(19) United States
(12) Patent Application Publication
(10) Pub. No.: US 2022/0115594 A1
(43) Pub. Date: Apr. 14, 2022

(54) ORGANIC ELECTROLUMINESCENCE DEVICE AND AMINE COMPOUND FOR ORGANIC ELECTROLUMINESCENCE DEVICE

(71) Applicant: Samsung Display Co., Ltd., Yongin-si (KR)

(72) Inventors: HANKYU PAK, Suwon-si (KR); EUNJAE JEONG, Hwaseong-si (KR); SANGHYUN HAN, Hwaseong-si (KR); DONGJUN KIM, Suwon-si (KR); MINJI KIM, Hwaseong-si (KR); SOHEE JO, Cheonan-si (KR)

(21) Appl. No.: 17/343,650

(22) Filed: Jun. 9, 2021

(30) Foreign Application Priority Data
Oct. 8, 2020 (KR) 10-2020-0130505

(51) Int. Cl. H01L 51/00; C07D 209/88; C09K 11/06

(52) U.S. Cl. CPC H01L 51/006; C07D 209/88; H01L 51/5012; H01L 51/0061; C09K 11/06

(57) ABSTRACT

Provided is an organic electroluminescence device according to an embodiment of the present disclosure including a first electrode, a second electrode facing the first electrode, an organic layer between the first electrode and the second electrode, and the organic layer includes an amine compound represented by Formula 1, and thus may exhibit high luminous efficiency.

### CROSS-REFERENCE TO RELATED APPLICATION

This application claims priority to and the benefit of Korean Patent Application No. 10-2020-0130505, filed on Oct. 8, 2020, the entire contents of which are hereby incorporated by reference.

### BACKGROUND

### 1. Field

Embodiments of the present disclosure herein relate to an organic electroluminescence device and an amine compound for the organic electroluminescence device.

### 2. Description of Related Art

Recently, the development of an organic electroluminescence display as an image display is being actively conducted. Unlike a liquid crystal display and the like, the organic electroluminescence display is so-called a self-luminescent display in which holes and electrons injected from a first electrode and a second electrode recombine in an emission layer so that a light-emitting material including an organic compound in the emission layer emits light to achieve display.

In the application of an organic electroluminescence device to a display, the organic electroluminescence device is desired to have low driving voltage, high luminous efficiency and long service life, and development of a material for an organic electroluminescence device that can stably achieve the requirements is continuously being investigated.

### SUMMARY

Embodiments of the present disclosure provide an organic electroluminescence device and an amine compound for an organic electroluminescence device, and, for example, provide a high efficiency organic electroluminescence device and an amine compound included in a hole transport region of the organic electroluminescence device.

An embodiment of the present disclosure provides an organic electroluminescence device including a first electrode, a second electrode facing the first electrode, and an organic layer between the first electrode and the second electrode, wherein the organic layer includes an amine compound represented by Formula 1 below.

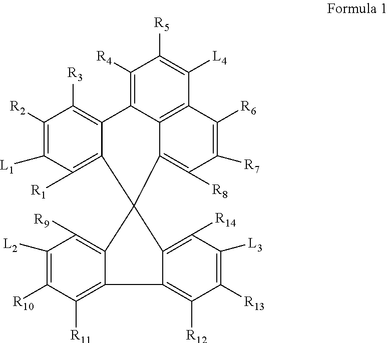

In Formula 1 above, R₁ to R₁₄ are each independently a hydrogen atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms, and L₁ to L₄ are each independently a hydrogen atom, a halogen atom, a substituted or unsubstituted alkyl group having 1 to 20 carbon atoms, a substituted or unsubstituted ring-forming aryl group having 6 to 30 carbon atoms, a substituted or unsubstituted ring-forming heteroaryl group having 2 to 30 carbon atoms, or a group represented by Formula 2 below where at least one selected from among L1 to L4 is represented by Formula 2 below.

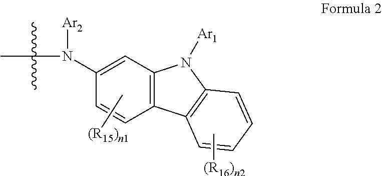

In Formula 2 above, R₁₅ and R₁₆ are each independently a hydrogen atom, a deuterium atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms, Ar₁ and Ar₂ are each independently a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms, n1 is an integer of 0 to 3, and n2 is an integer of 0 to 4.

In an embodiment, the organic layer may include a hole transport region on the first electrode, an emission layer on the hole transport region, and an electron transport region on the emission layer, wherein the hole transport region includes an amine compound represented by Formula 1 above.

In an embodiment, the hole transport region may include a hole injection layer on the first electrode, and a hole transport layer on the hole injection layer, wherein the hole injection layer or the hole transport layer includes an amine compound represented by Formula 1 above.

In an embodiment, the hole transport region may include a hole transport layer on the first electrode, and an electron blocking layer on the hole transport layer, wherein the electron blocking layer includes an amine compound represented by Formula 1 above.

In an embodiment, R₁ to R₁₄, Ar₁, and Ar₂ may not include a substituted or unsubstituted amine group in the amine compound represented by Formula 1 above.

In an embodiment, Formula 1 above may be represented by any one selected from among Formula 3-1 to Formula 3-4 below.

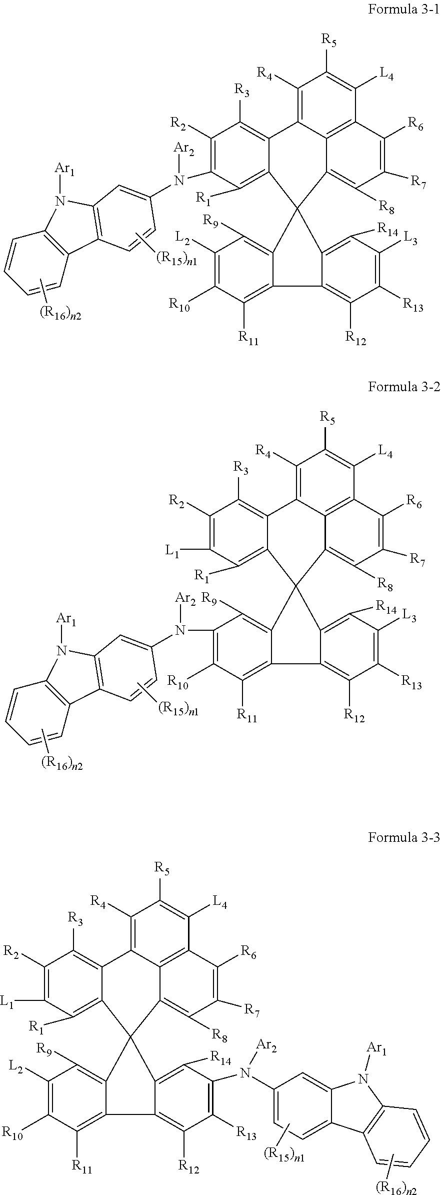

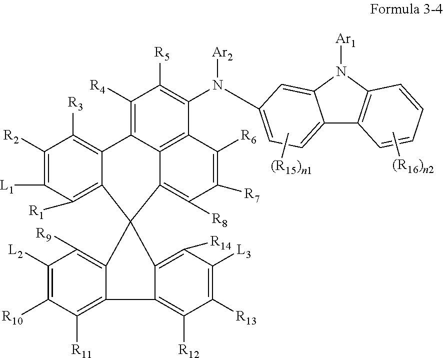

In Formula 3-1 to Formula 3-4 above, R₁ to R₁₆, L₁ to L₄, Ar₁, Ar₂, n1, and n2 are the same as defined with respect to in Formula 1 and Formula 2.

In an embodiment, the Ar₁ and Ar₂ may be each independently a substituted or unsubstituted ring-forming aryl group having 6 to 18 carbon atoms.

In an embodiment, the Ar₁ and Ar₂ may be each independently represented by any one selected from among Formula 4-1 to Formula 4-5 below.

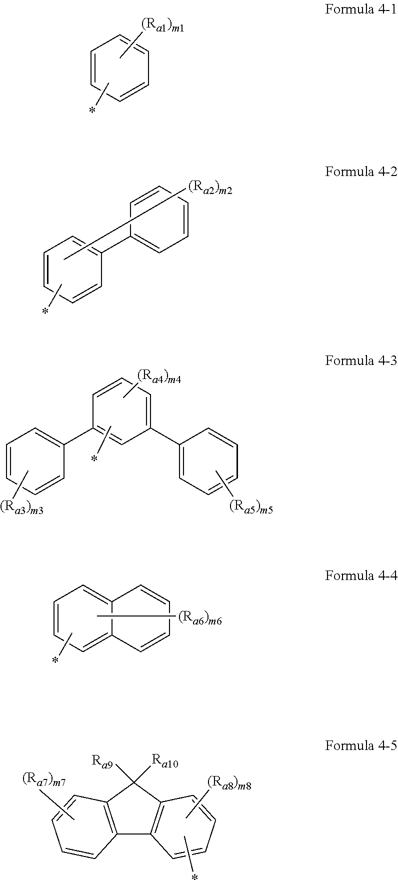

In Formula 4-1 to Formula 4-5 above, Rₐ₁ to Rₐ₁₀ are each independently a hydrogen atom, a deuterium atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms, m1, m3, and m5 are each independently an integer of 0 to 5, m2 is an integer of 0 to 9, m4 and m8 are an integer of 0 to 3, m6 is an integer of 0 to 7, and m7 is an integer of 0 to 4.

In an embodiment, the Ar₁ and Ar₂ may be each independently a substituted or unsubstituted dibenzohetero group.

In an embodiment, the Ar₁ and Ar₂ may be each independently represented by Formula 5-1 or Formula 5-2 below.

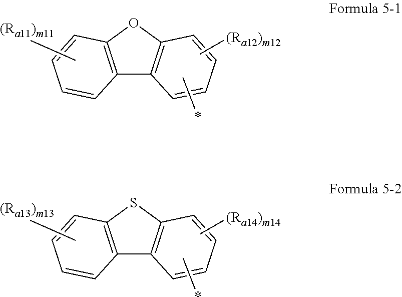

In Formula 5-1 and Formula 5-2 above, Rₐ₁₁ to Rₐ₁₄ are each independently a hydrogen atom, a deuterium atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms, m11 and m13 are each independently an integer of 0 to 4, and m12 and m14 are each independently an integer of 0 to 3.

In an embodiment, the emission layer may include a compound represented by Formula E-1 below.

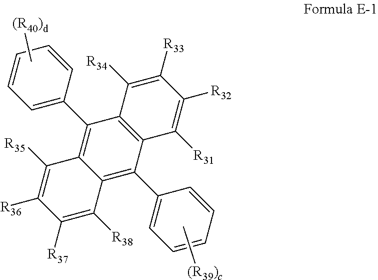

In Formula E-1, R₃₁ to R₄₀ are each independently a hydrogen atom, a deuterium atom, a halogen atom, a substituted or unsubstituted silyl group, a substituted or unsubstituted alkyl group having 1 to 10 carbon atoms, a substituted or unsubstituted ring-forming aryl group having 6 to 30 carbon atoms, or a substituted or unsubstituted ring-forming heteroaryl group having 2 to 30 carbon atoms, or combined with an adjacent group to form a ring, and c and d are each independently an integer of 0 to 5.

In an embodiment, the amine compound represented by Formula 1 above may be any one selected from among the compounds represented in Compound Group 1 below.

In an embodiment of the present disclosure, there is provided an amine compound represented by Formula 1.

### BRIEF DESCRIPTION OF THE FIGURES

The accompanying drawings are included to provide a further understanding of the subject matter of the present disclosure, and are incorporated in and constitute a part of this specification. The drawings illustrate example embodiments of the present disclosure and, together with the description, serve to explain principles of the present disclosure. In the drawings:

FIG. 1 is a plan view illustrating a display apparatus according to an embodiment of the present disclosure;

FIG. 2 is a cross-sectional view illustrating a display apparatus according to an embodiment of the present disclosure;

FIG. 3 is a cross-sectional view schematically illustrating an organic electroluminescence device according to an embodiment of the present disclosure;

FIG. 4 is a cross-sectional view schematically illustrating an organic electroluminescence device according to an embodiment of the present disclosure;

FIG. 5 is a cross-sectional view schematically illustrating an organic electroluminescence device according to an embodiment of the present disclosure;

FIG. 6 is a cross-sectional view schematically illustrating an organic electroluminescence device according to an embodiment of the present disclosure;

FIG. 7 is a cross-sectional view illustrating a display apparatus according to an embodiment of the present disclosure; and

FIG. 8 is a cross-sectional view illustrating a display apparatus according to an embodiment of the present disclosure.

### DETAILED DESCRIPTION

An embodiment of the present disclosure is susceptible to various modifications and alternative forms, and specific embodiments thereof are illustrated by way of example in the drawings and will be described herein in more detail. However, it should be understood that the subject matter of the present disclosure is not intended to be limited by the specific forms disclosed, and all modifications, equivalents, and alternatives are included within the spirit and scope of the present disclosure.

In describing each drawing, like reference numerals are used for like components. In the accompanying drawings, the dimensions of structures may be shown in enlarged scale for clarity of illustration. It will be understood that, although the terms first, second, etc. may be used herein to describe various components, these components should not be limited by these terms. These terms are only used to distinguish one component from another component. For example, a first component may be termed a second component without departing from the scope of the present disclosure, and similarly, a second component may be termed a first component. The singular forms, “a”, “an” and “the” are intended to include the plural forms as well, unless the context clearly indicates otherwise.

It will be further understood that the terms “include” or “have” etc., when used in this application, specify the presence of stated features, integers, steps, operations, components, or parts, but do not preclude the presence or addition of one or more other features, integers, steps, operations, components, parts, or combination thereof.

In this application, when a part such as a layer, a film, a region, a plate is referred to as being “on” or “above” the other part, it may be “directly on” the other part, or an intervening part may also be present. In contrast, when a part such as a layer, a film, a region, a plate is referred to as being “under” or “below” the other part, it may be “directly under” the other part, or an intervening part may also be present. In addition, in this application, when a part is referred to as being “on” the other part, it may be on the upper part or the lower part as well.

Hereinafter, embodiments of the present disclosure will be explained further with reference to the drawings.

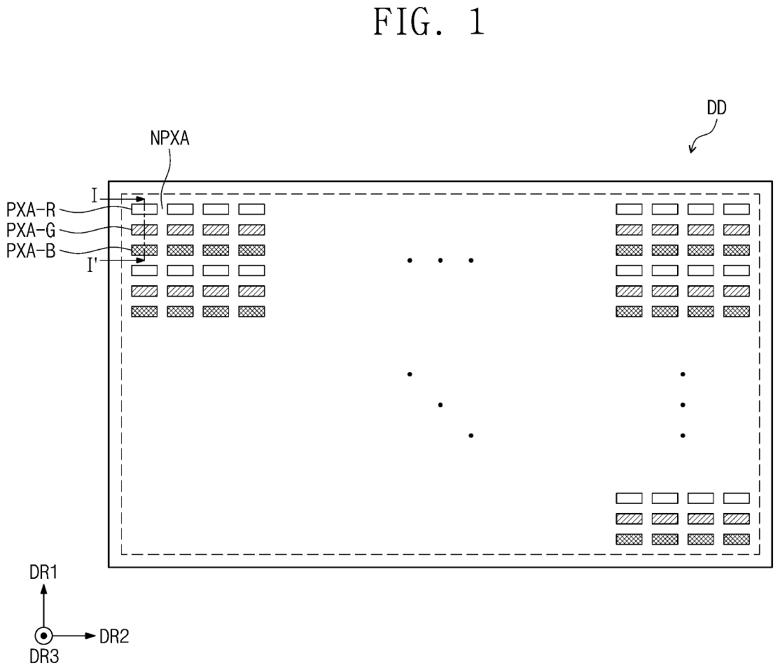

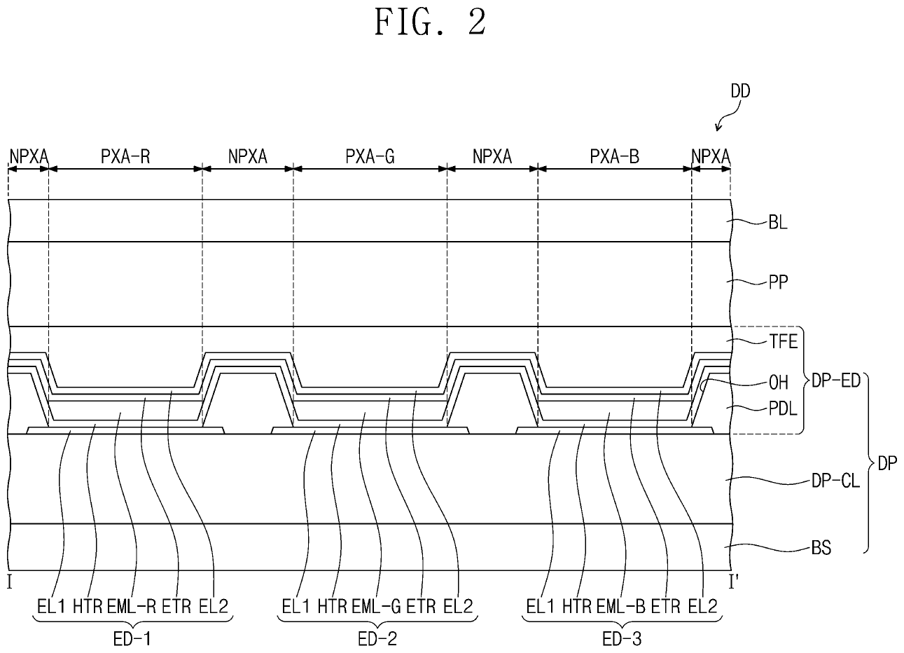

FIG. 1 is a plan view illustrating an embodiment of the present disclosure of a display apparatus DD. FIG. 2 is a cross-sectional view of a display apparatus DD according to an embodiment of the present disclosure. FIG. 2 is a cross-sectional view showing a portion corresponding to line I-I′ in FIG. 1.

The display apparatus DD may include a display panel DP and an optical layer PP on the display panel DP. The display panel DP includes organic electroluminescence devices ED-1, ED-2, and ED-3. The display apparatus DD may include a plurality of organic electroluminescence devices ED-1, ED-2, and ED-3. The optical layer PP may be on the display panel DP and control the reflected light by external light on the display panel DP. The optical layer PP may include, for example, a polarization layer or a color filter layer. In one or more embodiments, the optical layer PP may be omitted in the display apparatus DD according to an embodiment of the present disclosure.

A base substrate BL may be on the optical layer PP. The base substrate BL may be a member providing a base surface on which the optical layer pp is located. The base substrate BL may be a glass substrate, a metal substrate, a plastic substrate, and/or the like. However, the embodiment of the present disclosure is not limited thereto, and the base substrate BL may be an inorganic layer, an organic layer, or a composite material layer including an inorganic material and an organic material. In addition, unlike the illustration, the base substrate BL may be omitted in an embodiment of the present disclosure.

The display apparatus DD according to an embodiment of the present disclosure may further include a filling layer. The filling layer may be between a display device layer DP-ED and a base substrate BL. The filling layer may be an organic material layer. The filling layer may include at least one of acrylic-based resin, silicone-based resin, and/or epoxy-based resin.

The display panel DP may include a base layer BS, a circuit layer DP-CL provided on the base layer BS, and a display device layer DP-ED. The display device layer DP-ED may include a pixel-defining film PDL, organic electroluminescence devices ED-1, ED-2, and ED-3 between the pixel-defining film PDL, and an encapsulating layer TFE on the organic electroluminescence devices ED-1, ED-2, and ED-3.

The base layer BS may be a member that provides a base surface on which the display device layer DP-ED is located. The base layer BS may be a glass substrate, a metal substrate, a plastic substrate, and/or the like. However, the embodiment of the present disclosure is not limited thereto, and the base layer BS may be an inorganic layer, an organic layer, or a composite material layer including an inorganic material and an organic material.

In an embodiment of the present disclosure, the circuit layer DP-CL may be on the base layer BS, and the circuit layer DP-CL may include a plurality of transistors. The transistors may each include a control electrode, an input electrode, and an output electrode. For example, the circuit layer DP-CL may include a switching transistor and a driving transistor for driving the organic electroluminescence devices ED-1, ED-2, and ED-3 of the display device layer DP-ED.

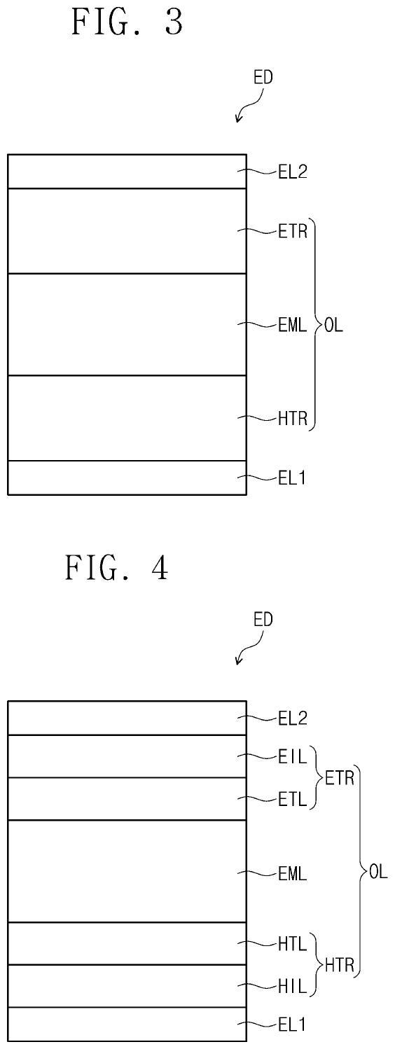

Each of the organic electroluminescence devices ED-1, ED-2, and ED-3 may have a structure of an organic electroluminescence device ED according to an embodiment of the present disclosure of FIGS. 3 to 6 to be described below. Each of the organic electroluminescence devices ED-1, ED-2, and ED-3 may include a first electrode EL1, a hole transport region HTR, emission layers EML-R, EML-G, and EML-B, an electron transport region ETR, and a second electrode EL2.

In FIG. 2, the emission layers EML-R, EML-G, and EML-B of the organic electroluminescence devices ED-1, ED-2, and ED-3 are in an opening OH defined with respect to the pixel-defining film PDL, and the hole transport region HTR, the electron transport region ETR, and the second electrode EL2 are provided as common layers in all of the organic electroluminescence devices ED-1, ED-2, and ED-3. However, the embodiment of the present disclosure is not limited thereto. In one or more embodiments of the present disclosure, the hole transport region HTR and the electron transport region ETR may be patterned and provided in the opening OH defined with respect to the pixel-defining film PDL. For example, in an embodiment of the present disclosure, the hole transport region HTR, the emission layers EML-R, EML-G, and EML-B, and the electron transport region ETR, etc. of the organic electroluminescence devices ED-1, ED-2, and ED-3 may be patterned and provided by an inkjet printing method.

The encapsulating layer TFE may cover the organic electroluminescence devices ED-1, ED-2, and ED-3. The encapsulating layer TFE may seal the display device layer DP-ED. The encapsulating layer TFE may be a thin film encapsulating layer. The encapsulating layer TFE may be a single layer or a stacked layer of a plurality of layers. The encapsulating layer TFE includes at least one insulating layer. The encapsulating layer TFE according to an embodiment of the present disclosure may include at least one inorganic film (hereinafter, an encapsulating inorganic film). In addition, the encapsulating layer TFE according to an embodiment of the present disclosure may include at least one organic film (hereinafter, an encapsulating organic film) and at least one encapsulating inorganic film.

The encapsulating inorganic film protects the display device layer DP-ED from moisture/oxygen, and the encapsulating organic film protects the display device layer DP-ED from foreign materials such as dust particles. The encapsulating inorganic film may include silicon nitride, silicon oxy nitride, silicon oxide, titanium oxide, aluminum oxide, or the like, but the embodiment of the present disclosure is not particularly limited thereto. The encapsulating organic film may include an acrylic-based compound, an epoxy-based compound, and/or the like. The encapsulating organic film may include an organic material capable of photopolymerization, but the embodiment of the present disclosure is not particularly limited thereto.

The encapsulating layer TFE may be on the second electrode EL2 and may be disposed (or formed or deposited) while charging the opening OH.

Referring to FIG. 1 and FIG. 2, the display apparatus DD may include a non-light emitting region NPXA and light-emitting regions PXA-R, PXA-G, and PXA-B. The light-emitting regions PXA-R, PXA-G, and PXA-B may be regions which emit light generated from the organic electroluminescence devices ED-1, ED-2, and ED-3, respectively. The light-emitting regions PXA-R, PXA-G, and PXA-B may be spaced apart from each other on a plane.

Each of the light-emitting regions PXA-R, PXA-G, and PXA-B may be a region separated by a pixel-defining film PDL. The non-light emitting regions NPXA may be regions interposed between the neighboring light-emitting regions PXA-R, PXA-G, and PXA-B, and may be regions corresponding to the pixel-defining film PDL. In one or more embodiments, the light-emitting regions PXA-R, PXA-G, and PXA-B may respectively correspond to pixels. The pixel-defining film PDL may separate the organic electroluminescence devices ED-1, ED-2 and ED-3. Emission layers EML-R, EML-G and EML-B of the organic electroluminescence devices ED-1, ED-2 and ED-3 may be in the opening OH defined with respect to the pixel-defining film PDL and separated from each other.

The light-emitting regions PXA-R, PXA-G, and PXA-B may be classified into a plurality of groups according to the color of light generated from the organic electroluminescence devices ED-1, ED-2, and ED-3. In the display apparatus DD according to an embodiment of the present disclosure shown in FIG. 1 and FIG. 2, three light-emitting regions PXA-R, PXA-G, and PXA-B respectively emitting red light, green light, and blue light are illustrated by way of example. For example, the display apparatus DD according to an embodiment of the present disclosure may include a red light-emitting region PXA-R, a green light-emitting region PXA-G, and a blue light-emitting region PXA-B, which are distinguished from each other.

In the display apparatus DD according to an embodiment of the present disclosure, a plurality of organic electroluminescence devices ED-1, ED-2, and ED-3 may emit light having different wavelength regions. For example, in an embodiment of the present disclosure, the display apparatus DD may include a first organic electroluminescence device ED-1 emitting red light, a second organic electroluminescence device ED-2 emitting green light, and a third organic electroluminescence device ED-3 emitting blue light. In one or more embodiments, the red light-emitting region PXA-R, the green light-emitting region PXA-G, and the blue light-emitting region PXA-B of the display apparatus DD may correspond to the first organic electroluminescence device ED-1, the second organic electroluminescence device ED-2, and the third organic electroluminescence device ED-3, respectively.

However, the embodiment of the present disclosure is not limited thereto, and the first to third organic electroluminescence devices ED-1, ED-2, and ED-3 may emit light of the same wavelength region, or at least one thereof may emit light of a different wavelength region. For example, all of the first to third organic electroluminescence devices ED-1, ED-2, and ED-3 may emit blue light.

The light-emitting regions PXA-R, PXA-G, and PXA-B in the display apparatus DD according to an embodiment of the present disclosure may be arranged in a stripe shape. Referring to FIG. 1, a plurality of red light-emitting regions PXA-R, a plurality of green light-emitting regions PXA-G, and a plurality of blue light-emitting regions PXA-B may be arranged respectively along a second direction axis DR2. In addition, the red light-emitting region PXA-R, the green light-emitting region PXA-G, and the blue light-emitting region PXA-B may be alternatively arranged in order along a first direction axis DR1.

FIG. 1 and FIG. 2 illustrate that all the light-emitting regions PXA-R, PXA-G, and PXA-B have similar areas, but the embodiment of the present disclosure is not limited thereto. The areas of the light-emitting regions PXA-R, PXA-G, and PXA-B may be different from each other depending on the wavelength region of the emitted light. In one or more embodiments, the areas of the light-emitting regions PXA-R, PXA-G, and PXA-B may indicate areas as viewed on a plane defined by the first direction axis DR1 and the second direction axis DR2.

In one or more embodiments, the arrangement of the light-emitting regions PXA-R, PXA-G, and PXA-B is not limited to the configuration illustrated in FIG. 1, and the arrangement order of the red light-emitting region PXA-R, the green light-emitting region PXA-G, and the blue light-emitting region PXA-B may be provided in various combinations depending on the characteristics of display quality required for the display apparatus DD. For example, the light-emitting regions PXA-R, PXA-G, and PXA-B may be arranged in a PENTILE® arrangement structure (e.g., an RGBG matrix, RGBG structure, or RGBG matrix structure) or a diamond configuration. PENTILE® is a duly registered trademark of Samsung Display Co., Ltd.

In addition, the areas of the light-emitting regions PXA-R, PXA-G, and PXA-B may be different from each other. For example, in an embodiment of the present disclosure, the area of the green light-emitting region PXA-G may be smaller than the area of the blue light-emitting region PXA-B, but the embodiment of the present disclosure is not limited thereto.

Hereinafter, FIGS. 3 to 6 are cross-sectional views schematically illustrating an organic electroluminescence device according to an embodiment of the present disclosure. Referring to FIGS. 3 to 6, in an organic electroluminescence device ED according to an embodiment, a first electrode EU and a second electrode EL2 face each other, and an organic layer OL may be between the first electrode EL1 and the second electrode EL2.

In one or more embodiments, the organic layer OL according to an embodiment of the present disclosure may include a plurality of functional layers. The plurality of functional layers may include a hole transport region HTR, an emission layer EML, and an electron transport region ETR. For example, the organic electroluminescence device ED according to an embodiment of the present disclosure may include a first electrode EL1, a hole transport region HTR, an emission layer EML, an electron transport region ETR, and a second electrode EL2 sequentially stacked. A capping layer CPL may be further on the second electrode EL2.

The organic electroluminescence device ED of an embodiment of the present disclosure may include an amine compound according to an embodiment of the present disclosure to be described further below in the organic layer OL between the first electrode EL1 and the second electrode EL2. For example, the organic electroluminescence device ED of an embodiment of the present disclosure may include an amine compound according to an embodiment of the present disclosure to be described below in the hole transport region HTR between the first electrode EU and the second electrode EL2. However, the embodiment of the present disclosure is not limited thereto. In addition to the hole transport region HTR, the organic electroluminescence device ED of an embodiment of the present disclosure may include the amine compound according to an embodiment of the present disclosure to be described below in at least one functional layer included in the emission layer EML and the hole transport region ETR which are a plurality of functional layers between the first electrode EL1 and the second electrode EL2. In one or more embodiments, a capping layer CPL on the second electrode EL2 may include the amine compound according to an embodiment of the present disclosure to be described below.

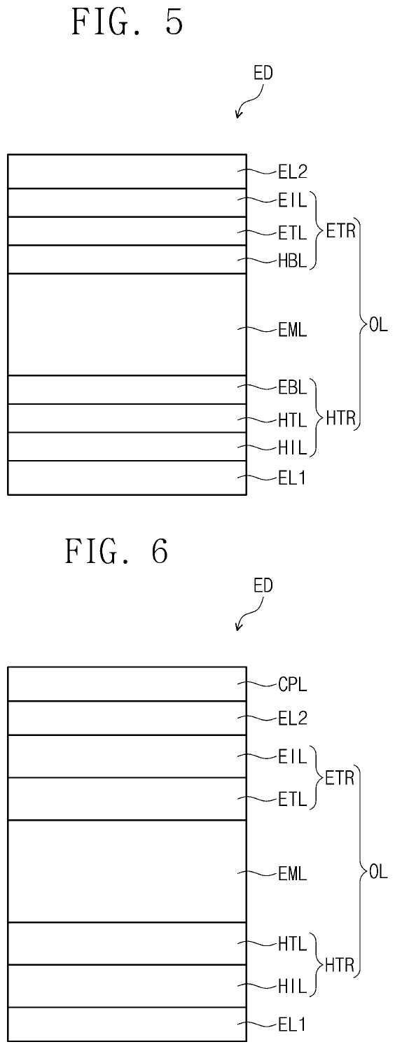

When compared with FIG. 3, FIG. 4 shows a cross-sectional view of an organic electroluminescence device ED according to an embodiment of the present disclosure, wherein a hole transport region HTR includes a hole injection layer HIL and a hole transport layer HTL, and an electron transport region ETR includes an electron injection layer EIL and an electron transport layer ETL. In addition, when compared with FIG. 3, FIG. 5 shows a cross-sectional view of an organic electroluminescence device ED according to an embodiment of the present disclosure, wherein a hole transport region HTR includes a hole injection layer HIL, a hole transport layer HTL, and an electron blocking layer EBL, and an electron transport region ETR includes an electron injection layer EIL, an electron transport layer ETL, and/or a hole blocking layer HBL. In an embodiment of the present disclosure, the hole injection layer HIL, the hole transport layer HTL, and/or the electron blocking layer EBL may include the amine compound according to an embodiment of the present disclosure to be described below. In another embodiment of the present disclosure, the electron injection layer EIL, the electron transport layer ETL, and/or the hole blocking layer HBL may include the amine compound according to an embodiment of the present disclosure to be described below.

When compared with FIG. 4, FIG. 6 shows a cross-sectional view of an organic electroluminescence device ED according to an embodiment of the present disclosure including a capping layer CPL on the second electrode EL2.

The first electrode EL1 has conductivity (e.g., electrical conductivity). The first electrode EL1 may be formed using a metal material, a metal alloy, and/or a conductive compound. The first electrode EU may be an anode or a cathode. However, the embodiment of the present disclosure is not limited thereto. In one or more embodiments, the first electrode EL1 may be a pixel electrode. The first electrode EU may be a transmissive electrode, a transflective electrode, or a reflective electrode. If the first electrode EU is the transmissive electrode, the first electrode EL1 may include transparent metal oxide such as indium tin oxide (ITO), indium zinc oxide (IZO), zinc oxide (ZnO), indium tin zinc oxide (ITZO), and/or the like. If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca, LiF/Al, Mo, Ti, a compound thereof, or a mixture thereof (for example, a mixture of Ag and Mg). In one or more embodiments, the first electrode EL1 may have a multilayered structure including a reflective film or a transflective film formed using the above-described materials and a transparent conductive film formed using indium tin oxide (ITO), indium zinc oxide (IZO), zinc oxide (ZnO), indium tin zinc oxide (ITZO), and/or the like. For example, the first electrode EL1 may have a three-layer structure of ITO/Ag/ITO, but the embodiment of the present disclosure is not limited thereto. In addition, the embodiment of the present disclosure is not limited thereto, and the first electrode EL1 may include the above-described metal material, a combination of two or more metal materials selected from the above-described metal materials, and/or oxides of the above-described metal materials. The thickness of the first electrode EL1 may be about 700 Å to about 10000 Å. For example, the thickness of the first electrode EL1 may be about 1000 Å to about 3000 Å.

The hole transport region HTR may be provided on the first electrode EL1. The hole transport region HTR may include at least one of a hole injection layer HIL, a hole transport layer HTL, a buffer layer and/or a light-emitting auxiliary layer, and/or an electron blocking layer EBL. A thickness of the hole transport region HTR may be, for example, about 50 Å to about 15,000 Å.

The hole transport region HTR may have a single layer structure formed using a single material, a single layer structure formed using a plurality of different materials, or a multilayer structure having a plurality of layers formed using a plurality of different materials.

For example, the hole transport regions HTR may have the structure of a single layer of a hole injection layer HIL or a hole transport layer HTL, and may have a structure of a single layer formed using a hole injection material and a hole transport material. Further, the hole transport regions HTR may have a structure of a single layer formed using a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in order from the first electrode EL1, but the embodiment of the present disclosure is not limited thereto.

The hole transport region HTR of the organic electroluminescence device ED according to an embodiment of the present disclosure includes an amine compound according to an embodiment of the present disclosure.

In one or more embodiments, the term “substituted or unsubstituted” corresponds to substituted or unsubstituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In addition, each of the described substituents may be substituted or unsubstituted. For example, a biphenyl group may be interpreted as an aryl group or a phenyl group substituted with a phenyl group.

In the description, the phrase “combining with an adjacent group to form a ring” may mean combining with an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and heterocycle may be a monocyclic ring or a polycyclic ring. In addition, the ring formed via the binding with an adjacent group may be bonded with another ring to form a spiro structure.

In the description, the term “adjacent group” may mean a substituent substituted for an atom which is directly combined with an atom substituted with a corresponding substituent, another substituent substituted for an atom which is substituted with a corresponding substituent, or a substituent sterically positioned at the nearest position to a corresponding substituent. For example, in 1,2-dimethylbenzene, two methyl groups may be interpreted as “adjacent groups” to each other, and in 1,1-diethylcyclopentene, two ethyl groups may be interpreted as “adjacent groups” to each other. In addition, in 4,5-dimethylphenanthrene, two methyl groups may be interpreted as “adjacent groups” to each other.

In the description, examples of the halogen atom may include a fluorine atom, a chlorine atom, a bromine atom, or an iodine atom.

In the description, the alkyl group may be a linear, branched, or cyclic type (e.g., a linear alkyl group, a branched alkyl group, or a cyclic alkyl group). The number of carbon atoms of the alkyl may be 1 to 50, 1 to 30, 1 to 20, 1 to 10, or 1 to 6. Examples of the alkyl group may include, but are not limited to, methyl, ethyl, n-propyl, isopropyl, n-butyl, s-butyl, t-butyl, i-butyl, 2-ethylbutyl, 3,3-dimethylbutyl, n-pentyl, pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, and the like.

In the description, the term “hydrocarbon ring group” means an optional functional group or a substituent derived from an aliphatic hydrocarbon ring. The hydrocarbon ring group may be a saturated ring-forming hydrocarbon ring group having 5 to 20 carbon atoms.

In the description, the term “aryl group” means an optional functional group or substituent derived from an aromatic hydrocarbon ring. The aryl group may be a monocyclic aryl group or a polycyclic aryl group. The number of ring-forming carbon atoms of the aryl group may be 6 to 30, 6 to 20, or 6 to 15. Examples of the aryl group may include, but are not limited to, phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysenyl, and so on.

In the description, the heteroaryl group may include one or more selected from among B, O, N, P, Si, and S as a heteroatom. If the heteroaryl group includes two or more heteroatoms, two or more heteroatoms may be the same as or different from each other. The heteroaryl group may be a monocyclic heterocyclic group or a polycyclic heterocyclic group. The number of ring-forming carbon atoms of the heteroaryl group may be 2 to 30, 2 to 20, or 2 to 10. Examples of the heteroaryl group include, but are not limited to thiophene, furan, pyrrole, imidazole, triazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzoimidazole, benzothiazole, benzocarbazole, benzothiophene, dibenzothiophene, thienothiophene, benzofuran, phenanthroline, thiazole, isoxazole, oxazole, oxadiazole, thiadiazole, phenothiazine, dibenzosilole, dibenzofuran, and the like.

In the description, the number of carbon atoms of the amine group may be 1 to 30, but is not particularly limited thereto. The amine group may include alkyl amine group and aryl amine group. Examples of amine group include, but are not limited to methylamine, dimethylamine, phenylamine, diphenylamine, naphthylamine, 9-methyl-anthracenylamine group, triphenylamine, and the like.

In one or more embodiments of the present description

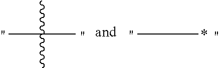

means a position to be connected.

An amine compound according to an embodiment of the present disclosure is presented by Formula 1 below.

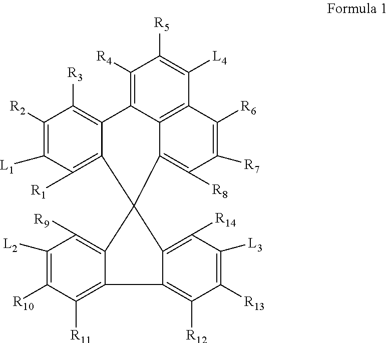

In formula 1, R₁ to R₁₄ are each independently a hydrogen atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms.

In formula 1, L₁ to L₄ are each independently a hydrogen atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms, or a group represented by Formula 2 below where at least one selected from among L1 to L4 is represented by Formula 2 below.

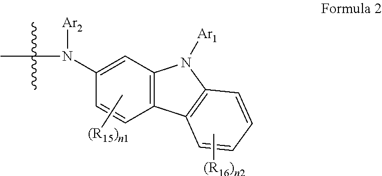

In Formula 2, R₁₅ and R₁₆ are each independently a hydrogen atom, a deuterium atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms.

In Formula 2, Ar₁ and Ar₂ are each independently a substituted or unsubstituted ring-forming aryl group having 6 to 30 carbon atoms, or a substituted or unsubstituted ring-forming heteroaryl group having 2 to 30 carbon atoms.

In Formula 2, n1 is an integer of 0 to 3. In one or more embodiments, if n1 is 2 or more, a plurality of R₁₅ are the same as or different from each other.

In Formula 2, n2 is an integer of 0 to 4. In one or more embodiments, if n2 is 2 or more, a plurality of R₁₆ are the same as or different from each other.

In an embodiment of the present disclosure, only one of L₁ to L₄ in Formula 1 may be represented by Formula 2. In another embodiment of the present disclosure, R₁ to R₁₄, Ar₁, and Ar₂ may not include a substituted or unsubstituted amine group in the amine compound represented by Formula 1. For example, the amine compound represented by Formula 1 may be a monoamine compound that does not include an amine group other than the amine group represented by Formula 2.

In an embodiment of the present disclosure, Formula 1 may be represented by any one selected from among Formula 3-1 to Formula 3-4 below.

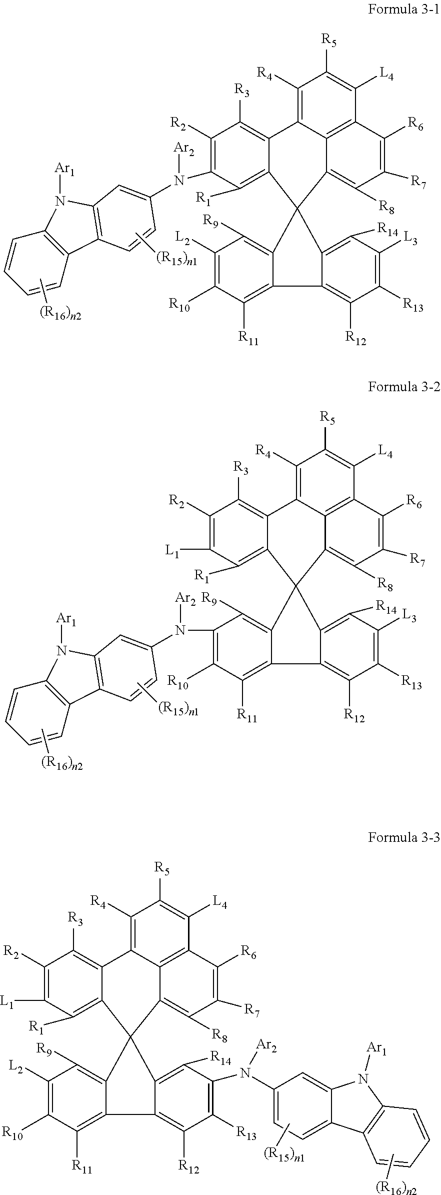

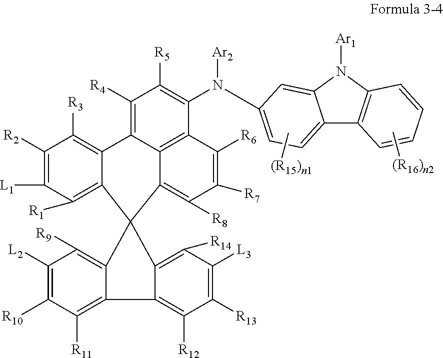

In Formula 3-1 to Formula 3-4, R₁ to R₁₆, L₁ to L₄, Ar₁, Ar₂, n1, and n2 are the same as defined with respect to Formula 1 and Formula 2.

In an embodiment of the present disclosure, Ar₁ and Ar₂ of an amine compound according to an embodiment of the present disclosure may be each independently a substituted or unsubstituted ring-forming aryl group having 6 to 18 carbon atoms.

In an embodiment of the present disclosure, Ar₁ and Ar₂ of an amine compound according to an embodiment of the present disclosure may be each independently represented by any one selected from among Formula 4-1 to Formula 4-5 below.

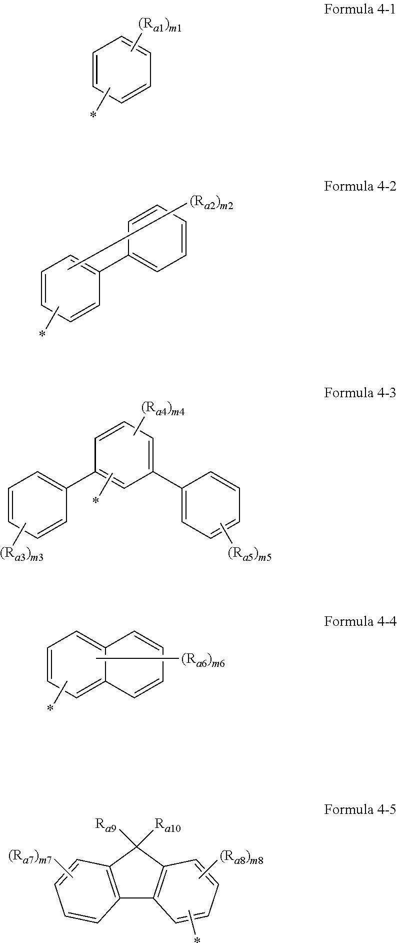

In Formula 4-1 to Formula 4-5, Rₐ₁ to Rₐ₁₀ are each independently a hydrogen atom, a deuterium atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms.

In Formula 4-1, m1 is an integer of 0 to 5. In one or more embodiments, if m1 is 2 or more, a plurality of Rₐ₁ are the same as or different from each other.

In Formula 4-2, m2 is an integer of 0 to 9. In one or more embodiments, if m2 is 2 or more, a plurality of Rₐ₂ are the same as or different from each other.

In Formula 4-3, m3 is an integer of 0 to 5. In one or more embodiments, if m3 is 2 or more, a plurality of Rₐ₃ are the same as or different from each other.

In Formula 4-3, m4 is an integer of 0 to 3. In one or more embodiments, if m4 is 2 or more, a plurality of Rₐ₄ are the same as or different from each other.

In Formula 4-3, m5 is an integer of 0 to 5. In one or more embodiments, if m5 is 2 or more, a plurality of Rₐ₅ are the same as or different from each other.

In Formula 4-4, m6 is an integer of 0 to 7. In one or more embodiments, if m6 is 2 or more, a plurality of Rₐ₆ are the same as or different from each other.

In Formula 4-5, m7 is an integer of 0 to 4. In one or more embodiments, if m7 is 2 or more, a plurality of Rₐ₇ are the same as or different from each other.

In Formula 4-5, m8 is an integer of 0 to 3. In one or more embodiments, if m8 is 2 or more, a plurality of Rₐ₈ are the same as or different from each other.

In an embodiment of the present disclosure, Ar₁ and Ar₂ of an amine compound according to an embodiment of the present disclosure may be each independently a substituted or unsubstituted dibenzohetero group.

In an embodiment of the present disclosure, Ar₁ and Ar₂ of an amine compound according to an embodiment of the present disclosure may be each independently represented by Formula 5-1 or Formula 5-2 below.

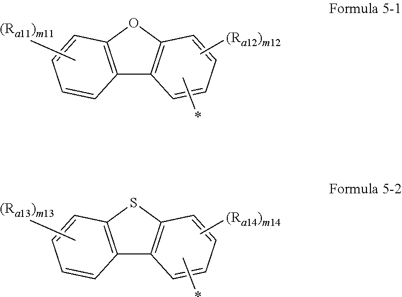

In Formula 5-1 and Formula 5-2, Rₐ₁₁ to Rₐ₁₄ may be each independently a hydrogen atom, a deuterium atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms.

In Formula 5-1, m11 is an integer of 0 to 4. In one or more embodiments, if m11 is 2 or more, a plurality of Rₐ₁₁ are the same as or different from each other.

In Formula 5-1, m12 is an integer of 0 to 3. In one or more embodiments, if m12 is 2 or more, a plurality of Rₐ₁₂ are the same as or different from each other.

In Formula 5-2, m13 is an integer of 0 to 4. In one or more embodiments, if m13 is 2 or more, a plurality of Rₐ₁₃ are the same as or different from each other.

In Formula 5-2, m14 is an integer of 0 to 3. In one or more embodiments, if m14 is 2 or more, a plurality of Rₐ₁₄ are the same as or different from each other.

In an embodiment of the present disclosure, Ar₁ and Ar₂ of an amine compound according to an embodiment of the present disclosure may be the same as each other. However, the embodiment of the present disclosure is not limited thereto, and Ar₁ and Ar₂ may be different from each other.

The amine compound represented by Formula 1 according to an embodiment of the present disclosure may be any one selected from the compounds represented in Compound Group 1 below. However, the embodiment of the present disclosure is not limited thereto.

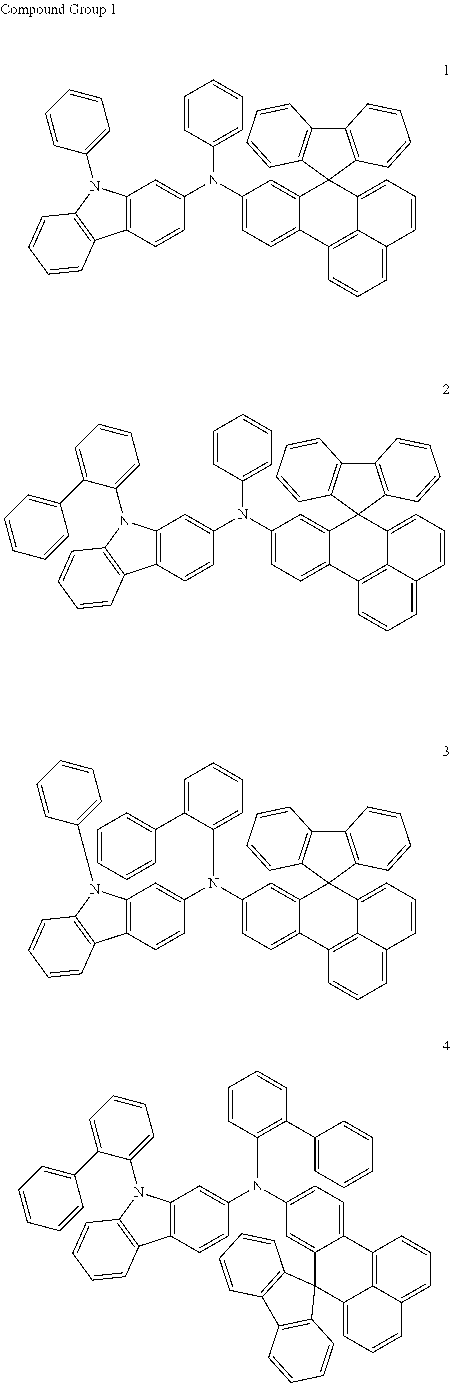 [SMILES: c1ccc(N(c2ccc3c(c2)C2(c4ccccc4-c4ccccc42)c2cccc4cccc-3c24)c2ccc3c4ccccc4n(-c4ccccc4)c3c2)cc1 ; c1ccc(-c2ccccc2-n2c3ccccc3c3ccc(N(c4ccccc4)c4ccc5c(c4)C4(c6ccccc6-c6ccccc64)c4cccc6cccc-5c46)cc32)cc1 ; c1ccc(Cn2c3ccccc3c3ccc(N(c4ccc5c(c4)C4(c6ccccc6-c6ccccc64)c4cccc6cccc-5c46)c4ccccc4-c4ccccc4)cc32)cc1 ; c1ccc(-c2ccccc2N(c2ccc3c(c2)C2(c4ccccc4-c4ccccc42)c2cccc4cccc-3c24)c2ccc3c4ccccc4n(-c4ccccc4-c4ccccc4)c3c2)cc1]

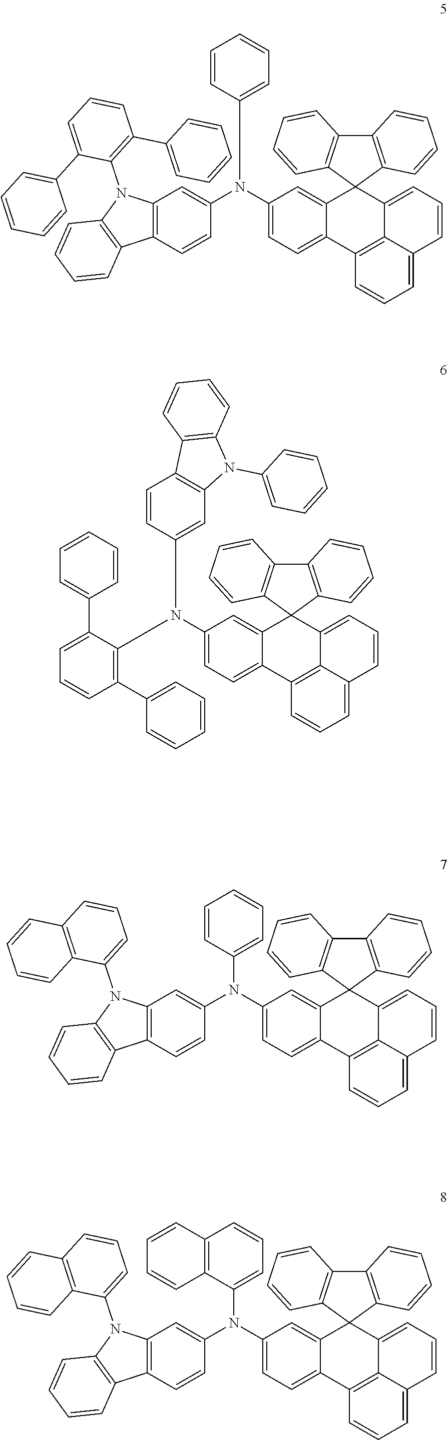 [SMILES: c1ccc(-c2cccc(-c3ccccc3)c2-n2c3ccccc3c3ccc(N(c4ccccc4)c4ccc5c(c4)C4(c6ccccc6-c6ccccc64)c4cccc6cccc-5c46)cc32)cc1 ; c1ccc(-c2cccc(-c3ccccc3)c2N(c2ccc3c(c2)C2(c4ccccc4-c4ccccc42)c2cccc4cccc-3c24)c2ccc3c4ccccc4n(-c4ccccc4)c3c2)cc1 ; c1ccc(N(c2ccc3c(c2)C2(c4ccccc4-c4ccccc42)c2cccc4cccc-3c24)c2ccc3c4ccccc4n(-c4cccc5ccccc45)c3c2)cc1 ; c1ccc2c(c1)-c1ccccc1C21c2cc(N(c3ccc4c5ccccc5n(-c5cccc6ccccc56)c4c3)c3cccc4ccccc34)ccc2-c2cccc3cccc1c23]

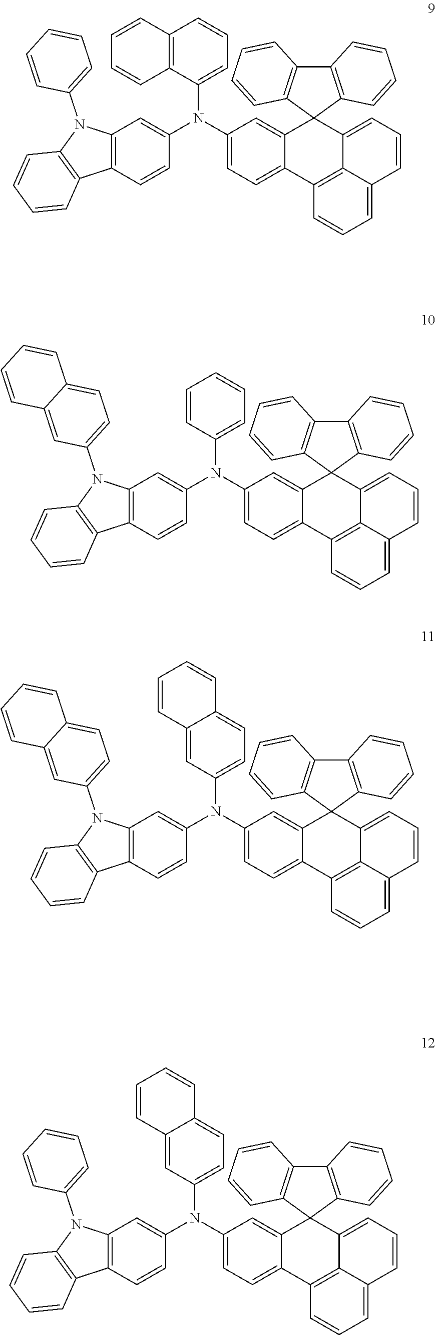 [SMILES: c1ccc(-n2c3ccccc3c3ccc(N(c4ccc5c(c4)C4(c6ccccc6-c6ccccc64)c4cccc6cccc-5c46)c4cccc5ccccc45)cc32)cc1 ; c1ccc(N(c2ccc3c(c2)C2(c4ccccc4-c4ccccc42)c2cccc4cccc-3c24)c2ccc3c4ccccc4n(-c4ccc5ccccc5c4)c3c2)cc1 ; c1ccc2c(c1)-c1ccccc1C21c2cc(N(c3ccc4ccccc4c3)c3ccc4c5ccccc5n(-c5ccc6ccccc6c5)c4c3)ccc2-c2cccc3cccc1c23 ; c1ccc(-n2c3ccccc3c3ccc(N(c4ccc5c(c4)C4(c6ccccc6-c6ccccc64)c4cccc6cccc-5c46)c4ccc5ccccc5c4)cc32)cc1]

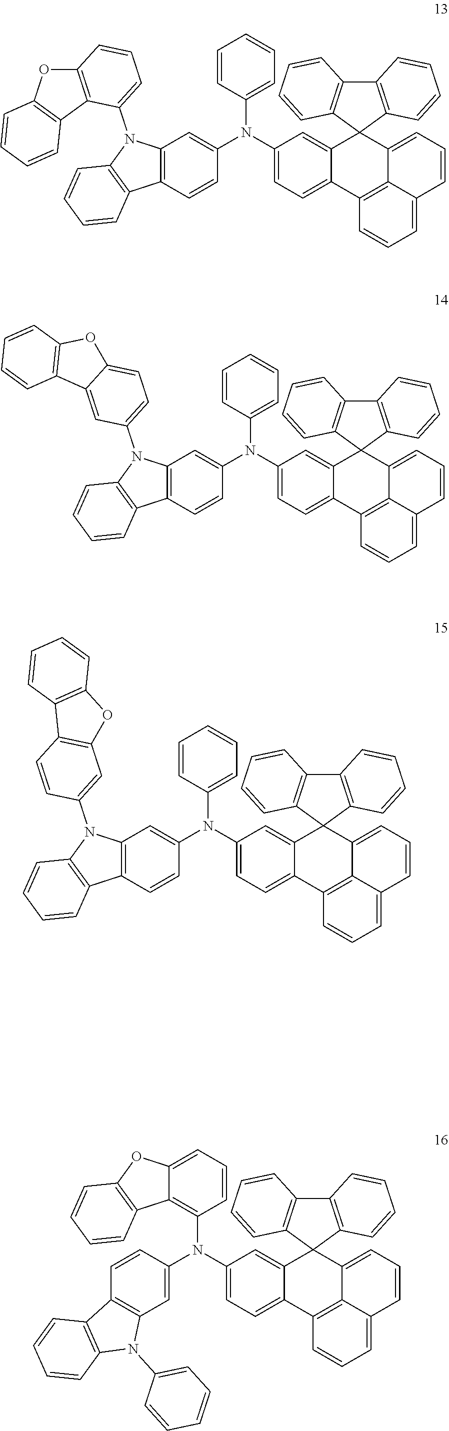 [SMILES: c1ccc(N(c2ccc3c(c2)C2(c4ccccc4-c4ccccc42)c2cccc4cccc-3c24)c2ccc3c4ccccc4n(-c4cccc5oc6ccccc6c45)c3c2)cc1 ; c1ccc(N(c2ccc3c(c2)C2(c4ccccc4-c4ccccc42)c2cccc4cccc-3c24)c2ccc3c4ccccc4n(-c4ccc5oc6ccccc6c5c4)c3c2)cc1 ; c1ccc(N(c2ccc3c(c2)C2(c4ccccc4-c4ccccc42)c2cccc4cccc-3c24)c2ccc3c4ccccc4n(-c4ccc5c(c4)oc4ccccc45)c3c2)cc1 ; c1ccc(-n2c3ccccc3c3ccc(N(c4ccc5c(c4)C4(c6ccccc6-c6ccccc64)c4cccc6cccc-5c46)c4cccc5oc6ccccc6c45)cc32)cc1]

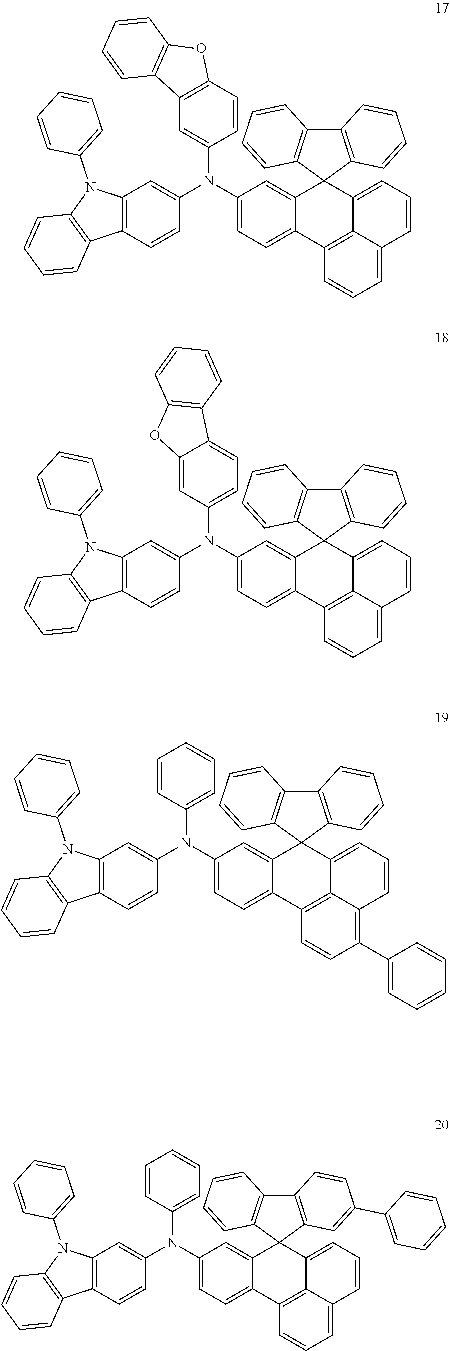 [SMILES: c1ccc(-n2c3ccccc3c3ccc(N(c4ccc5c(c4)C4(c6ccccc6-c6ccccc64)c4cccc6cccc-5c46)c4ccc5oc6ccccc6c5c4)cc32)cc1 ; c1ccc(-n2c3ccccc3c3ccc(N(c4ccc5c(c4)C4(c6ccccc6-c6ccccc64)c4cccc6cccc-5c46)c4ccc5c(c4)oc4ccccc45)cc32)cc1 ; c1ccc(-c2ccc3c4c(cccc24)C2(c4ccccc4-c4ccccc42)c2cc(N(c4ccccc4)c4ccc5c6ccccc6n(-c6ccccc6)c5c4)ccc2-3)cc1 ; c1ccc(-c2ccc3c(c2)C2(c4ccccc4-3)c3cc(N(c4ccccc4)c4ccc5c6ccccc6n(-c6ccccc6)c5c4)ccc3-c3cccc4cccc2c34)cc1]

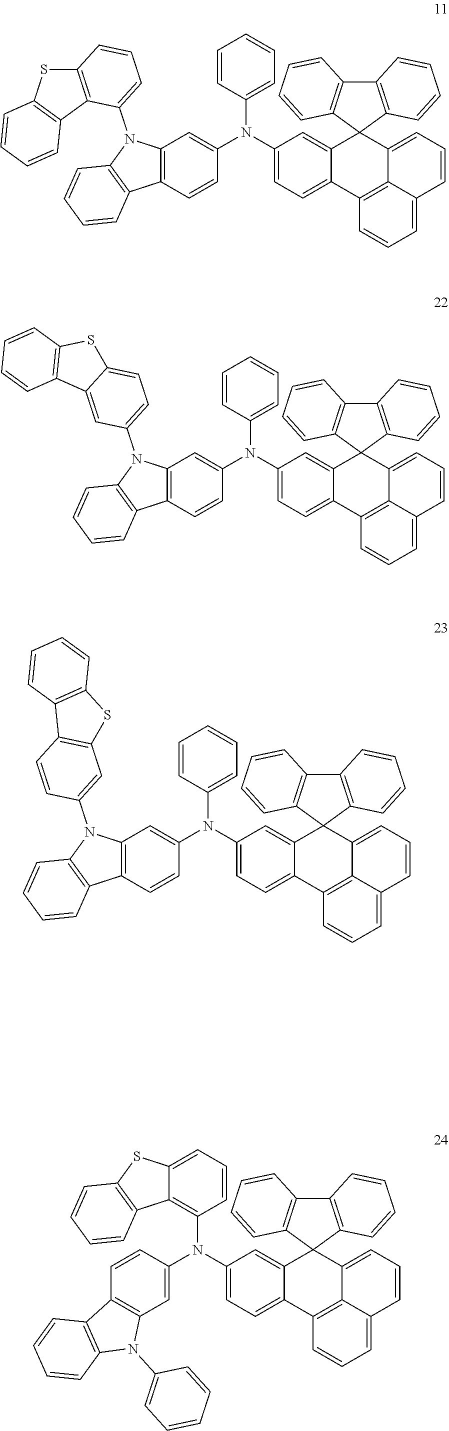 [SMILES: c1ccc(N(c2ccc3c(c2)C2(c4ccccc4-c4ccccc42)c2cccc4cccc-3c24)c2ccc3c4ccccc4n(-c4cccc5sc6ccccc6c45)c3c2)cc1 ; c1ccc(N(c2ccc3c(c2)C2(c4ccccc4-c4ccccc42)c2cccc4cccc-3c24)c2ccc3c4ccccc4n(-c4ccc5sc6ccccc6c5c4)c3c2)cc1 ; c1ccc(N(c2ccc3c(c2)C2(c4ccccc4-c4ccccc42)c2cccc4cccc-3c24)c2ccc3c4ccccc4n(-c4ccc5c(c4)sc4ccccc45)c3c2)cc1 ; c1ccc(-n2c3ccccc3c3ccc(N(c4ccc5c(c4)C4(c6ccccc6-c6ccccc64)c4cccc6cccc-5c46)c4cccc5sc6ccccc6c45)cc32)cc1]

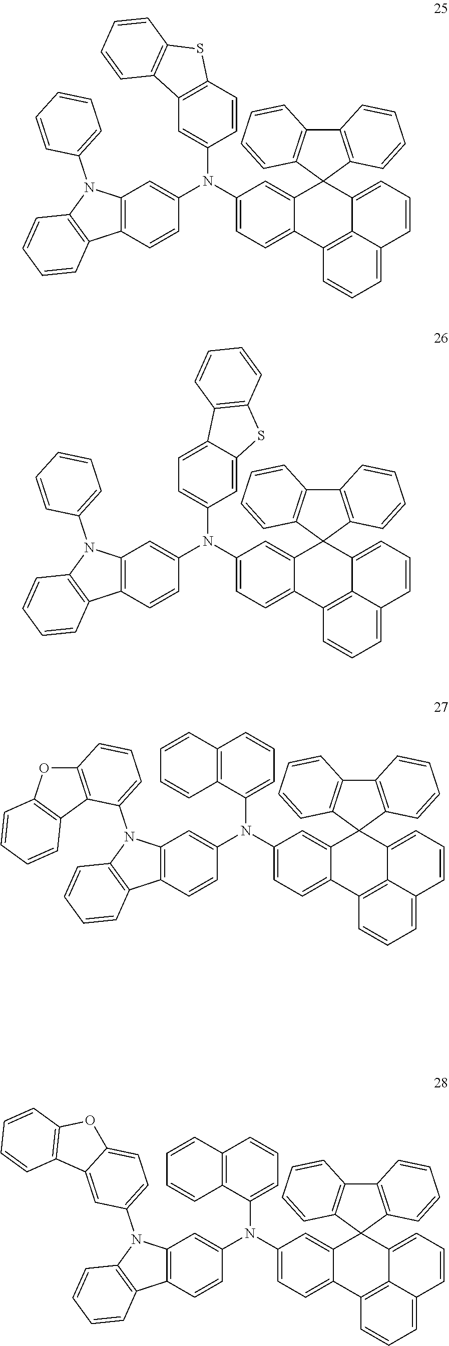 [SMILES: c1ccc(-n2c3ccccc3c3ccc(N(c4ccc5c(c4)C4(c6ccccc6-c6ccccc64)c4cccc6cccc-5c46)c4ccc5sc6ccccc6c5c4)cc32)cc1 ; c1ccc(-n2c3ccccc3c3ccc(N(c4ccc5c(c4)C4(c6ccccc6-c6ccccc64)c4cccc6cccc-5c46)c4ccc5c(c4)sc4ccccc45)cc32)cc1 ; c1ccc2c(c1)-c1ccccc1C21c2cc(N(c3ccc4c5ccccc5n(-c5cccc6oc7ccccc7c56)c4c3)c3cccc4ccccc34)ccc2-c2cccc3cccc1c23 ; c1ccc2c(c1)-c1ccccc1C21c2cc(N(c3ccc4c5ccccc5n(-c5ccc6oc7ccccc7c6c5)c4c3)c3cccc4ccccc34)ccc2-c2cccc3cccc1c23]

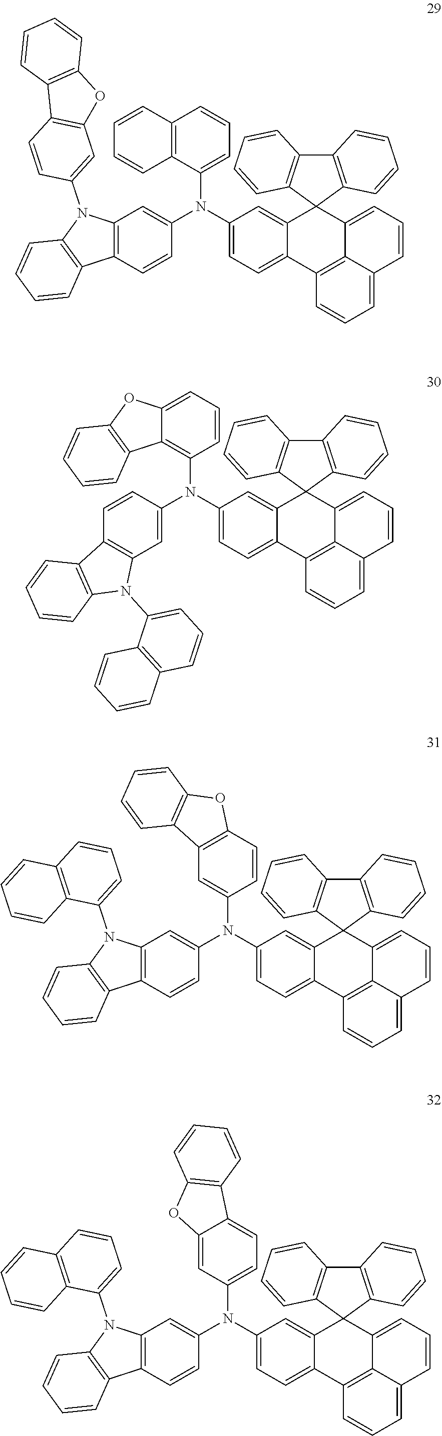 [SMILES: c1ccc2c(c1)-c1ccccc1C21c2cc(N(c3ccc4c5ccccc5n(-c5ccc6c(c5)oc5ccccc56)c4c3)c3cccc4ccccc34)ccc2-c2cccc3cccc1c23 ; c1ccc2c(c1)-c1ccccc1C21c2cc(N(c3ccc4c5ccccc5n(-c5cccc6ccccc56)c4c3)c3cccc4oc5ccccc5c34)ccc2-c2cccc3cccc1c23 ; c1ccc2c(c1)-c1ccccc1C21c2cc(N(c3ccc4oc5ccccc5c4c3)c3ccc4c5ccccc5n(-c5cccc6ccccc56)c4c3)ccc2-c2cccc3cccc1c23 ; c1ccc2c(c1)-c1ccccc1C21c2cc(N(c3ccc4c(c3)oc3ccccc34)c3ccc4c5ccccc5n(-c5cccc6ccccc56)c4c3)ccc2-c2cccc3cccc1c23]

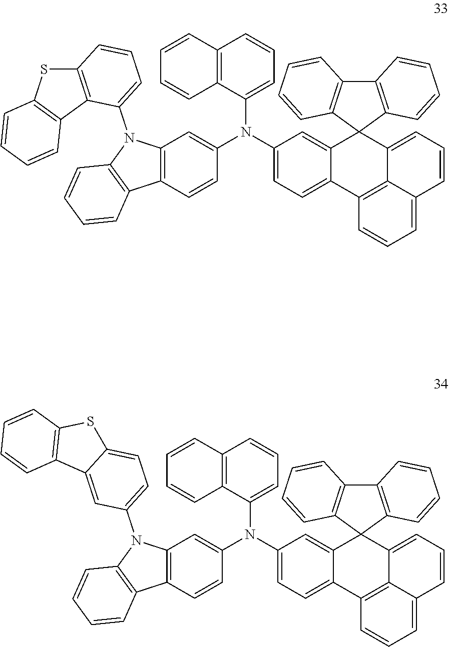 [SMILES: c1ccc2c(c1)-c1ccccc1C21c2cc(N(c3ccc4c5ccccc5n(-c5cccc6sc7ccccc7c56)c4c3)c3cccc4ccccc34)ccc2-c2cccc3cccc1c23 ; c1ccc2c(c1)-c1ccccc1C21c2cc(N(c3ccc4c5ccccc5n(-c5ccc6sc7ccccc7c6c5)c4c3)c3cccc4ccccc34)ccc2-c2cccc3cccc1c23]

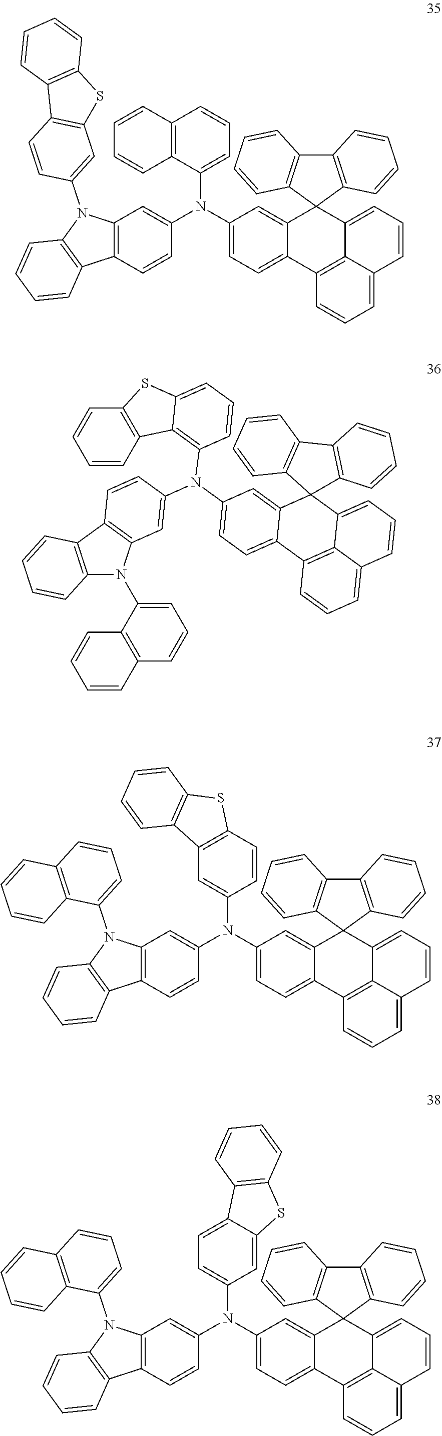 [SMILES: c1ccc2c(c1)-c1ccccc1C21c2cc(N(c3ccc4c5ccccc5n(-c5ccc6c(c5)sc5ccccc56)c4c3)c3cccc4ccccc34)ccc2-c2cccc3cccc1c23 ; c1ccc2c(c1)-c1ccccc1C21c2cc(N(c3ccc4c5ccccc5n(-c5cccc6ccccc56)c4c3)c3cccc4sc5ccccc5c34)ccc2-c2cccc3cccc1c23 ; c1ccc2c(c1)-c1ccccc1C21c2cc(N(c3ccc4sc5ccccc5c4c3)c3ccc4c5ccccc5n(-c5cccc6ccccc56)c4c3)ccc2-c2cccc3cccc1c23 ; c1ccc2c(c1)-c1ccccc1C21c2cc(N(c3ccc4c(c3)sc3ccccc34)c3ccc4c5ccccc5n(-c5cccc6ccccc56)c4c3)ccc2-c2cccc3cccc1c23]

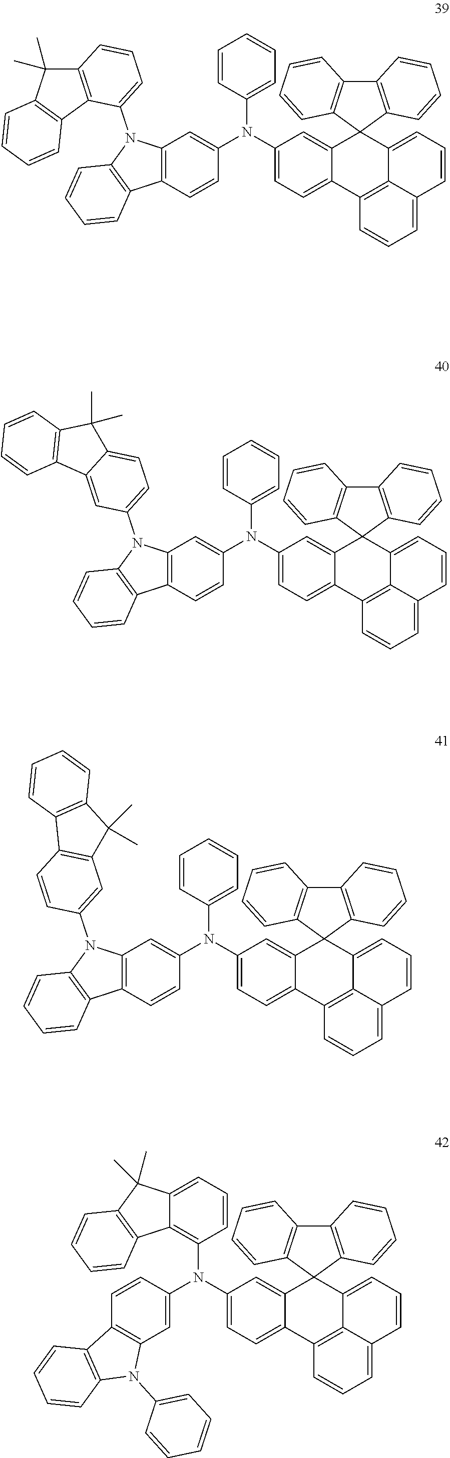 [SMILES: CC1(C)c2ccccc2-c2c(-n3c4ccccc4c4ccc(N(c5ccccc5)c5ccc6c(c5)C5(c7ccccc7-c7ccccc75)c5cccc7cccc-6c57)cc43)cccc21 ; CC1(C)c2ccccc2-c2cc(-n3c4ccccc4c4ccc(N(c5ccccc5)c5ccc6c(c5)C5(c7ccccc7-c7ccccc75)c5cccc7cccc-6c57)cc43)ccc21 ; CC1(C)c2ccccc2-c2ccc(-n3c4ccccc4c4ccc(N(c5ccccc5)c5ccc6c(c5)C5(c7ccccc7-c7ccccc75)c5cccc7cccc-6c57)cc43)cc21 ; CC1(C)c2ccccc2-c2c(N(c3ccc4c(c3)C3(c5ccccc5-c5ccccc53)c3cccc5cccc-4c35)c3ccc4c5ccccc5n(-c5ccccc5)c4c3)cccc21]

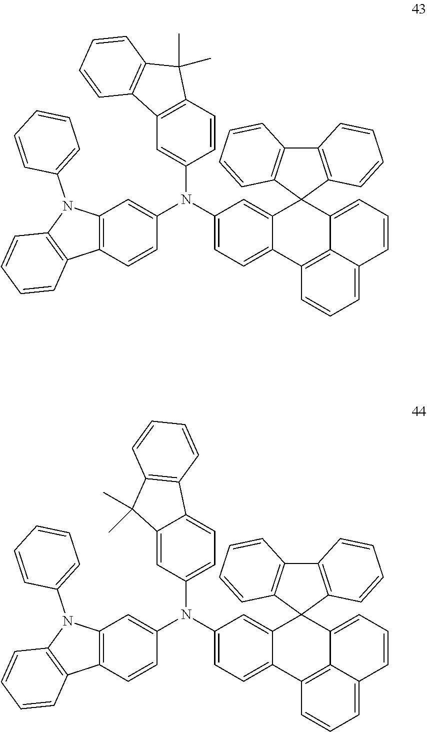 [SMILES: CC1(C)c2ccccc2-c2cc(N(c3ccc4c(c3)C3(c5ccccc5-c5ccccc53)c3cccc5cccc-4c35)c3ccc4c5ccccc5n(-c5ccccc5)c4c3)ccc21 ; CC1(C)c2ccccc2-c2ccc(N(c3ccc4c(c3)C3(c5ccccc5-c5ccccc53)c3cccc5cccc-4c35)c3ccc4c5ccccc5n(-c5ccccc5)c4c3)cc21]

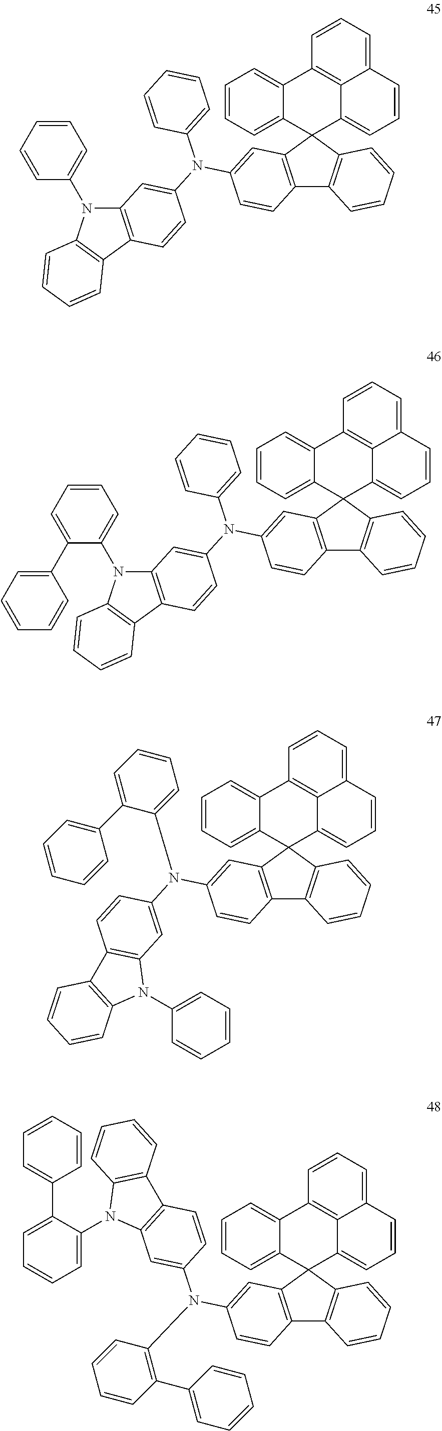 [SMILES: c1ccc(N(c2ccc3c(c2)C2(c4ccccc4-3)c3ccccc3-c3cccc4cccc2c34)c2ccc3c4ccccc4n(-c4ccccc4)c3c2)cc1 ; c1ccc(-c2ccccc2-n2c3ccccc3c3ccc(N(c4ccccc4)c4ccc5c(c4)C4(c6ccccc6-5)c5ccccc5-c5cccc6cccc4c56)cc32)cc1 ; c1ccc(-c2ccccc2N(c2ccc3c(c2)C2(c4ccccc4-3)c3ccccc3-c3cccc4cccc2c34)c2ccc3c4ccccc4n(-c4ccccc4)c3c2)cc1 ; c1ccc(-c2ccccc2N(c2ccc3c(c2)C2(c4ccccc4-3)c3ccccc3-c3cccc4cccc2c34)c2ccc3c4ccccc4n(-c4ccccc4-c4ccccc4)c3c2)cc1]

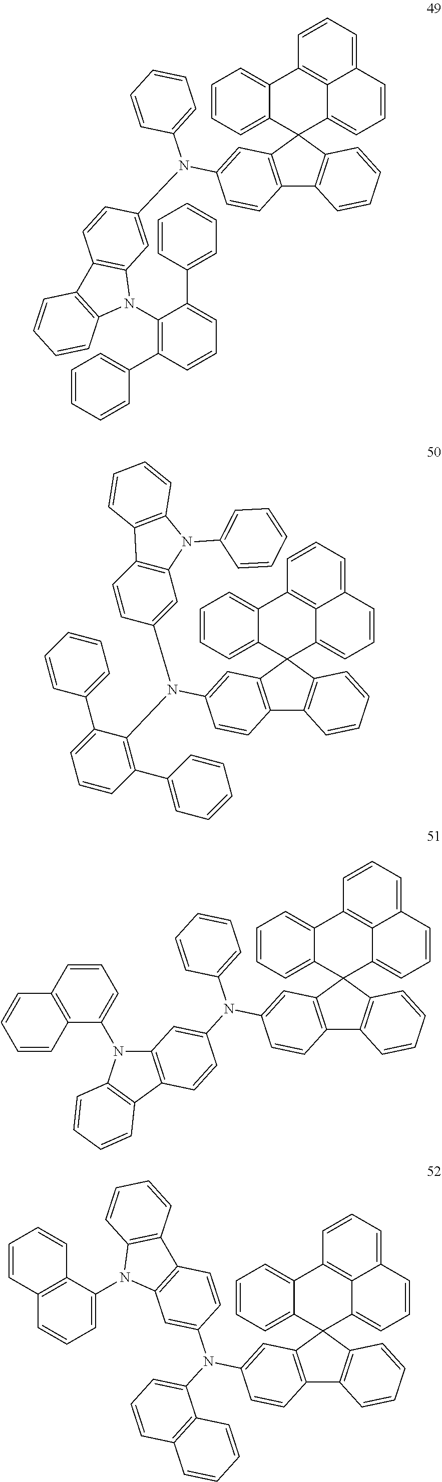

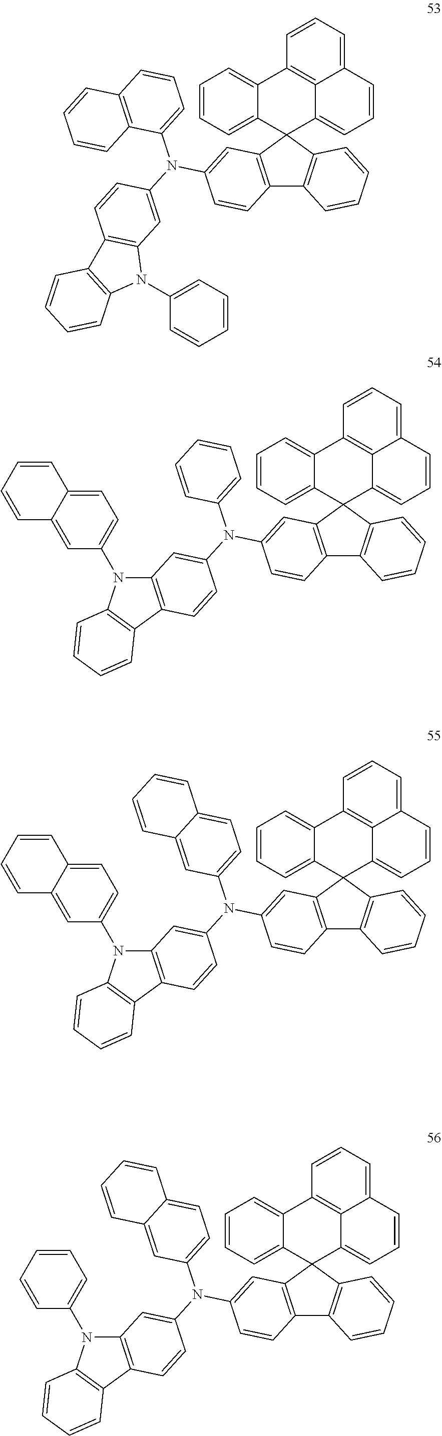 [SMILES: c1ccc(-n2c3ccccc3c3ccc(N(c4ccc5c(c4)C4(c6ccccc6-5)c5ccccc5-c5cccc6cccc4c56)c4cccc5ccccc45)cc32)cc1 ; c1ccc(N(c2ccc3c(c2)C2(c4ccccc4-3)c3ccccc3-c3cccc4cccc2c34)c2ccc3c4ccccc4n(-c4ccc5ccccc5c4)c3c2)cc1 ; c1ccc2c(c1)-c1ccc(N(c3ccc4ccccc4c3)c3ccc4c5ccccc5n(-c5ccc6ccccc6c5)c4c3)cc1C21c2ccccc2-c2cccc3cccc1c23 ; c1ccc(-n2c3ccccc3c3ccc(N(c4ccc5c(c4)C4(c6ccccc6-5)c5ccccc5-c5cccc6cccc4c56)c4ccc5ccccc5c4)cc32)cc1]

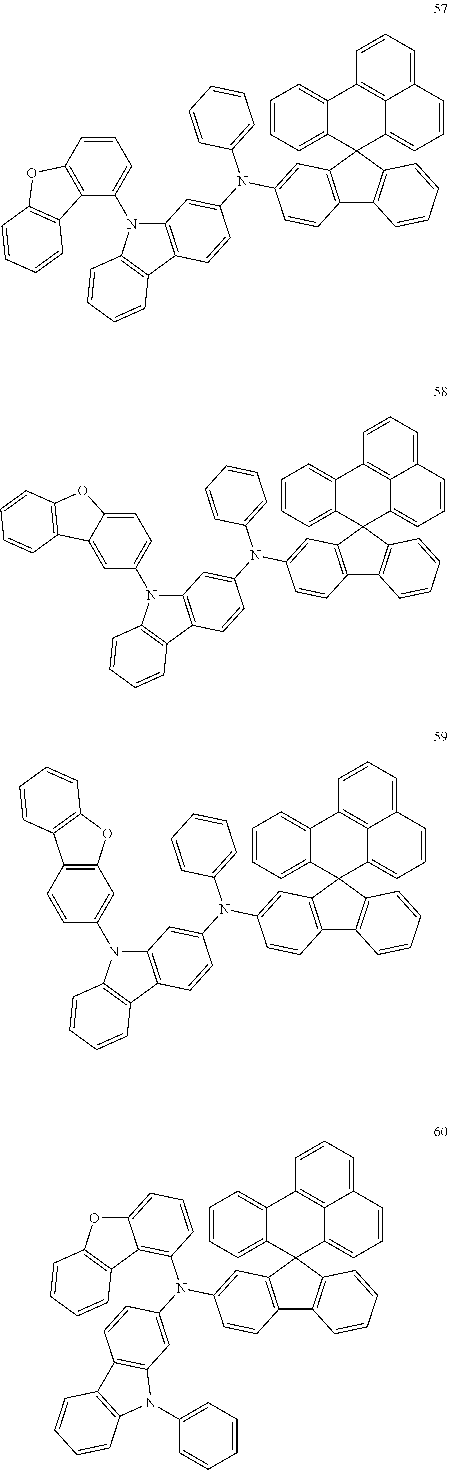 [SMILES: c1ccc(N(c2ccc3c(c2)C2(c4ccccc4-3)c3ccccc3-c3cccc4cccc2c34)c2ccc3c4ccccc4n(-c4cccc5oc6ccccc6c45)c3c2)cc1 ; c1ccc(N(c2ccc3c(c2)C2(c4ccccc4-3)c3ccccc3-c3cccc4cccc2c34)c2ccc3c4ccccc4n(-c4ccc5oc6ccccc6c5c4)c3c2)cc1 ; c1ccc(N(c2ccc3c(c2)C2(c4ccccc4-3)c3ccccc3-c3cccc4cccc2c34)c2ccc3c4ccccc4n(-c4ccc5c(c4)oc4ccccc45)c3c2)cc1 ; c1ccc(-n2c3ccccc3c3ccc(N(c4ccc5c(c4)C4(c6ccccc6-5)c5ccccc5-c5cccc6cccc4c56)c4cccc5oc6ccccc6c45)cc32)cc1]

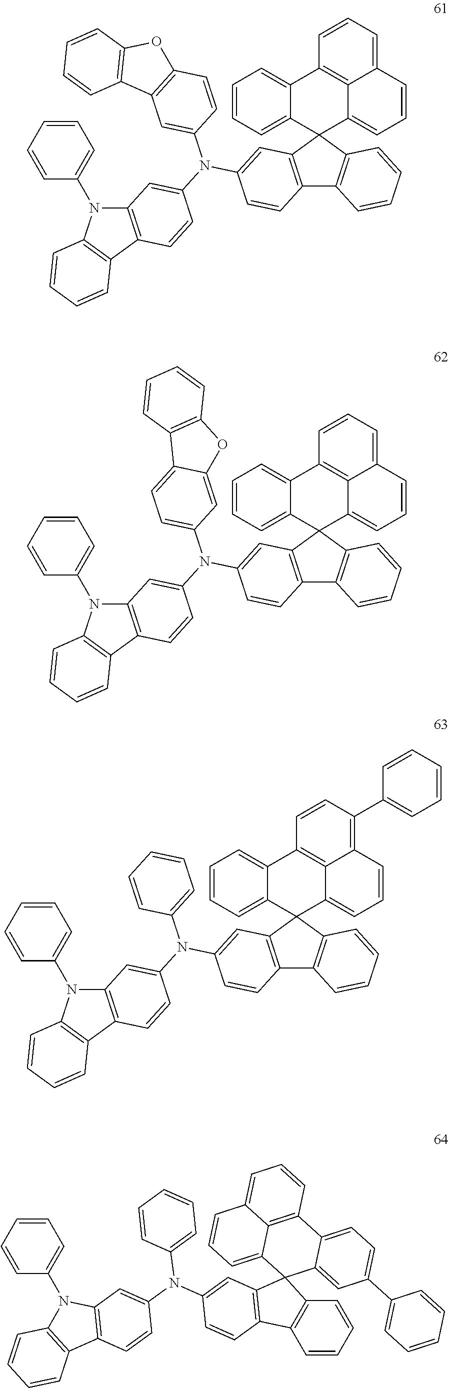 [SMILES: c1ccc(-n2c3ccccc3c3ccc(N(c4ccc5c(c4)C4(c6ccccc6-5)c5ccccc5-c5cccc6cccc4c56)c4ccc5oc6ccccc6c5c4)cc32)cc1 ; c1ccc(-n2c3ccccc3c3ccc(N(c4ccc5c(c4)C4(c6ccccc6-5)c5ccccc5-c5cccc6cccc4c56)c4ccc5c(c4)oc4ccccc45)cc32)cc1 ; c1ccc(-c2ccc3c4c(cccc24)C2(c4ccccc4-c4ccc(N(c5ccccc5)c5ccc6c7ccccc7n(-c7ccccc7)c6c5)cc42)c2ccccc2-3)cc1 ; c1ccc(-c2ccc3c(c2)C2(c4ccccc4-c4ccc(N(c5ccccc5)c5ccc6c7ccccc7n(-c7ccccc7)c6c5)cc42)c2cccc4cccc-3c24)cc1]

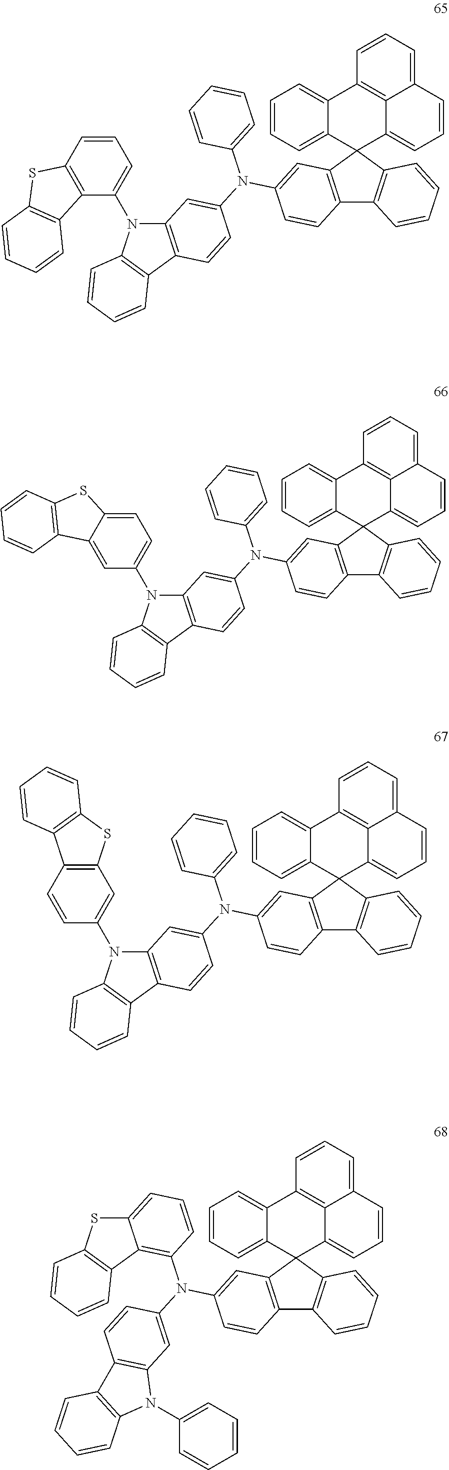 [SMILES: c1ccc(N(c2ccc3c(c2)C2(c4ccccc4-3)c3ccccc3-c3cccc4cccc2c34)c2ccc3c4ccccc4n(-c4cccc5sc6ccccc6c45)c3c2)cc1 ; c1ccc(N(c2ccc3c(c2)C2(c4ccccc4-3)c3ccccc3-c3cccc4cccc2c34)c2ccc3c4ccccc4n(-c4ccc5sc6ccccc6c5c4)c3c2)cc1 ; c1ccc(N(c2ccc3c(c2)C2(c4ccccc4-3)c3ccccc3-c3cccc4cccc2c34)c2ccc3c4ccccc4n(-c4ccc5c(c4)sc4ccccc45)c3c2)cc1 ; c1ccc(-n2c3ccccc3c3ccc(N(c4ccc5c(c4)C4(c6ccccc6-5)c5ccccc5-c5cccc6cccc4c56)c4cccc5sc6ccccc6c45)cc32)cc1]

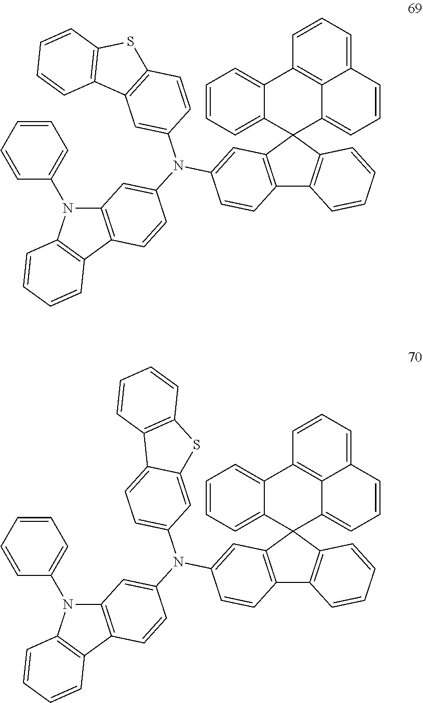 [SMILES: c1ccc(-n2c3ccccc3c3ccc(N(c4ccc5c(c4)C4(c6ccccc6-5)c5ccccc5-c5cccc6cccc4c56)c4ccc5sc6ccccc6c5c4)cc32)cc1 ; c1ccc(-n2c3ccccc3c3ccc(N(c4ccc5c(c4)C4(c6ccccc6-5)c5ccccc5-c5cccc6cccc4c56)c4ccc5c(c4)sc4ccccc45)cc32)cc1]

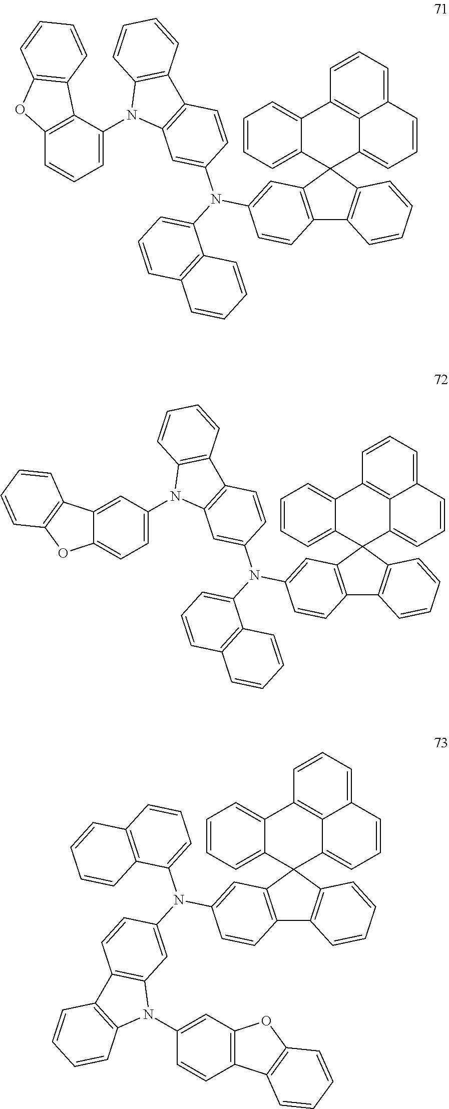 [SMILES: c1ccc2c(c1)-c1ccc(N(c3ccc4c5ccccc5n(-c5cccc6oc7ccccc7c56)c4c3)c3cccc4ccccc34)cc1C21c2ccccc2-c2cccc3cccc1c23 ; c1ccc2c(c1)-c1ccc(N(c3ccc4c5ccccc5n(-c5ccc6oc7ccccc7c6c5)c4c3)c3cccc4ccccc34)cc1C21c2ccccc2-c2cccc3cccc1c23 ; c1ccc2c(c1)-c1ccc(N(c3ccc4c5ccccc5n(-c5ccc6c(c5)oc5ccccc56)c4c3)c3cccc4ccccc34)cc1C21c2ccccc2-c2cccc3cccc1c23]

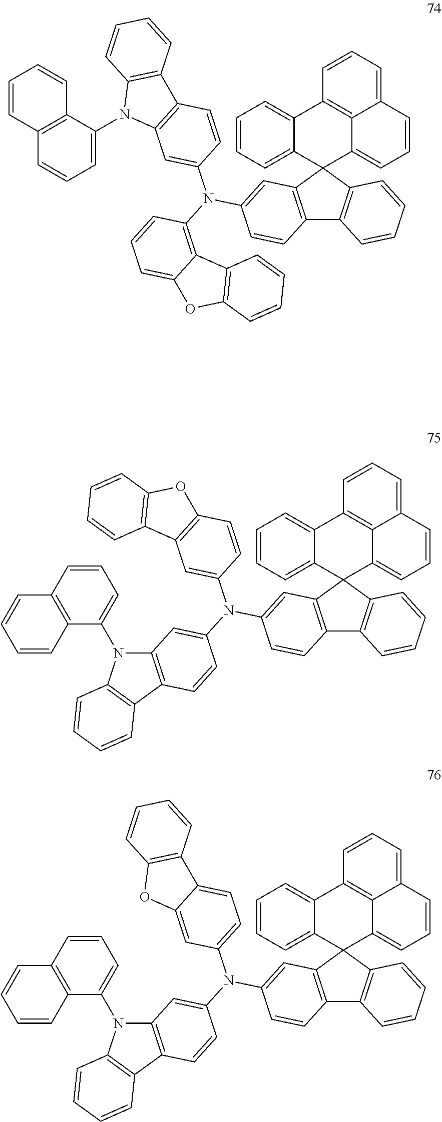 [SMILES: c1ccc2c(c1)-c1ccc(N(c3ccc4c5ccccc5n(-c5cccc6ccccc56)c4c3)c3cccc4oc5ccccc5c34)cc1C21c2ccccc2-c2cccc3cccc1c23 ; c1ccc2c(c1)-c1ccc(N(c3ccc4oc5ccccc5c4c3)c3ccc4c5ccccc5n(-c5cccc6ccccc56)c4c3)cc1C21c2ccccc2-c2cccc3cccc1c23 ; c1ccc2c(c1)-c1ccc(N(c3ccc4c(c3)oc3ccccc34)c3ccc4c5ccccc5n(-c5cccc6ccccc56)c4c3)cc1C21c2ccccc2-c2cccc3cccc1c23]

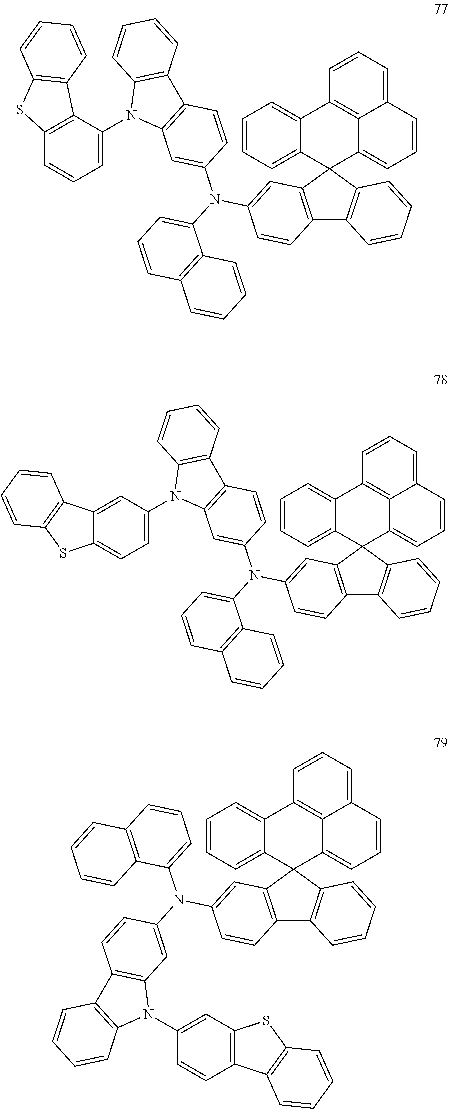 [SMILES: c1ccc2c(c1)-c1ccc(N(c3ccc4c5ccccc5n(-c5cccc6sc7ccccc7c56)c4c3)c3cccc4ccccc34)cc1C21c2ccccc2-c2cccc3cccc1c23 ; c1ccc2c(c1)-c1ccc(N(c3ccc4c5ccccc5n(-c5ccc6sc7ccccc7c6c5)c4c3)c3cccc4ccccc34)cc1C21c2ccccc2-c2cccc3cccc1c23 ; c1ccc2c(c1)-c1ccc(N(c3ccc4c5ccccc5n(-c5ccc6c(c5)sc5ccccc56)c4c3)c3cccc4ccccc34)cc1C21c2ccccc2-c2cccc3cccc1c23]

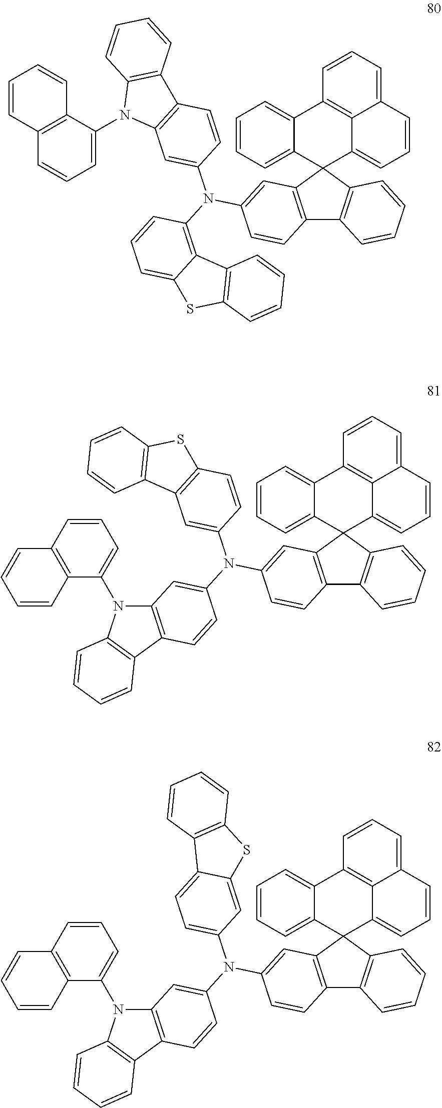 [SMILES: c1ccc2c(c1)-c1ccc(N(c3ccc4c5ccccc5n(-c5cccc6ccccc56)c4c3)c3cccc4sc5ccccc5c34)cc1C21c2ccccc2-c2cccc3cccc1c23 ; c1ccc2c(c1)-c1ccc(N(c3ccc4sc5ccccc5c4c3)c3ccc4c5ccccc5n(-c5cccc6ccccc56)c4c3)cc1C21c2ccccc2-c2cccc3cccc1c23 ; c1ccc2c(c1)-c1ccc(N(c3ccc4c(c3)sc3ccccc34)c3ccc4c5ccccc5n(-c5cccc6ccccc56)c4c3)cc1C21c2ccccc2-c2cccc3cccc1c23]

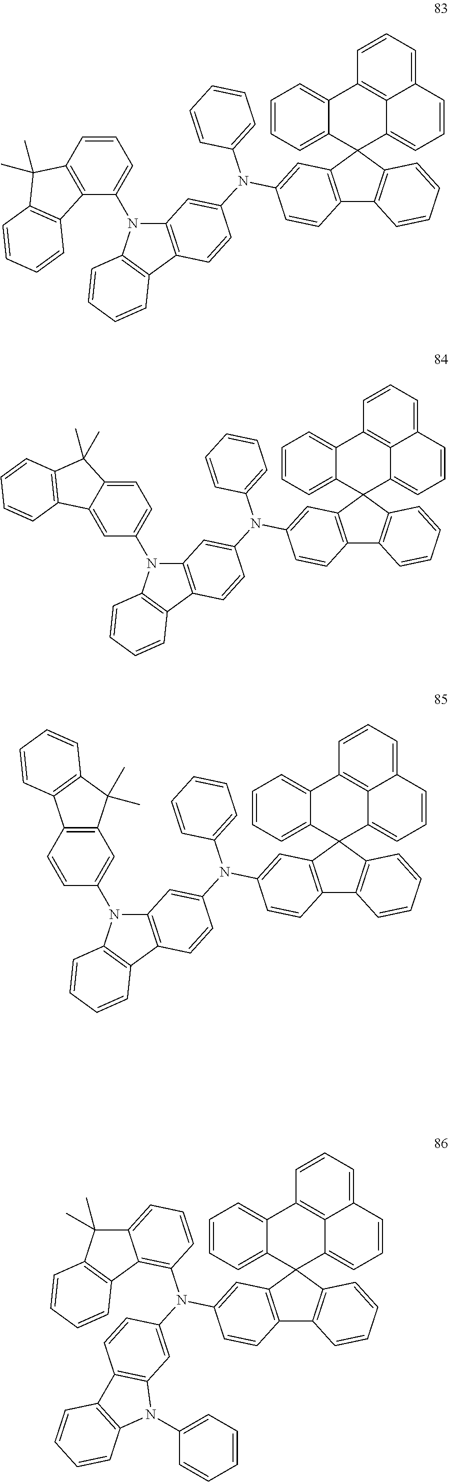 [SMILES: CC1(C)c2ccccc2-c2c(-n3c4ccccc4c4ccc(N(c5ccccc5)c5ccc6c(c5)C5(c7ccccc7-6)c6ccccc6-c6cccc7cccc5c67)cc43)cccc21 ; CC1(C)c2ccccc2-c2cc(-n3c4ccccc4c4ccc(N(c5ccccc5)c5ccc6c(c5)C5(c7ccccc7-6)c6ccccc6-c6cccc7cccc5c67)cc43)ccc21 ; CC1(C)c2ccccc2-c2ccc(-n3c4ccccc4c4ccc(N(c5ccccc5)c5ccc6c(c5)C5(c7ccccc7-6)c6ccccc6-c6cccc7cccc5c67)cc43)cc21 ; CC1(C)c2ccccc2-c2c(N(c3ccc4c(c3)C3(c5ccccc5-4)c4ccccc4-c4cccc5cccc3c45)c3ccc4c5ccccc5n(-c5ccccc5)c4c3)cccc21]

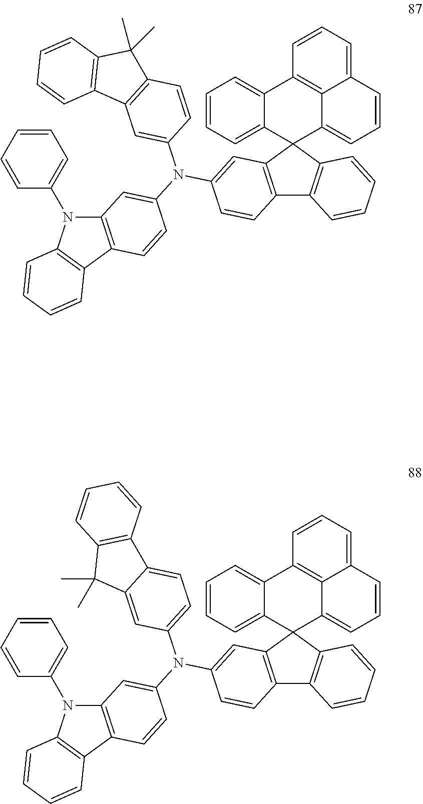 [SMILES: CC1(C)c2ccccc2-c2cc(N(c3ccc4c(c3)C3(c5ccccc5-4)c4ccccc4-c4cccc5cccc3c45)c3ccc4c5ccccc5n(-c5ccccc5)c4c3)ccc21 ; CC1(C)c2ccccc2-c2ccc(N(c3ccc4c(c3)C3(c5ccccc5-4)c4ccccc4-c4cccc5cccc3c45)c3ccc4c5ccccc5n(-c5ccccc5)c4c3)cc21]

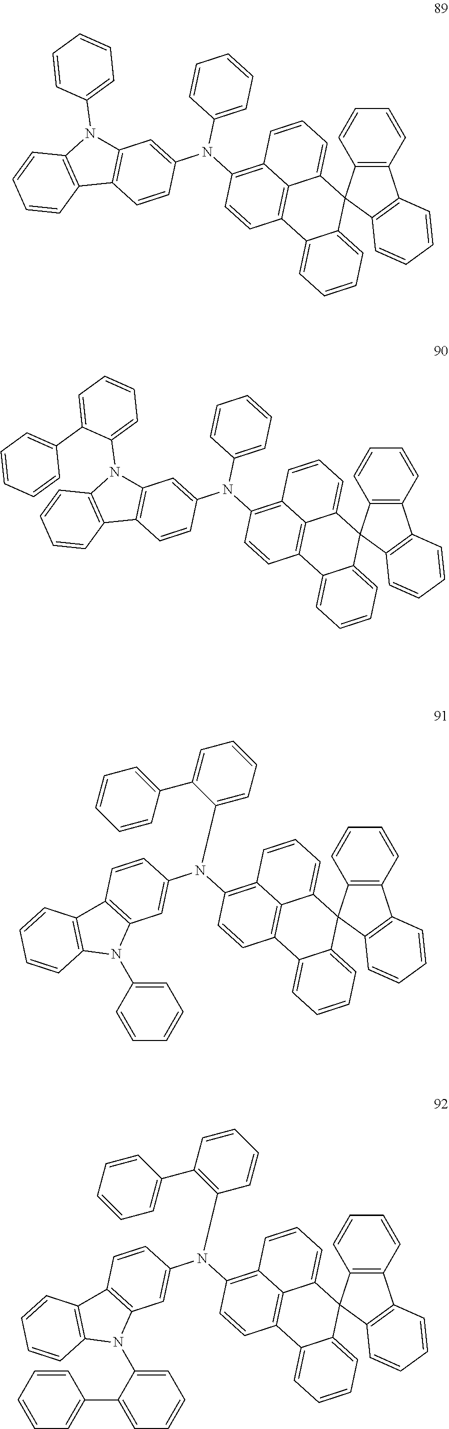 [SMILES: c1ccc(N(c2ccc3c4ccccc4n(-c4ccccc4)c3c2)c2ccc3c4c(cccc24)C2(c4ccccc4-c4ccccc42)c2ccccc2-3)cc1 ; c1ccc(-c2ccccc2-n2c3ccccc3c3ccc(N(c4ccccc4)c4ccc5c6c(cccc46)C4(c6ccccc6-c6ccccc64)c4ccccc4-5)cc32)cc1 ; c1ccc(-c2ccccc2N(c2ccc3c4ccccc4n(-c4ccccc4)c3c2)c2ccc3c4c(cccc24)C2(c4ccccc4-c4ccccc42)c2ccccc2-3)cc1 ; c1ccc(-c2ccccc2N(c2ccc3c4ccccc4n(-c4ccccc4-c4ccccc4)c3c2)c2ccc3c4c(cccc24)C2(c4ccccc4-c4ccccc42)c2ccccc2-3)cc1]

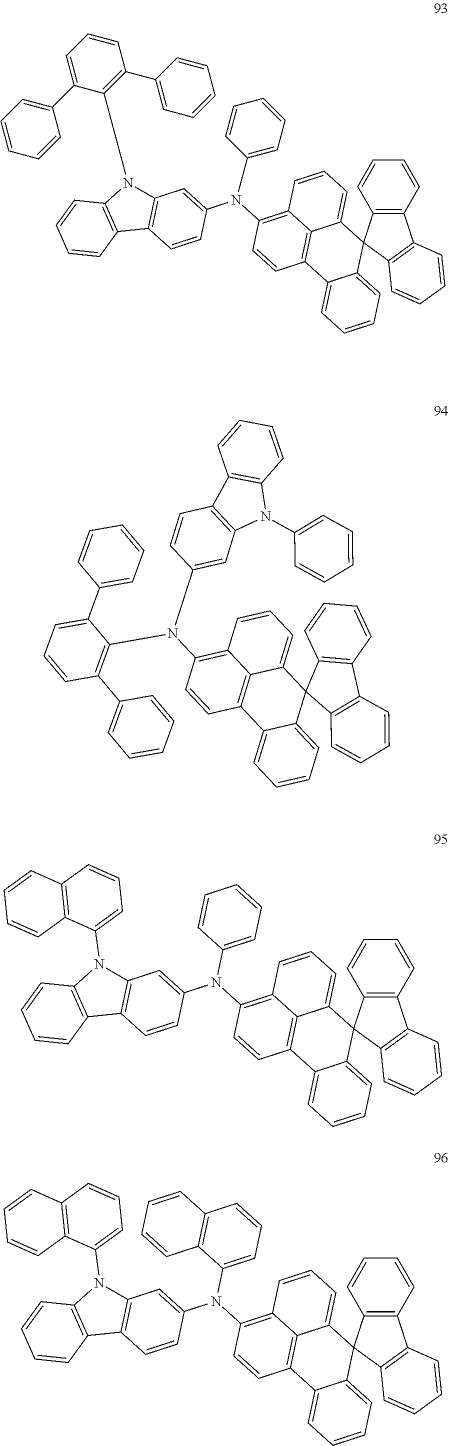 [SMILES: c1ccc(-c2cccc(-c3ccccc3)c2-n2c3ccccc3c3ccc(N(c4ccccc4)c4ccc5c6c(cccc46)C4(c6ccccc6-c6ccccc64)c4ccccc4-5)cc32)cc1 ; c1ccc(-c2cccc(-c3ccccc3)c2N(c2ccc3c4ccccc4n(-c4ccccc4)c3c2)c2ccc3c4c(cccc24)C2(c4ccccc4-c4ccccc42)c2ccccc2-3)cc1 ; c1ccc(N(c2ccc3c4ccccc4n(-c4cccc5ccccc45)c3c2)c2ccc3c4c(cccc24)C2(c4ccccc4-c4ccccc42)c2ccccc2-3)cc1 ; c1ccc2c(c1)-c1ccccc1C21c2ccccc2-c2ccc(N(c3ccc4c5ccccc5n(-c5cccc6ccccc56)c4c3)c3cccc4ccccc34)c3cccc1c23]

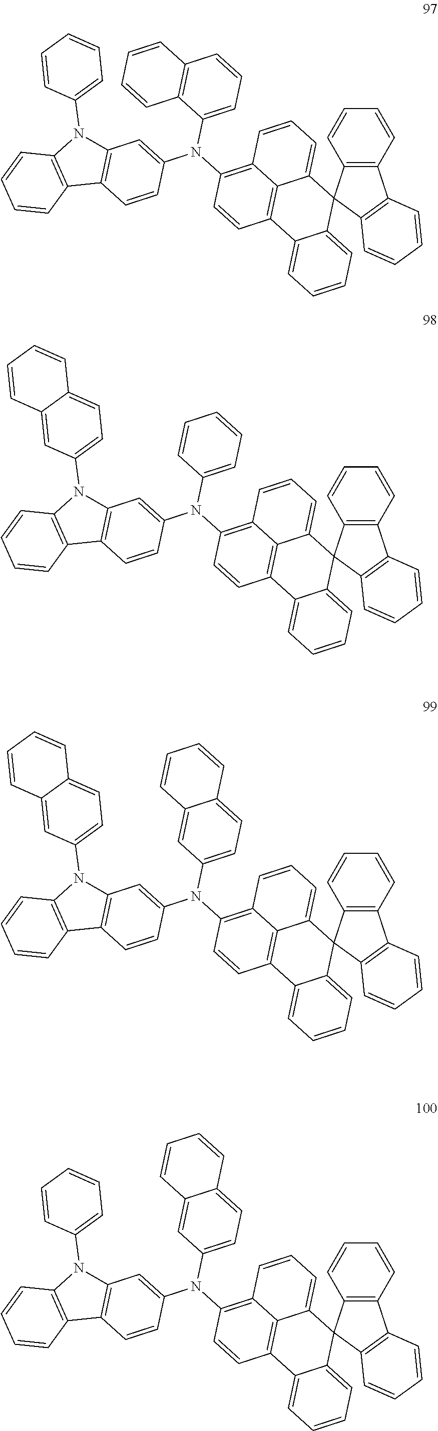 [SMILES: c1ccc(-n2c3ccccc3c3ccc(N(c4cccc5ccccc45)c4ccc5c6c(cccc46)C4(c6ccccc6-c6ccccc64)c4ccccc4-5)cc32)cc1 ; c1ccc(N(c2ccc3c4ccccc4n(-c4ccc5ccccc5c4)c3c2)c2ccc3c4c(cccc24)C2(c4ccccc4-c4ccccc42)c2ccccc2-3)cc1 ; c1ccc2c(c1)-c1ccccc1C21c2ccccc2-c2ccc(N(c3ccc4ccccc4c3)c3ccc4c5ccccc5n(-c5ccc6ccccc6c5)c4c3)c3cccc1c23 ; c1ccc(-n2c3ccccc3c3ccc(N(c4ccc5ccccc5c4)c4ccc5c6c(cccc46)C4(c6ccccc6-c6ccccc64)c4ccccc4-5)cc32)cc1]

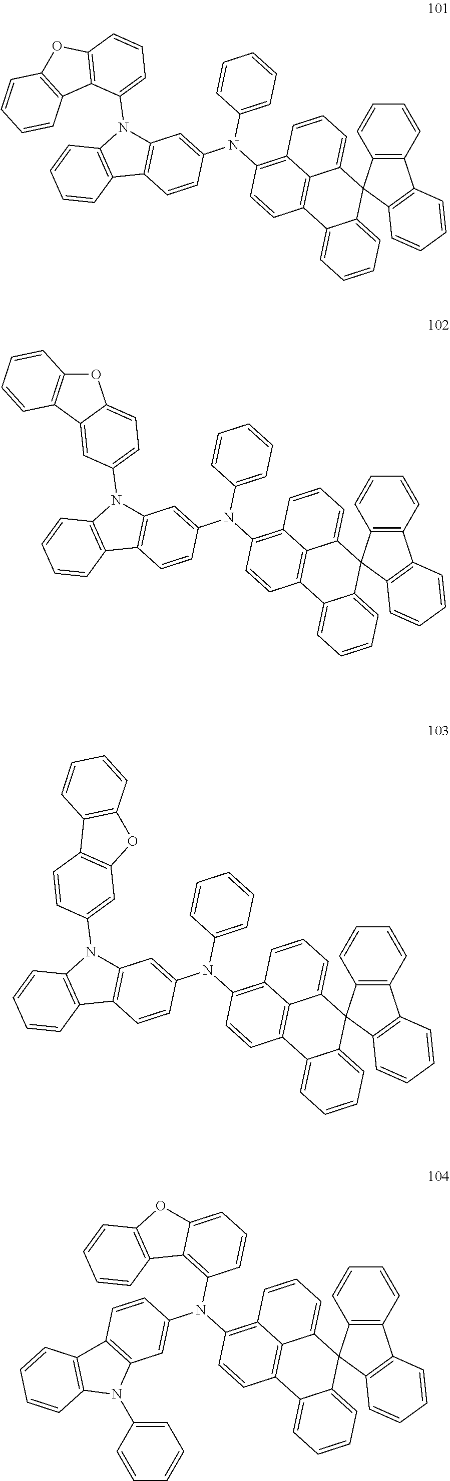 [SMILES: c1ccc(N(c2ccc3c4ccccc4n(-c4cccc5oc6ccccc6c45)c3c2)c2ccc3c4c(cccc24)C2(c4ccccc4-c4ccccc42)c2ccccc2-3)cc1 ; c1ccc(N(c2ccc3c4ccccc4n(-c4ccc5oc6ccccc6c5c4)c3c2)c2ccc3c4c(cccc24)C2(c4ccccc4-c4ccccc42)c2ccccc2-3)cc1 ; c1ccc(N(c2ccc3c4ccccc4n(-c4ccc5c(c4)oc4ccccc45)c3c2)c2ccc3c4c(cccc24)C2(c4ccccc4-c4ccccc42)c2ccccc2-3)cc1 ; c1ccc(-n2c3ccccc3c3ccc(N(c4ccc5c6c(cccc46)C4(c6ccccc6-c6ccccc64)c4ccccc4-5)c4cccc5oc6ccccc6c45)cc32)cc1]

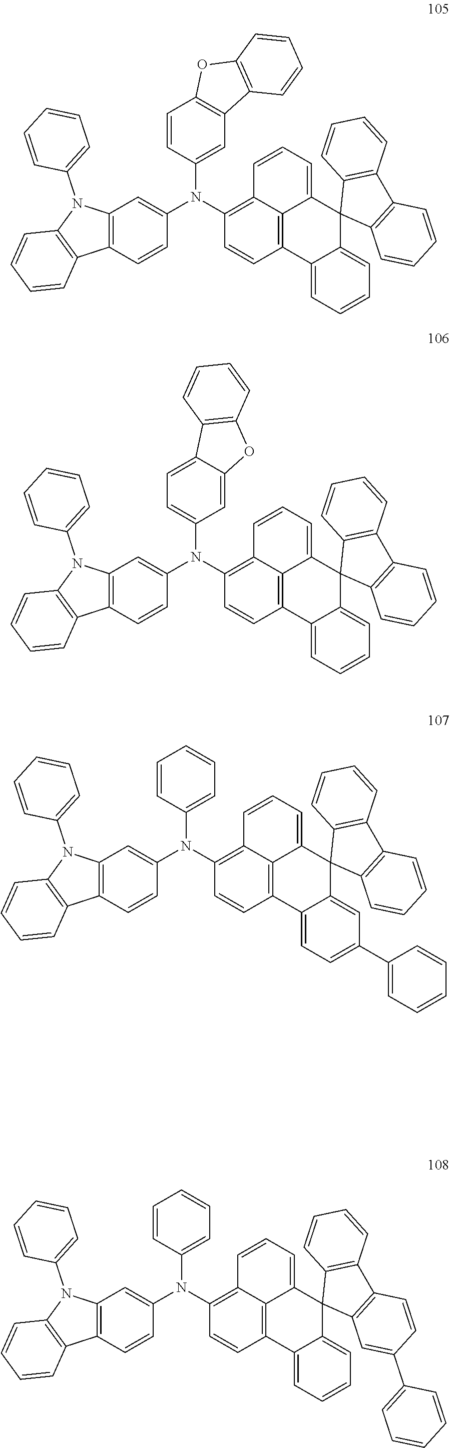 [SMILES: c1ccc(-n2c3ccccc3c3ccc(N(c4ccc5oc6ccccc6c5c4)c4ccc5c6c(cccc46)C4(c6ccccc6-c6ccccc64)c4ccccc4-5)cc32)cc1 ; c1ccc(-n2c3ccccc3c3ccc(N(c4ccc5c(c4)oc4ccccc45)c4ccc5c6c(cccc46)C4(c6ccccc6-c6ccccc64)c4ccccc4-5)cc32)cc1 ; c1ccc(-c2ccc3c(c2)C2(c4ccccc4-c4ccccc42)c2cccc4c(N(c5ccccc5)c5ccc6c7ccccc7n(-c7ccccc7)c6c5)ccc-3c24)cc1 ; c1ccc(-c2ccc3c(c2)C2(c4ccccc4-3)c3ccccc3-c3ccc(N(c4ccccc4)c4ccc5c6ccccc6n(-c6ccccc6)c5c4)c4cccc2c34)cc1]

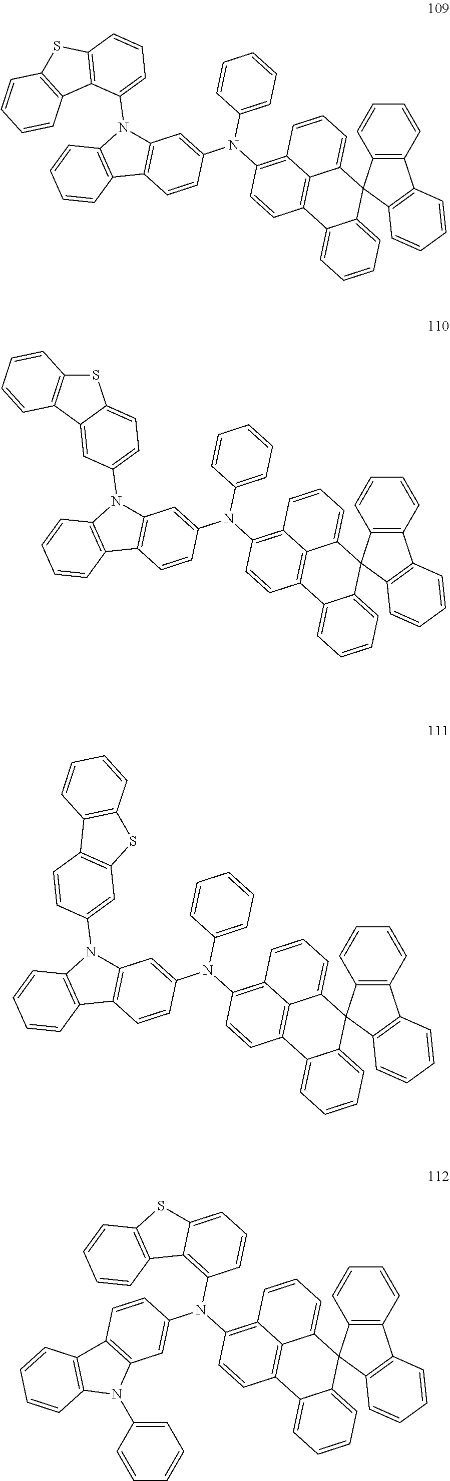 [SMILES: c1ccc(N(c2ccc3c4ccccc4n(-c4cccc5sc6ccccc6c45)c3c2)c2ccc3c4c(cccc24)C2(c4ccccc4-c4ccccc42)c2ccccc2-3)cc1 ; c1ccc(N(c2ccc3c4ccccc4n(-c4ccc5sc6ccccc6c5c4)c3c2)c2ccc3c4c(cccc24)C2(c4ccccc4-c4ccccc42)c2ccccc2-3)cc1 ; c1ccc(N(c2ccc3c4ccccc4n(-c4ccc5c(c4)sc4ccccc45)c3c2)c2ccc3c4c(cccc24)C2(c4ccccc4-c4ccccc42)c2ccccc2-3)cc1 ; c1ccc(-n2c3ccccc3c3ccc(N(c4ccc5c6c(cccc46)C4(c6ccccc6-c6ccccc64)c4ccccc4-5)c4cccc5sc6ccccc6c45)cc32)cc1]

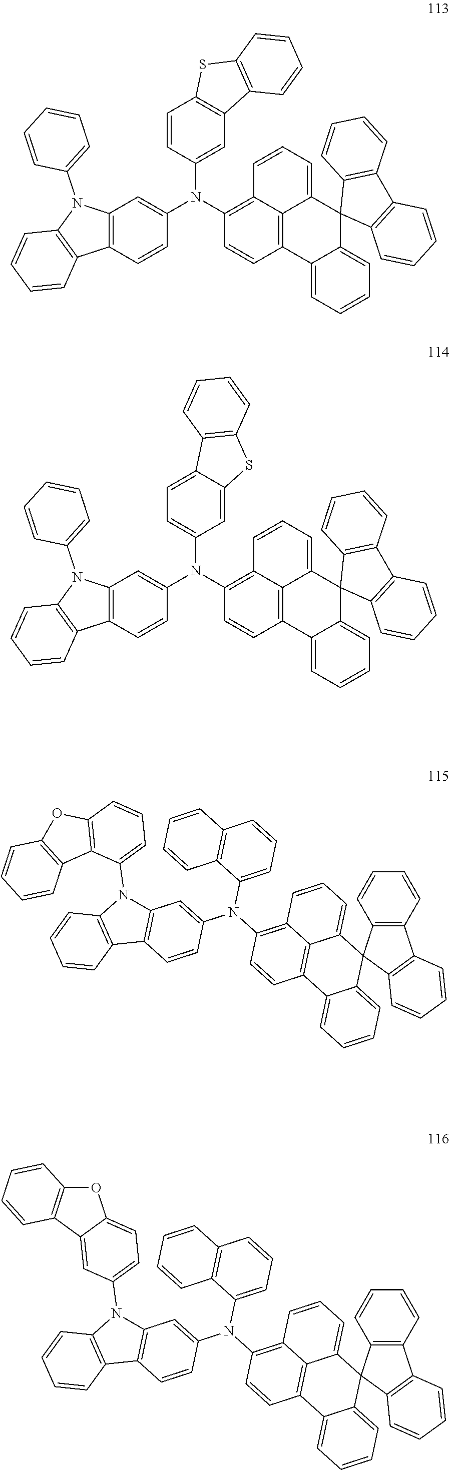 [SMILES: c1ccc(-n2c3ccccc3c3ccc(N(c4ccc5sc6ccccc6c5c4)c4ccc5c6c(cccc46)C4(c6ccccc6-c6ccccc64)c4ccccc4-5)cc32)cc1 ; c1ccc(-n2c3ccccc3c3ccc(N(c4ccc5c(c4)sc4ccccc45)c4ccc5c6c(cccc46)C4(c6ccccc6-c6ccccc64)c4ccccc4-5)cc32)cc1 ; c1ccc2c(c1)-c1ccccc1C21c2ccccc2-c2ccc(N(c3ccc4c5ccccc5n(-c5cccc6oc7ccccc7c56)c4c3)c3cccc4ccccc34)c3cccc1c23 ; c1ccc2c(c1)-c1ccccc1C21c2ccccc2-c2ccc(N(c3ccc4c5ccccc5n(-c5ccc6oc7ccccc7c6c5)c4c3)c3cccc4ccccc34)c3cccc1c23]

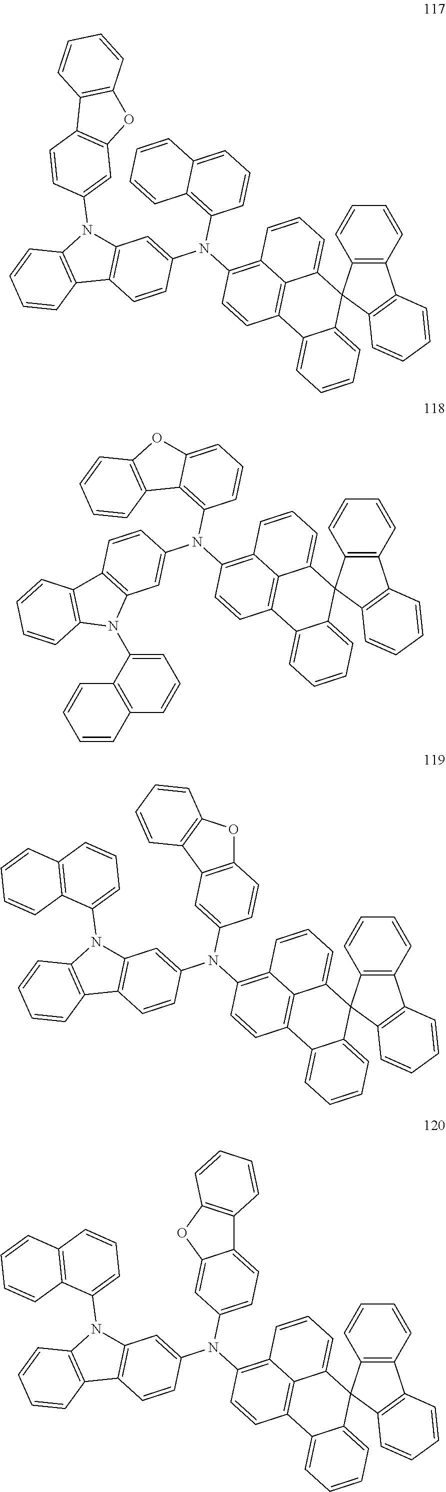 [SMILES: c1ccc2c(c1)-c1ccccc1C21c2ccccc2-c2ccc(N(c3ccc4c5ccccc5n(-c5ccc6c(c5)oc5ccccc56)c4c3)c3cccc4ccccc34)c3cccc1c23 ; c1ccc2c(c1)-c1ccccc1C21c2ccccc2-c2ccc(N(c3ccc4c5ccccc5n(-c5cccc6ccccc56)c4c3)c3cccc4oc5ccccc5c34)c3cccc1c23 ; c1ccc2c(c1)-c1ccccc1C21c2ccccc2-c2ccc(N(c3ccc4oc5ccccc5c4c3)c3ccc4c5ccccc5n(-c5cccc6ccccc56)c4c3)c3cccc1c23 ; c1ccc2c(c1)-c1ccccc1C21c2ccccc2-c2ccc(N(c3ccc4c(c3)oc3ccccc34)c3ccc4c5ccccc5n(-c5cccc6ccccc56)c4c3)c3cccc1c23]

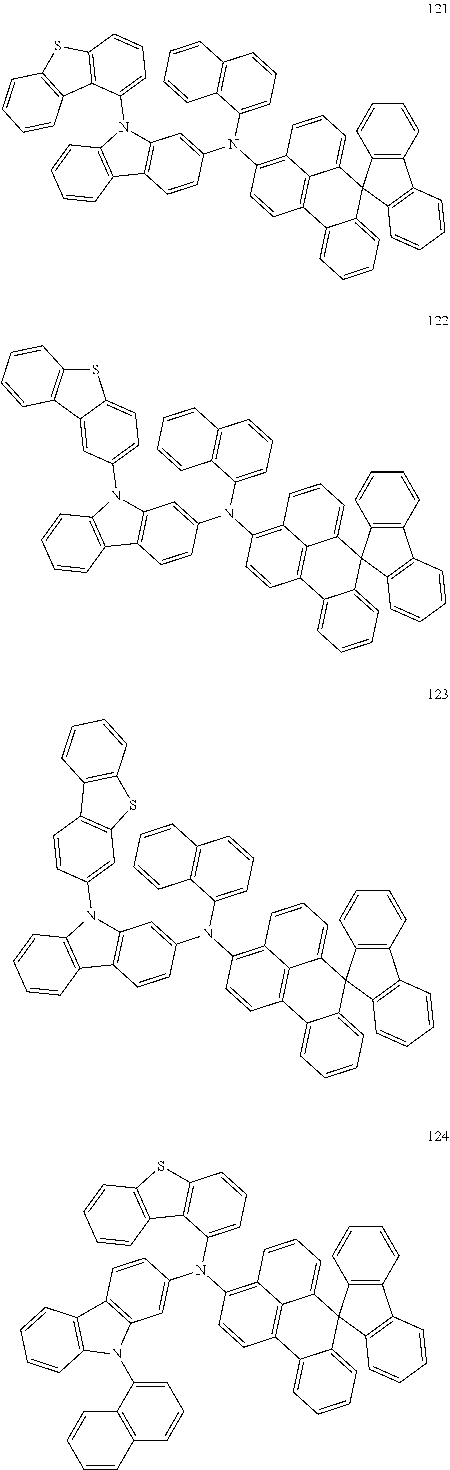 [SMILES: c1ccc2c(c1)-c1ccccc1C21c2ccccc2-c2ccc(N(c3ccc4c5ccccc5n(-c5cccc6sc7ccccc7c56)c4c3)c3cccc4ccccc34)c3cccc1c23 ; c1ccc2c(c1)-c1ccccc1C21c2ccccc2-c2ccc(N(c3ccc4c5ccccc5n(-c5ccc6sc7ccccc7c6c5)c4c3)c3cccc4ccccc34)c3cccc1c23 ; c1ccc2c(c1)-c1ccccc1C21c2ccccc2-c2ccc(N(c3ccc4c5ccccc5n(-c5ccc6c(c5)sc5ccccc56)c4c3)c3cccc4ccccc34)c3cccc1c23 ; c1ccc2c(c1)-c1ccccc1C21c2ccccc2-c2ccc(N(c3ccc4c5ccccc5n(-c5cccc6ccccc56)c4c3)c3cccc4sc5ccccc5c34)c3cccc1c23]

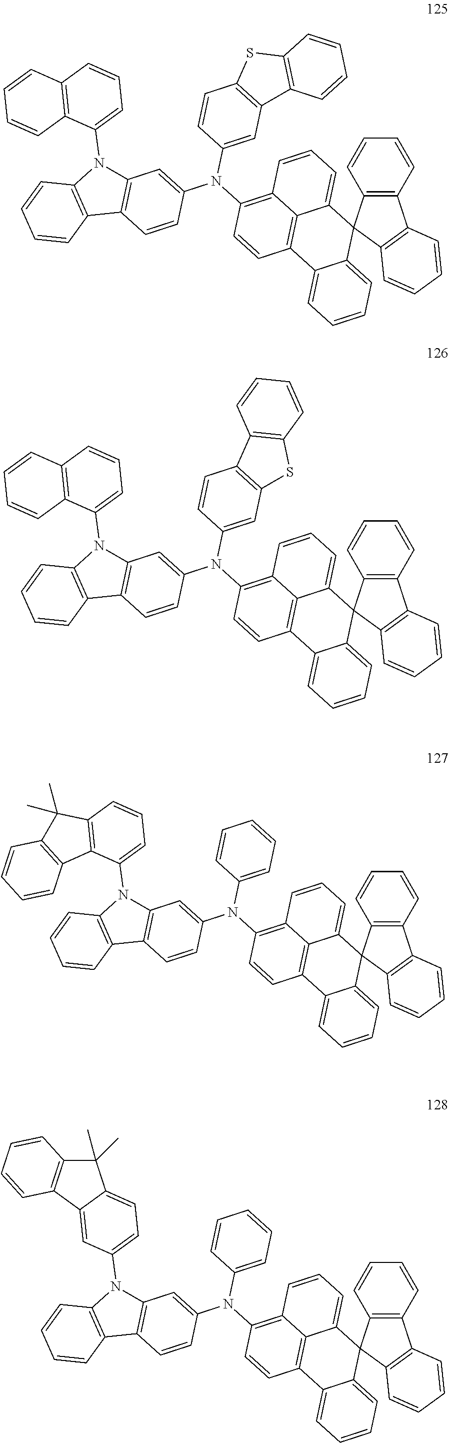 [SMILES: c1ccc2c(c1)-c1ccccc1C21c2ccccc2-c2ccc(N(c3ccc4sc5ccccc5c4c3)c3ccc4c5ccccc5n(-c5cccc6ccccc56)c4c3)c3cccc1c23 ; c1ccc2c(c1)-c1ccccc1C21c2ccccc2-c2ccc(N(c3ccc4c(c3)sc3ccccc34)c3ccc4c5ccccc5n(-c5cccc6ccccc56)c4c3)c3cccc1c23 ; CC1(C)c2ccccc2-c2c(-n3c4ccccc4c4ccc(N(c5ccccc5)c5ccc6c7c(cccc57)C5(c7ccccc7-c7ccccc75)c5ccccc5-6)cc43)cccc21 ; CC1(C)c2ccccc2-c2cc(-n3c4ccccc4c4ccc(N(c5ccccc5)c5ccc6c7c(cccc57)C5(c7ccccc7-c7ccccc75)c5ccccc5-6)cc43)ccc21]

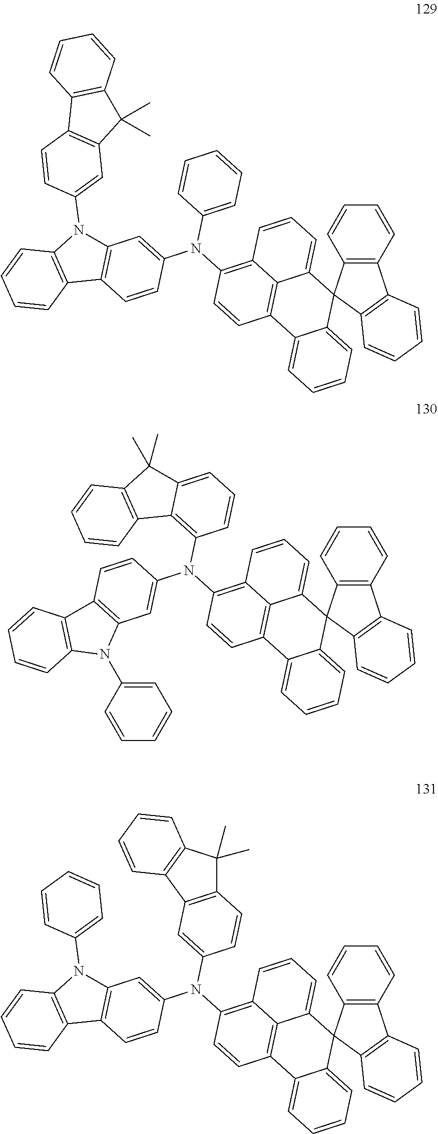 [SMILES: CC1(C)c2ccccc2-c2ccc(-n3c4ccccc4c4ccc(N(c5ccccc5)c5ccc6c7c(cccc57)C5(c7ccccc7-c7ccccc75)c5ccccc5-6)cc43)cc21 ; CC1(C)c2ccccc2-c2c(N(c3ccc4c5ccccc5n(-c5ccccc5)c4c3)c3ccc4c5c(cccc35)C3(c5ccccc5-c5ccccc53)c3ccccc3-4)cccc21 ; CC1(C)c2ccccc2-c2cc(N(c3ccc4c5ccccc5n(-c5ccccc5)c4c3)c3ccc4c5c(cccc35)C3(c5ccccc5-c5ccccc53)c3ccccc3-4)ccc21]

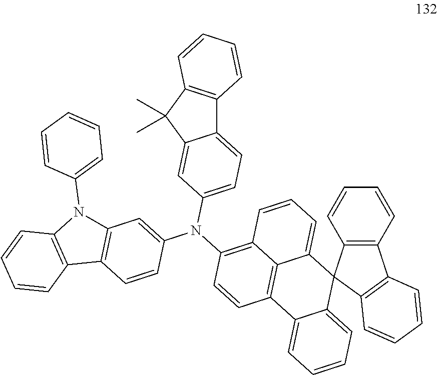 [SMILES: CC1(C)c2ccccc2-c2ccc(N(c3ccc4c5ccccc5n(-c5ccccc5)c4c3)c3ccc4c5c(cccc35)C3(c5ccccc5-c5ccccc53)c3ccccc3-4)cc21]

An amine compound according to an embodiment of the present disclosure represented by Formula 1 has a molecular structure in which an amine derivative is bonded to a spiro structure of a condensed ring and a carbazole group, and has a high glass transition temperature and a high melting point as a result of an introduction of the spiro structure of the condensed ring so that the amine compound may exhibit excellent characteristics of heat resistance and durability. In addition, hole transport characteristics may be further improved by having a low Highest Occupied Molecular Orbital (HOMO) energy level due to its structure in which an amine group is bonded to carbon number 2 of a carbazole group. When this amine compound according to an embodiment of the present disclosure is used in the hole transport region, hole transportability may be increased, and accordingly, the probability of recombination of holes and electrons in an emission layer may increase, thereby improving luminous efficiency.

An organic electroluminescence device ED according to an embodiment of the present disclosure is explained with reference back to FIGS. 3 to 6. As described above, the hole transport region HTR includes the aforementioned amine compound according to an embodiment of the present disclosure. For example, the hole transport region HTR includes an amine compound represented by Formula 1.

If the hole transport region HTR has a multilayer structure having a plurality of layers, any one of a plurality of layers may include the amine compound represented by Formula 1. For example, the hole transport region HTR may include a hole injection layer HIL on a first electrode EL1, and a hole transport layer HTL on the hole injection layer HIL, and the hole injection layer HIL or the hole transport layer HTL may include the amine compound represented by Formula 1. In addition, the hole transport region HTR includes a hole transport layer HTL on the first electrode EL1, and an electron blocking layer EBL on the hole transport layer HTL, and the electron blocking layer EBL may include the amine compound represented by Formula 1.

The hole transport region HTR may be formed by using various suitable methods such as a vacuum deposition method, a spin coating method, a cast method, a Langmuir-Blodgett (LB) method, an inkjet printing method, a laser printing method, and/or a laser induced thermal imaging (LITI) method.

The hole transport region HTR may further include a compound represented by Formula H-1 below.

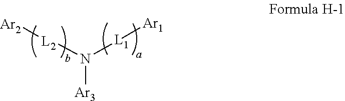

In Formula H-1 above, L₁ and L₂ may be each independently a direct linkage, a substituted or unsubstituted ring-forming arylene group having 6 to 30 carbon atoms, or a substituted or unsubstituted ring-forming heteroarylene group having 2 to 30 carbon atoms. “a” and “b” may be each independently an integer of 0 to 10. In one or more embodiments, if “a” or “b” is an integer of 2 or more, a plurality of L₁ and L₂ may be each independently a substituted or unsubstituted ring-forming arylene group having 6 to 30 carbon atoms, or a substituted or unsubstituted ring-forming heteroarylene group having 2 to 30 carbon atoms.

In Formula H-1, Ar₁ and Ar₂ may be each independently a substituted or unsubstituted ring-forming aryl group having 6 to 30 carbon atoms, or a substituted or unsubstituted ring-forming heteroaryl group having 2 to 30 carbon atoms. In addition, in Formula H-1, Ar₃ may be a substituted or unsubstituted ring-forming aryl group having 6 to 30 carbon atoms.

The compound represented by Formula H-1 above may be a monoamine compound. In one or more embodiments, the compound represented by Formula H-1 above may be a diamine compound in which at least one selected from among Ar₁ to Ar₃ includes an amine group as a substituent. In addition, the compound represented by Formula H-1 above may be a carbazole-based compound including a substituted or unsubstituted carbazole group in at least one of Ar₁ or Ar₂, or a fluorene-based compound including a substituted or unsubstituted fluorene group in at least one of Ar₁ or Ar₂.

The compound represented by Formula H-1 may be represented by any one selected from among the compounds in Compound Group H below. However, the compounds listed in Compound Group H below are illustrative examples, and the compound represented by Formula H-1 is not limited to those represented in Compound Group H below.

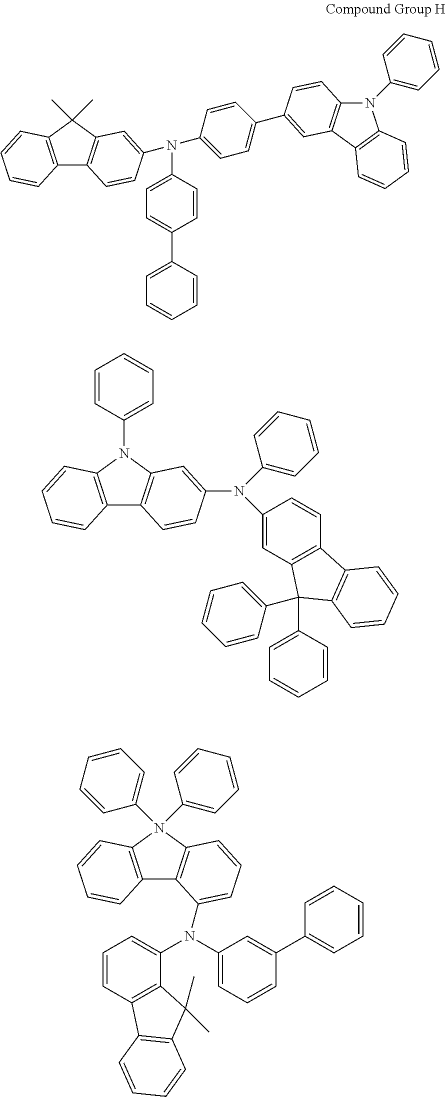

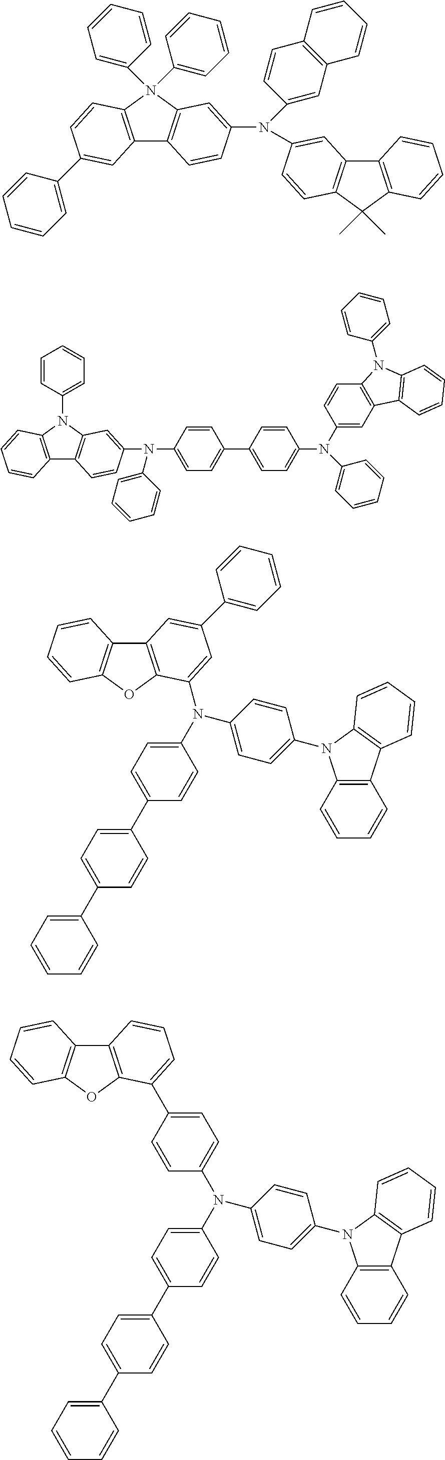

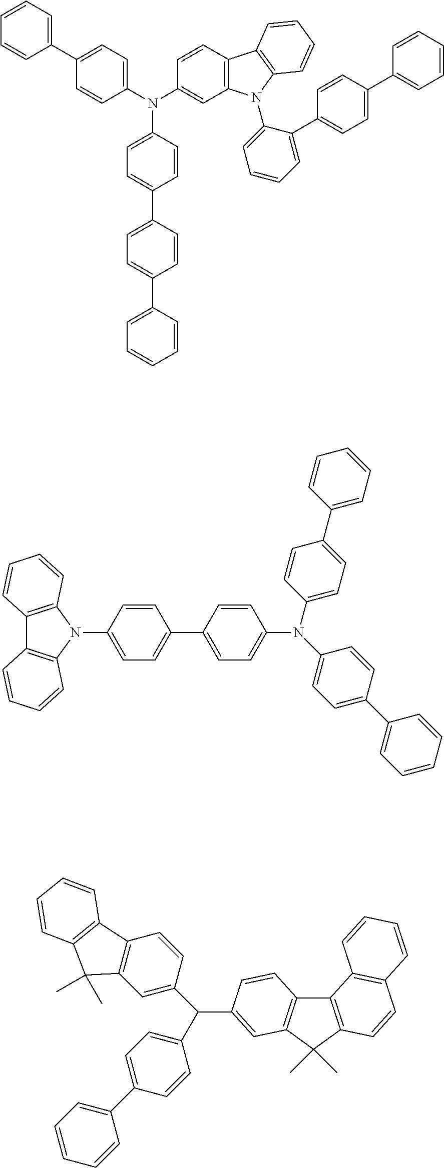

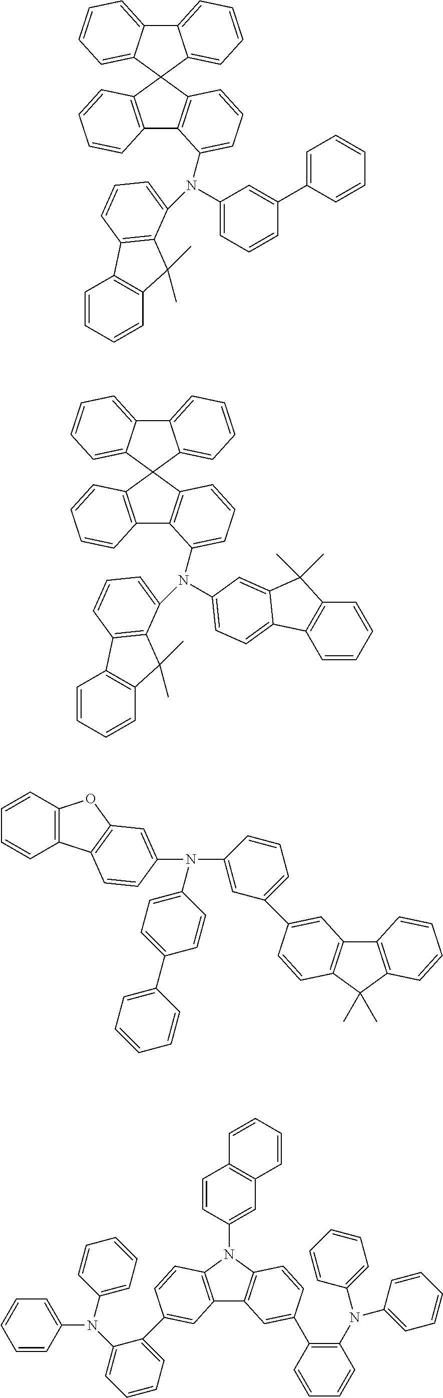 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21 ; c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1]

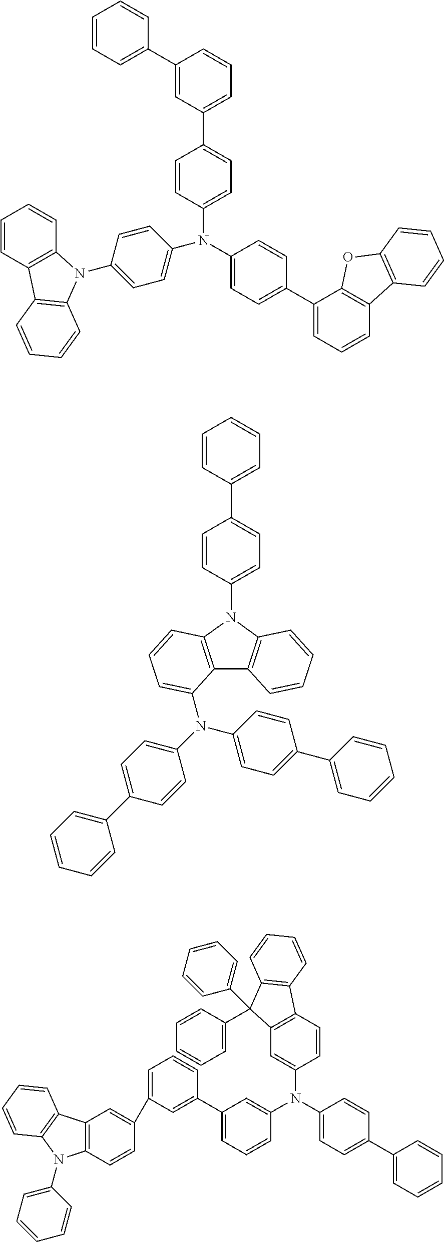 [SMILES: c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1]

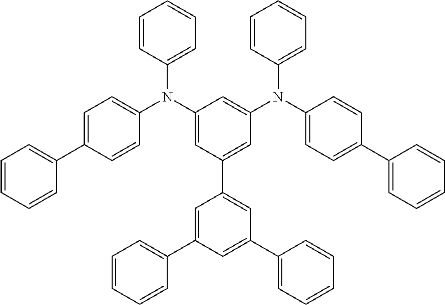 [SMILES: c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1]

The hole transport region HTR may further include a phthalocyanine compound such as copper phthalocyanine, N¹,N¹′-([1,1′-biphenyl]-4,4′-diyl)bis(N1-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′4″-Tris(N,N-diphenylamino)triphenylamine (TDATA), 4,4′,4″-tris[N(1-naphthyl)-N-phenylamino]-triphenylamine (1-TNATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonicacid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h] quinoxaline-2,3,6,7,10,11-hexacarbonitrile (HATCN), and/or the like.

The hole transport region HTR may further include a carbazole-based derivative such as N-phenyl carbazole and polyvinyl carbazole, a fluorene-based derivative, N,N′-bis(3-methylphenyl)-N,N′-diphenyl-[1,1-biphenyl]-4,4′-diamine (TPD), a triphenylamine-based derivative such as 4,4′,4″-tris(N-carbazolyl)triphenylamine (TCTA), N,N′-di(naphthalene-1-yl)-N,N′-diphenyl-benzidine (NPB), 4,4′-cyclohexylidene bis[N,N-bis(4-methylphenyl)benzeneamine] (TAPC), 4,4′-bis[N,N′-(3-tolyl)amino]-3,3′-dimethylbiphenyl (HMTPD), 1,3-bis(N-carbazolyl)benzene (mCP), and/or the like.

In addition, the hole transport region HTR may further include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl) benzene (mDCP), and/or the like.

The hole transport region HTR may include the aforementioned compounds of the hole transport region in at least one of the hole injection layer HIL, the hole transport layer HTL, and/or the electron blocking layer EBL.

A thickness of the hole transport region HTR may be about 100 Å to about 10000 Å, for example, about 100 Å to about 5000 Å. If the hole transport region HTR includes the hole injection layer HIL, a thickness of the hole injection layer HIL may be, for example, about 30 Å to about 1000 Å. If the hole transport region HTR includes the hole transport layer HTL, a thickness of the hole transport layer HTL may be about 30 Å to about 1000 Å. For example, if the hole transport region HTR includes the electron blocking layer EBL, a thickness of the electron blocking layer EBL may be about 10 Å to about 1000 Å. If the thicknesses of the hole transport regions HTR, the hole injection layer HIL, the hole transport layer HTL, and the electron blocking layer EBL satisfy the above-described ranges, suitable or satisfactory hole transport characteristics may be achieved without substantial increase of a driving voltage.

The hole transport region HTR may further include a charge generating material in addition to the above-described materials to improve conductivity (e.g., electrical conductivity). The charge generating material may be dispersed uniformly or non-uniformly in the hole transport region HTR. The charge generating material may be, for example, a p-dopant. The p-dopant may include at least one selected from among halogenated metal compounds, quinone derivatives, metal oxides, and cyano group-containing compounds, but the embodiment of the present disclosure is not limited thereto. For example, p-dopant may include a halogenated metal compound such as CuI and/or RbI, a quinone derivative such as tetracyanoquinodimethane (TCNQ) and/or 2,3,5,6-tetrafluoro-7,7,8,8-tetracyanoquinodimethane (F4-TCNQ), metal oxide such as tungsten oxide and/or molybdenum oxide, but the embodiment of the present disclosure is not limited thereto.

As described above, the hole transport region HTR may further include at least one of the buffer layer and/or the electron blocking layer EBL in addition to the hole injection layer HIL and the hole transport layer HTL. The buffer layer may compensate an optical resonance distance according to the wavelength of light emitted from the emission layer EML to increase light emission efficiency. Materials which may be included in the hole transport region HTR may be used as materials included in the buffer layer. The electron blocking layer EBL is a layer playing the role of preventing or reducing the injection of electrons from the electron transport region ETR to the hole transport region HTR.

The emission layer is provided on the hole transport region HTR. The emission layer EML may have a thickness of, for example about 100 Å to about 1000 Å, or about 100 Å to about 300 Å. The emission layer EML may have a single layer structure formed using a single material, a single layer structure formed using a plurality of different materials, or a multilayer structure having a plurality of layers formed using a plurality of different materials.

In the organic electroluminescence device ED according to an embodiment of the present disclosure, the emission layer EML may include an anthracene derivative, a pyrene derivative, a fluoranthene derivative, a chrysene derivative, a dihydrobenz anthracene derivative, and/or a triphenylene derivative. For example, the emission layer EML may include an anthracene derivative or a pyrene derivative.

In the organic electroluminescence devices ED according to an embodiment of the present disclosure shown in FIGS. 3 to 6, the emission layer EML may include a host and a dopant, and the emission layer EML may include the compound represented by Formula E-1 below. The compound represented by Formula E-1 below may be used as a fluorescent host material.

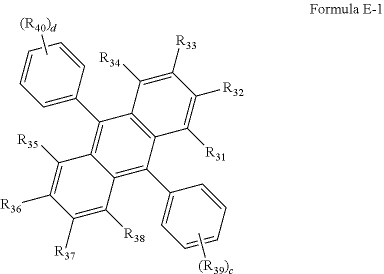

In Formula E-1, R₃₁ to R₄₀ may be each independently a hydrogen atom, a deuterium atom, a halogen atom, a substituted or unsubstituted silyl group, a substituted or unsubstituted alkyl group having 1 to 10 carbon atoms, a substituted or unsubstituted ring-forming aryl group having 6 to 30 carbon atoms, a substituted or unsubstituted ring-forming heteroaryl group having 2 to 30 carbon atoms, and/or combined with an adjacent group to form a ring. In one or more embodiments, R₃₁ to R₄₀ may be combined with an adjacent group to form a saturated hydrocarbon ring or an unsaturated hydrocarbon ring.

In Formula E-1, c and d may be each independently an integer of 0 to 5.

Formula E-1 may be represented by any one selected from among Compound E1 to Compound E19 below.

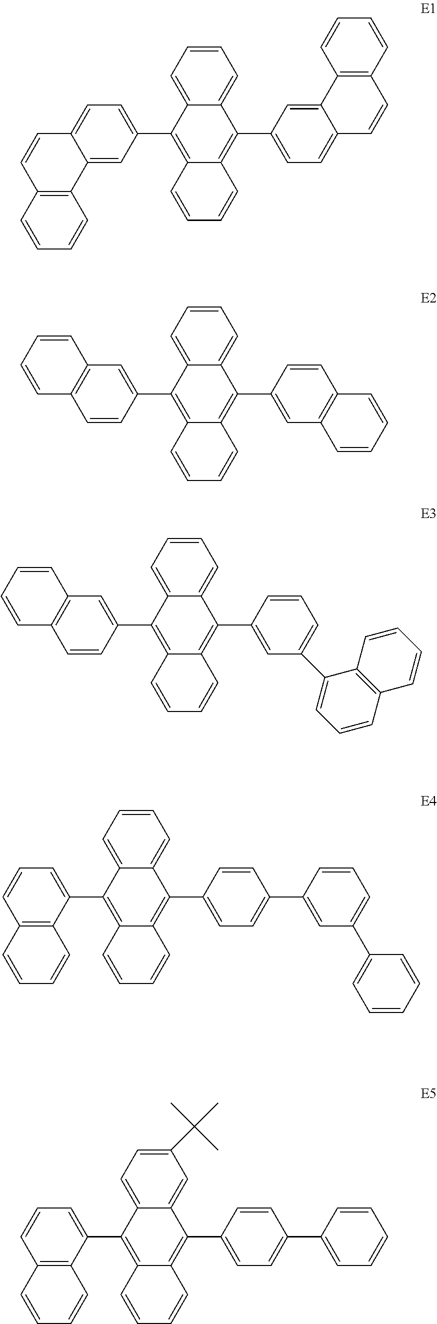

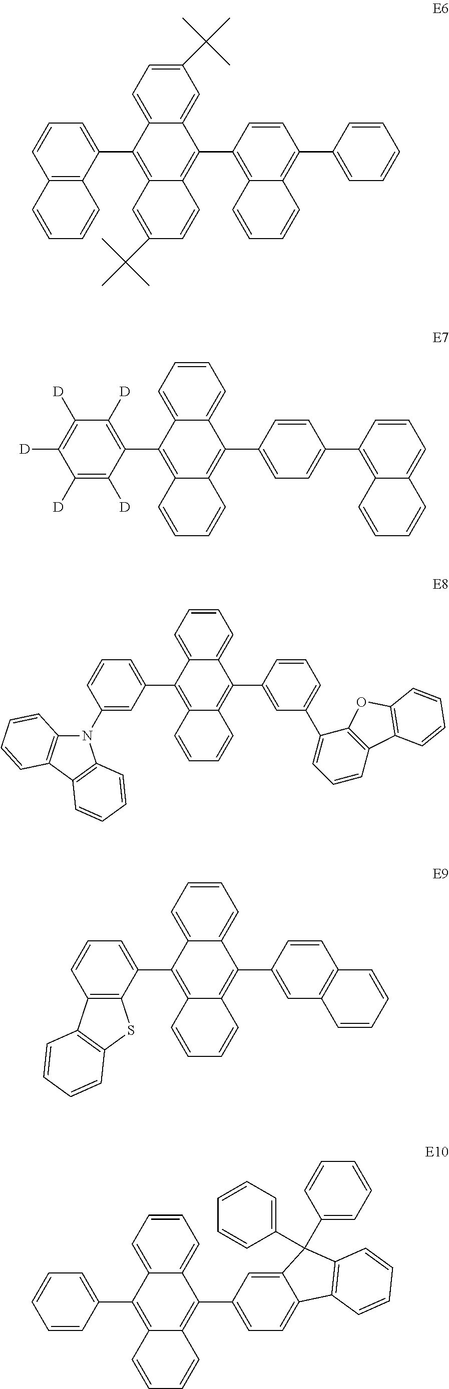

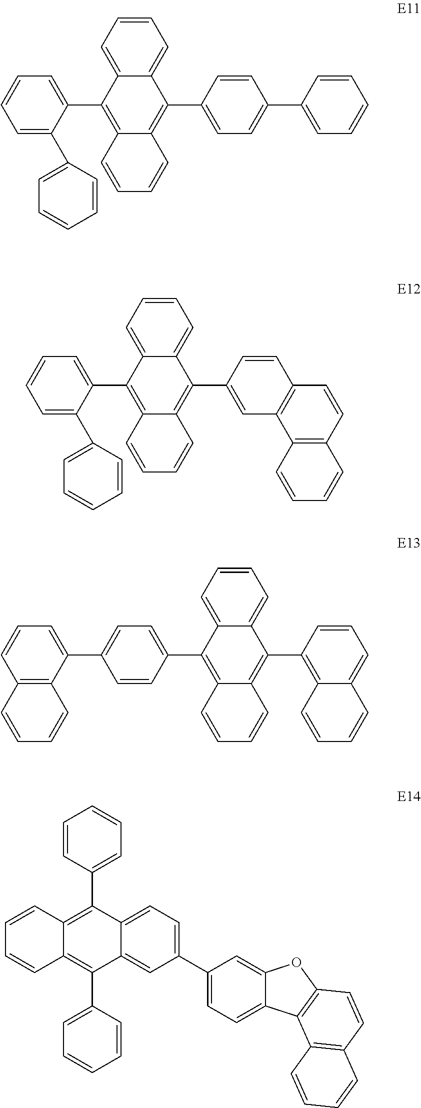

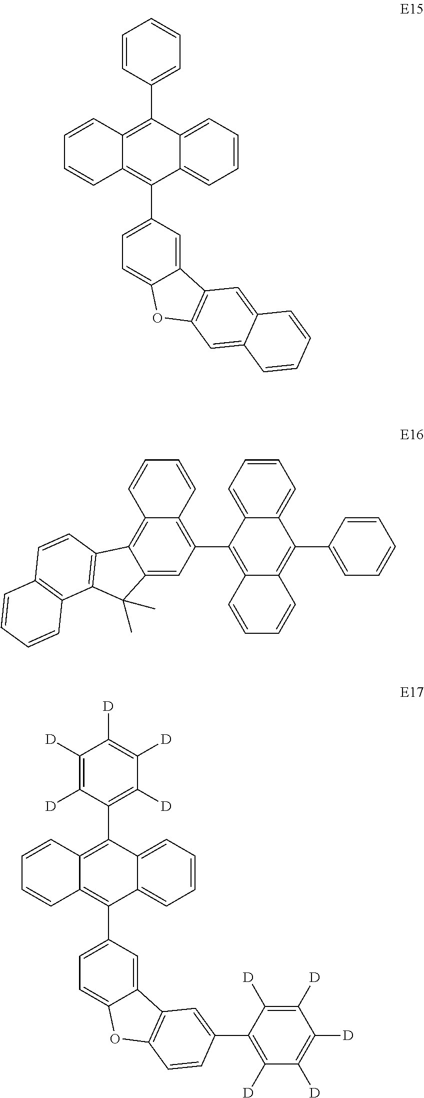

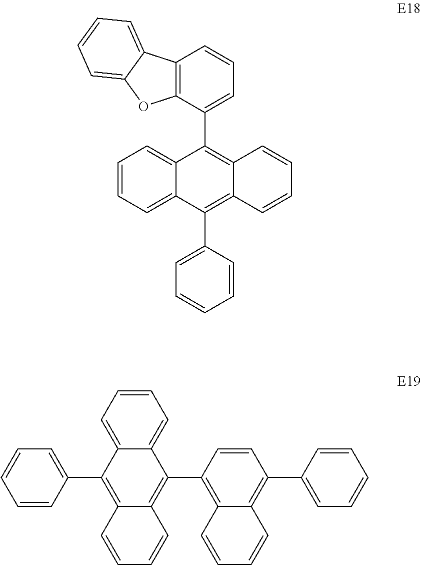

In an embodiment of the present disclosure, the emission layer EML may include the compound represented by Formula E-2a or Formula E-2b below. The compound represented by Formula E-2a or Formula E-2b below may be used as a phosphorescent host material.

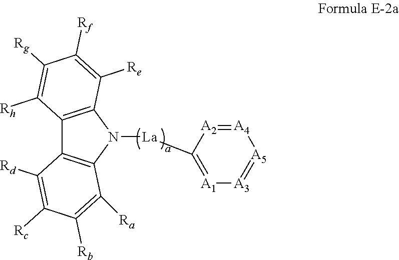

In Formula E-2a, a may be an integer of 0 to 10, and La may be a direct linkage, a substituted or unsubstituted ring-forming arylene group having 6 to 30 carbon atoms, or a substituted or unsubstituted ring-forming heteroarylene group having 2 to 30 carbon atoms. In one or more embodiments, if a is an integer of 2 or more, a plurality of Lₐ may be each independently a substituted or unsubstituted ring-forming arylene group having 6 to 30 carbon atoms, or a substituted or unsubstituted ring-forming heteroarylene group having 2 to 30 carbon atoms.

In addition, in Formula E-2a, A₁ to A₅ may be each independently N or CRᵢ. Rₐ to Rᵢ may be each independently a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted ring-forming aryl group having 6 to 30 carbon atoms, a substituted or unsubstituted ring-forming heteroaryl group having 2 to 30 carbon atoms, and/or combined with an adjacent group to form a ring. Rₐ to Rᵢ may combined with an adjacent group to from hydrocarbon ring or hetero ring including N, O, S, etc. as a ring-forming atom.

In one or more embodiments, in Formula E-2a, two or three selected from among A₁ to A₅ may be N, and the remainder may be CRᵢ.

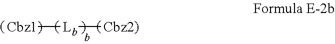

In Formula E-2b, Cbz1 and Cbz2 may be each independently an unsubstituted carbazole group, or a carbazole group substituted with a ring-forming aryl group having 6 to 30 carbon atoms. L_{b} may be a direct linkage, a substituted or unsubstituted ring-forming arylene group having 6 to 30 carbon atoms, or a substituted or unsubstituted ring-forming heteroarylene group having 2 to 30 carbon atoms. b may be an integer of 0 to 10, and if b is an integer of 2 or more, a plurality of L_{b} may be each independently a substituted or unsubstituted ring-forming arylene group having 6 to 30 carbon atoms, or a substituted or unsubstituted ring-forming heteroarylene group having 2 to 30 carbon atoms.

The compound represented by Formula E-2a or Formula E-2b may be represented by any one selected from among the compounds in Compound Group E-2 below. However, the compounds listed in Compound Group E-2 below are illustrative, and the compound represented by Formula E-2a or Formula E-2b is not limited to those represented in Compound Group E-2 below.

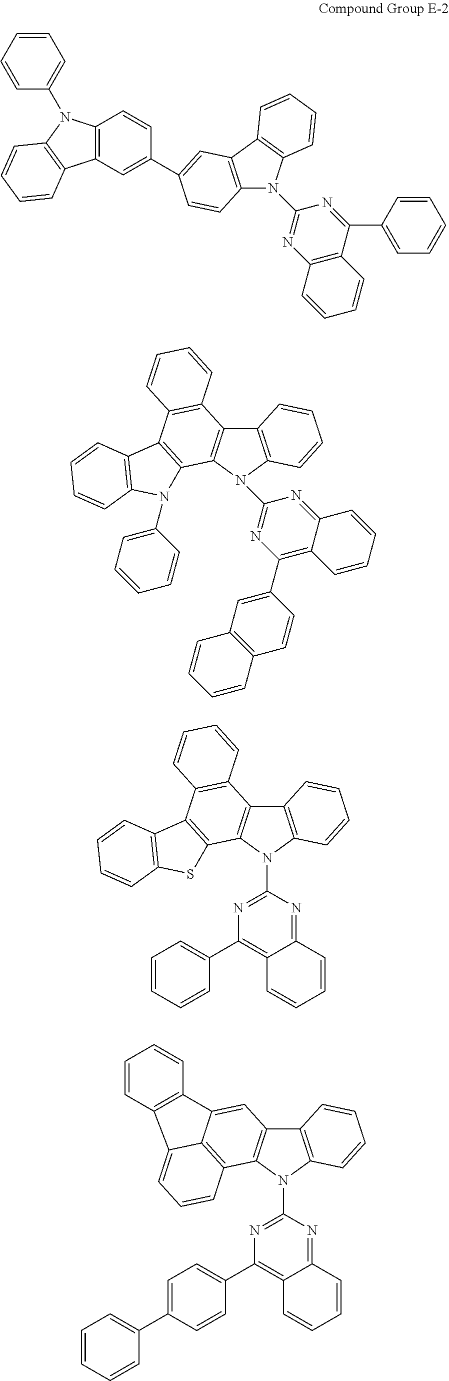

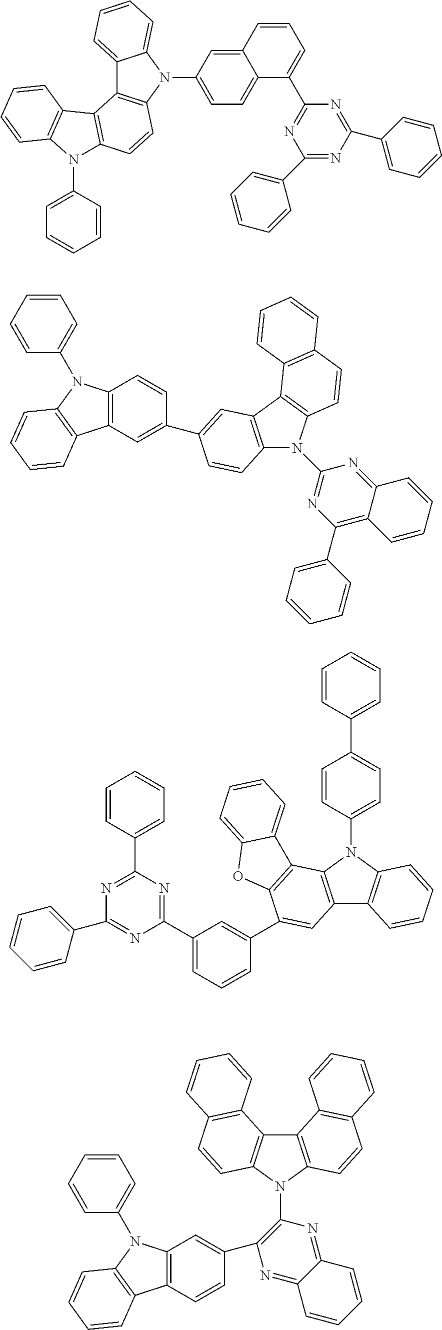

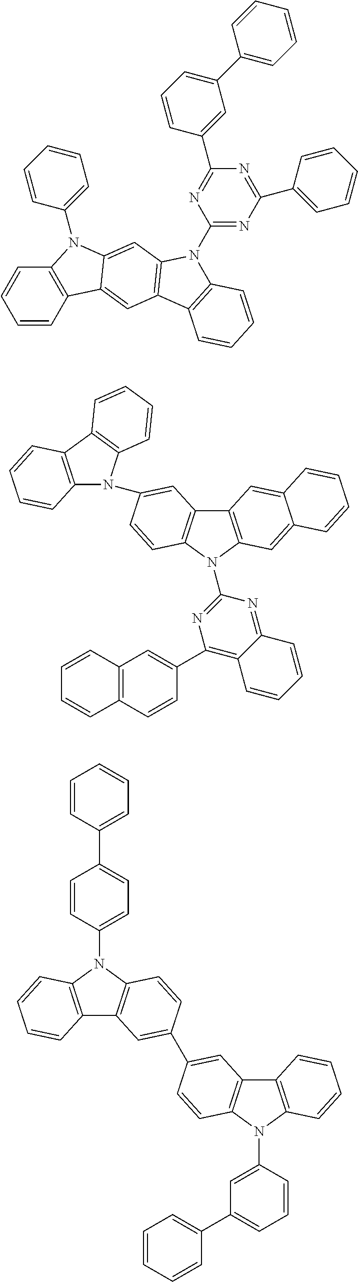 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1]

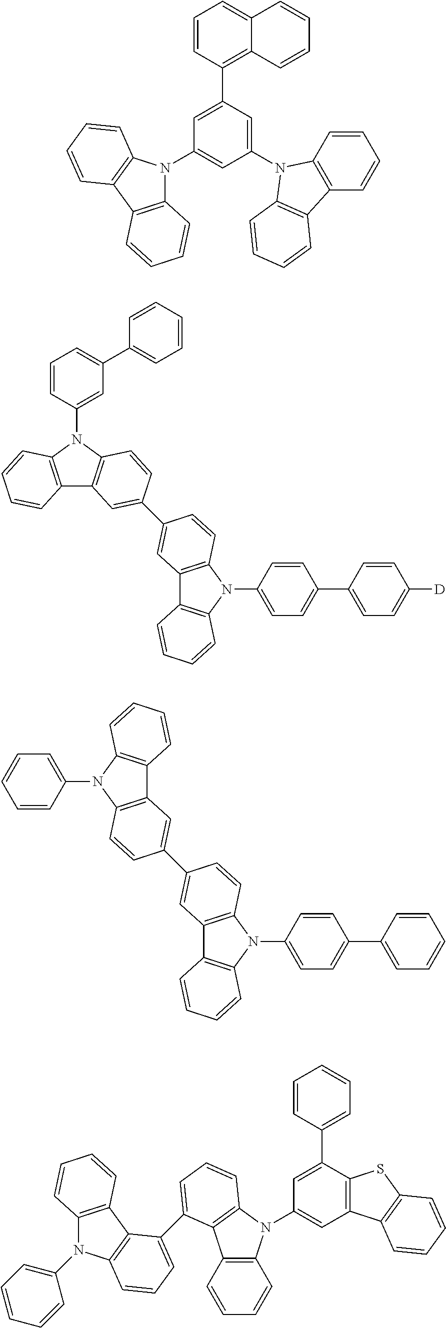

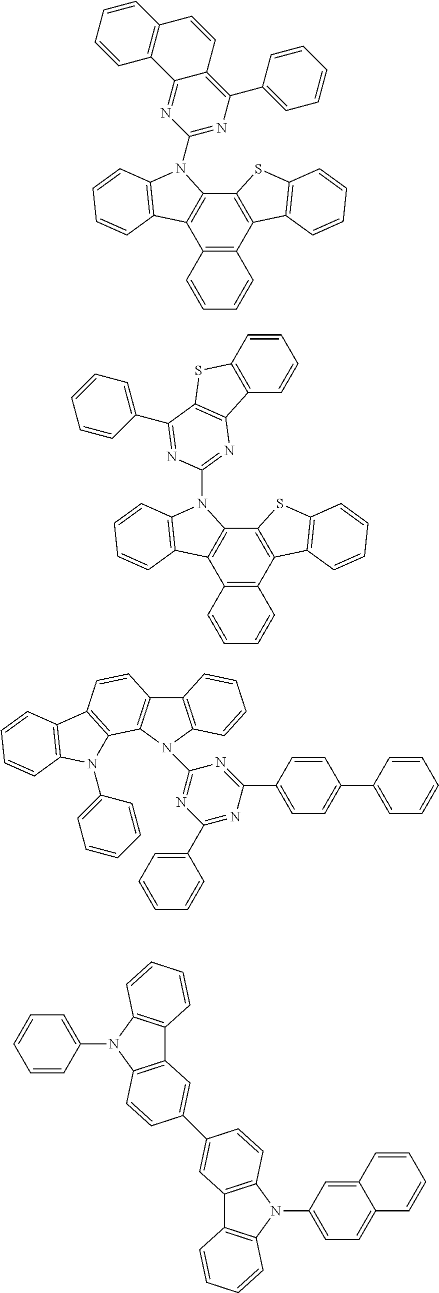

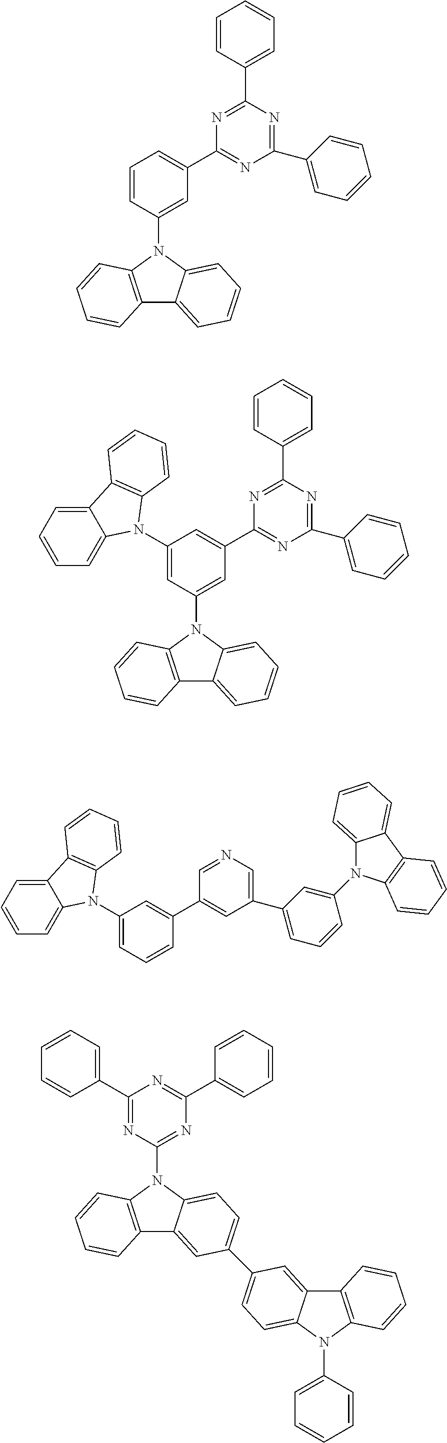 [SMILES: c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may include any suitable material available in the art as a host material. For example, the emission layer EML may include at least one selected from among bis[2-(diphenylphosphino)phenyl] ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), and 1,3,5-tris(1-phenyl-1H-benzo[d]imidazole-2-yl)benzene (TPBi) as the host material. However, the embodiment of the present disclosure is not limited thereto, and for example, tris(8-hydroxyquinolino)aluminum (Alq₃), 4,4′-bis(N-carbazolyl)-1,1′-biphenyl (CBP), poly(N-vinylcarbazole) (PVK), 9,10-di(naphthalene-2-yl)anthracene (ADN), 4,4′,4″-tris(carbazol-9-yl)-triphenylamine (TCTA), 1,3,5-tris(N-phenylbenzimidazole-2-yl)benzene (TPBi), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), 2,8-bis(diphenylphosphoryl)dibenzofuran (PPF), and/or the like may be used as the host material.

The emission layer EML may include the compound represented by Formula M-a or Formula M-b below. The compound represented by Formula M-a or Formula M-b below may be used as a phosphorescent dopant material.

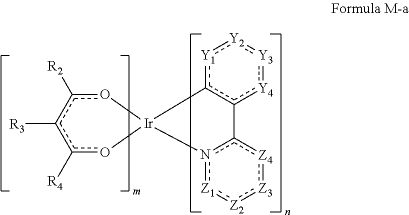

In Formula M-a above, Y₁ to Y₄, and Z₁ to Z₄ may be each independently CR₁ or N, and R₁ to R₄ may be each independently a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted ring-forming aryl group having 6 to 30 carbon atoms, a substituted or unsubstituted ring-forming heteroaryl group having 2 to 30 carbon atoms, and/or combined with an adjacent group to form a ring. In Formula M-a, m is 0 or 1, and n is 2 or 3. In Formula M-a, when m is 0, n is 3, and when m is 1, n is 2.

The compound represented by Formula M-a may be used as a red phosphorescent dopant or a green phosphorescent dopant.

The compound represented by Formula M-a may be represented by any one selected from among Compounds M-a1 to M-a19 below. However, Compounds M-a1 to M-a19 below are illustrative examples, and the compound represented by Formula M-a is not limited to those represented by Compounds M-a1 to M-a19 below.

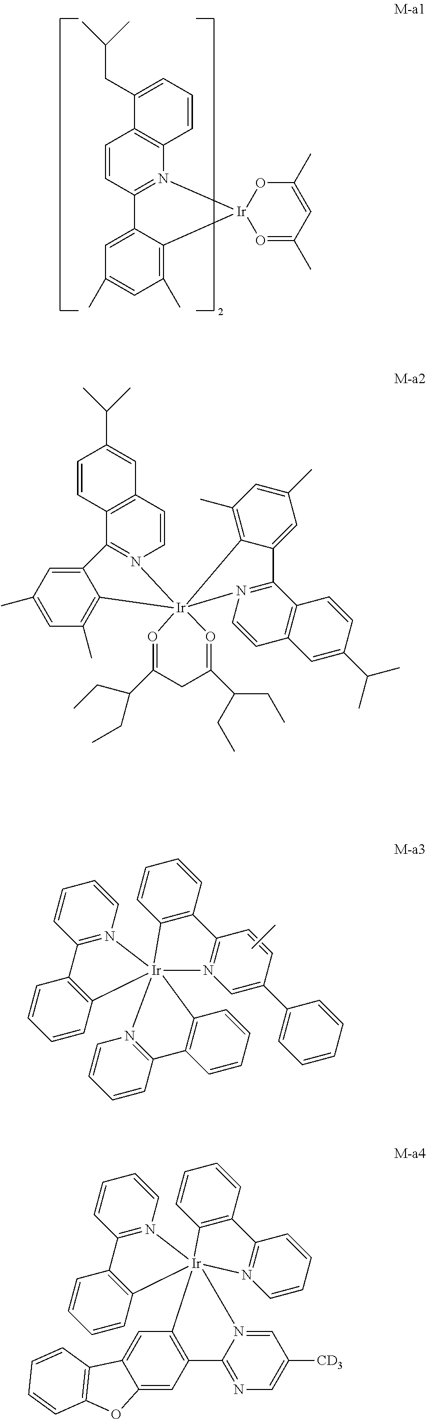

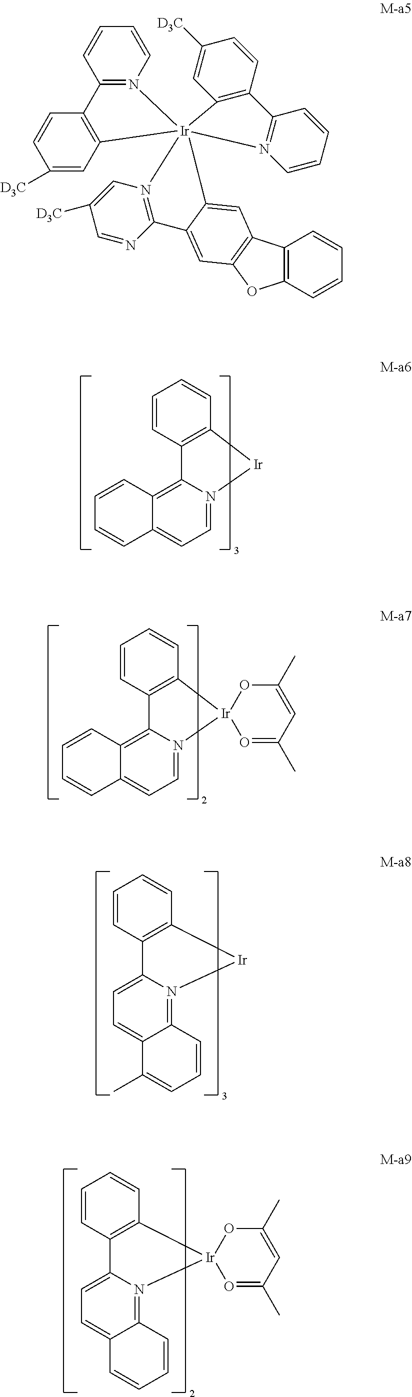

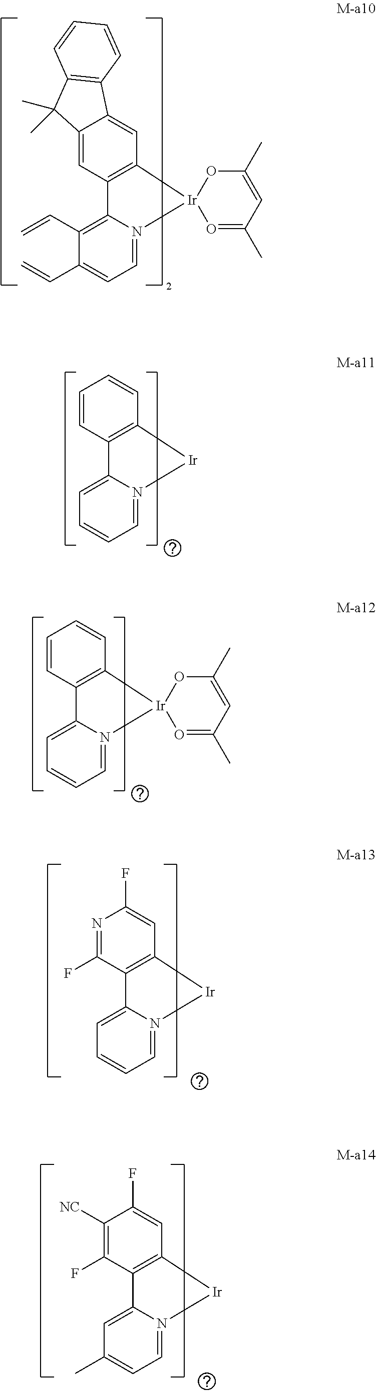

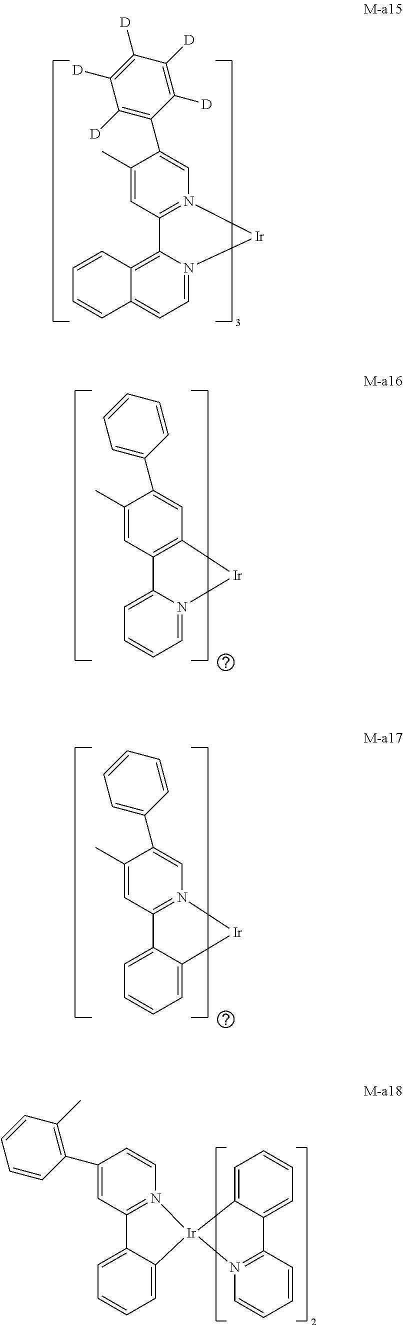

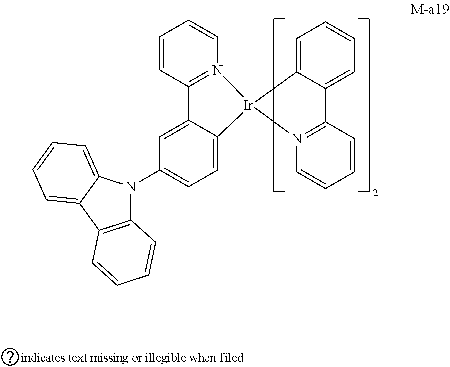

Compound M-a1 and Compound M-a2 may be used as a red dopant material, and Compounds M-a3 to M-a5 may be used as a green dopant material.

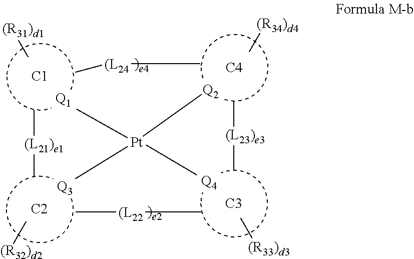

In Formula M-b, Q₁ to Q₄ are each independently C or N, and C₁ to C₄ are each independently a substituted or unsubstituted ring-forming hydrocarbon ring having 5 to 30 carbon atoms, or a substituted or unsubstituted ring-forming heterocycle having 2 to 30 carbon atoms. L₂₁ to L₂₄ are each independently a direct linkage,

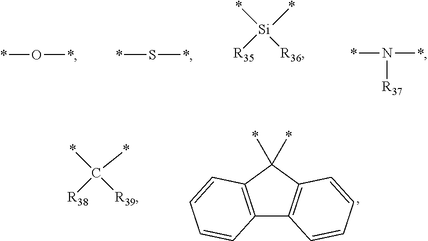

a substituted or unsubstituted divalent alkyl group having 1 to 20 carbon atoms, a substituted or unsubstituted ring-forming arylene group having 6 to 30 carbon atoms, or a substituted or unsubstituted ring-forming heteroarylene group having 2 to 30 carbon atoms, and e1 to e4 are each independently 0 or 1. R₃₁ to R₃₉ are each independently a hydrogen atom, a deuterium atom, a cyano group, a substituted or unsubstituted amine group, a substituted or unsubstituted alkyl group having 1 to 20 carbon atoms, a substituted or unsubstituted ring-forming aryl group having 6 to 30 carbon atoms, a substituted or unsubstituted ring-forming heteroaryl group having 2 to 30 carbon atoms, and/or combined with an adjacent group to form a ring, and d1 to d4 are each independently an integer of 0 to 4.

The compound represented by Formula M-b may be used as a blue phosphorescent dopant or a green phosphorescent dopant.

The compound represented by Formula M-b may be represented by any one selected from among the compounds below. However, the compounds below are illustrative examples, and the compound represented by Formula M-b is not limited to those represented in the compounds below.

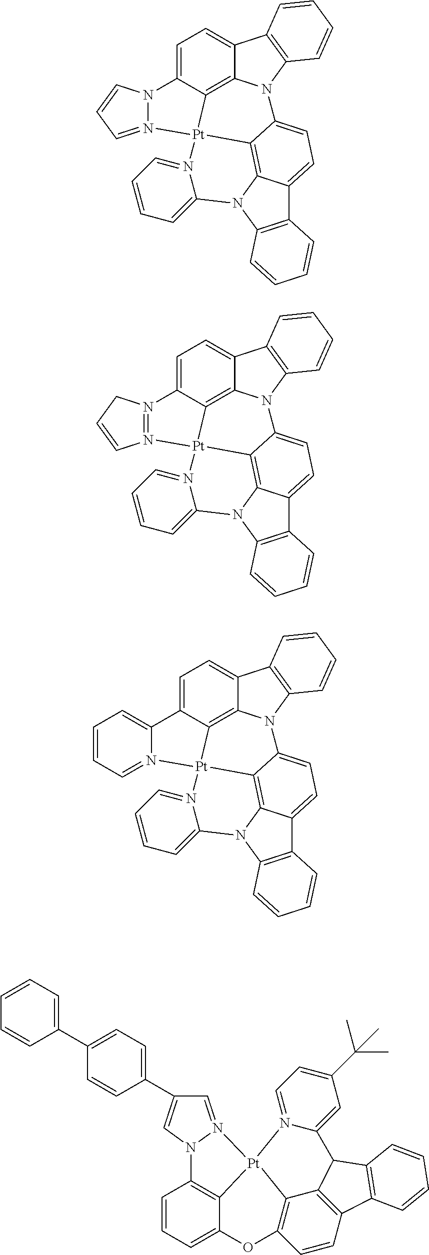

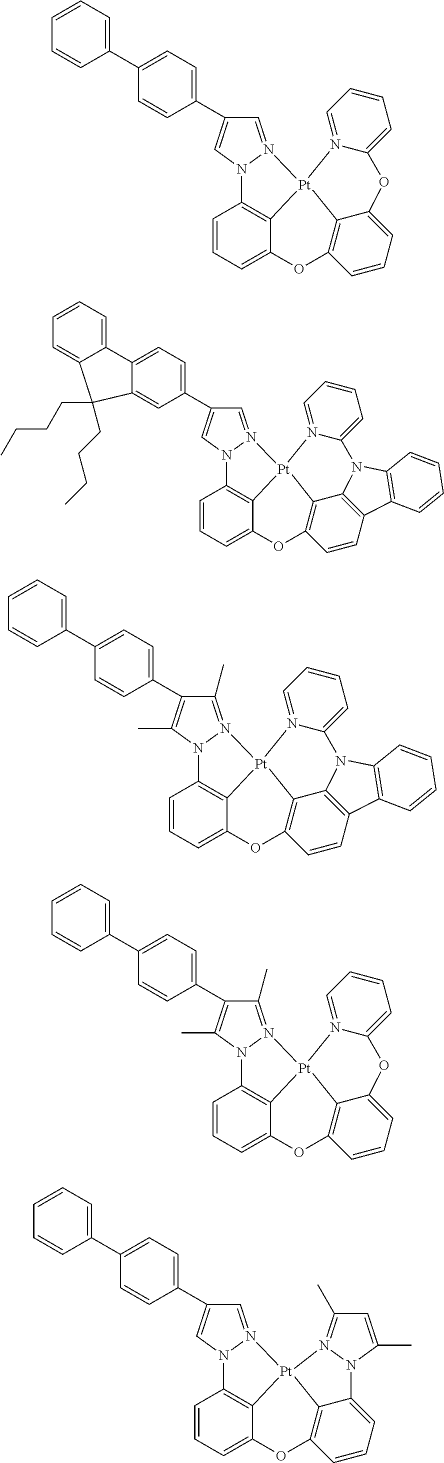

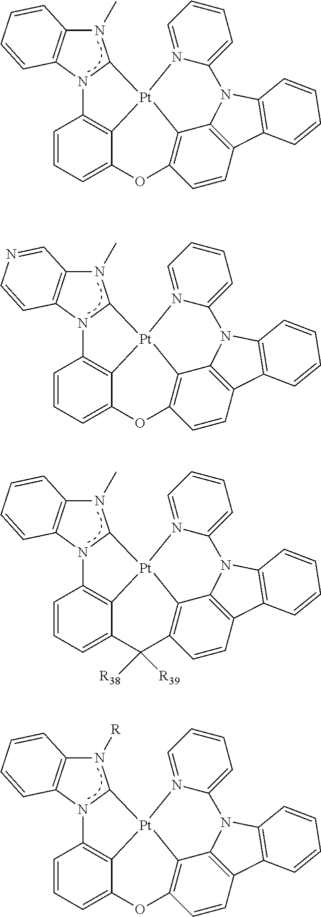

In the above compounds, R, R₃₈, and R₃₉ may be each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms.

The emission layer EML may include the compound represented by any one selected from among Formula F-a to Formula F-c below. The compound represented by Formula F-a to Formula F-c below may be used as a fluorescent dopant material.

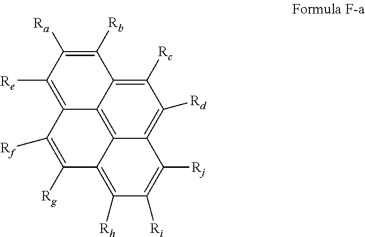

In Formula F-a above, two selected from among Rₐ to Rⱼ may be each independently substituted with

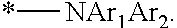

The remainder selected from among Rₐ to Rⱼ that are not substituted with

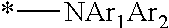

may be each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms. In

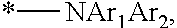

Ar₁ and Ar₂ may be each independently a substituted or unsubstituted ring-forming aryl group having 6 to 30 carbon atoms, or a substituted or unsubstituted ring-forming heteroaryl group having 2 to 30 carbon atoms. For example, at least one of Ar₁ or Ar₂ may be a heteroaryl group including O or S as a ring-forming atom.

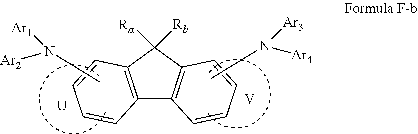

In Formula F-b above, Rₐ and R_{b} may be each independently a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted ring-forming aryl group having 6 to 30 carbon atoms, a substituted or unsubstituted ring-forming heteroaryl group having 2 to 30 carbon atoms, and/or combined with an adjacent group to form a ring.

In Formula F-b, U and V may be each independently a substituted or unsubstituted ring-forming hydrocarbon ring having 5 to 30 carbon atoms, or a substituted or unsubstituted ring-forming heterocycle having 2 to 30 carbon atoms.

In Formula F-b, the number of ring represented by U and V may be each independently 0 or 1. For example, in Formula F-b, if the number of U or V is 1, one ring constitutes a condensed ring in a portion indicated as U or V, and if the number of U or V is 0, it means that a ring indicated as U or V does not exist. In one or more embodiments, if the number of U is 0 and the number of V is 1, or the number of U is 1 and the number of V is 0, the condensed ring having a fluorene core of Formula F-b may be a tetracyclic compound. In addition, if the number of U and V are all 0, the condensed ring of Formula F-b may be a tricyclic compound. In addition, if the number of U and V are all 1, the condensed ring having a fluorene core of Formula F-b may be a pentacyclic compound.

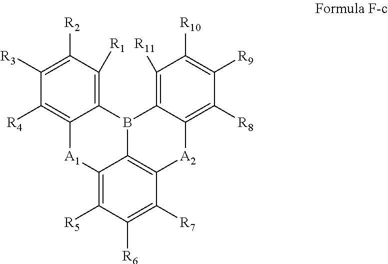

In Formula F-c, A₁ and A₂ may be each independently O, S, Se, or NRₘ, and Rₘ may be a hydrogen atom, a deuterium atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms. R₁ to R₁₁ are each independently a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted ring-forming aryl group having 6 to 30 carbon atoms, a substituted or unsubstituted ring-forming heteroaryl group having 2 to 30 carbon atoms, and/or combined with an adjacent group to form a ring.

In Formula F-c, A₁ and A₂ may be each independently combined with substituents of an adjacent ring to form a condensed ring. For example, when A₁ and A₂ are each independently NRₘ, A₁ may be combined with R₄ or R₅ to form a ring. In addition, A₂ may be combined with R₇ or R₈ to form a ring.

In an embodiment of the present disclosure, the emission layer EML may further include, as any suitable dopant material available in the art, for example, a styryl derivative (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi)), 4,4′-bis[2-(4-(N,N-diphenylamino)phenyl)vinyl]biphenyl (DPAVBi), perylene and/or a derivative thereof (for example, 2,5,8,11-tetra-t-butylperylene (TBP)), pyrene and/or a derivative thereof (for example, 1,1-dipyrene, 1,4-dipyrenylbenzene, 1,4-bis(N,N-diphenylamino)pyrene), and/or the like.

The emission layer EML may further include any suitable phosphorescent dopant material available in the art. For example, a metal complex including iridium (Ir), platinum (Pt), osmium (Os), gold (Au), titanium (Ti), zirconium (Zr), hafnium (Hf), europium (Eu), terbium (Tb), and/or thulium (Tm) may be used as a phosphorescent dopant. In one or more embodiments, iridium (III) bis(4,6-difluorophenylpyridinato-N,C2′) (FIrpic), bis(2,4-difluorophenylpyridinato)-tetrakis(1-pyrazolyl)borate iridium (III) (Fir6), and/or platinum octaethyl porphyrin (PtOEP) may be used as a phosphorescent dopant. However, the embodiment of the present disclosure is not limited thereto.

The emission layer EML may include a quantum dot material. The core of the quantum dot may be selected from Group II-VI compounds, Group III-V compounds, Group IV-VI compounds, Group IV elements, Group IV compounds, and a combination thereof.

Group II-VI compounds may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and a mixture thereof.

Group III-VI compounds may include a binary compound such as In₂S₃ and/or In₂Se₃, a ternary compound such as InGaS₃ and/or InGaSe₃, or any combination thereof.

Group I-III-VI compounds may be selected from a ternary compound selected from the group consisting of AgInS, AgInS₂, CuInS, CuInS₂, AgGaS₂, CuGaS₂, CuGaO₂, AgGaO₂, AgAlO₂, and a mixture thereof, and/or a quaternary compound such as AgInGaS₂ and/or CuInGaS₂.

Group III-V compounds may be selected from the group consisting of a binary compound selected from the group consisting of GaN, GaP, GaAs, GaSb, AlN, AlP, AlAs, AlSb, InN, InP, InAs, InSb, and a mixture thereof; a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof; and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and a mixture thereof. In one or more embodiments, the Group III-V compounds may further include a Group II metal. For example, InZnP, and/or the like may be selected as Group V compounds.

Group IV-VI compounds may be selected from the group consisting of a binary compound selected from the group consisting of SnS, SnSe, SnTe, PbS, PbSe, PbTe, and a mixture thereof; a ternary compound selected from the group consisting of SnSeS, SnSeTe, SnSTe, PbSeS, PbSeTe, PbSTe, SnPbS, SnPbSe, SnPbTe, and a mixture thereof; and a quaternary compound selected from the group consisting of SnPbSSe, SnPbSeTe, SnPbSTe, and a mixture thereof. Group IV elements may be selected from the group consisting of Si, Ge, and a mixture thereof. Group IV compounds may be a binary compound selected from the group consisting of SiC, SiGe, and a mixture thereof.

In this case, the binary compound, the ternary compound, or the quaternary compound may be present in the particle at a uniform (e.g., substantially uniform) concentration, or may be present in the same particle while being divided to have partially different concentration distributions. In addition, they may have a core/shell structure in which one quantum dot surrounds another quantum dot. An interface between the core and the shell may have a concentration gradient such that a concentration of an element present in the shell gradually decreases along a direction toward the core.

In some examples, the quantum dot may have a core-shell structure including a core including the aforementioned nanocrystal and a shell surrounding the core. The shell of the quantum dot may serve as a protective layer for maintaining characteristics of a semiconductor by preventing or reducing chemical modification of the core and/or a charging layer for imparting electrophoretic characteristics to the quantum dot. The shell may be a single layer or multiples layers. An interface between the core and the shell may have a concentration gradient such that a concentration of an element present in the shell gradually decreases along a direction toward the center of the interface. Examples of the shell of the quantum dot may include metal and/or non-metal oxide, a semiconductor compound, or a combination thereof.

For example, the metal and/or non-metal oxide may be illustrated as a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO, and/or a ternary compound such as MgAl₂O₄, CoFe₂O₄, NiFe₂O₄, and/or CoMn₂O₄, but the embodiment of the present disclosure is not limited thereto.

In addition, the semiconductor compound may be illustrated as, for example, CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, and/or the like, but the embodiment of the present disclosure is not limited thereto.

The quantum dot may have a full width of half maximum (FWHM) of an emission wavelength spectrum of about 45 nm or less, about 40 nm or less, or, for example, about 30 nm or less, and color purity or color gamut may be improved in this range. In addition, as light emitted through this quantum dot is emitted in all directions, a wide viewing angle may be improved.

In addition, the shape of the quantum dot is a shape generally used in the art, and is not particularly limited. For example, spherical, pyramidal, multi-arm, and/or cubic nanoparticles, nanotubes, nanowires, nanofibers, plate-shaped nanoparticles, and/or the like may be used.

The quantum dot may control the color of emitted light according to the particle size, and thus, the quantum dot may have various suitable light-emitting colors such as blue, red, green, and/or the like.

In the organic electroluminescence devices ED according to an embodiment of the present disclosure shown in FIGS. 3 to 6, the electron transport region ETR is provided on the emission layer EML. The electron transport region ETR may include at least one of the hole blocking layer HBL, the electron transport layer ETL, and/or the electron injection layer EIL, but the embodiment of the present disclosure is not limited thereto.

The electron transport region ETR may have a single layer structure formed using a single material, a single layer structure formed using a plurality of different materials, or a multilayer structure having a plurality of layers formed using a plurality of different materials.

For example, the electron transport region ETR may have the structure of a single layer of the electron injection layer EIL or the electron transport layer ETL, and may have a structure of a single layer formed using an electron injection material and an electron transport material. Further, the electron transport regions ETR may have a single layer structure formed using a plurality of different materials, or a structure stacked from the emission layer EML of electron transport layer ETL/electron injection layer EIL, or hole blocking layer HBL/electron transport layer ETL/electron injection layer EIL, but the embodiment of the present disclosure is not limited thereto. A thickness of the electron transport region ETR may be, for example, about 1000 Å to about 1500 Å.

The electron transport region ETR may be formed by using various suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-1 below.

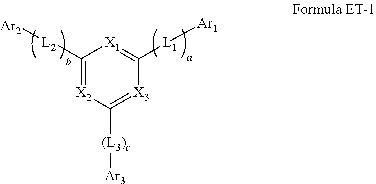

In Formula ET-1, at least one selected from among X₁ to X₃ is N, and the remainder are CRₐ. Rₐ may be each independently a hydrogen atom, a deuterium atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms. Ar₁ to Ar₃ may be each independently a hydrogen atom, a deuterium atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms.

In Formula ET-1, a to c may be each independently an integer of 0 to 10. In Formula ET-1, L₁ to L₃ may be each independently a direct linkage, a substituted or unsubstituted ring-forming arylene group having 6 to 30 carbon atoms, or a substituted or unsubstituted ring-forming heteroarylene group having 2 to 30 carbon atoms. In one or more embodiments, if a to c are an integer of 2 or more, L₁ to L₃ may be each independently a substituted or unsubstituted ring-forming arylene group having 6 to 30 carbon atoms, or a substituted or unsubstituted ring-forming heteroarylene group having 2 to 30 carbon atoms.

The electron transport region ETR may further include an anthracene-based compound. However, the embodiment of the present disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-Tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), beryllium bis(benzoquinolin-10-olate) (Bebq₂), 9,10-di(naphthalene-2-yl)anthracene (ADN), 1,3-bis[3,5-di(pyridin-3-yl)phenyl]benzene (BmPyPhB), or a mixture thereof.

In addition, the electron transport region ETR may include a halogenated metal such as LiF, NaCl, CsF, RbCl, RbI, CuI, KI, a lanthanide metal such as Yb, and/or a co-deposited material of the above-described halogenated metal and lanthanide metals as well. For example, the electron transport region ETR may include KI:Yb, RbI:Yb, and/or the like as a co-deposited material. In one or more embodiments, the electron transport region ETR may include metal oxide such as Li₂O, BaO, and/or Liq(8-hydroxyl-lithium quinolate), but the embodiment of the present disclosure is not limited thereto. Also, the electron transport region ETR may be formed using a mixture material of an electron transport material and an insulating organo metal salt. The organo metal salt may be a material having an energy band gap of about 4 eV or more. In one or more embodiments, the organo metal salt may include, for example, metal acetates, metal benzoates, metal acetoacetates, metal acetylacetonates, and/or metal stearates, but the embodiment of the present disclosure is not limited thereto.

The electron transport region ETR may include the aforementioned compounds of the electron transport region in at least one of the electron injection layer EIL, the electron transport layer ETL, and/or the hole blocking layer HBL.

If the electron transport region ETR includes the electron transport layer ETL, a thickness of the electron transport layer ETL may be about 100 Å to about 1000 Å, for example, about 150 Å to about 500 Å. If the thickness of the electron transport layer ETL satisfies the above-described range, suitable or satisfactory electron transport characteristics may be achieved without substantial increase of a driving voltage. If the electron transport region ETR includes the electron transport layer ETL, a thickness of the electron injection layer EIL may be about 1 Å to about 100 Å, for example, about 3 Å to about 90 Å. If the thickness of the electron injection layer EIL satisfies the above-described range, suitable or satisfactory electron injection characteristics may be achieved without substantial increase of a driving voltage.

The second electrode EL2 is provided on the electron transport region ETR. The second electrode EL2 may be a common electrode. The second electrode EL2 may be a cathode or an anode, but the embodiment of the present disclosure is not limited thereto. For example, if the first electrode EL1 is an anode, the second electrode EL2 may be a cathode, and if the first electrode EL1 is a cathode, the second electrode EL2 may be an anode.

The second electrode EL2 may be a transmissive electrode, a transflective electrode, or a reflective electrode. If the second electrode EL2 is the transmissive electrode, the second electrode EL2 may include transparent metal oxide such as indium tin oxide (ITO), indium zinc oxide (IZO), zinc oxide (ZnO), indium tin zinc oxide (ITZO), and/or the like.

If the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, a compound thereof, or a mixture containing thereof (for example, AgMg, AgYb, and/or MgAg). In one or more embodiments, the second electrode EL2 may have a multilayered structure including a reflective film or a transflective film formed using the aforementioned materials and a transparent conductive film formed using indium tin oxide (ITO), indium zinc oxide (IZO), zinc oxide (ZnO), indium tin zinc oxide (ITZO), and/or the like. For example, the second electrode EL2 may include the aforementioned metal material, a combination of two or more metal materials selected from the aforementioned metal materials, and/or an oxide of the aforementioned metal materials.

In one or more embodiments, the second electrode EL2 may be coupled with an auxiliary electrode. If the second electrode EL2 is coupled with the auxiliary electrode, the resistance of the second electrode EL2 may decrease.

In one or more embodiments, a capping layer CPL may be further on the second electrode EL2 of the organic electroluminescence device ED according to an embodiment of the present disclosure. The capping layer CPL may include multiple layers or a single layer.

In an embodiment of the present disclosure, the capping layer CPL may be an organic layer and/or an inorganic layer. For example, if the capping layer CPL includes an inorganic material, the inorganic material may include alkali metal compound such as LiF, alkaline earth metal compound such as MgF₂, SiON, SiNₓ, SiO_{y}, and/or the like.

For example, if the capping layer CPL includes an organic material, the organic material may include α-NPD, NPB, TPD, m-MTDATA, Alq₃, CuPc, N4,N4,N4′,N4′-tetra (biphenyl-4-yl) biphenyl-4,4′-diamine (TPD15), 4,4′,4″-tris (carbazol sol-9-yl) triphenylamine (TCTA), etc., epoxy resin, and/or acrylate such as methacrylate. However, the embodiment of the present disclosure is not limited thereto, and the capping layer CPL may include at least one selected from among Compounds P1 to P5 below.

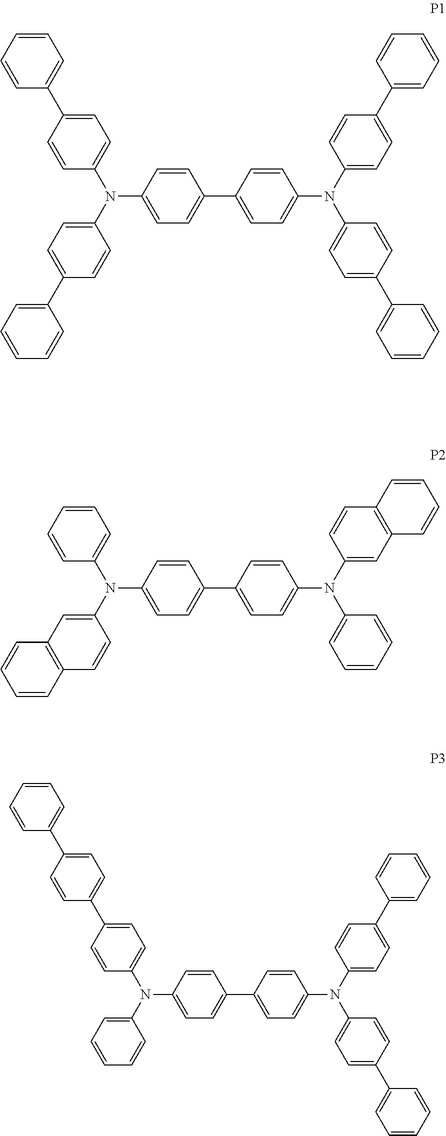 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

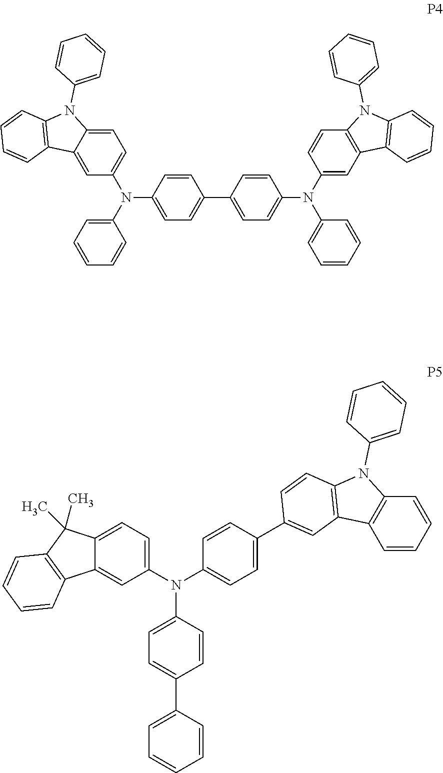 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In one or more embodiments, the refractive index of the capping layer CPL may be 1.6 or more. For example, for light in the wavelength range of about 550 nm to about 660 nm, the refractive index of the capping layer CPL may be about 1.6 or more.

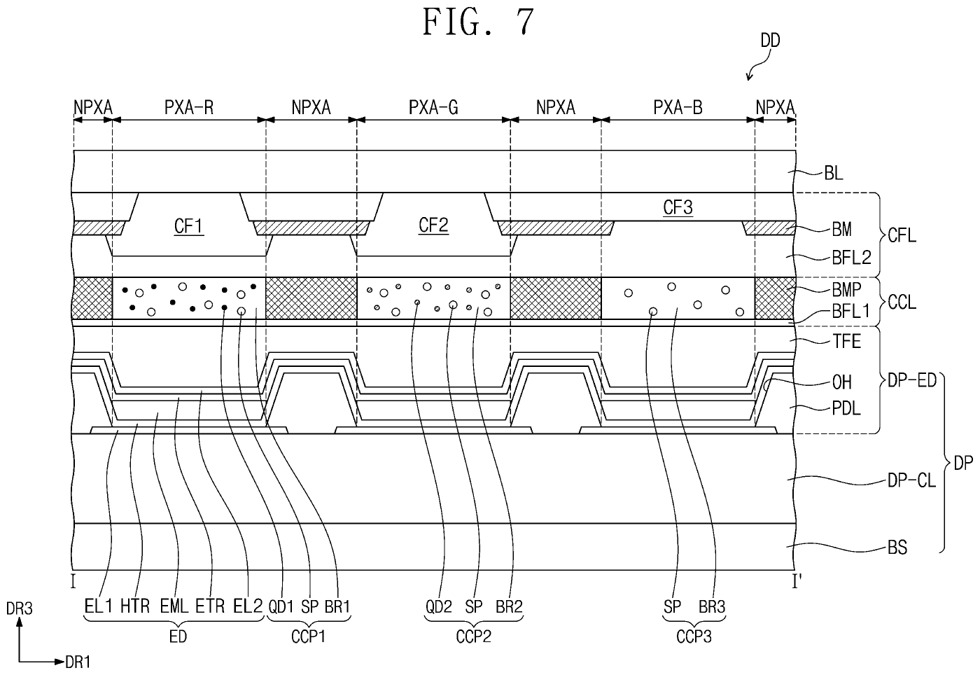

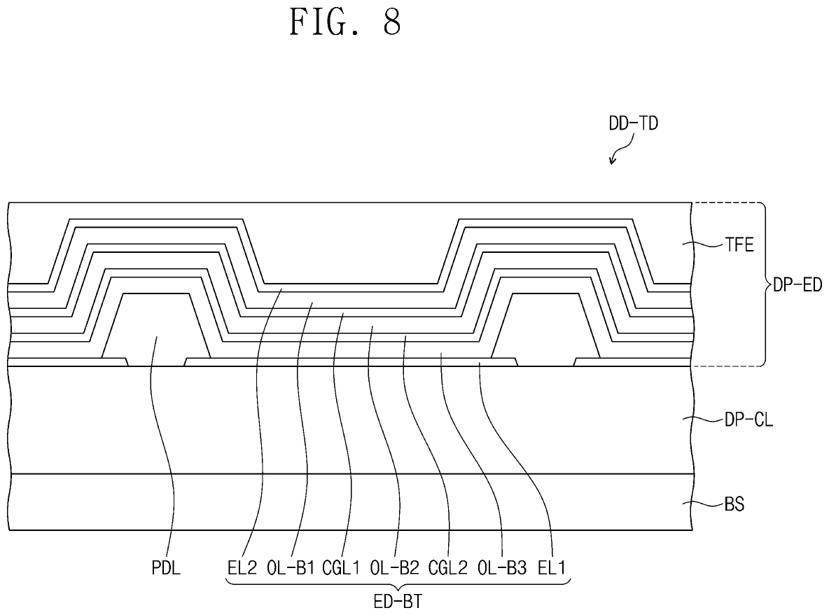

FIG. 7 and FIG. 8 are each a cross-sectional view showing a display apparatus according to an embodiment of the present disclosure. In the description of the display apparatus according to an embodiment of the present disclosure described with reference to FIG. 7 and FIG. 8, the contents overlapping with those described in FIGS. 1 to 6 will not be described again, and differences will be mainly described.

Referring to FIG. 7, a display apparatus DD according to an embodiment of the present disclosure may include a display panel DP including a display device layer DP-ED, a light control layer CCL on the display panel DP, and a color filter layer CFL.

In an embodiment of the present disclosure illustrated in FIG. 7, the display panel DP may include a base layer BS, a circuit layer DP-CL provided on the base layer BS, and a display device layer DP-ED, and the display device layer DP-ED may include an organic electroluminescence device ED.

The organic electroluminescence device ED may include a first electrode EL1, a hole transport region HTR on the first electrode EL1, an emission layer EML on the hole transport region HTR, an electron transport region ETR on the emission layer EML, and a second electrode EL2 on the electron transport region ETR. In one or more embodiments, the structure of the organic electroluminescence device ED illustrated in FIG. 7 may be equally applicable to the structure of the organic electroluminescence device in FIGS. 4 to 6 described above.

Referring to FIG. 7, the emission layer EML may be in an opening OH defined with respect to a pixel-defining film PDL. For example, the emission layer EML separated by the pixel-defining film PDL and provided corresponding to each of light-emitting regions PXA-R, PXA-G, and PXA-B may emit light in the same wavelength region. In a display apparatus DD according to an embodiment of the present disclosure, the emission layer EML may emit blue light. In one or more embodiments, in an embodiment of the present disclosure, the emission layer EML may be provided as a common layer to all of the light-emitting regions PXA-R, PXA-G, and PXA-B.

The light control layer CCL may be on the display panel DP. The light control layer CCL may include a light conversion body. The light conversion body may be a quantum dot and/or a phosphor. The light conversion body may convert the wavelength of received light to emit. For example, the light control layer CCL may be a layer including a quantum dot and/or a layer including a phosphor.

The light control layer CCL may include a plurality of light control portions CCP1, CCP2, and CCP3. The light control portions CCP1, CCP2, and CCP3 may be spaced apart from each other.

Referring to FIG. 7, a division pattern BMP may be between the light control portions CCP1, CCP2, and CCP3 spaced apart from each other, but the embodiment of the present disclosure is not limited thereto. In FIG. 7, the division pattern BMP is illustrated to be non-overlapping with the light control portions CCP1, CCP2, and CCP3, edges of the light control portions CCP1, CCP2, and CCP3 may at least partially overlap with the division pattern BMP.

The light control layer CCL may include a first light control portion CCP1 including a first quantum dot QD1 that converts a first color light provided in the organic electroluminescence device ED into a second color light, a second light control portion CCP2 including a second quantum dot QD2 that converts the first color light into a third color light, and a third light control portion CCP3 that transmits the first color light.

In an embodiment of the present disclosure, the first light control portion CCP1 may provide red light, which is a second color light, and the second light control portion CCP2 may provide green light, which is a third color light. The third light control portion CCP3 may transmit and provide blue light, which is the first light provided in the organic electroluminescence device ED. For example, the first quantum dot QD1 may be a red quantum dot, and the second quantum dot QD2 may be a green quantum dot. The same description as described above may be applied to the quantum dots QD1 and QD2.

In addition, the light control layer CCL may further include a scatterer SP (e.g., a light scatterer SP). The first light control portion CCP1 may include the first quantum dot QD1 and the scatterer SP, the second light control portion CCP2 may include the second quantum dot QD2 and the scatterer SP, and the third light control portion CCP3 may not include a quantum dot but may include the scatterer SP.

The scatterer SP may be an inorganic particle. For example, the scatterer SP may include at least one of TiO₂, ZnO, Al₂O₃, SiO₂, and/or hollow silica. The scatterer SP may include at least one of TiO₂, ZnO, Al₂O₃, SiO₂, and/or hollow silica, and/or may be a mixture of two or more materials selected from TiO₂, ZnO, Al₂O₃, SiO₂, and hollow silica.

Each of the first light control portion CCP1, the second light control portion CCP2, and the third light control portion CCP3 may include base resins BR1, BR2, and BR3, respectively, to disperse the quantum dots QD1 and QD2 and the scatterer SP. In an embodiment of the present disclosure, the first light control portion CCP1 may include the first quantum dot QD1 and the scatterer SP dispersed in the first base resin BR1, the second light control portion CCP2 may include the second quantum dot QD2 and scatterer SP dispersed in the second base resin BR2, and the third light control portion CCP3 may include the scatterer SP dispersed in the third base resin BR3. The base resins BR1, BR2, and BR3 are media in which the quantum dots QD1 and QD2 and the scatterer SP are dispersed, and may be formed of various suitable resin compositions which may be generally referred to as binders. For example, the base resins BR1, BR2, and BR3 may be acrylic resins, urethane-based resins, acrylic-based resins, epoxy-based resins, and/or the like. The base resins BR1, BR2, and BR3 may be transparent resins. In an embodiment of the present disclosure, each of the first base resin BR1, the second base resin BR2, and the third base resin BR3 may be the same as or different from each other.

The light control layer CCL may include a barrier layer BFL1. The barrier layer BFL1 may serve to prevent or reduce penetration of moisture and/or oxygen (which may be referred to hereinafter as “moisture/oxygen”). The barrier layer BFL1 may be on the light control portions CCP1, CCP2, and CCP3 to prevent or reduce exposure of the light control portions CCP1, CCP2, and CCP3 to moisture/oxygen. In one or more embodiments, the barrier layer BFL1 may cover the light control portions CCP1, CCP2, and CCP3. In addition, the barrier layer BFL2 may be provided between the light control portions CCP1, CCP2, and CCP3 and the color filter layer CFL as well.

The barrier layers BFL1 and BFL2 may include at least one inorganic layer. In one or more embodiments, the barrier layers BFL1 and BFL2 may be formed including an inorganic material. For example, the barrier layers BFL1 and BFL2 may be formed including silicon nitride, aluminum nitride, zirconium nitride, titanium nitride, hafnium nitride, tantalum nitride, silicon oxide, aluminum oxide, titanium oxide, tin oxide, cerium oxide, and/or silicon oxynitride, and/or a metal thin film wherein light transmittance is secured. In one or more embodiments, the barrier layers BFL1 and BFL2 may further include an organic film. The barrier layers BFL1 and BFL2 may be comprised of a single layer or a plurality of layers.

In a display apparatus DD according to an embodiment of the present disclosure, the color filter layer CFL may be on the light control layer CCL. For example, the color filter layer CFL may be directly on the light control layer CCL. In this case, the barrier layer BFL2 may be omitted.

The color filter layer CFL may include a light-shielding portion BM and filters CF-B, CF-G, and CF-R. The color filter layer CFL may include a first filter CF1 to transmit a second color light, a second filter CF2 to transmit a third color light, and a third filter CF3 to transmit a first color light. For example, the first filter CF1 may be a red filter, the second filter CF2 may be a green filter, and the third filter CF3 may be a blue filter. Each of the filters CF1, CF2, and CF3 may include polymer photosensitive resin and a pigment and/or a dye. The first filter CF1 may include a red pigment and/or dye, the second filter CF2 may include a green pigment and/or dye, and the third filter CF3 may include a blue pigment and/or dye. In one or more embodiments, the embodiment of the present disclosure is not limited thereto, and the third filter CF3 may not include a pigment or dye. The third filter CF3 may include polymer photosensitive resin and may not include a pigment or dye. The third filter CF3 may be transparent. The third filter CF3 may be formed of transparent photosensitive resin.

In addition, in an embodiment of the present disclosure, the first filter CF1 and the second filter CF2 may be a yellow filter. The first filter CF1 and the second filter CF2 may not be distinguished from each other and provided integrally.

The light-shielding portion BM may be a black matrix. The light-shielding portion BM may be formed by including an organic light-shielding material and/or an inorganic light-shielding material including a black pigment and/or a black dye. The light-shielding portion BM may prevent or reduce light leakage, and separate the boundary between the adjacent filters CF1, CF2, and CF3. In addition, in an embodiment of the present disclosure, the light-shielding portion BM may be formed of a blue filter.

Each of the first to the third filters CF1, CF2, and CF3 may respectively correspond to a red light-emitting region PXA-R, a green light-emitting region PXA-G, and a blue light-emitting region PXA-B.

A base substrate BL may be on the color filter layer CFL. The base substrate BL may be a member that provides a base surface on which the color filter layer CFL and the light control layer CCL are located. The base substrate BL may be a glass substrate, a metal substrate, a plastic substrate, and/or the like. However, the embodiment of the present disclosure is not limited thereto, and the base substrate BL may be an inorganic layer, an organic layer, or a composite material layer including an inorganic material and an organic material. In one or more embodiments, the base substrate BL may be omitted in an embodiment of the present disclosure.

FIG. 8 is a cross-sectional view illustrating a portion of a display apparatus according to an embodiment of the present disclosure. FIG. 8 illustrates a cross-sectional view of a portion corresponding to the display panel DP of FIG. 7. In the display apparatus DD-TD according to an embodiment of the present disclosure, the organic electroluminescence device ED-BT may include a plurality of light-emitting structures OL-B1, OL-B2, and OL-B3. The organic electroluminescence device ED-BT may include a first electrode EL1 and a second electrode EL2 that face each other, and a plurality of light-emitting structures OL-B1, OL-B2, and OL-B3 that are provided by sequentially stacking in a thickness direction between the first electrode EL1 and the second electrode EL2. Each of the light-emitting structures OL-B1, OL-B2, and OL-B3 may include the emission layer EML (FIG. 7), and the hole transport region HTR and the electron transport region ETR between the emission layer EML (FIG. 7).

The organic electroluminescence device ED-BT included in the display apparatus DD-TD according to an embodiment of the present disclosure may be an organic electroluminescence device having a tandem structure including a plurality of emission layers.

In an embodiment of the present disclosure illustrated in FIG. 8, all of the light emitted from each of the light-emitting structures OL-B1, OL-B2, and OL-B3 may be blue light. However, the embodiment of the present disclosure is not limited thereto, and the wavelength ranges of light emitted from each of the light-emitting structures OL-B1, OL-B2, and OL-B3 may be different from each other. For example, the organic electroluminescence device ED-BT including the plurality of light-emitting structures OL-B1, OL-B2, and OL-B3 that emit light having different wavelength regions may emit white light.

A charge generating layer CGL may be between adjacent light-emitting structures OL-B1, OL-B2, and OL-B3. The charge generating layer CGL may include a p-type charge generating layer and/or an n-type charge generating layer.

Hereinafter, the present application will be described in more detail with reference to specific examples and comparative examples. The following examples are only illustrations to assist the understanding of the present disclosure, and the scope of the present disclosure is not limited thereto.

### Synthesis Example

A compound according to an embodiment of the present disclosure may be synthesized, for example, as follows. However, a method for synthesizing a compound according to an embodiment of the present disclosure is not limited thereto.

### 1. Synthesis of Compound 1

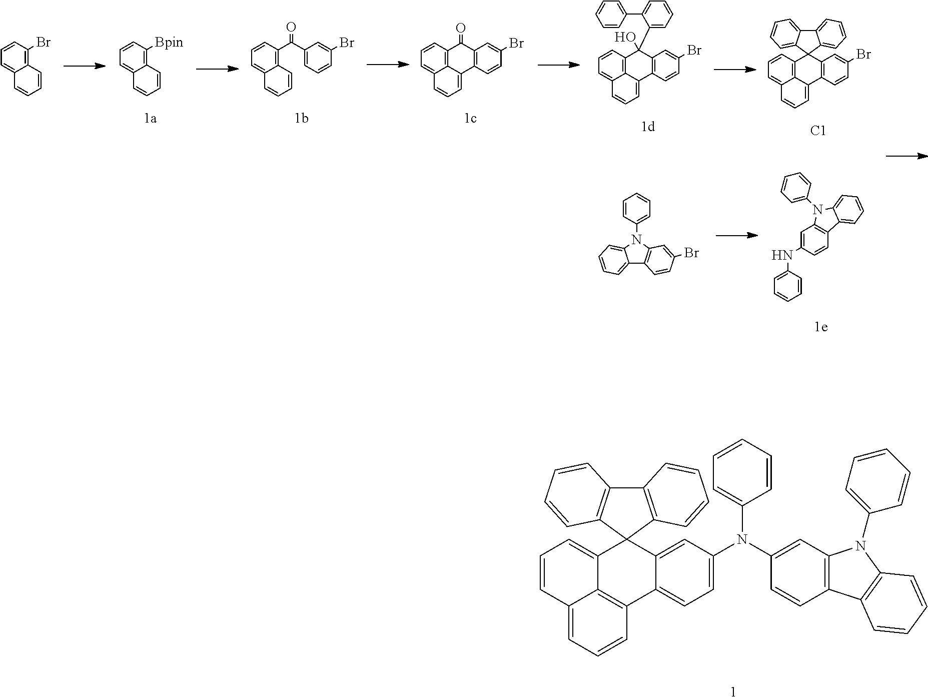

### 1-1. Synthesis of Intermediate 1a

1-bromonaphthalene (1.0 eq.), bis(pinacolato)diboron (2.0 eq.), potassium acetate (4.0 eq.), and palladium acetate (0.05 eq.) were dissolved in 1,4-dioxane, followed by stirring under a nitrogen atmosphere at about 80° C. for about 3 hours. After cooling and then washing with ethyl acetate and water three times, the resulting organic layer was dried over MgSO₄, followed by drying under reduced pressure. Intermediate 1a was obtained by column chromatography. (yield: 85%)

### 1-2. Synthesis of Intermediate 1b

Intermediate 1a (1.0 eq.), 3-bromobenzoyl chloride (1.5 eq.), tetrakis(triphenylphosphine)palladium (0.05 eq.), and potassium carbonate (2.0 eq.) were dissolved in THF:H₂O in a volume ratio of 4:1, and then stirred at about 80° C. for about 12 hours under a nitrogen atmosphere. After cooling and then washing with ethyl acetate and water three times, the resulting organic layer was dried over MgSO₄, followed by drying under reduced pressure. Intermediate 1 b was obtained by column chromatography. (yield: 66%)

### 1-3. Synthesis of Intermediate 1c

Intermediate 1 b (1.0 eq.), palladium acetate (0.01 eq.), and silver(I) oxide (1.5 eq.) were dissolved in trifluoroacetic acid, and then stirred at about 130° C. for about 36 hours under a nitrogen atmosphere. After cooling and then washing with ethyl acetate and water three times, the resulting organic layer was dried over MgSO₄, followed by drying under reduced pressure. Intermediate 1c was obtained by column chromatography. (yield: 70%)

### 1-4. Synthesis of Intermediate 1d

Anhydrous diethyl ether was added dropwise to 2-bromo-1,1′-biphenyl (1.0 eq.), magnesium (5.0 eq.), and dichloroethane (0.01 eq.), and then stirred at about 40° C. for about an hour under a nitrogen atmosphere, followed by cooling to about 0° C. The resultant solution was slowly added dropwise to the Intermediate 1c solution dissolved in THF, and stirred at about 40° C. for about an hour. After cooling, an ammonium chloride solution was slowly added dropwise thereto, and washed with ethyl acetate and water three times, and then the resulting organic layer was dried over MgSO₄, followed by drying under reduced pressure. Intermediate 1d was obtained by column chromatography. (yield: 75%)

### 1-5. Synthesis of Intermediate C1

Intermediate 1d (1.0 eq.) was dissolved in 9:1 volume ratio of acetic acid:hydrochloric acid, and then stirred at about 80° C. for about 2 hours under a nitrogen atmosphere. After cooling and then washing with ethyl acetate and water three times, the resulting organic layer was dried over MgSO₄, followed by drying under reduced pressure. Intermediate C1 was obtained by column chromatography. (yield: 69%)

### 1-6. Synthesis of Intermediate 1e

2-bromo-9-phenyl-9H-carbazole (1 eq.), aniline (1.1 eq.), tris(dibenzylideneacetone)dipalladium(0) (0.03 eq.), tri-tert-butylphosphine (0.06 eq.), and sodium tert-butoxide (2.0 eq.) were dissolved in toluene, and then stirred at about 80° C. for about 2 hours under a nitrogen atmosphere. After cooling and then washing with ethyl acetate and water three times, the resulting organic layer was dried over MgSO₄, followed by drying under reduced pressure. Intermediate 1e was obtained by column chromatography. (yield: 85%)

### 1-7. Synthesis of Compound 1

Intermediate C1 (1.0 eq.), Intermediate 1e (1.1 eq.), tris(dibenzylideneacetone)dipalladium(0) (0.05 eq.), tri-tert-butylphosphine (0.10 eq.), and sodium tert-butoxide (2.0 eq.) were dissolved in toluene, and then stirred at about 90° C. for about 2 hours under a nitrogen atmosphere. After cooling and then washing with ethyl acetate and water three times, the resulting organic layer was dried over MgSO₄, followed by drying under reduced pressure. Compound 1 was obtained by column chromatography. (yield: 82%)

### 2. Synthesis of Compound 9

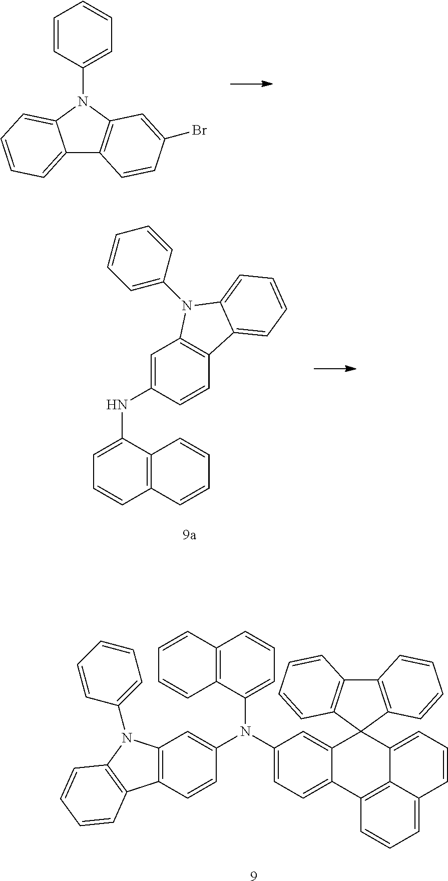 [SMILES: Brc1ccc2c3ccccc3n(-c3ccccc3)c2c1 ; c1ccc(-n2c3ccccc3c3ccc(Nc4cccc5ccccc45)cc32)cc1 ; c1ccc(-n2c3ccccc3c3ccc(N(c4ccc5c(c4)C4(c6ccccc6-c6ccccc64)c4cccc6cccc-5c46)c4cccc5ccccc45)cc32)cc1 ; "9"]

### 2-1. Synthesis of Intermediate 9a

2-bromo-9-phenyl-9H-carbazole (1 eq.), naphthalen-1-amine (1.1 eq.), tris(dibenzylideneacetone)dipalladium(0) (0.03 eq.), tri-tert-butylphosphine (0.06 eq.), and sodium tert-butoxide (2.0 eq.) were dissolved in toluene, and then stirred at about 80° C. for about 2 hours under a nitrogen atmosphere. After cooling and then washing with ethyl acetate and water three times, the resulting organic layer was dried over MgSO₄, followed by drying under reduced pressure. Intermediate 9a was obtained by column chromatography. (yield: 88%)

### 2-2. Synthesis of Compound 9

Intermediate C1 (1.0 eq.), Intermediate 9a (1.1 eq.), tris(dibenzylideneacetone)dipalladium(0) (0.05 eq.), tri-tert-butylphosphine (0.10 eq.), and sodium tert-butoxide (2.0 eq.) were dissolved in toluene, and then stirred at about 90° C. for about 2 hours under a nitrogen atmosphere. After cooling and then washing with ethyl acetate and water three times, the resulting organic layer was dried over MgSO₄, followed by drying under reduced pressure. Compound 9 was obtained by column chromatography. (yield: 81%)

### 3. Synthesis of Compound 12

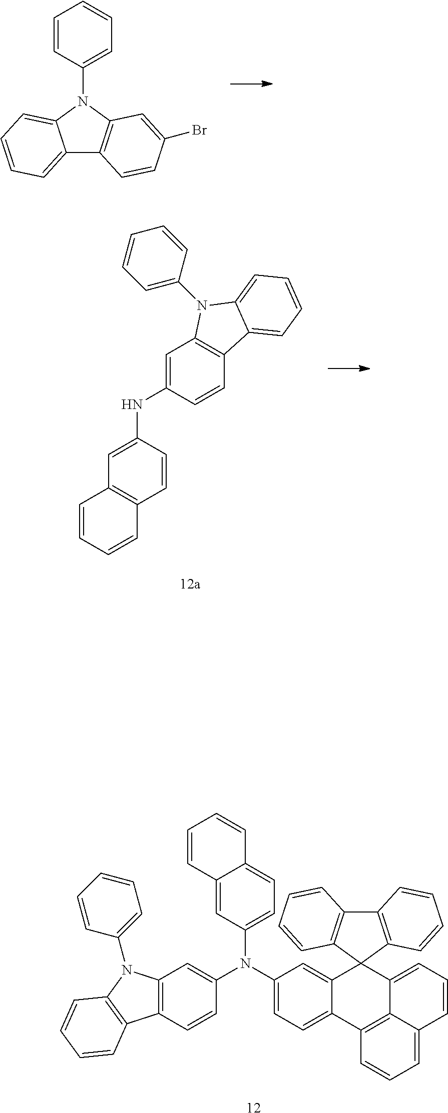 [SMILES: Brc1ccc2c3ccccc3n(-c3ccccc3)c2c1 ; c1ccc(-n2c3ccccc3c3ccc(Nc4ccc5ccccc5c4)cc32)cc1 ; c1ccc(-n2c3ccccc3c3ccc(N(c4ccc5c(c4)C4(c6ccccc6-c6ccccc64)c4cccc6cccc-5c46)c4ccc5ccccc5c4)cc32)cc1 ; "12"]

### 3-1. Synthesis of Intermediate 12a

2-bromo-9-phenyl-9H-carbazole (1 eq.), naphthalen-2-amine (1.1 eq.), tris(dibenzylideneacetone)dipalladium(0) (0.03 eq.), tri-tert-butylphosphine (0.06 eq.), and sodium tert-butoxide (2.0 eq.) were dissolved in toluene, and then stirred at about 80° C. for about 2 hours under a nitrogen atmosphere. After cooling and then washing with ethyl acetate and water three times, the resulting organic layer was dried over MgSO₄, followed by drying under reduced pressure. Intermediate 12a was obtained by column chromatography. (yield: 91%)

### 3-2. Synthesis of Compound 12

Intermediate C1 (1.0 eq.), Intermediate 12a (1.1 eq.), tris(dibenzylideneacetone)dipalladium(0) (0.05 eq.), tri-tert-butylphosphine (0.10 eq.), and sodium tert-butoxide (2.0 eq.) were dissolved in toluene, and then stirred at about 90° C. for about 2 hours under a nitrogen atmosphere. After cooling and then washing with ethyl acetate and water three times, the resulting organic layer was dried over MgSO₄, followed by drying under reduced pressure. Compound 12 was obtained by column chromatography. (yield: 81%)

### 4. Synthesis of Compound 20

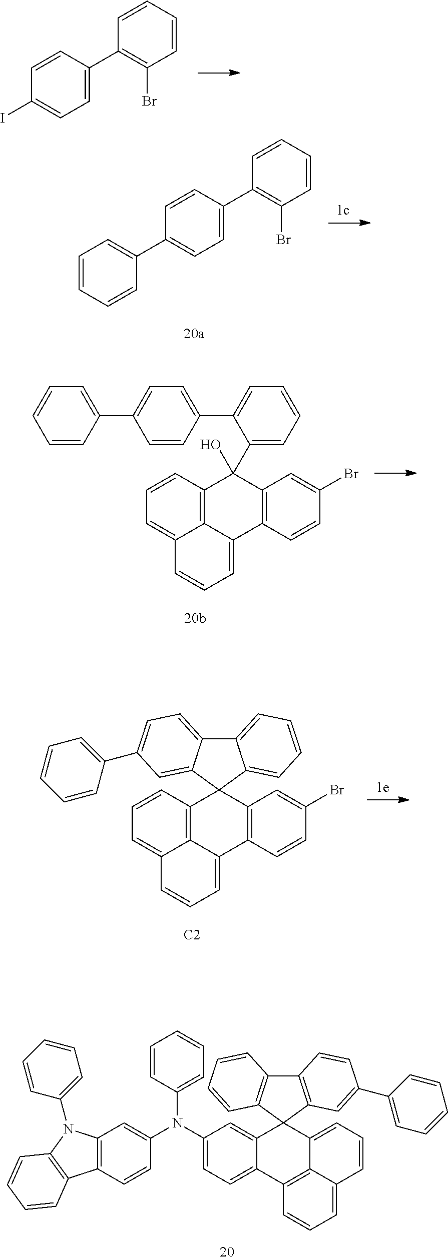

### 4-1. Synthesis of Intermediate 20a

2-bromo-4′-iodo-1,1′-biphenyl (1.0 eq.), phenylboronic acid (1.0 eq.), tetrakis(triphenylphosphine)palladium (0.05 eq.), and potassium carbonate (2.0 eq.) were dissolved in THF:H₂O in a volume ratio of 4:1, and then stirred at about 80° C. about 12 hours under a nitrogen atmosphere. After cooling and then washing with ethyl acetate and water three times, the resulting organic layer was dried over MgSO₄, followed by drying under reduced pressure. Intermediate 20a was obtained by column chromatography. (yield: 75%)

### 4-2. Synthesis of Intermediate 20b

Anhydrous diethyl ether was added dropwise to Intermediate 20a (1.0 eq.), magnesium (5.0 eq.), and dichloroethane (0.01 eq.), and then stirred at about 40° C. for about an hour under a nitrogen atmosphere, followed by cooling to about 0° C. The resultant solution was slowly added dropwise to the Intermediate 1c solution dissolved in THF, and stirred at about 40° C. for about an hour. After cooling, an ammonium chloride solution was slowly added dropwise thereto, and washed with ethyl acetate and water three times. Then, the resulting organic layer was dried over MgSO₄, followed by drying under reduced pressure. Intermediate 20b was obtained by column chromatography. (yield: 75%)

### 4-3. Synthesis of Intermediate C2

Intermediate 20b (1.0 eq.) was dissolved in 9:1 volume ratio of acetic acid:hydrochloric acid, and then stirred at about 80° C. for about 2 hours under a nitrogen atmosphere. After cooling and then washing with ethyl acetate and water three times, the resulting organic layer was dried over MgSO₄, followed by drying under reduced pressure. Intermediate C2 was obtained by column chromatography. (yield: 69%)

### 4-4. Synthesis of Compound 20

Intermediate C2 (1.0 eq.), Intermediate 1e (1.1 eq.), tris(dibenzylideneacetone)dipalladium(0) (0.05 eq.), tri-tert-butylphosphine (0.10 eq.), and sodium tert-butoxide (2.0 eq.) were dissolved in toluene, and then stirred at about 90° C. for about 2 hours under a nitrogen atmosphere. After cooling and then washing with ethyl acetate and water three times, the resulting organic layer was dried over MgSO₄, followed by drying under reduced pressure. Compound 20 was obtained by column chromatography. (yield: 85%)

### 5. Synthesis of Compound 45

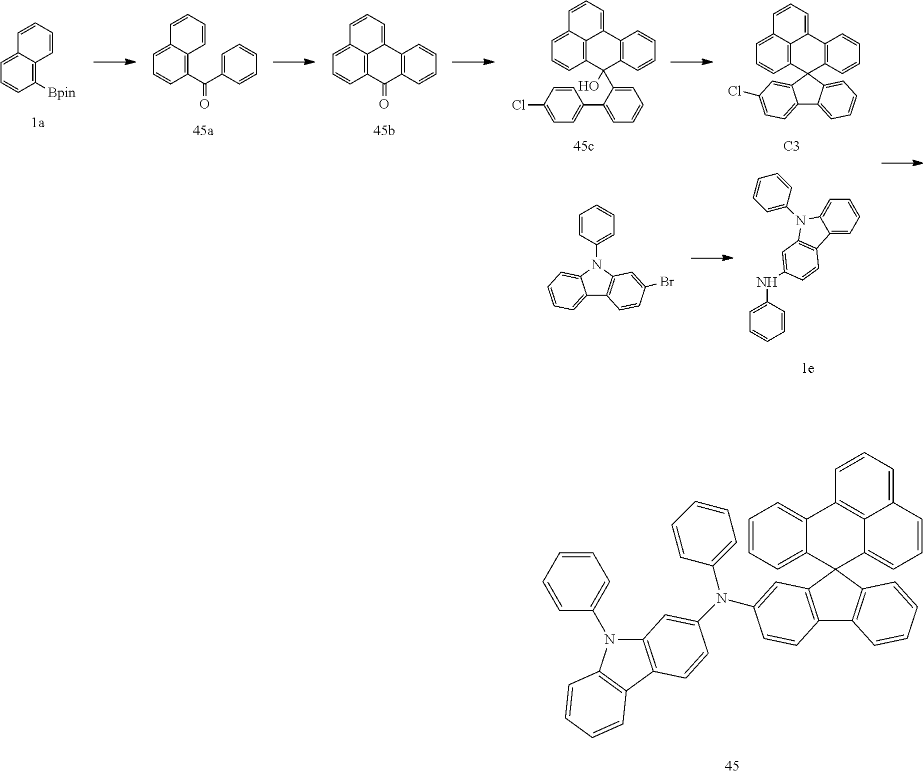

### 5-1. Synthesis of Intermediate 45a

Intermediate 1a (1.0 eq.), benzoyl chloride (1.5 eq.), tetrakis(triphenylphosphine)palladium (0.05 eq.), and potassium carbonate (2.0 eq.) were dissolved in THF:H₂O in a volume ratio of 4:1, and then stirred at about 80° C. for about 12 hours under a nitrogen atmosphere. After cooling and then washing with ethyl acetate and water three times, the resulting organic layer was dried over MgSO₄, followed by drying under reduced pressure. Intermediate 45a was obtained by column chromatography. (yield: 63%)

### 5-2. Synthesis of Intermediate 45b

Intermediate 1 b (1.0 eq.), palladium acetate (0.01 eq.), and silver(I) oxide (1.5 eq.) were dissolved in trifluoroacetic acid, and then stirred at about 130° C. for about 36 hours under a nitrogen atmosphere. After cooling and then washing with ethyl acetate and water three times, the resulting organic layer was dried over MgSO₄, followed by drying under reduced pressure. Intermediate 145b was obtained by column chromatography. (yield: 70%)

### 5-3. Synthesis of Intermediate 45c

Anhydrous diethyl ether was added dropwise to 2-bromo-4′-chloro-1,1′-biphenyl (1.0 eq.), magnesium (5.0 eq.), and dichloroethane (0.01 eq.), and then stirred at about 40° C. for about an hour under a nitrogen atmosphere, followed by cooling to about 0° C. This solution was slowly added dropwise to the Intermediate 1c solution dissolved in THF, and stirred at about 40° C. for about an hour. After cooling, an ammonium chloride solution was slowly added dropwise, and washed with ethyl acetate and water three times. Then, the resulting organic layer was dried over MgSO₄, followed by drying under reduced pressure. Intermediate 45c was obtained by column chromatography. (yield: 70%)

### 5-4. Synthesis of Intermediate C3

Intermediate 45c (1.0 eq.) was dissolved in 9:1 volume ratio of acetic acid:hydrochloric acid, and then stirred at about 80° C. for about 2 hours under a nitrogen atmosphere. After cooling and then washing with ethyl acetate and water three times, the resulting organic layer was dried over MgSO₄, followed by drying under reduced pressure. Intermediate C3 was obtained by column chromatography. (yield: 69%)

### 5-5. Synthesis of Compound 45

Intermediate C3 (1.0 eq.), Intermediate 1e (1.1 eq.), tris(dibenzylideneacetone)dipalladium(0) (0.05 eq.), tri-tert-butylphosphine (0.10 eq.), and sodium tert-butoxide (2.0 eq.) were dissolved in toluene, and then stirred at about 90° C. for about 2 hours under a nitrogen atmosphere. After cooling and then washing with ethyl acetate and water three times, the resulting organic layer was dried over MgSO₄, followed by drying under reduced pressure. Compound 45 was obtained by column chromatography. (yield: 92%)

### 6. Synthesis of Compound 53

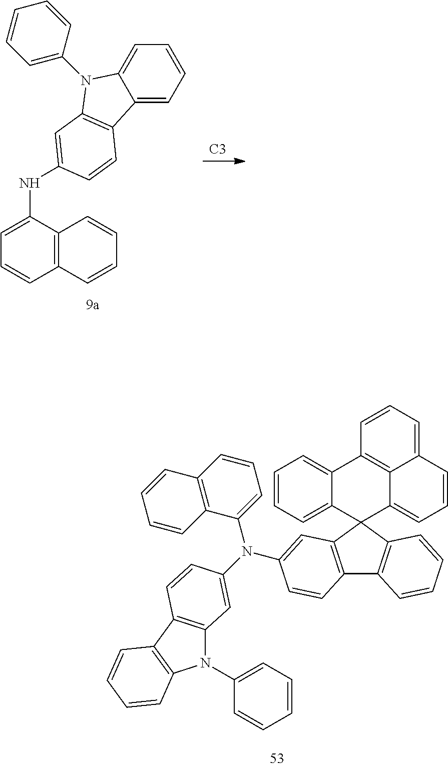 [SMILES: c1ccc(-n2c3ccccc3c3ccc(Nc4cccc5ccccc45)cc32)cc1 ; "C3" ; c1ccc(-n2c3ccccc3c3ccc(N(c4ccc5c(c4)C4(c6ccccc6-5)c5ccccc5-c5cccc6cccc4c56)c4cccc5ccccc45)cc32)cc1]

Intermediate C3 (1.0 eq.), Intermediate 9a (1.1 eq.), tris(dibenzylideneacetone)dipalladium(0) (0.05 eq.), tri-tert-butylphosphine (0.10 eq.), and sodium tert-butoxide (2.0 eq.) were dissolved in toluene, and then stirred at about 90° C. for about 2 hours under a nitrogen atmosphere. After cooling and then washing with ethyl acetate and water three times, the resulting organic layer was dried over MgSO₄, followed by drying under reduced pressure. Compound 53 was obtained by column chromatography. (yield: 84%)

### 7. Synthesis of Compound 56

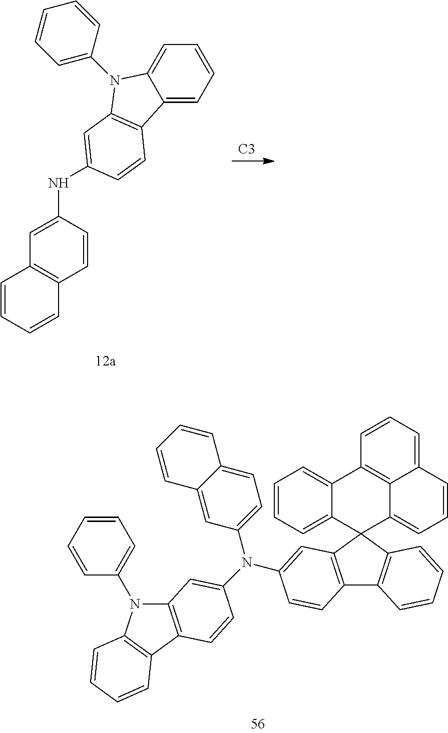 [SMILES: c1ccc(-n2c3ccccc3c3ccc(Nc4ccc5ccccc5c4)cc32)cc1 ; "C3" ; c1ccc(-n2c3ccccc3c3ccc(N(c4ccc5c(c4)C4(c6ccccc6-5)c5ccccc5-c5cccc6cccc4c56)c4ccc5ccccc5c4)cc32)cc1]

Intermediate C3 (1.0 eq.), Intermediate 12a (1.1 eq.), tris(dibenzylideneacetone)dipalladium(0) (0.05 eq.), tri-tert-butylphosphine (0.10 eq.), and sodium tert-butoxide (2.0 eq.) were dissolved in toluene, and then stirred at about 90° C. for about 2 hours under a nitrogen atmosphere. After cooling and then washing with ethyl acetate and water three times, the resulting organic layer was dried over MgSO₄, followed by drying under reduced pressure. Compound 56 was obtained by column chromatography. (yield: 81%)

### 8. Synthesis of Compound 64

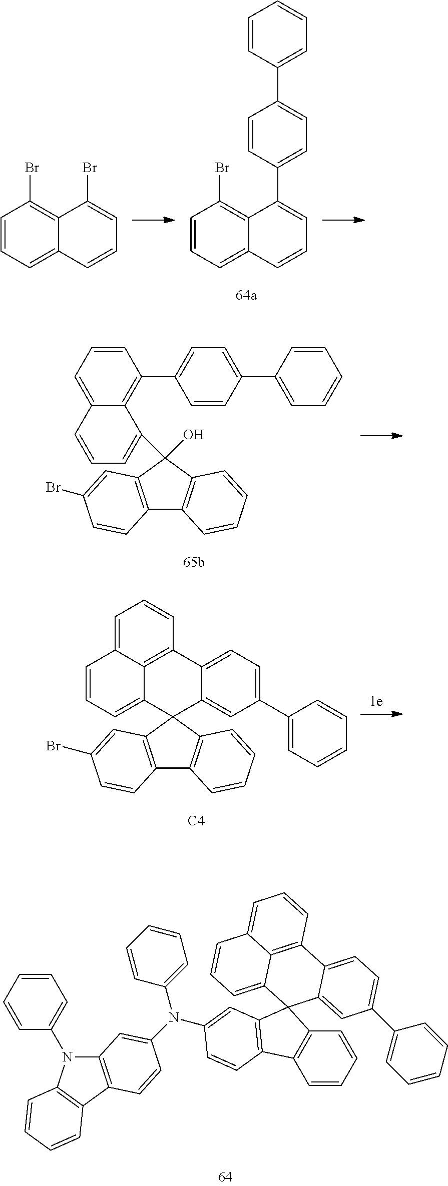

### 8-1. Synthesis of Intermediate 64a

1,8-dibromonaphthalene (1.0 eq.), [1,1′-biphenyl]-4-ylboronic acid (1.0 eq.), tetrakis(triphenylphosphine)palladium (0.05 eq.), and potassium carbonate (2.0 eq.) were dissolved in THF:H₂O in a volume ratio of 4:1, and then stirred at about 80° C. for about 12 hours under a nitrogen atmosphere. After cooling and then washing with ethyl acetate and water three times, the resulting organic layer was dried over MgSO₄, followed by drying under reduced pressure. Intermediate 64a was obtained by column chromatography. (yield: 61%)

### 8-2. Synthesis of Intermediate 65b

Anhydrous diethyl ether was added dropwise to Intermediate 64a (1.0 eq.), magnesium (5.0 eq.), and dichloroethane (0.01 eq.), and then stirred at about 40° C. for about an hour under a nitrogen atmosphere. The resultant solution was cooled to about 0° C., and then slowly added dropwise to 2-bromo-9H-fluoren-9-one (1.0 eq.) solution dissolved in THF, and stirred at about 40° C. for about an hour. After cooling, an ammonium chloride solution was slowly added dropwise thereto, and washed with ethyl acetate and water three times. Then, the resulting organic layer was dried over MgSO₄, followed by drying under reduced pressure. Intermediate 65b was obtained by column chromatography. (yield: 65%)

### 8-3. Synthesis of Intermediate C4

Intermediate 64b (1.0 eq.) was dissolved in 9:1 volume ratio of acetic acid:hydrochloric acid, and then stirred at about 80° C. for about 2 hours under a nitrogen atmosphere. After cooling and then washing with ethyl acetate and water three times, the resulting organic layer was dried over MgSO₄, followed by drying under reduced pressure. Intermediate C4 was obtained by column chromatography. (yield: 69%)

### 8-4. Synthesis of Compound 64

Intermediate C4 (1.0 eq.), Intermediate 1e (1.1 eq.), tris(dibenzylideneacetone)dipalladium(0) (0.05 eq.), tri-tert-butylphosphine (0.10 eq.), and sodium tert-butoxide (2.0 eq.) were dissolved in toluene, and then stirred at about 90° C. for about 2 hours under a nitrogen atmosphere. After cooling and then washing with ethyl acetate and water three times, the resulting organic layer was dried over MgSO₄, followed by drying under reduced pressure. Compound 64 was obtained by column chromatography. (yield: 77%)

### 9. Synthesis of Compound 89

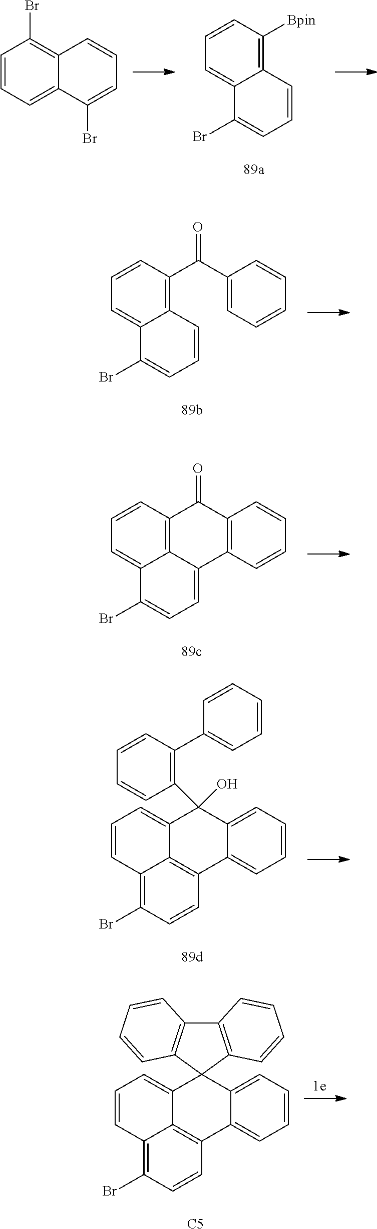

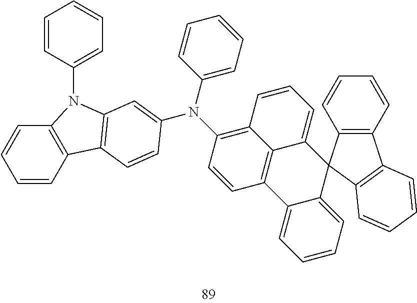 [SMILES: c1ccc(N(c2ccc3c4ccccc4n(-c4ccccc4)c3c2)c2ccc3c4c(cccc24)C2(c4ccccc4-c4ccccc42)c2ccccc2-3)cc1]

### 9-1. Synthesis of Intermediate 89a

1,5-dibromonaphthalene (1.0 eq.), bis(pinacolato)diboron (2.0 eq.), potassium acetate (4.0 eq.), and palladium acetate (0.05 eq.) were dissolved in 1,4-dioxane, followed by stirring at about 80° C. for about 3 hours under a nitrogen atmosphere. After cooling and then washing with ethyl acetate and water three times, the resulting organic layer was dried over MgSO₄, followed by drying under reduced pressure. Intermediate 89a was obtained by column chromatography. (yield: 83%)

### 9-2. Synthesis of Intermediate 89b

Intermediate 89a (1.0 eq.), benzoyl chloride (1.5 eq.), tetrakis(triphenylphosphine)palladium (0.05 eq.), and potassium carbonate (2.0 eq.) were dissolved in THF:H₂O in a volume ratio of 4:1, and then stirred at about 80° C. for about 12 hours under a nitrogen atmosphere. After cooling and then washing with ethyl acetate and water three times, the resulting organic layer was dried over MgSO₄, followed by drying under reduced pressure. Intermediate 89b was obtained by column chromatography. (yield: 64%)

### 9-3. Synthesis of Intermediate 89c

Intermediate 89b (1.0 eq.), palladium acetate (0.01 eq.), and silver(I) oxide (1.5 eq.) were dissolved in trifluoroacetic acid, and then stirred at about 130° C. for about 36 hours under a nitrogen atmosphere. After cooling and then washing with ethyl acetate and water three times, the resulting organic layer was dried over MgSO₄, followed by drying under reduced pressure. Intermediate 89c was obtained by column chromatography. (yield: 68%)

### 9-4. Synthesis of Intermediate 89d

Anhydrous diethyl ether was added dropwise to 2-bromo-1,1′-biphenyl (1.0 eq.), magnesium (5.0 eq.), and dichloroethane (0.01 eq.), and then stirred at about 40° C. for about an hour under a nitrogen atmosphere, followed by cooling to about 0° C. The resultant solution was slowly added dropwise to the Intermediate 89c solution dissolved in THF, and stirred at about 40° C. for about an hour. After cooling, an ammonium chloride solution was slowly added dropwise thereto, and washed with ethyl acetate and water three times. Then, the resulting organic layer was dried over MgSO₄, followed by drying under reduced pressure. Intermediate 89d was obtained by column chromatography. (yield: 75%)

### 9-5. Synthesis of Intermediate C5

Intermediate 89d (1.0 eq.) was dissolved in 9:1 volume ratio of acetic acid:hydrochloric acid, and then stirred at about 80° C. for about 2 hours under a nitrogen atmosphere. After cooling and then washing with ethyl acetate and water three times, the resulting organic layer was dried over MgSO₄, followed by drying under reduced pressure. Intermediate C5 was obtained by column chromatography. (yield: 78%)

### 9-6. Synthesis of Compound 89

Intermediate C5 (1.0 eq.), Intermediate 1e (1.1 eq.), tris(dibenzylideneacetone)dipalladium(0) (0.05 eq.), tri-tert-butylphosphine (0.10 eq.), and sodium tert-butoxide (2.0 eq.) were dissolved in toluene, and then stirred at about 90° C. for about 2 hours under a nitrogen atmosphere. After cooling and then washing with ethyl acetate and water three times, the resulting organic layer was dried over MgSO₄, followed by drying under reduced pressure. Compound 89 was obtained by column chromatography. (yield: 85%)

### 10. Synthesis of Compound 97

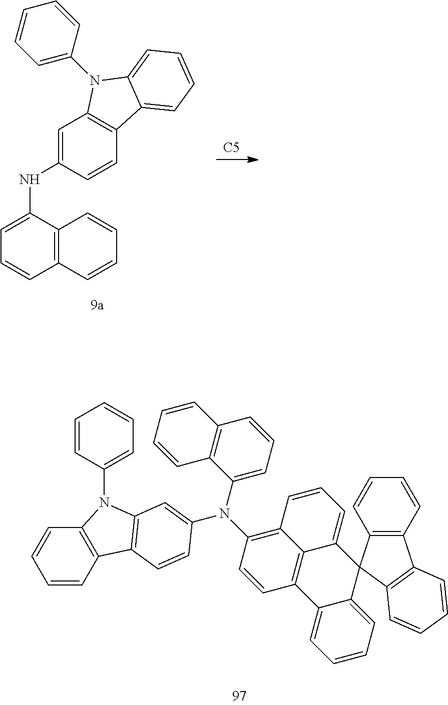

Intermediate C5 (1.0 eq.), Intermediate 9a (1.1 eq.), tris(dibenzylideneacetone)dipalladium(0) (0.05 eq.), tri-tert-butylphosphine (0.10 eq.), and sodium tert-butoxide (2.0 eq.) were dissolved in toluene, and then stirred at about 90° C. for about 2 hours under a nitrogen atmosphere. After cooling and then washing with ethyl acetate and water three times, the resulting organic layer was dried over MgSO₄, followed by drying under reduced pressure. Compound 97 was obtained by column chromatography. (yield: 84%)

### 11. Synthesis of Compound 100

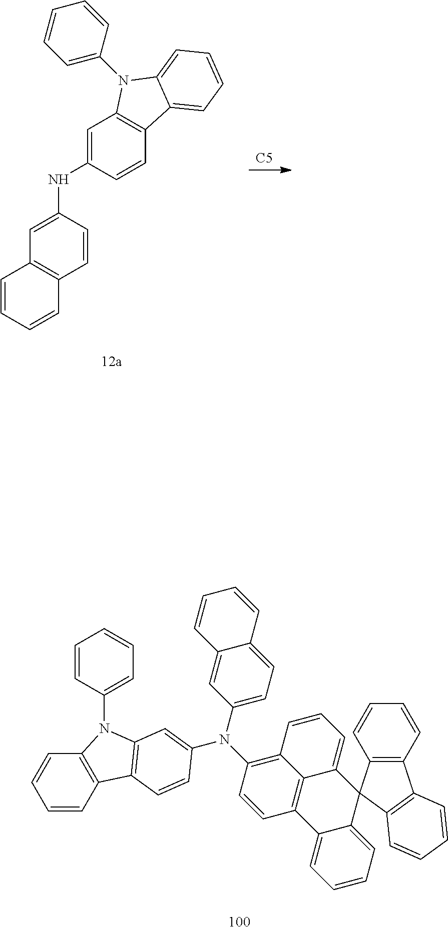 [SMILES: c1ccc(-n2c3ccccc3c3ccc(Nc4ccc5ccccc5c4)cc32)cc1 ; "C5" ; c1ccc(-n2c3ccccc3c3ccc(N(c4ccc5ccccc5c4)c4ccc5c6c(cccc46)C4(c6ccccc6-c6ccccc64)c4ccccc4-5)cc32)cc1]

Intermediate C5 (1.0 eq.), Intermediate 12a (1.1 eq.), tris(dibenzylideneacetone)dipalladium(0) (0.05 eq.), tri-tert-butylphosphine (0.10 eq.), and sodium tert-butoxide (2.0 eq.) were dissolved in toluene, and then stirred at about 90° C. for about 2 hours under a nitrogen atmosphere. After cooling and then washing with ethyl acetate and water three times, the resulting organic layer was dried over MgSO₄, followed by drying under reduced pressure. Compound 100 was obtained by column chromatography. (yield: 84%)

### 12. Synthesis of Compound 108

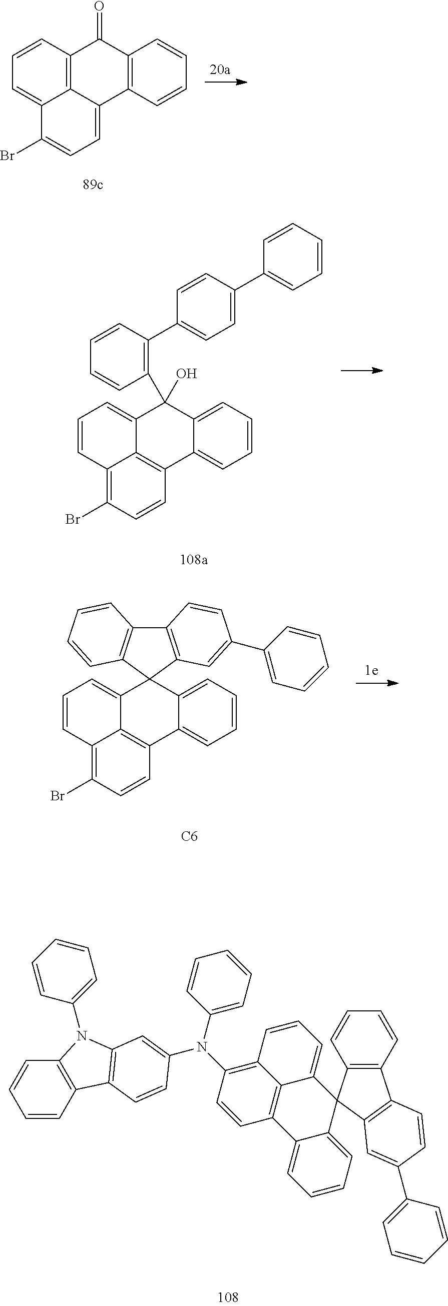

### 12-1. Synthesis of Intermediate 108a

Anhydrous diethyl ether was added dropwise to Intermediate 20a (1.0 eq.), magnesium (5.0 eq.), and dichloroethane (0.01 eq.), and then stirred at about 40° C. for about an hour under a nitrogen atmosphere, followed by cooling to about 0° C. The resultant solution was slowly added dropwise to the Intermediate 89c solution dissolved in THF, and stirred at about 40° C. for about an hour. After cooling, an ammonium chloride solution was slowly added dropwise thereto, and washed with ethyl acetate and water three times. Then, the resulting organic layer was dried over MgSO₄, followed by drying under reduced pressure. Intermediate 108a was obtained by column chromatography. (yield: 75%)

### 12-2. Synthesis of Intermediate C6

Intermediate 108a (1.0 eq.) was dissolved in 9:1 volume ratio of acetic acid:hydrochloric acid, and then stirred at about 80° C. for about 2 hours under a nitrogen atmosphere. After cooling and then washing with ethyl acetate and water three times, the resulting organic layer was dried over MgSO₄, followed by drying under reduced pressure. Intermediate C6 was obtained by column chromatography. (yield: 82%)

### 12-3. Synthesis of Compound 108

Intermediate C6 (1.0 eq.), Intermediate 1e (1.1 eq.), tris(dibenzylideneacetone)dipalladium(0) (0.05 eq.), tri-tert-butylphosphine (0.10 eq.), and sodium tert-butoxide (2.0 eq.) were dissolved in toluene, and then stirred at about 90° C. for about 2 hours under a nitrogen atmosphere. After cooling and then washing with ethyl acetate and water three times, the resulting organic layer was dried over MgSO₄, followed by drying under reduced pressure. Compound 108 was obtained by column chromatography. (yield: 89%)

### Device Fabrication Example

Organic electroluminescence devices were fabricated using the Example compounds and the Comparative Example compounds, as described below, as a material of a hole transport region.

### Example Compound

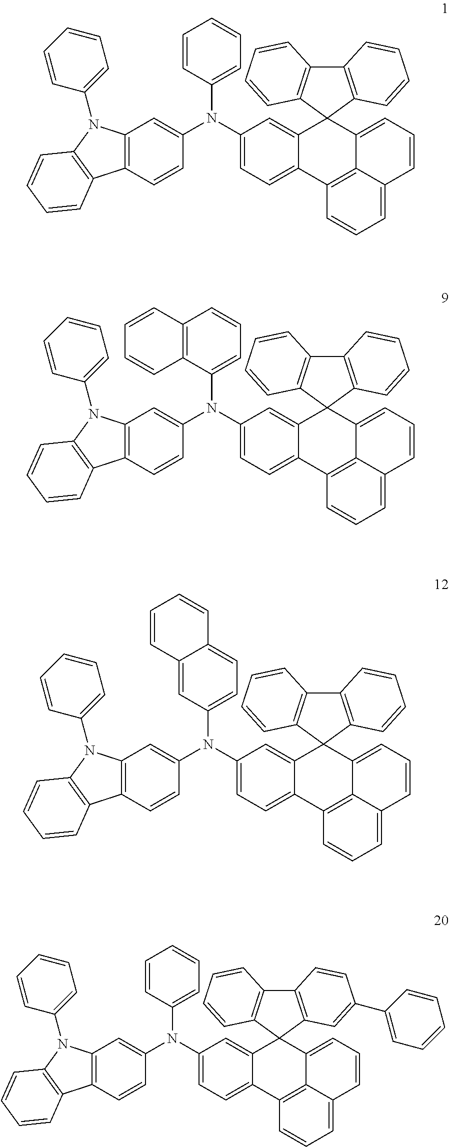 [SMILES: c1ccc(N(c2ccc3c(c2)C2(c4ccccc4-c4ccccc42)c2cccc4cccc-3c24)c2ccc3c4ccccc4n(-c4ccccc4)c3c2)cc1 ; c1ccc(-n2c3ccccc3c3ccc(N(c4ccc5c(c4)C4(c6ccccc6-c6ccccc64)c4cccc6cccc-5c46)c4cccc5ccccc45)cc32)cc1 ; c1ccc(-n2c3ccccc3c3ccc(N(c4ccc5c(c4)C4(c6ccccc6-c6ccccc64)c4cccc6cccc-5c46)c4ccc5ccccc5c4)cc32)cc1 ; c1ccc(-c2ccc3c(c2)C2(c4ccccc4-3)c3cc(N(c4ccccc4)c4ccc5c6ccccc6n(-c6ccccc6)c5c4)ccc3-c3cccc4cccc2c34)cc1]

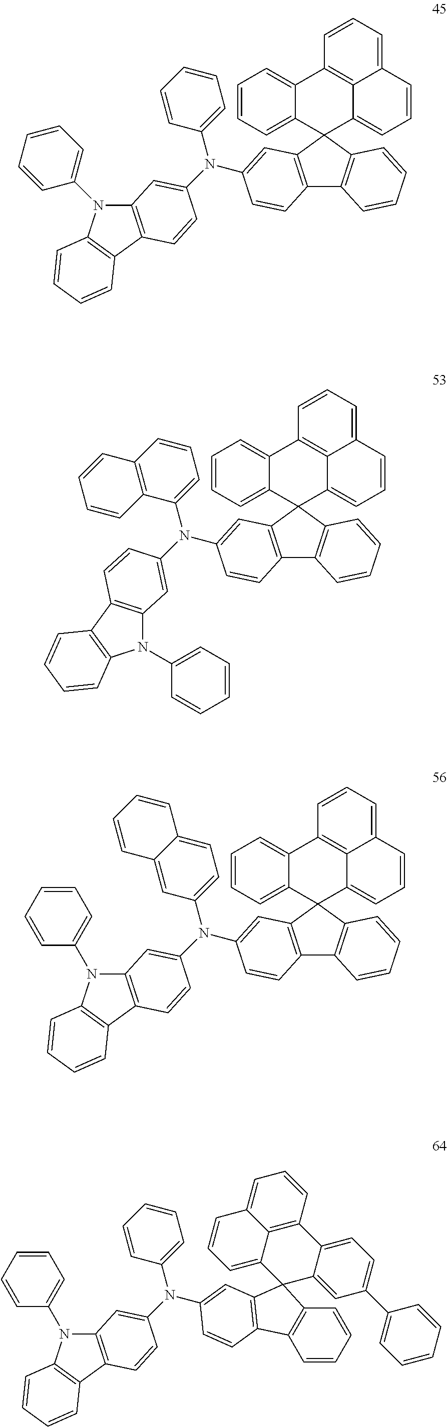 [SMILES: c1ccc(N(c2ccc3c(c2)C2(c4ccccc4-3)c3ccccc3-c3cccc4cccc2c34)c2ccc3c4ccccc4n(-c4ccccc4)c3c2)cc1 ; c1ccc(-n2c3ccccc3c3ccc(N(c4ccc5c(c4)C4(c6ccccc6-5)c5ccccc5-c5cccc6cccc4c56)c4cccc5ccccc45)cc32)cc1 ; c1ccc(-n2c3ccccc3c3ccc(N(c4ccc5c(c4)C4(c6ccccc6-5)c5ccccc5-c5cccc6cccc4c56)c4ccc5ccccc5c4)cc32)cc1 ; c1ccc(-c2ccc3c(c2)C2(c4ccccc4-c4ccc(N(c5ccccc5)c5ccc6c7ccccc7n(-c7ccccc7)c6c5)cc42)c2cccc4cccc-3c24)cc1]

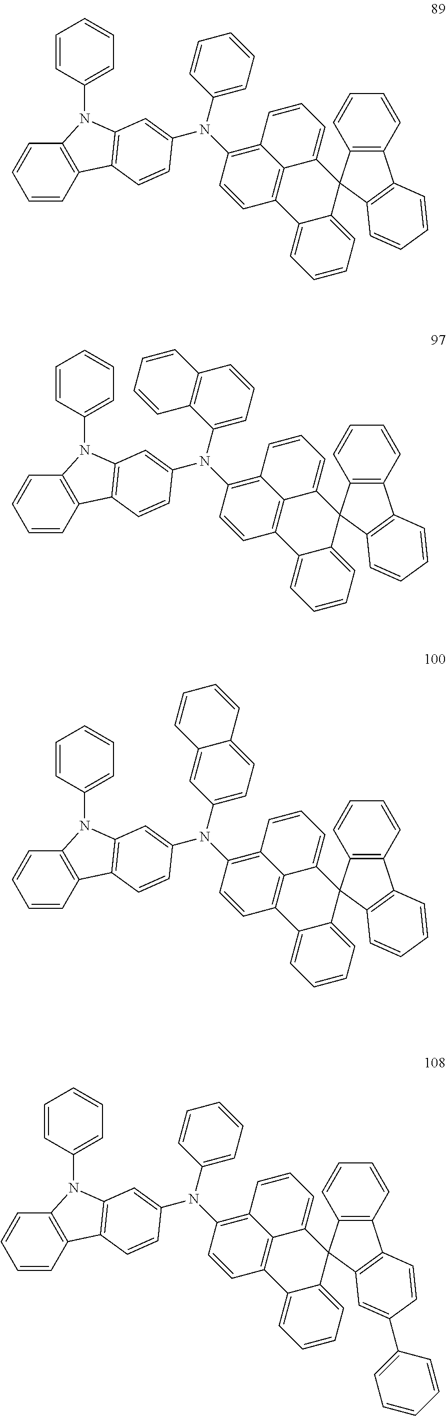 [SMILES: c1ccc(N(c2ccc3c4ccccc4n(-c4ccccc4)c3c2)c2ccc3c4c(cccc24)C2(c4ccccc4-c4ccccc42)c2ccccc2-3)cc1 ; c1ccc(-n2c3ccccc3c3ccc(N(c4cccc5ccccc45)c4ccc5c6c(cccc46)C4(c6ccccc6-c6ccccc64)c4ccccc4-5)cc32)cc1 ; c1ccc(-n2c3ccccc3c3ccc(N(c4ccc5ccccc5c4)c4ccc5c6c(cccc46)C4(c6ccccc6-c6ccccc64)c4ccccc4-5)cc32)cc1 ; c1ccc(-c2ccc3c(c2)C2(c4ccccc4-3)c3ccccc3-c3ccc(N(c4ccccc4)c4ccc5c6ccccc6n(-c6ccccc6)c5c4)c4cccc2c34)cc1]

### Comparative Example Compound

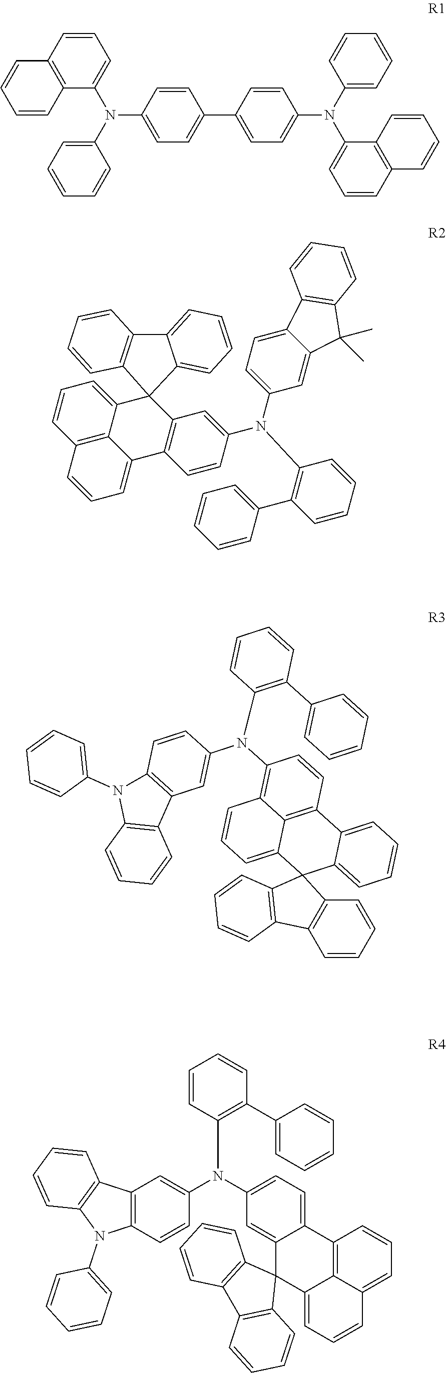 [SMILES: c1ccc(N(c2ccc(-c3ccc(N(c4ccccc4)c4cccc5ccccc45)cc3)cc2)c2cccc3ccccc23)cc1 ; CC1(C)c2ccccc2-c2ccc(N(c3ccc4c(c3)C3(c5ccccc5-c5ccccc53)c3cccc5cccc-4c35)c3ccccc3-c3ccccc3)cc21 ; c1ccc(-c2ccccc2N(c2ccc3c(c2)c2ccccc2n3-c2ccccc2)c2ccc3c4c(cccc24)C2(c4ccccc4-c4ccccc42)c2ccccc2-3)cc1 ; c1ccc(-c2ccccc2N(c2ccc3c(c2)C2(c4ccccc4-c4ccccc42)c2cccc4cccc-3c24)c2ccc3c(c2)c2ccccc2n3-c2ccccc2)cc1]

Organic electroluminescence devices of the Examples and the Comparative Examples were fabricated by the following method. ITO was patterned on a glass substrate having a thickness of about 120 nm, then washed with ultrapure water and treated with UV ozone to form a first electrode. Then, 2-TNATA was deposited to a thickness of about 60 nm, and a hole transport layer having a thickness of about 30 nm was formed using the compound of Examples or Comparative Examples. Next, an emission layer having a thickness of about 30 nm was formed in 9,10-di(naphthalen-2-yl)anthracene (DNA) doped with about 2% DPAVBi, and an electron transport region was formed by forming an about 30 nm thick layer of Alq₃ and an about 1 nm thick layer of LiF on the emission layer. Next, a second electrode having a thickness of about 300 nm was formed of aluminum (Al). Each layer was formed by a vacuum deposition method.

### Evaluation of Light Emitting Element Characteristics

Evaluation results of the light emitting elements of Examples 1 to 12 and Comparative Examples 1 to 4 are listed in Table 1. Driving voltage, luminance, luminous efficiency and a service life of the light emitting elements are listed in comparison in Table 1. In the evaluation results of the characteristics for the Examples and the Comparative Examples listed in Table 1, the luminous efficiency shows an efficiency value at a current density of 50 mA/cm², and the service life shows a brightness half-life at 100 mA/cm².

TABLE 1

| | | Driving | Current | | Luminous | Service |
|---|---|---|---|---|---|---|
| | Hole transport | voltage | Density | Luminance | efficacy | life |
| | layer | (V) | (mA/cm²) | (cd/m²) | (cd/A) | LT50 (h) |
| Example 1 | Example | 4.20 | 50 | 3650 | 7.30 | 359 |
| | Compound 1 | | | | | |
| Example 2 | Example | 4.21 | 50 | 3680 | 7.40 | 355 |
| | Compound 9 | | | | | |
| Example 3 | Example | 4.35 | 50 | 3520 | 7.20 | 350 |
| | Compound 12 | | | | | |
| Example 4 | Example | 4.15 | 50 | 3640 | 7.20 | 348 |
| | Compound 20 | | | | | |
| Example 5 | Example | 4.18 | 50 | 3750 | 7.50 | 376 |
| | Compound 45 | | | | | |
| Example 6 | Example | 4.20 | 50 | 3755 | 7.51 | 375 |
| | Compound 53 | | | | | |
| Example 7 | Example | 4.40 | 50 | 3650 | 7.18 | 360 |
| | Compound 56 | | | | | |
| Example 8 | Example | 4.15 | 50 | 3720 | 7.35 | 340 |
| | Compound 64 | | | | | |
| Example 9 | Example | 4.41 | 50 | 3605 | 7.40 | 372 |
| | Compound 89 | | | | | |
| Example 10 | Example | 4.51 | 50 | 3615 | 7.45 | 365 |
| | Compound 97 | | | | | |
| Example 11 | Example | 4.60 | 50 | 3510 | 7.30 | 355 |
| | Compound 100 | | | | | |
| Example 12 | Example | 4.22 | 50 | 3590 | 7.35 | 350 |
| | Compound 108 | | | | | |
| Comparative | Comparative | 7.01 | 50 | 2645 | 5.29 | 258 |
| Example 1 | Example | | | | | |
| | Compound R1 | | | | | |
| Comparative | Comparative | 4.42 | 50 | 2448 | 7.10 | 234 |
| Example 2 | Example | | | | | |
| | Compound R2 | | | | | |
| Comparative | Comparative | 4.65 | 50 | 2945 | 7.00 | 221 |
| Example 3 | Example | | | | | |
| | Compound R3 | | | | | |
| Comparative | Comparative | 4.62 | 50 | 3501 | 6.90 | 215 |
| Example 4 | Example | | | | | |
| | Compound R4 | | | | | |

Referring to Table 1, it may be seen that all of Examples 1 to 12 achieve low voltage, high luminance, high efficiency, and long service life at the same time compared to Comparative Examples 1 to 4.

Referring to Table 1, it may be seen that the Example compounds exhibit long service life and high efficiency characteristics compared with Comparative Example compounds R1 and R2, by having a molecular structure in which a spiro structure of a condensed ring and a carbazole group are bonded to an amine derivative at the same time.

In addition, while the present application is not bound by any particular mechanism or theory, it is believed that although Comparative Example compounds R3 and R4 have a similar molecular structure to that of the Example compounds, wherein a spiro structure of a condensed ring and a carbazole group are bonded to an amine derivative, the Example compounds have a lower HOMO (Highest Occupied Molecular Orbital) energy level than Comparative Example compounds R3 and R4 by having the amine derivative bonded to carbon number 2 of the carbazole group, so that hole transport characteristics may be improved, thereby exhibiting improved service life and luminous efficiency characteristics.

The amine compound according to an embodiment of the present disclosure is used in the hole transport region to contribute to low driving voltage, high efficiency, and long service life of the organic electroluminescence device. The amine compound according to an embodiment of the present disclosure is combined with a spiro(benzo[de]anthracene-7.9′-fluorene) structure. Accordingly, the amine compound according to an embodiment of the present disclosure may have a wide band value and a high glass transition temperature. Thus, hole transport characteristics may be improved, thereby increasing exciton generation efficiency to achieve high luminous efficiency.

The amine compound according to an embodiment of the present disclosure is used in the hole transport region to contribute to low driving voltage, high efficiency, and long service life of the organic electroluminescence device.

An organic electroluminescence device according to an embodiment of the present disclosure may have excellent efficiency.

An amine compound according to an embodiment of the present disclosure may be used as a material for a hole transport region of an organic electroluminescence device, and efficiency of an organic electroluminescence device may be improved by using the same.

Although example embodiments of the present disclosure have been described, it is understood that the present disclosure should not be limited to these example embodiments but various changes and modifications can be made by one ordinary skilled in the art within the spirit and scope of the present disclosure as defined by the appended claims, and equivalents thereof.

What is claimed is:

1. An organic electroluminescence device comprising:
a first electrode;
a second electrode facing the first electrode; and
an organic layer between the first electrode and the second electrode,
wherein the organic layer comprises an amine compound represented by Formula 1 below: 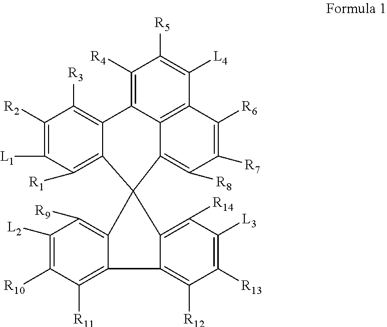
wherein, in Formula 1 above,
R₁ to R₁₄ are each independently a hydrogen atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms, and
L₁ to L₄ are each independently a hydrogen atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms, or a group represented by Formula 2 below,
where at least one selected from among L1 to L4 is represented by Formula 2 below: 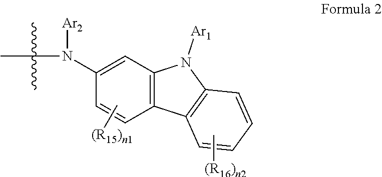
wherein, in Formula 2 above,
R₁₅ and R₁₆ are each independently a hydrogen atom, a deuterium atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms,
Ar₁ and Ar₂ are each independently a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms,
n1 is an integer of 0 to 3, and
n2 is an integer of 0 to 4.

2. The organic electroluminescence device of claim 1, wherein the organic layer comprises:
a hole transport region on the first electrode;
an emission layer on the hole transport region; and
an electron transport region on the emission layer,
wherein the hole transport region comprises an amine compound represented by Formula 1 above.

3. The organic electroluminescence device of claim 2, wherein the hole transport region comprises:
a hole injection layer on the first electrode; and
a hole transport layer on the hole injection layer,
wherein the hole injection layer or the hole transport layer comprises an amine compound represented by Formula 1 above.

4. The organic electroluminescence device of claim 2, wherein the hole transport region comprises:
a hole transport layer on the first electrode; and
an electron blocking layer on the hole transport layer,
wherein the electron blocking layer comprises an amine compound represented by Formula 1 above.

5. The organic electroluminescence device of claim 1, wherein R₁ to R₁₄, Ar₁, and Ar₂ do not comprise a substituted or unsubstituted amine group in the amine compound represented by Formula 1 above.

6. The organic electroluminescence device of claim 1, wherein Formula 1 above is represented by any one selected from among Formula 3-1 to Formula 3-4 below: 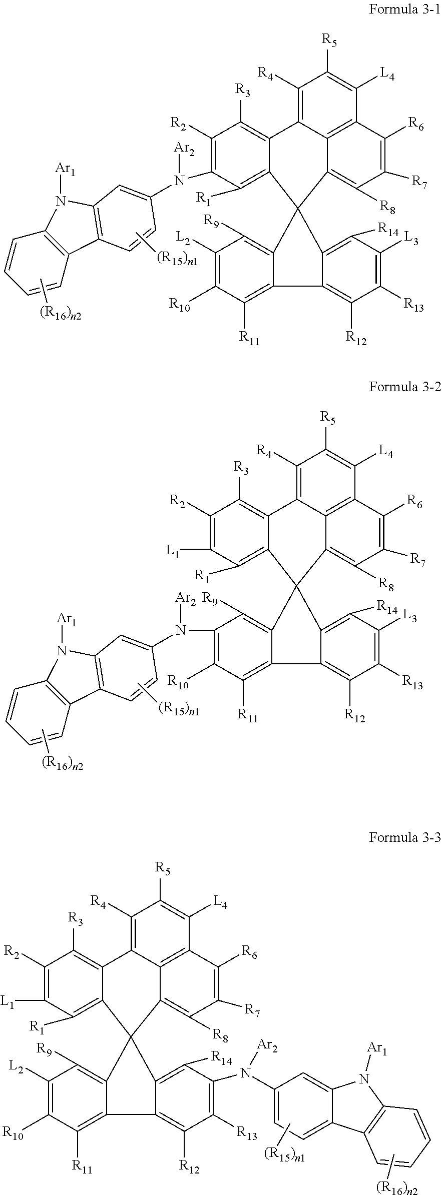 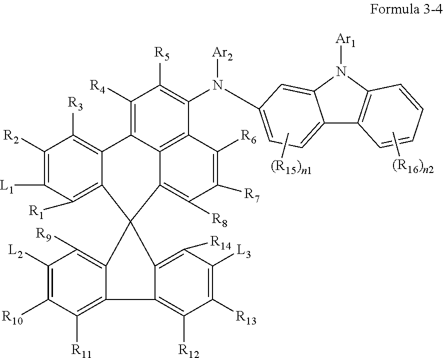
wherein, in Formula 3-1 to Formula 3-4 above,
R₁ to R₁₆, L₁ to L₄, An, Ar₂, n1, and n2 are the same as defined with respect to Formula 1 and Formula 2.

7. The organic electroluminescence device of claim 1, wherein the Ar₁ and Ar₂ are each independently a substituted or unsubstituted ring-forming aryl group having 6 to 18 carbon atoms.

8. The organic electroluminescence device of claim 7, wherein the Ar₁ and Ar₂ are each independently represented by any one selected from among Formula 4-1 to Formula 4-5 below: 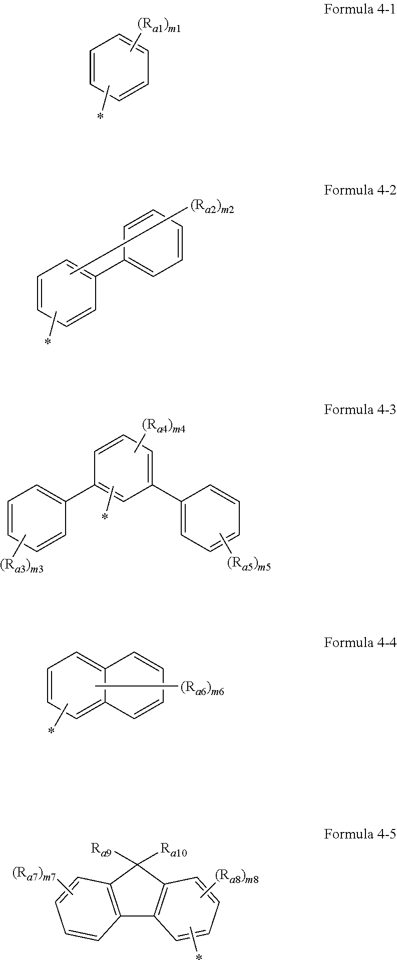
wherein, in Formula 4-1 to Formula 4-5 above,
Rₐ₁ to Rₐ₁₀ are each independently a hydrogen atom, a deuterium atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms,
m1, m3, and m5 are each independently an integer of 0 to 5,
m2 is an integer of 0 to 9,
m4 and m8 are each independently an integer of 0 to 3,
m6 is an integer of 0 to 7, and
m7 is an integer of 0 to 4

9. The organic electroluminescence device of claim 1, wherein the Ar₁ and Ar₂ are each independently a substituted or unsubstituted dibenzohetero group.

10. The organic electroluminescence device of claim 9, wherein the Ar₁ and Ar₂ are each independently represented by Formula 5-1 or Formula 5-2 below: 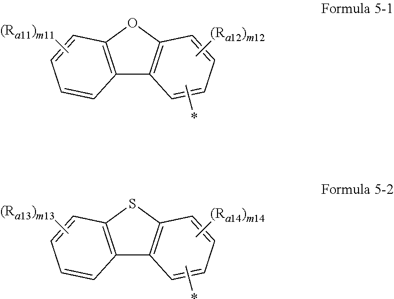
wherein, in Formula 5-1 and Formula 5-2 above,
Rₐ₁₁ to Rₐ₁₄ are each independently a hydrogen atom, a deuterium atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms,
m11 and m13 are each independently an integer of 0 to 4, and
m12 and m14 are each independently an integer of 0 to 3.

11. The organic electroluminescence device of claim 2, wherein the emission layer comprises a compound represented by Formula E-1 below: 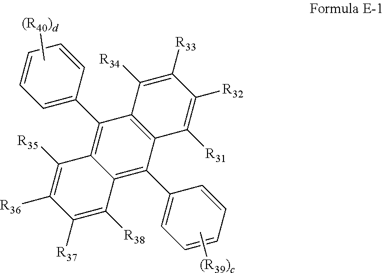
wherein, in Formula E-1 above,
R₃₁ to R₄₀ are each independently a hydrogen atom, a deuterium atom, a halogen atom, a substituted or unsubstituted silyl group, a substituted or unsubstituted alkyl group having 1 to 10 carbon atoms, a substituted or unsubstituted ring-forming aryl group having 6 to 30 carbon atoms, or a substituted or unsubstituted ring-forming heteroaryl group having 2 to 30 carbon atoms, or combined with an adjacent group to form a ring, and
c and d are each independently an integer of 0 to 5.

12. The organic electroluminescence device of claim 1, wherein the amine compound represented by Formula 1 above is any one selected from among the compounds represented in Compound Group 1 below:
Compound Group 1

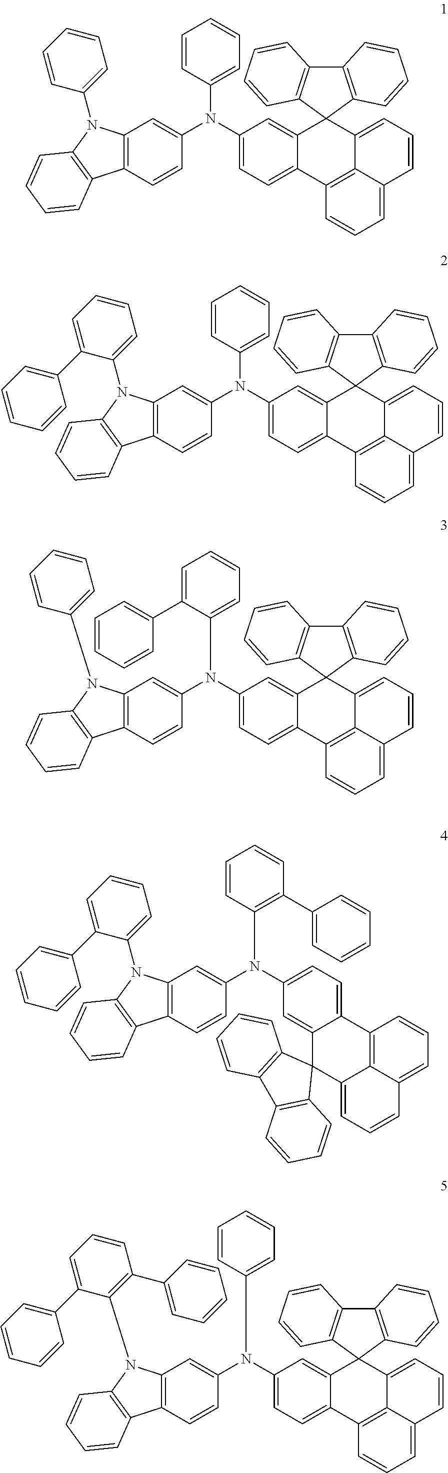

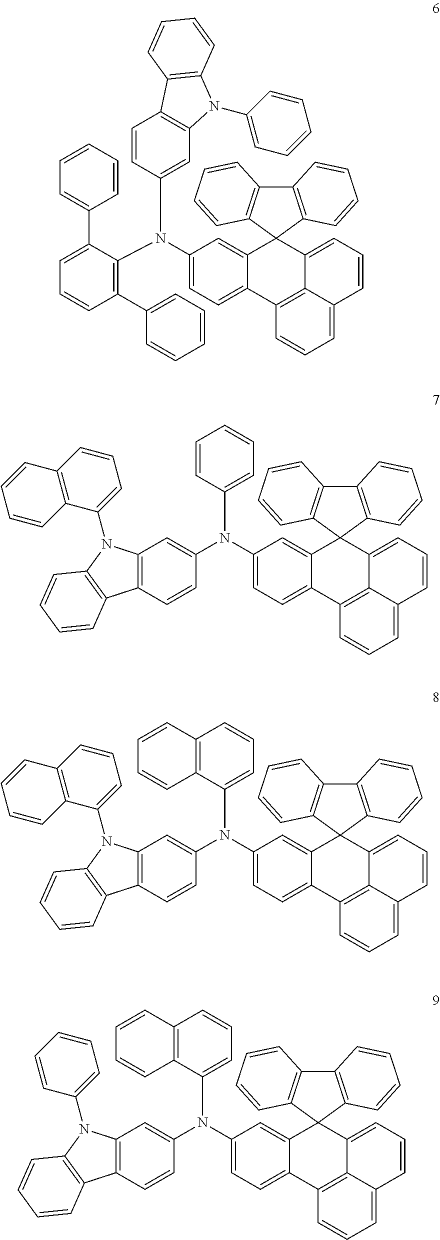 [SMILES: c1ccc(-c2cccc(-c3ccccc3)c2N(c2ccc3c(c2)C2(c4ccccc4-c4ccccc42)c2cccc4cccc-3c24)c2ccc3c4ccccc4n(-c4ccccc4)c3c2)cc1 ; c1ccc(N(c2ccc3c(c2)C2(c4ccccc4-c4ccccc42)c2cccc4cccc-3c24)c2ccc3c4ccccc4n(-c4cccc5ccccc45)c3c2)cc1 ; c1ccc2c(c1)-c1ccccc1C21c2cc(N(c3ccc4c5ccccc5n(-c5cccc6ccccc56)c4c3)c3cccc4ccccc34)ccc2-c2cccc3cccc1c23 ; c1ccc(-n2c3ccccc3c3ccc(N(c4ccc5c(c4)C4(c6ccccc6-c6ccccc64)c4cccc6cccc-5c46)c4cccc5ccccc45)cc32)cc1]

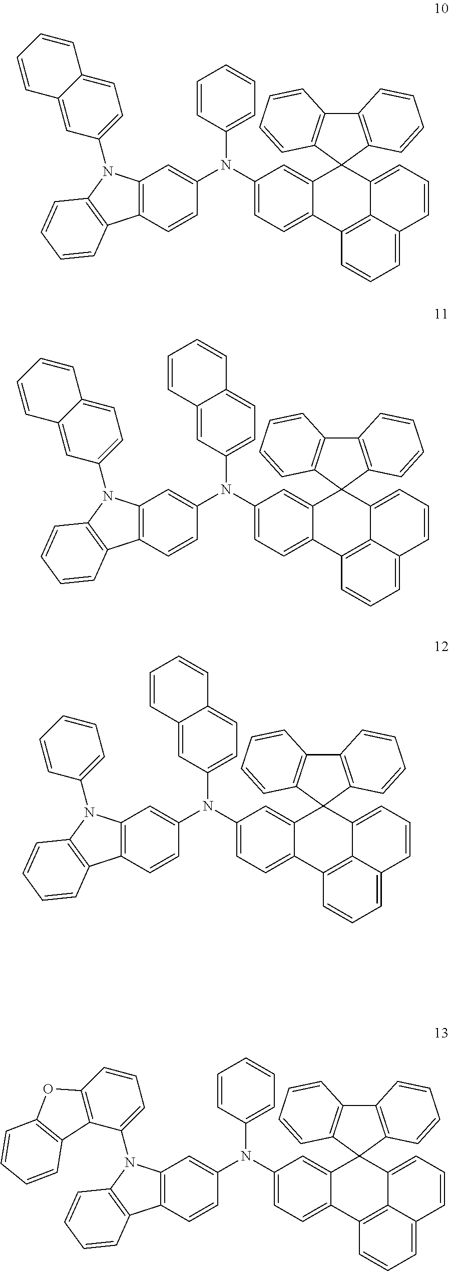 [SMILES: c1ccc(N(c2ccc3c(c2)C2(c4ccccc4-c4ccccc42)c2cccc4cccc-3c24)c2ccc3c4ccccc4n(-c4ccc5ccccc5c4)c3c2)cc1 ; c1ccc2c(c1)-c1ccccc1C21c2cc(N(c3ccc4ccccc4c3)c3ccc4c5ccccc5n(-c5ccc6ccccc6c5)c4c3)ccc2-c2cccc3cccc1c23 ; c1ccc(-n2c3ccccc3c3ccc(N(c4ccc5c(c4)C4(c6ccccc6-c6ccccc64)c4cccc6cccc-5c46)c4ccc5ccccc5c4)cc32)cc1 ; c1ccc(N(c2ccc3c(c2)C2(c4ccccc4-c4ccccc42)c2cccc4cccc-3c24)c2ccc3c4ccccc4n(-c4cccc5oc6ccccc6c45)c3c2)cc1]

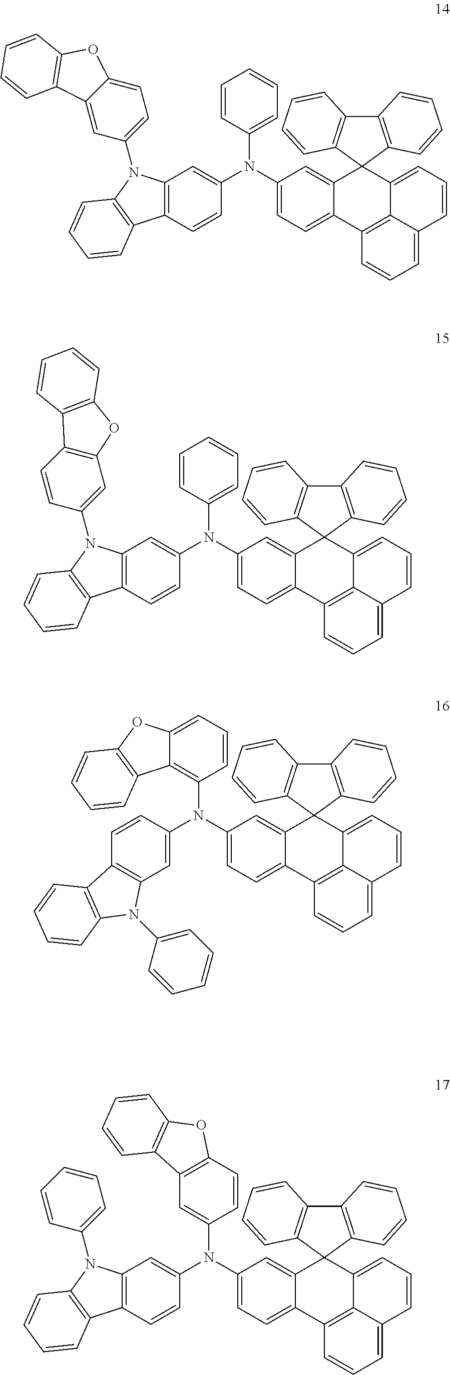 [SMILES: c1ccc(N(c2ccc3c(c2)C2(c4ccccc4-c4ccccc42)c2cccc4cccc-3c24)c2ccc3c4ccccc4n(-c4ccc5oc6ccccc6c5c4)c3c2)cc1 ; c1ccc(N(c2ccc3c(c2)C2(c4ccccc4-c4ccccc42)c2cccc4cccc-3c24)c2ccc3c4ccccc4n(-c4ccc5c(c4)oc4ccccc45)c3c2)cc1 ; c1ccc(-n2c3ccccc3c3ccc(N(c4ccc5c(c4)C4(c6ccccc6-c6ccccc64)c4cccc6cccc-5c46)c4cccc5oc6ccccc6c45)cc32)cc1 ; c1ccc(-n2c3ccccc3c3ccc(N(c4ccc5c(c4)C4(c6ccccc6-c6ccccc64)c4cccc6cccc-5c46)c4ccc5oc6ccccc6c5c4)cc32)cc1]

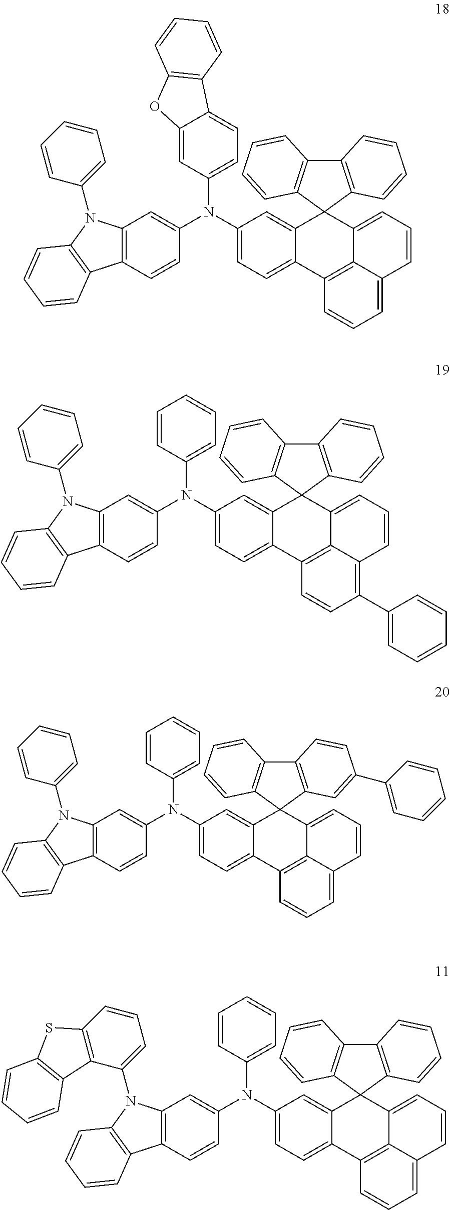 [SMILES: c1ccc(-n2c3ccccc3c3ccc(N(c4ccc5c(c4)C4(c6ccccc6-c6ccccc64)c4cccc6cccc-5c46)c4ccc5c(c4)oc4ccccc45)cc32)cc1 ; c1ccc(-c2ccc3c4c(cccc24)C2(c4ccccc4-c4ccccc42)c2cc(N(c4ccccc4)c4ccc5c6ccccc6n(-c6ccccc6)c5c4)ccc2-3)cc1 ; c1ccc(-c2ccc3c(c2)C2(c4ccccc4-3)c3cc(N(c4ccccc4)c4ccc5c6ccccc6n(-c6ccccc6)c5c4)ccc3-c3cccc4cccc2c34)cc1 ; c1ccc(N(c2ccc3c(c2)C2(c4ccccc4-c4ccccc42)c2cccc4cccc-3c24)c2ccc3c4ccccc4n(-c4cccc5sc6ccccc6c45)c3c2)cc1]

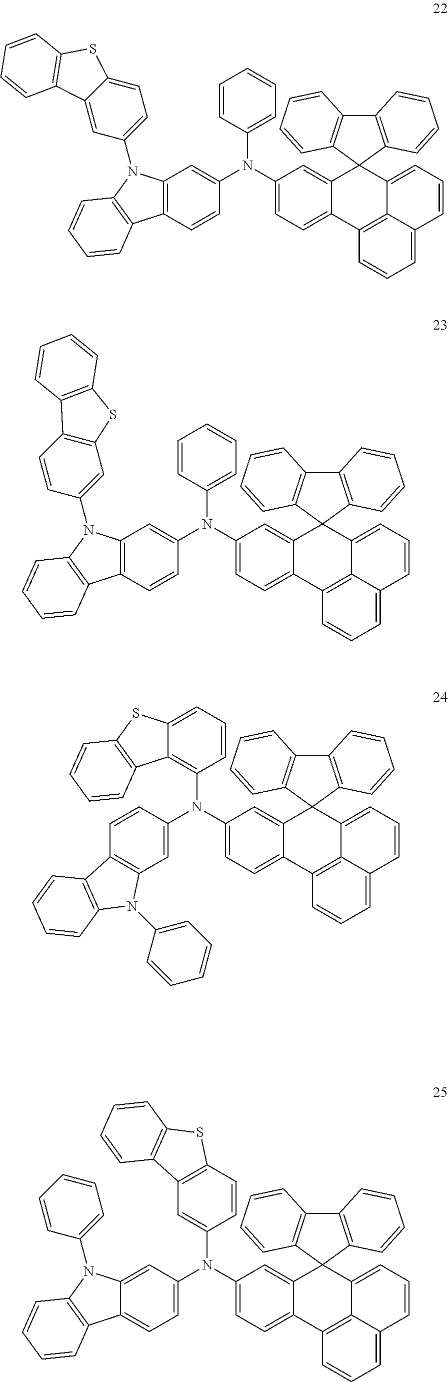 [SMILES: c1ccc(N(c2ccc3c(c2)C2(c4ccccc4-c4ccccc42)c2cccc4cccc-3c24)c2ccc3c4ccccc4n(-c4ccc5sc6ccccc6c5c4)c3c2)cc1 ; c1ccc(N(c2ccc3c(c2)C2(c4ccccc4-c4ccccc42)c2cccc4cccc-3c24)c2ccc3c4ccccc4n(-c4ccc5c(c4)sc4ccccc45)c3c2)cc1 ; c1ccc(-n2c3ccccc3c3ccc(N(c4ccc5c(c4)C4(c6ccccc6-c6ccccc64)c4cccc6cccc-5c46)c4cccc5sc6ccccc6c45)cc32)cc1 ; c1ccc(-n2c3ccccc3c3ccc(N(c4ccc5c(c4)C4(c6ccccc6-c6ccccc64)c4cccc6cccc-5c46)c4ccc5sc6ccccc6c5c4)cc32)cc1]

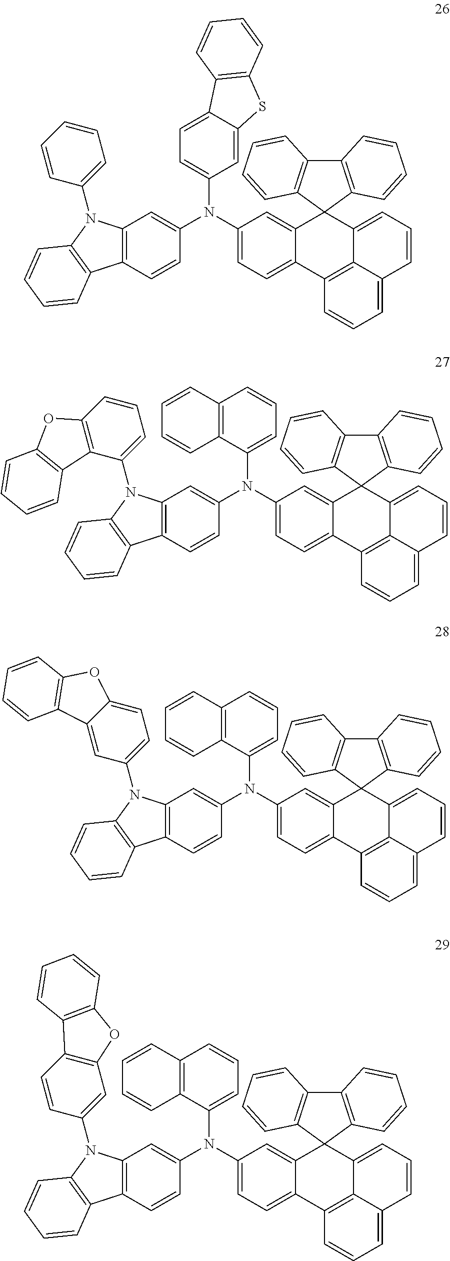 [SMILES: c1ccc(-n2c3ccccc3c3ccc(N(c4ccc5c(c4)C4(c6ccccc6-c6ccccc64)c4cccc6cccc-5c46)c4ccc5c(c4)sc4ccccc45)cc32)cc1 ; c1ccc2c(c1)-c1ccccc1C21c2cc(N(c3ccc4c5ccccc5n(-c5cccc6oc7ccccc7c56)c4c3)c3cccc4ccccc34)ccc2-c2cccc3cccc1c23 ; c1ccc2c(c1)-c1ccccc1C21c2cc(N(c3ccc4c5ccccc5n(-c5ccc6oc7ccccc7c6c5)c4c3)c3cccc4ccccc34)ccc2-c2cccc3cccc1c23 ; c1ccc2c(c1)-c1ccccc1C21c2cc(N(c3ccc4c5ccccc5n(-c5ccc6c(c5)oc5ccccc56)c4c3)c3cccc4ccccc34)ccc2-c2cccc3cccc1c23]

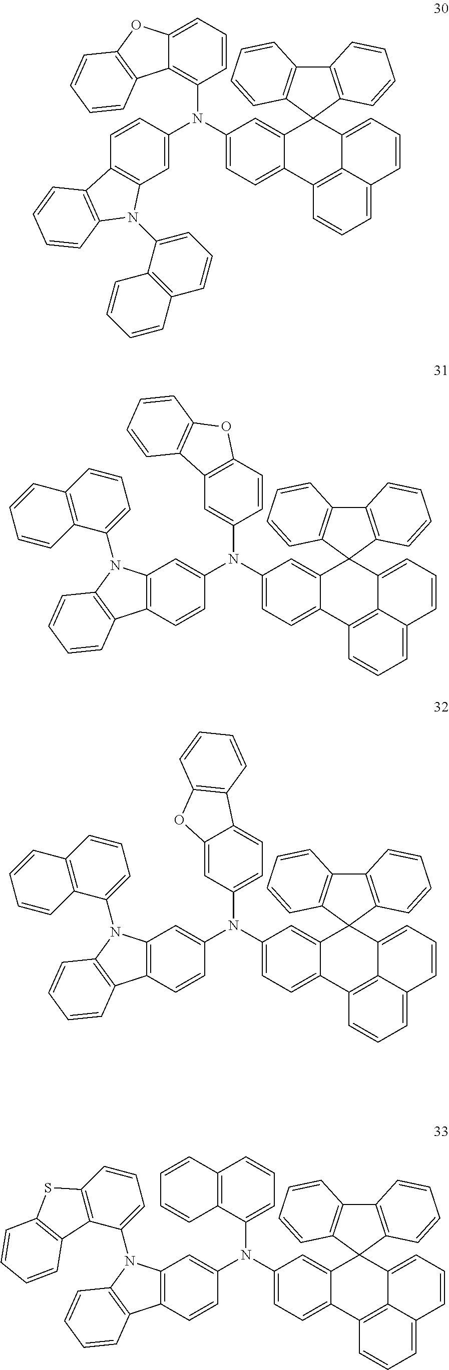 [SMILES: c1ccc2c(c1)-c1ccccc1C21c2cc(N(c3ccc4c5ccccc5n(-c5cccc6ccccc56)c4c3)c3cccc4oc5ccccc5c34)ccc2-c2cccc3cccc1c23 ; c1ccc2c(c1)-c1ccccc1C21c2cc(N(c3ccc4oc5ccccc5c4c3)c3ccc4c5ccccc5n(-c5cccc6ccccc56)c4c3)ccc2-c2cccc3cccc1c23 ; c1ccc2c(c1)-c1ccccc1C21c2cc(N(c3ccc4c(c3)oc3ccccc34)c3ccc4c5ccccc5n(-c5cccc6ccccc56)c4c3)ccc2-c2cccc3cccc1c23 ; c1ccc2c(c1)-c1ccccc1C21c2cc(N(c3ccc4c5ccccc5n(-c5cccc6sc7ccccc7c56)c4c3)c3cccc4ccccc34)ccc2-c2cccc3cccc1c23]

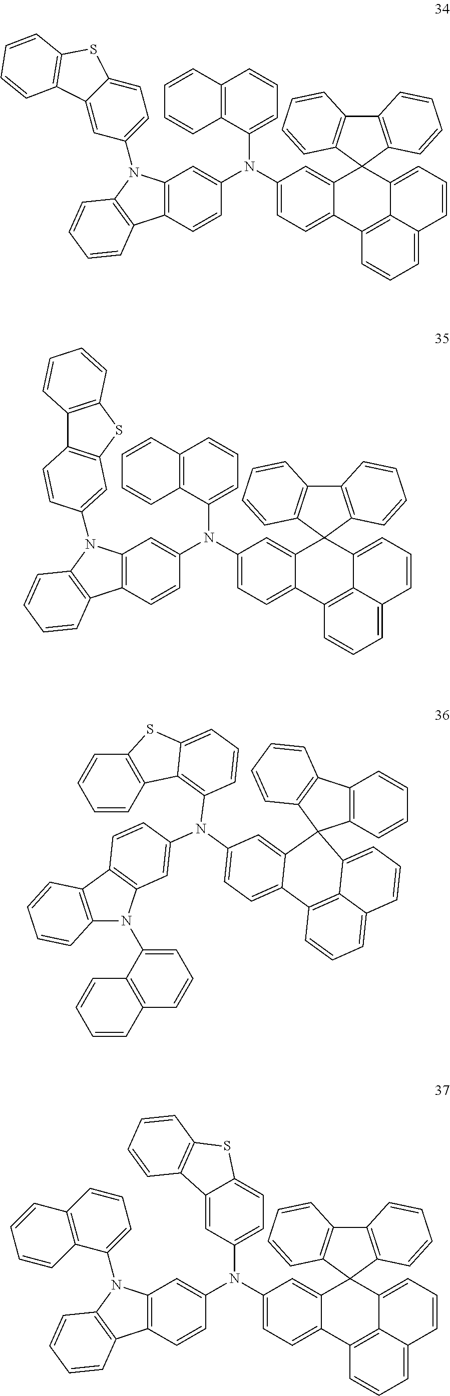 [SMILES: c1ccc2c(c1)-c1ccccc1C21c2cc(N(c3ccc4c5ccccc5n(-c5ccc6sc7ccccc7c6c5)c4c3)c3cccc4ccccc34)ccc2-c2cccc3cccc1c23 ; c1ccc2c(c1)-c1ccccc1C21c2cc(N(c3ccc4c5ccccc5n(-c5ccc6c(c5)sc5ccccc56)c4c3)c3cccc4ccccc34)ccc2-c2cccc3cccc1c23 ; c1ccc2c(c1)-c1ccccc1C21c2cc(N(c3ccc4c5ccccc5n(-c5cccc6ccccc56)c4c3)c3cccc4sc5ccccc5c34)ccc2-c2cccc3cccc1c23 ; c1ccc2c(c1)-c1ccccc1C21c2cc(N(c3ccc4sc5ccccc5c4c3)c3ccc4c5ccccc5n(-c5cccc6ccccc56)c4c3)ccc2-c2cccc3cccc1c23]

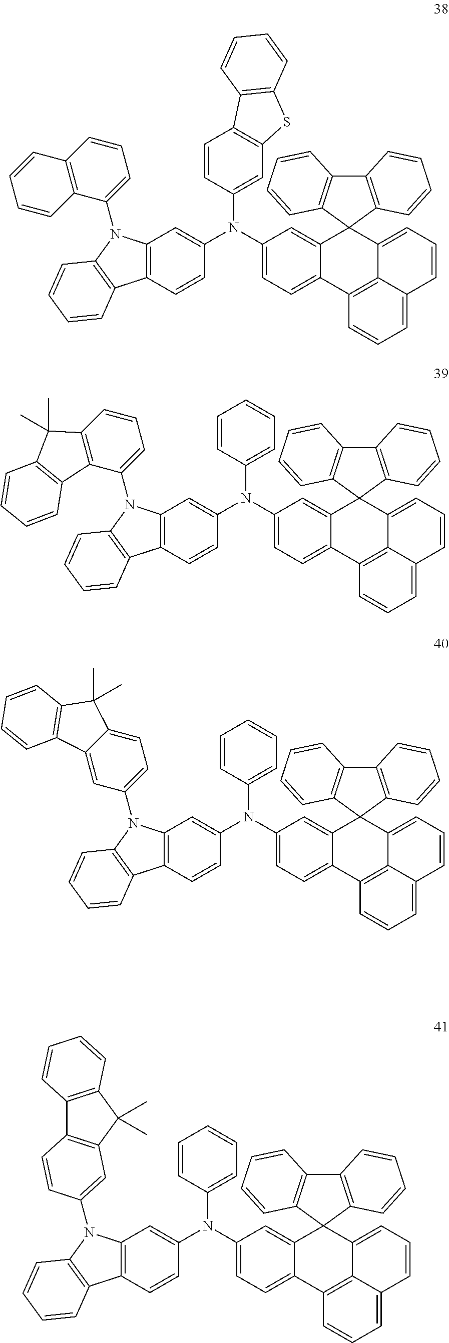 [SMILES: c1ccc2c(c1)-c1ccccc1C21c2cc(N(c3ccc4c(c3)sc3ccccc34)c3ccc4c5ccccc5n(-c5cccc6ccccc56)c4c3)ccc2-c2cccc3cccc1c23 ; CC1(C)c2ccccc2-c2c(-n3c4ccccc4c4ccc(N(c5ccccc5)c5ccc6c(c5)C5(c7ccccc7-c7ccccc75)c5cccc7cccc-6c57)cc43)cccc21 ; CC1(C)c2ccccc2-c2cc(-n3c4ccccc4c4ccc(N(c5ccccc5)c5ccc6c(c5)C5(c7ccccc7-c7ccccc75)c5cccc7cccc-6c57)cc43)ccc21 ; CC1(C)c2ccccc2-c2ccc(-n3c4ccccc4c4ccc(N(c5ccccc5)c5ccc6c(c5)C5(c7ccccc7-c7ccccc75)c5cccc7cccc-6c57)cc43)cc21]

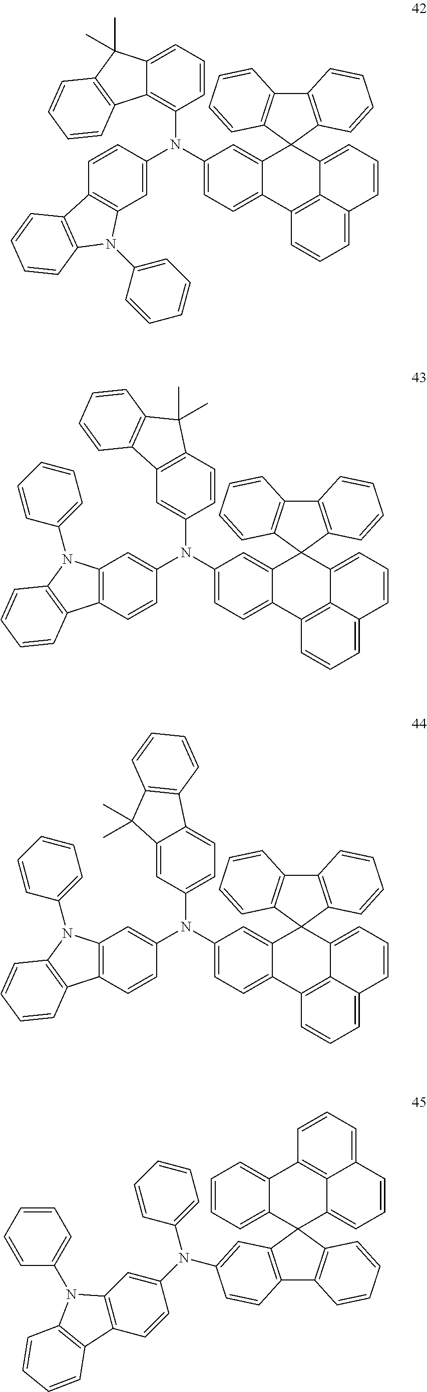 [SMILES: CC1(C)c2ccccc2-c2c(N(c3ccc4c(c3)C3(c5ccccc5-c5ccccc53)c3cccc5cccc-4c35)c3ccc4c5ccccc5n(-c5ccccc5)c4c3)cccc21 ; CC1(C)c2ccccc2-c2cc(N(c3ccc4c(c3)C3(c5ccccc5-c5ccccc53)c3cccc5cccc-4c35)c3ccc4c5ccccc5n(-c5ccccc5)c4c3)ccc21 ; CC1(C)c2ccccc2-c2ccc(N(c3ccc4c(c3)C3(c5ccccc5-c5ccccc53)c3cccc5cccc-4c35)c3ccc4c5ccccc5n(-c5ccccc5)c4c3)cc21 ; c1ccc(N(c2ccc3c(c2)C2(c4ccccc4-3)c3ccccc3-c3cccc4cccc2c34)c2ccc3c4ccccc4n(-c4ccccc4)c3c2)cc1]

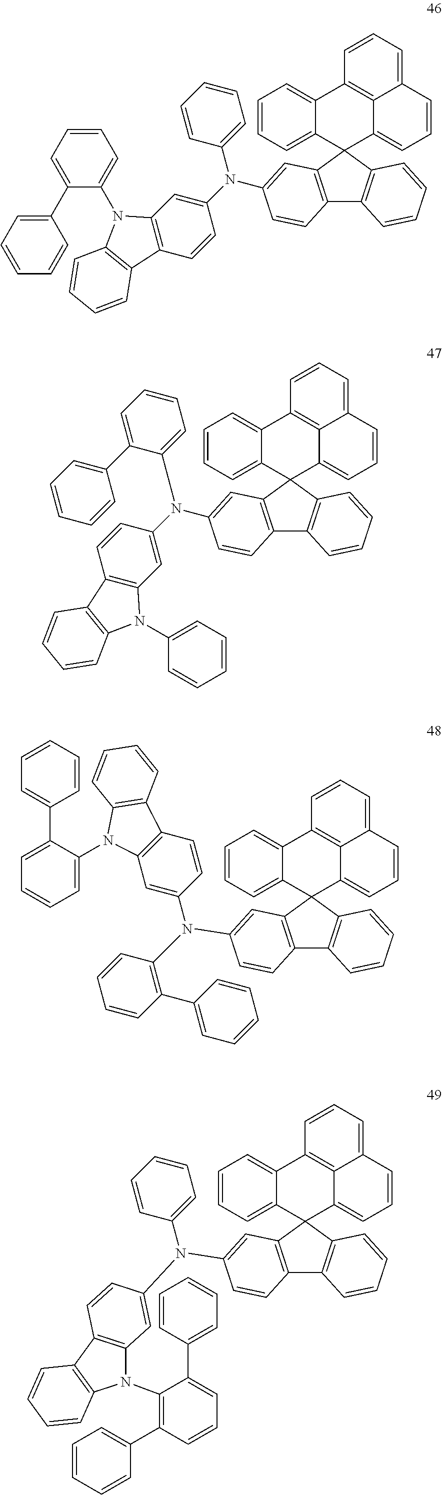 [SMILES: c1ccc(-c2ccccc2-n2c3ccccc3c3ccc(N(c4ccccc4)c4ccc5c(c4)C4(c6ccccc6-5)c5ccccc5-c5cccc6cccc4c56)cc32)cc1 ; c1ccc(-c2ccccc2N(c2ccc3c(c2)C2(c4ccccc4-3)c3ccccc3-c3cccc4cccc2c34)c2ccc3c4ccccc4n(-c4ccccc4)c3c2)cc1 ; c1ccc(-c2ccccc2N(c2ccc3c(c2)C2(c4ccccc4-3)c3ccccc3-c3cccc4cccc2c34)c2ccc3c4ccccc4n(-c4ccccc4-c4ccccc4)c3c2)cc1 ; c1ccc(-c2cccc(-c3ccccc3)c2-n2c3ccccc3c3ccc(N(c4ccccc4)c4ccc5c(c4)C4(c6ccccc6-5)c5ccccc5-c5cccc6cccc4c56)cc32)cc1]

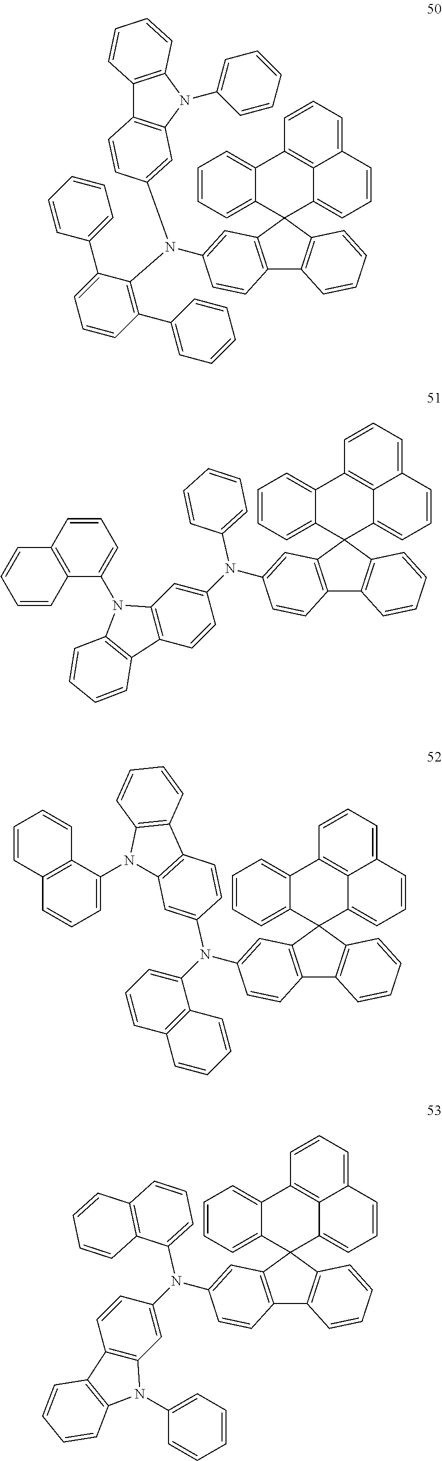 [SMILES: c1ccc(-c2cccc(-c3ccccc3)c2N(c2ccc3c(c2)C2(c4ccccc4-3)c3ccccc3-c3cccc4cccc2c34)c2ccc3c4ccccc4n(-c4ccccc4)c3c2)cc1 ; c1ccc(N(c2ccc3c(c2)C2(c4ccccc4-3)c3ccccc3-c3cccc4cccc2c34)c2ccc3c4ccccc4n(-c4cccc5ccccc45)c3c2)cc1 ; c1ccc2c(c1)-c1ccc(N(c3ccc4c5ccccc5n(-c5cccc6ccccc56)c4c3)c3cccc4ccccc34)cc1C21c2ccccc2-c2cccc3cccc1c23 ; c1ccc(-n2c3ccccc3c3ccc(N(c4ccc5c(c4)C4(c6ccccc6-5)c5ccccc5-c5cccc6cccc4c56)c4cccc5ccccc45)cc32)cc1]

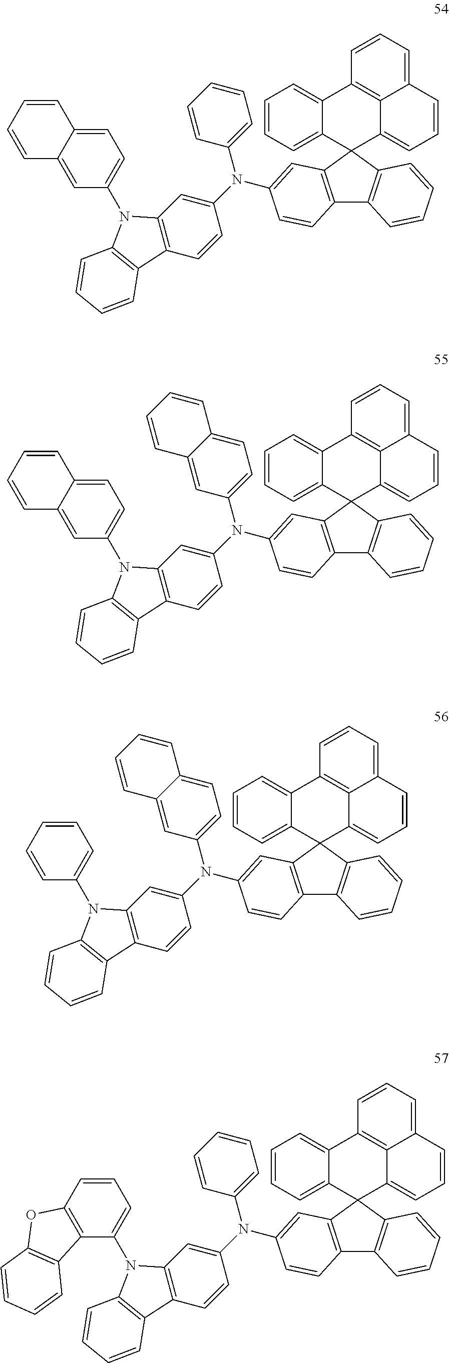 [SMILES: c1ccc(N(c2ccc3c(c2)C2(c4ccccc4-3)c3ccccc3-c3cccc4cccc2c34)c2ccc3c4ccccc4n(-c4ccc5ccccc5c4)c3c2)cc1 ; c1ccc2c(c1)-c1ccc(N(c3ccc4ccccc4c3)c3ccc4c5ccccc5n(-c5ccc6ccccc6c5)c4c3)cc1C21c2ccccc2-c2cccc3cccc1c23 ; c1ccc(-n2c3ccccc3c3ccc(N(c4ccc5c(c4)C4(c6ccccc6-5)c5ccccc5-c5cccc6cccc4c56)c4ccc5ccccc5c4)cc32)cc1 ; c1ccc(N(c2ccc3c(c2)C2(c4ccccc4-3)c3ccccc3-c3cccc4cccc2c34)c2ccc3c4ccccc4n(-c4cccc5oc6ccccc6c45)c3c2)cc1]

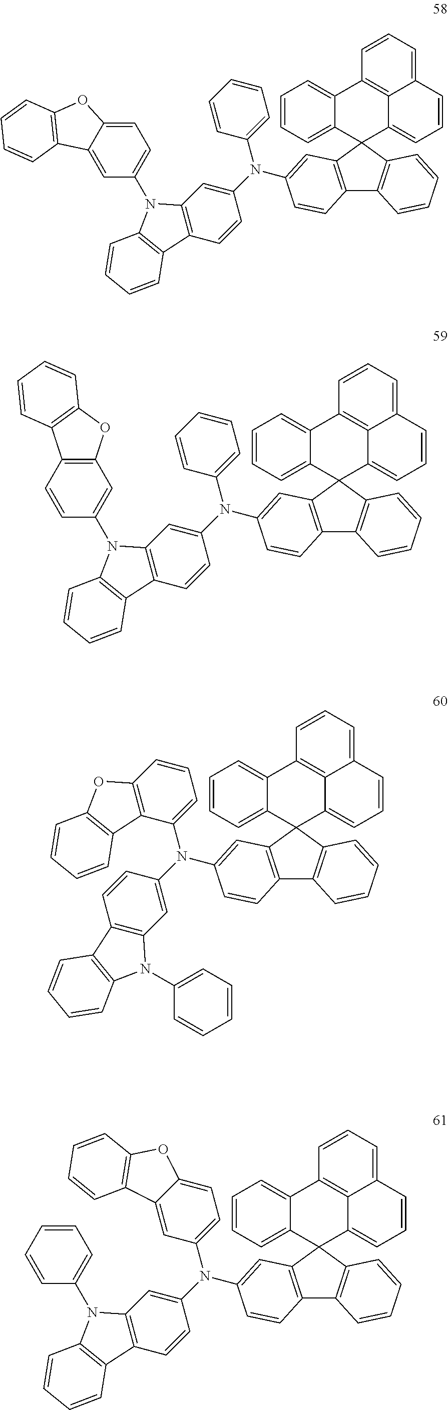 [SMILES: c1ccc(N(c2ccc3c(c2)C2(c4ccccc4-3)c3ccccc3-c3cccc4cccc2c34)c2ccc3c4ccccc4n(-c4ccc5oc6ccccc6c5c4)c3c2)cc1 ; c1ccc(N(c2ccc3c(c2)C2(c4ccccc4-3)c3ccccc3-c3cccc4cccc2c34)c2ccc3c4ccccc4n(-c4ccc5c(c4)oc4ccccc45)c3c2)cc1 ; c1ccc(-n2c3ccccc3c3ccc(N(c4ccc5c(c4)C4(c6ccccc6-5)c5ccccc5-c5cccc6cccc4c56)c4cccc5oc6ccccc6c45)cc32)cc1 ; c1ccc(-n2c3ccccc3c3ccc(N(c4ccc5c(c4)C4(c6ccccc6-5)c5ccccc5-c5cccc6cccc4c56)c4ccc5oc6ccccc6c5c4)cc32)cc1]

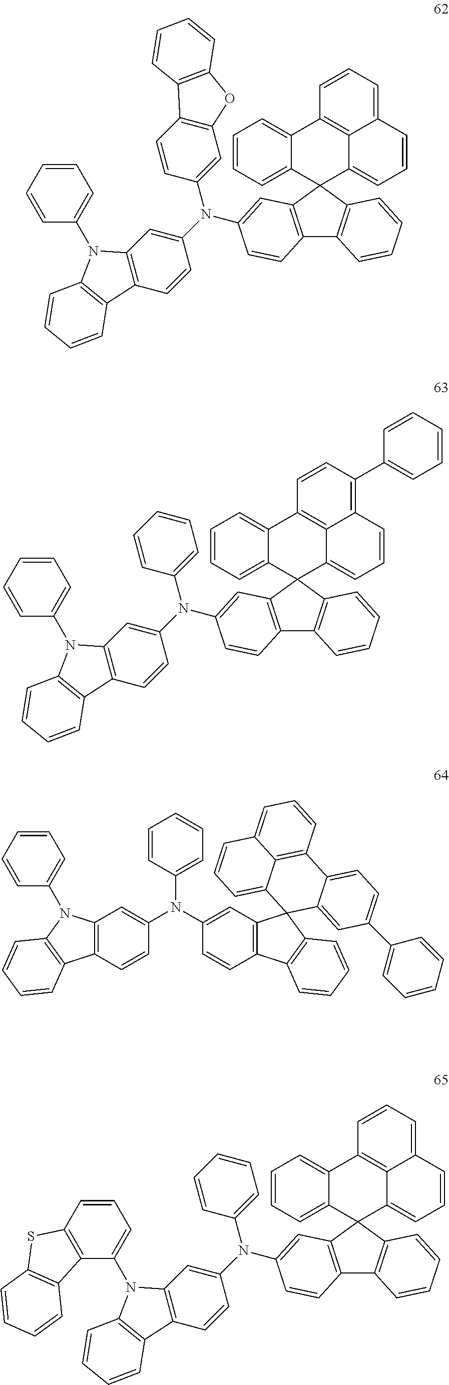 [SMILES: c1ccc(-n2c3ccccc3c3ccc(N(c4ccc5c(c4)C4(c6ccccc6-5)c5ccccc5-c5cccc6cccc4c56)c4ccc5c(c4)oc4ccccc45)cc32)cc1 ; c1ccc(-c2ccc3c4c(cccc24)C2(c4ccccc4-c4ccc(N(c5ccccc5)c5ccc6c7ccccc7n(-c7ccccc7)c6c5)cc42)c2ccccc2-3)cc1 ; c1ccc(-c2ccc3c(c2)C2(c4ccccc4-c4ccc(N(c5ccccc5)c5ccc6c7ccccc7n(-c7ccccc7)c6c5)cc42)c2cccc4cccc-3c24)cc1 ; c1ccc(N(c2ccc3c(c2)C2(c4ccccc4-3)c3ccccc3-c3cccc4cccc2c34)c2ccc3c4ccccc4n(-c4cccc5sc6ccccc6c45)c3c2)cc1]

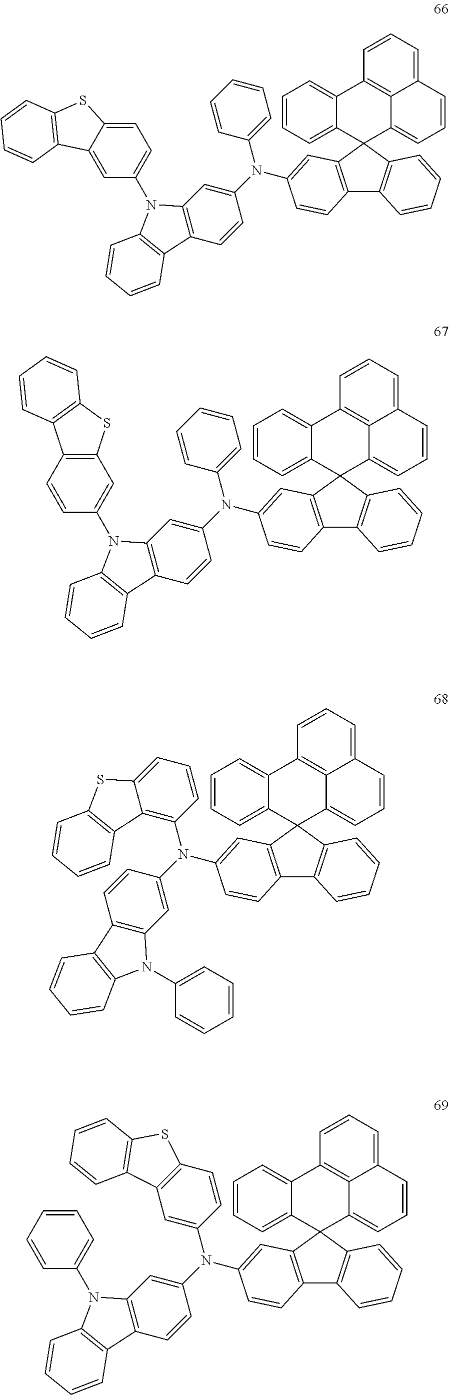 [SMILES: c1ccc(N(c2ccc3c(c2)C2(c4ccccc4-3)c3ccccc3-c3cccc4cccc2c34)c2ccc3c4ccccc4n(-c4ccc5sc6ccccc6c5c4)c3c2)cc1 ; c1ccc(N(c2ccc3c(c2)C2(c4ccccc4-3)c3ccccc3-c3cccc4cccc2c34)c2ccc3c4ccccc4n(-c4ccc5c(c4)sc4ccccc45)c3c2)cc1 ; c1ccc(-n2c3ccccc3c3ccc(N(c4ccc5c(c4)C4(c6ccccc6-5)c5ccccc5-c5cccc6cccc4c56)c4cccc5sc6ccccc6c45)cc32)cc1 ; c1ccc(-n2c3ccccc3c3ccc(N(c4ccc5c(c4)C4(c6ccccc6-5)c5ccccc5-c5cccc6cccc4c56)c4ccc5sc6ccccc6c5c4)cc32)cc1]

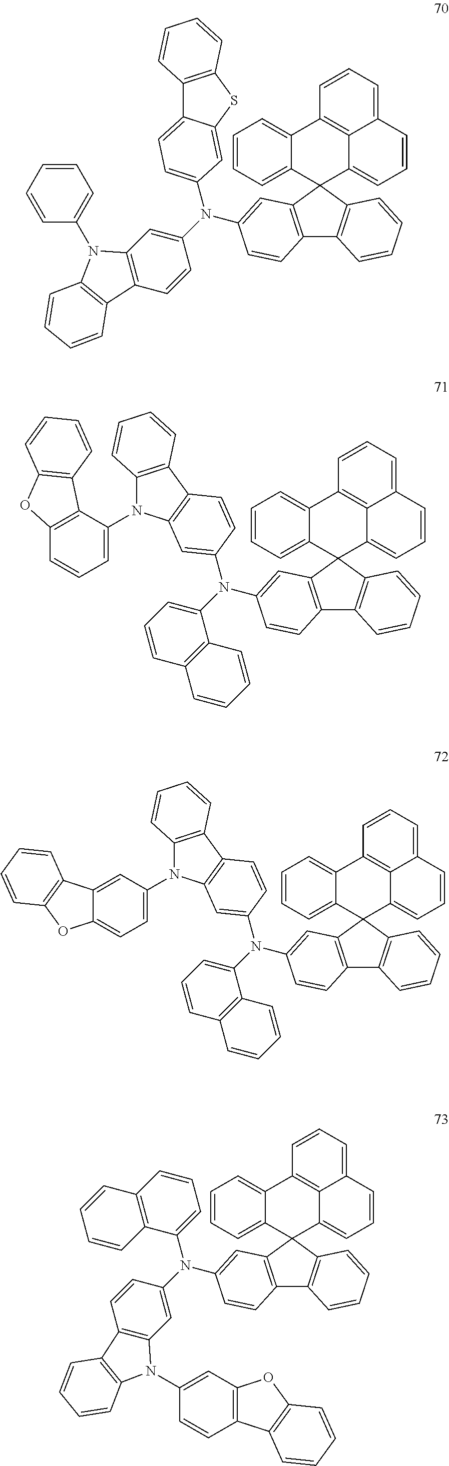 [SMILES: c1ccc(-n2c3ccccc3c3ccc(N(c4ccc5c(c4)C4(c6ccccc6-5)c5ccccc5-c5cccc6cccc4c56)c4ccc5c(c4)sc4ccccc45)cc32)cc1 ; c1ccc2c(c1)-c1ccc(N(c3ccc4c5ccccc5n(-c5cccc6oc7ccccc7c56)c4c3)c3cccc4ccccc34)cc1C21c2ccccc2-c2cccc3cccc1c23 ; c1ccc2c(c1)-c1ccc(N(c3ccc4c5ccccc5n(-c5ccc6oc7ccccc7c6c5)c4c3)c3cccc4ccccc34)cc1C21c2ccccc2-c2cccc3cccc1c23 ; c1ccc2c(c1)-c1ccc(N(c3ccc4c5ccccc5n(-c5ccc6c(c5)oc5ccccc56)c4c3)c3cccc4ccccc34)cc1C21c2ccccc2-c2cccc3cccc1c23]

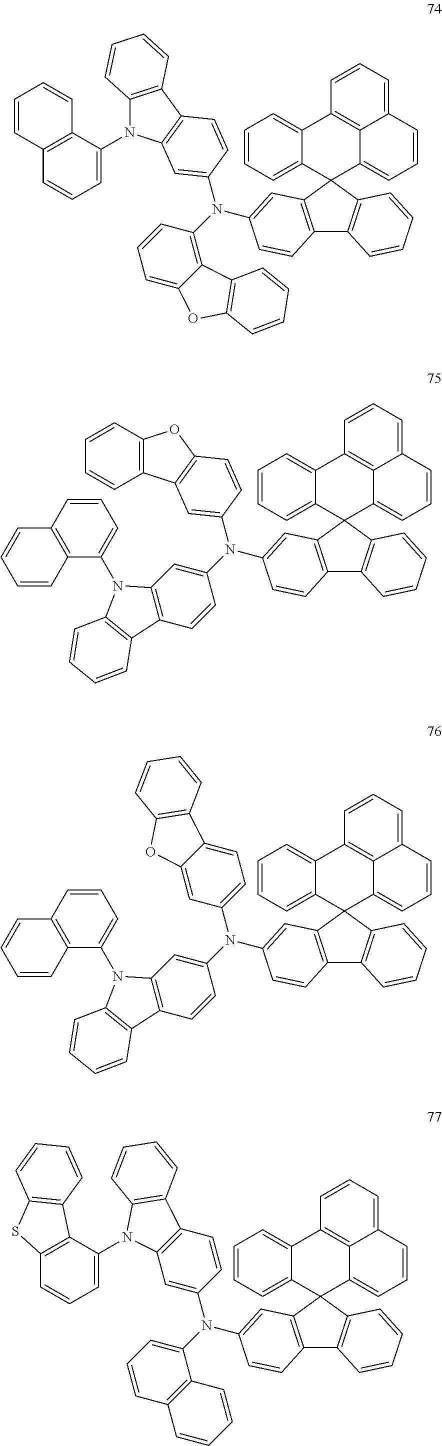 [SMILES: c1ccc2c(c1)-c1ccc(N(c3ccc4c5ccccc5n(-c5cccc6ccccc56)c4c3)c3cccc4oc5ccccc5c34)cc1C21c2ccccc2-c2cccc3cccc1c23 ; c1ccc2c(c1)-c1ccc(N(c3ccc4oc5ccccc5c4c3)c3ccc4c5ccccc5n(-c5cccc6ccccc56)c4c3)cc1C21c2ccccc2-c2cccc3cccc1c23 ; c1ccc2c(c1)-c1ccc(N(c3ccc4c(c3)oc3ccccc34)c3ccc4c5ccccc5n(-c5cccc6ccccc56)c4c3)cc1C21c2ccccc2-c2cccc3cccc1c23 ; c1ccc2c(c1)-c1ccc(N(c3ccc4c5ccccc5n(-c5cccc6sc7ccccc7c56)c4c3)c3cccc4ccccc34)cc1C21c2ccccc2-c2cccc3cccc1c23]

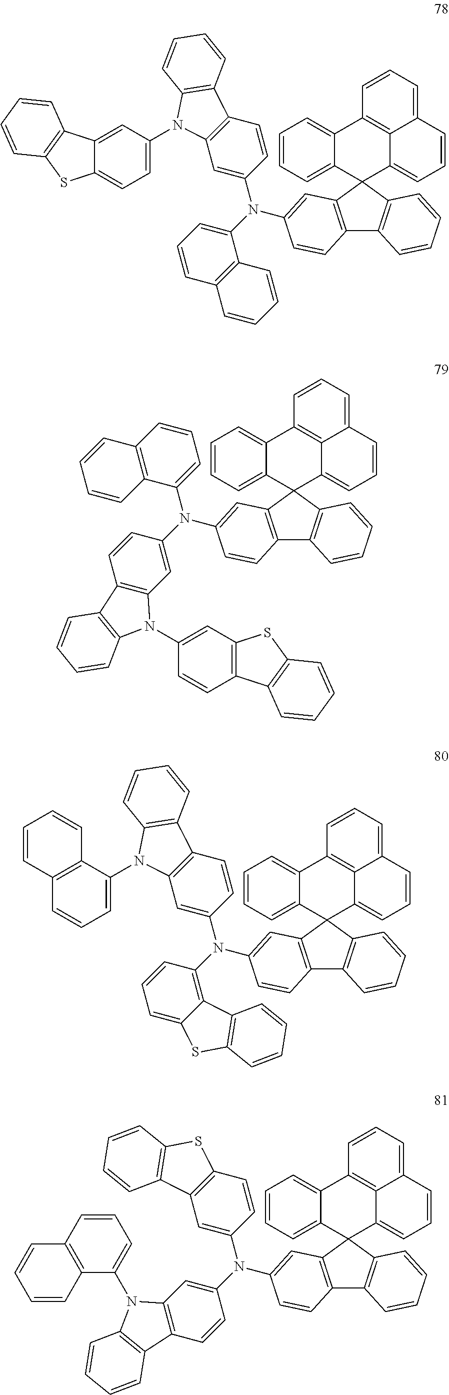 [SMILES: c1ccc2c(c1)-c1ccc(N(c3ccc4c5ccccc5n(-c5ccc6sc7ccccc7c6c5)c4c3)c3cccc4ccccc34)cc1C21c2ccccc2-c2cccc3cccc1c23 ; c1ccc2c(c1)-c1ccc(N(c3ccc4c5ccccc5n(-c5ccc6c(c5)sc5ccccc56)c4c3)c3cccc4ccccc34)cc1C21c2ccccc2-c2cccc3cccc1c23 ; c1ccc2c(c1)-c1ccc(N(c3ccc4c5ccccc5n(-c5cccc6ccccc56)c4c3)c3cccc4sc5ccccc5c34)cc1C21c2ccccc2-c2cccc3cccc1c23 ; c1ccc2c(c1)-c1ccc(N(c3ccc4sc5ccccc5c4c3)c3ccc4c5ccccc5n(-c5cccc6ccccc56)c4c3)cc1C21c2ccccc2-c2cccc3cccc1c23]

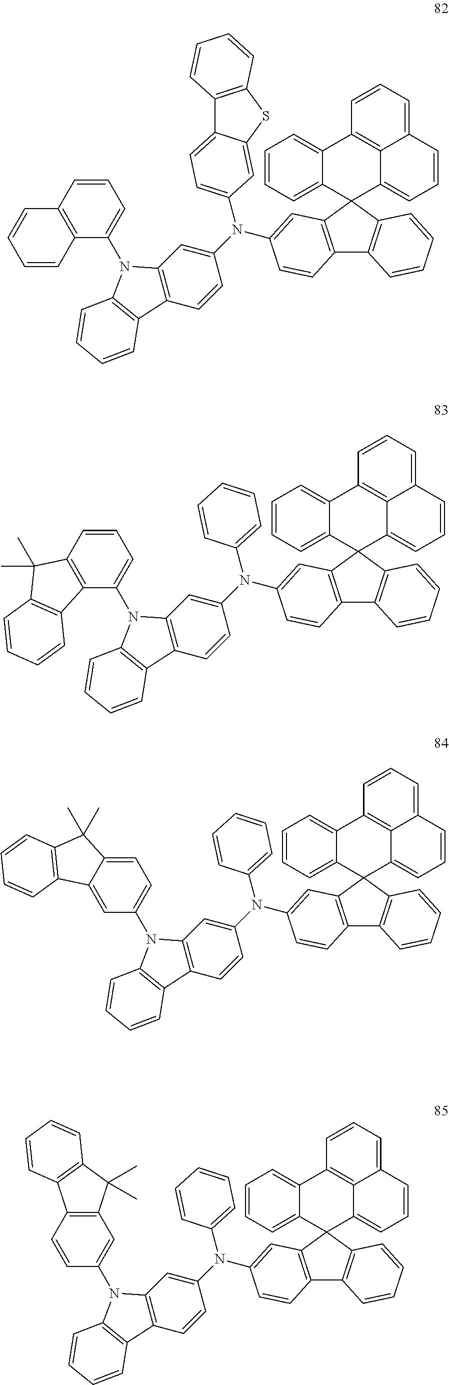 [SMILES: c1ccc2c(c1)-c1ccc(N(c3ccc4c(c3)sc3ccccc34)c3ccc4c5ccccc5n(-c5cccc6ccccc56)c4c3)cc1C21c2ccccc2-c2cccc3cccc1c23 ; CC1(C)c2ccccc2-c2c(-n3c4ccccc4c4ccc(N(c5ccccc5)c5ccc6c(c5)C5(c7ccccc7-6)c6ccccc6-c6cccc7cccc5c67)cc43)cccc21 ; CC1(C)c2ccccc2-c2cc(-n3c4ccccc4c4ccc(N(c5ccccc5)c5ccc6c(c5)C5(c7ccccc7-6)c6ccccc6-c6cccc7cccc5c67)cc43)ccc21 ; CC1(C)c2ccccc2-c2ccc(-n3c4ccccc4c4ccc(N(c5ccccc5)c5ccc6c(c5)C5(c7ccccc7-6)c6ccccc6-c6cccc7cccc5c67)cc43)cc21]

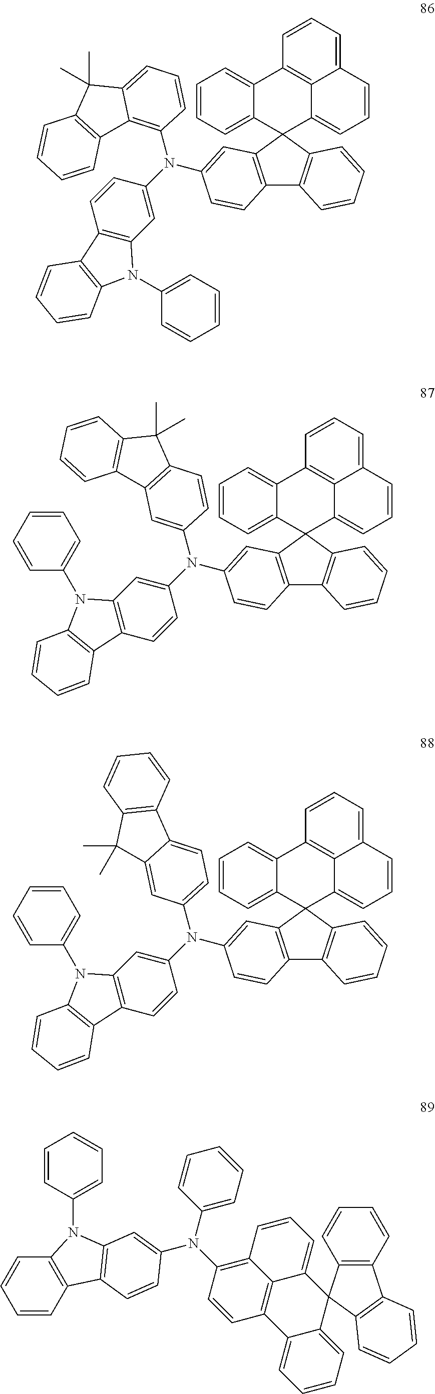 [SMILES: CC1(C)c2ccccc2-c2c(N(c3ccc4c(c3)C3(c5ccccc5-4)c4ccccc4-c4cccc5cccc3c45)c3ccc4c5ccccc5n(-c5ccccc5)c4c3)cccc21 ; CC1(C)c2ccccc2-c2cc(N(c3ccc4c(c3)C3(c5ccccc5-4)c4ccccc4-c4cccc5cccc3c45)c3ccc4c5ccccc5n(-c5ccccc5)c4c3)ccc21 ; CC1(C)c2ccccc2-c2ccc(N(c3ccc4c(c3)C3(c5ccccc5-4)c4ccccc4-c4cccc5cccc3c45)c3ccc4c5ccccc5n(-c5ccccc5)c4c3)cc21 ; c1ccc(N(c2ccc3c4ccccc4n(-c4ccccc4)c3c2)c2ccc3c4c(cccc24)C2(c4ccccc4-c4ccccc42)c2ccccc2-3)cc1]

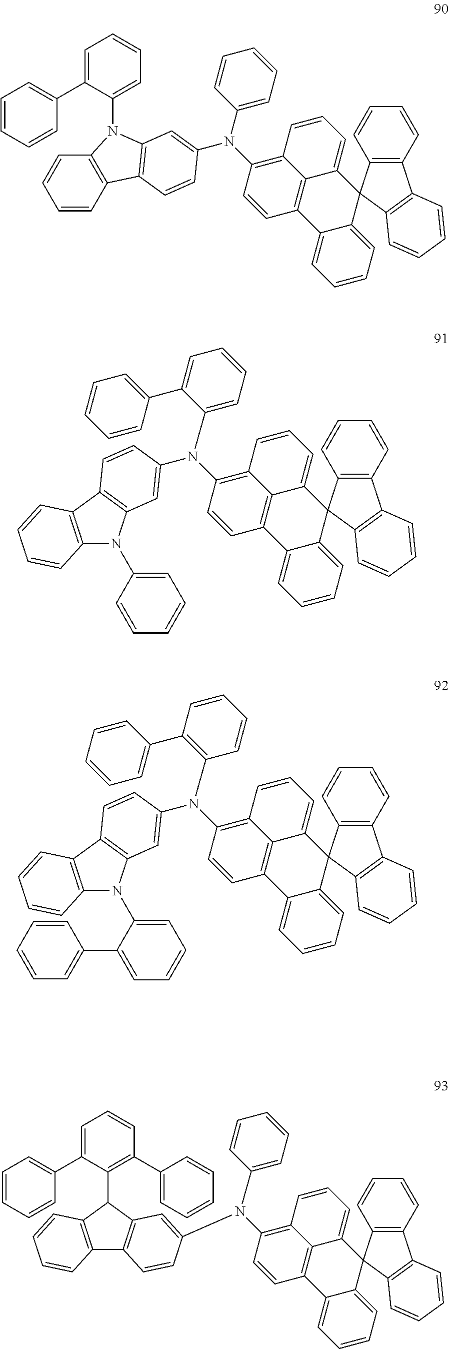 [SMILES: c1ccc(-c2ccccc2-n2c3ccccc3c3ccc(N(c4ccccc4)c4ccc5c6c(cccc46)C4(c6ccccc6-c6ccccc64)c4ccccc4-5)cc32)cc1 ; c1ccc(-c2ccccc2N(c2ccc3c4ccccc4n(-c4ccccc4)c3c2)c2ccc3c4c(cccc24)C2(c4ccccc4-c4ccccc42)c2ccccc2-3)cc1 ; c1ccc(-c2ccccc2N(c2ccc3c4ccccc4n(-c4ccccc4-c4ccccc4)c3c2)c2ccc3c4c(cccc24)C2(c4ccccc4-c4ccccc42)c2ccccc2-3)cc1 ; c1ccc(-c2cccc(-c3ccccc3)c2C2c3ccccc3-c3ccc(N(c4ccccc4)c4ccc5c6c(cccc46)C4(c6ccccc6-c6ccccc64)c4ccccc4-5)cc32)cc1]

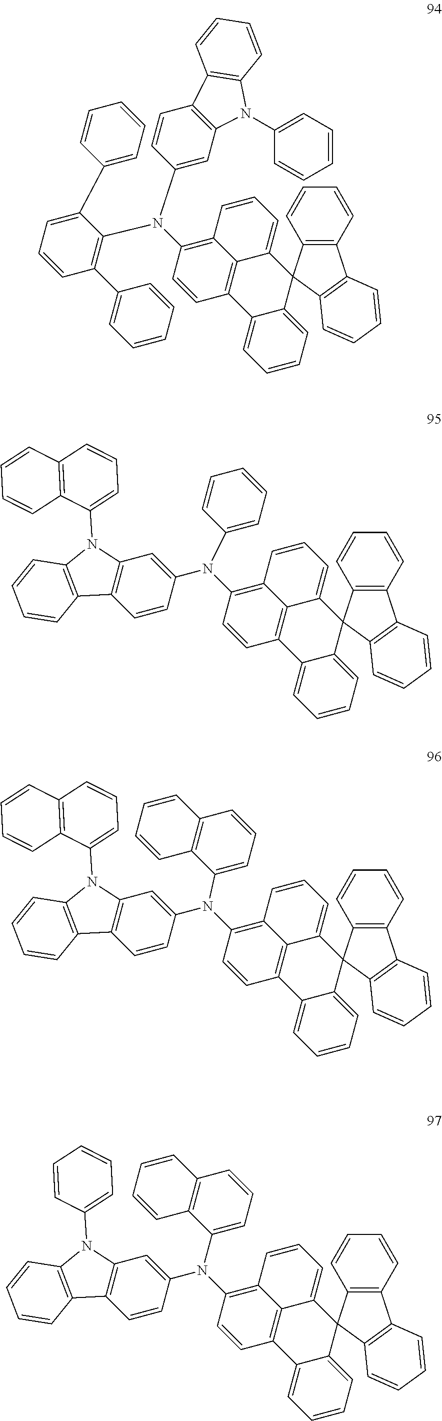 [SMILES: c1ccc(-c2cccc(-c3ccccc3)c2N(c2ccc3c4ccccc4n(-c4ccccc4)c3c2)c2ccc3c4c(cccc24)C2(c4ccccc4-c4ccccc42)c2ccccc2-3)cc1 ; c1ccc(N(c2ccc3c4ccccc4n(-c4cccc5ccccc45)c3c2)c2ccc3c4c(cccc24)C2(c4ccccc4-c4ccccc42)c2ccccc2-3)cc1 ; c1ccc2c(c1)-c1ccccc1C21c2ccccc2-c2ccc(N(c3ccc4c5ccccc5n(-c5cccc6ccccc56)c4c3)c3cccc4ccccc34)c3cccc1c23 ; c1ccc(-n2c3ccccc3c3ccc(N(c4cccc5ccccc45)c4ccc5c6c(cccc46)C4(c6ccccc6-c6ccccc64)c4ccccc4-5)cc32)cc1]

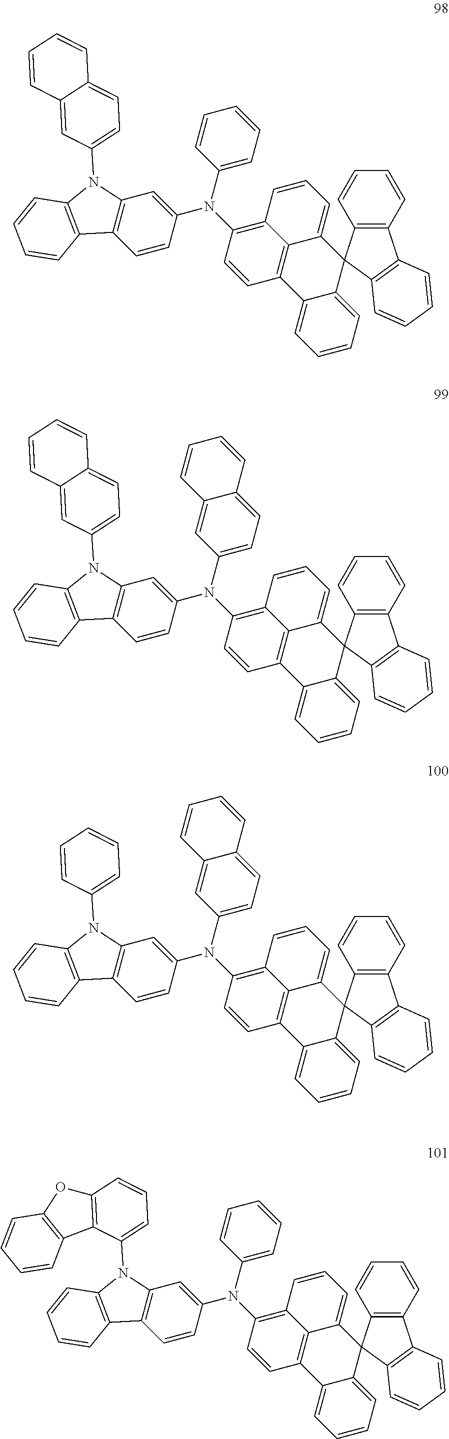 [SMILES: c1ccc(N(c2ccc3c4ccccc4n(-c4ccc5ccccc5c4)c3c2)c2ccc3c4c(cccc24)C2(c4ccccc4-c4ccccc42)c2ccccc2-3)cc1 ; c1ccc2c(c1)-c1ccccc1C21c2ccccc2-c2ccc(N(c3ccc4ccccc4c3)c3ccc4c5ccccc5n(-c5ccc6ccccc6c5)c4c3)c3cccc1c23 ; c1ccc(-n2c3ccccc3c3ccc(N(c4ccc5ccccc5c4)c4ccc5c6c(cccc46)C4(c6ccccc6-c6ccccc64)c4ccccc4-5)cc32)cc1 ; c1ccc(N(c2ccc3c4ccccc4n(-c4cccc5oc6ccccc6c45)c3c2)c2ccc3c4c(cccc24)C2(c4ccccc4-c4ccccc42)c2ccccc2-3)cc1]

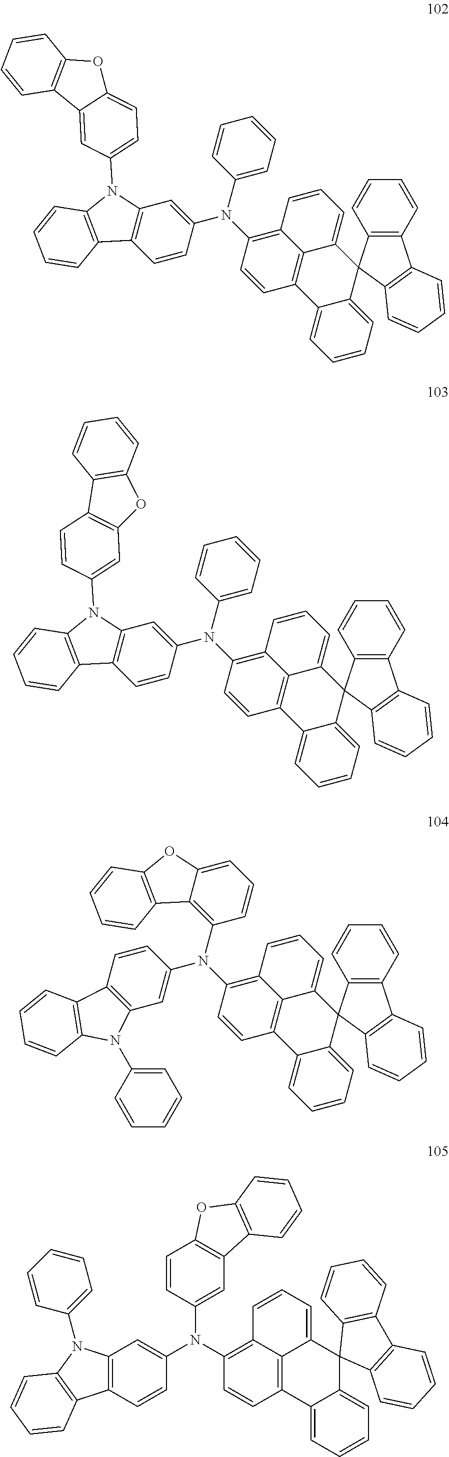 [SMILES: c1ccc(N(c2ccc3c4ccccc4n(-c4ccc5oc6ccccc6c5c4)c3c2)c2ccc3c4c(cccc24)C2(c4ccccc4-c4ccccc42)c2ccccc2-3)cc1 ; c1ccc(N(c2ccc3c4ccccc4n(-c4ccc5c(c4)oc4ccccc45)c3c2)c2ccc3c4c(cccc24)C2(c4ccccc4-c4ccccc42)c2ccccc2-3)cc1 ; c1ccc(-n2c3ccccc3c3ccc(N(c4ccc5c6c(cccc46)C4(c6ccccc6-c6ccccc64)c4ccccc4-5)c4cccc5oc6ccccc6c45)cc32)cc1 ; c1ccc(-n2c3ccccc3c3ccc(N(c4ccc5oc6ccccc6c5c4)c4ccc5c6c(cccc46)C4(c6ccccc6-c6ccccc64)c4ccccc4-5)cc32)cc1]

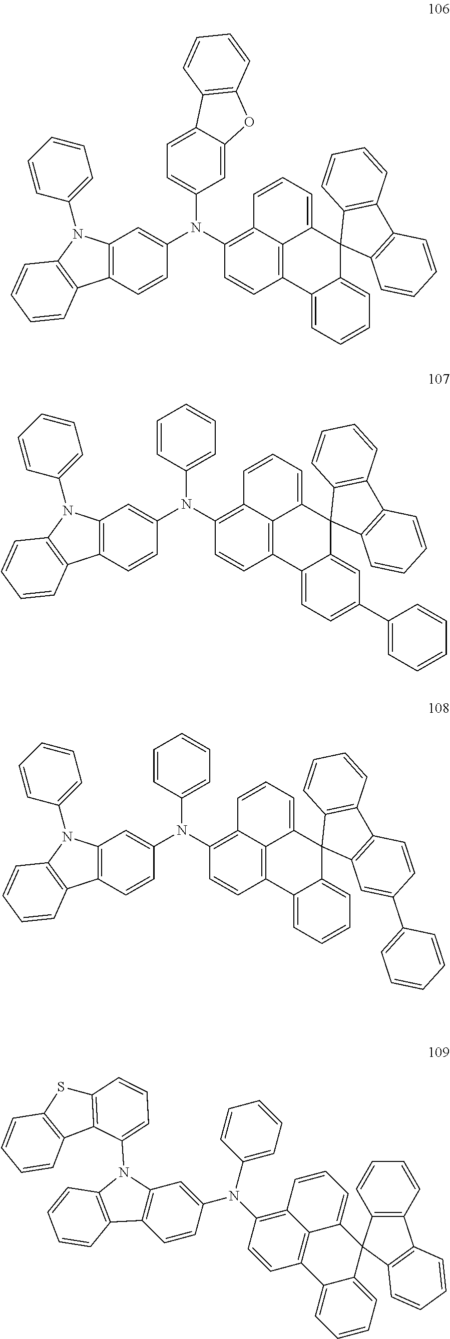 [SMILES: c1ccc(-n2c3ccccc3c3ccc(N(c4ccc5c(c4)oc4ccccc45)c4ccc5c6c(cccc46)C4(c6ccccc6-c6ccccc64)c4ccccc4-5)cc32)cc1 ; c1ccc(-c2ccc3c(c2)C2(c4ccccc4-c4ccccc42)c2cccc4c(N(c5ccccc5)c5ccc6c7ccccc7n(-c7ccccc7)c6c5)ccc-3c24)cc1 ; c1ccc(-c2ccc3c(c2)C2(c4ccccc4-3)c3ccccc3-c3ccc(N(c4ccccc4)c4ccc5c6ccccc6n(-c6ccccc6)c5c4)c4cccc2c34)cc1 ; c1ccc(N(c2ccc3c4ccccc4n(-c4cccc5sc6ccccc6c45)c3c2)c2ccc3c4c(cccc24)C2(c4ccccc4-c4ccccc42)c2ccccc2-3)cc1]

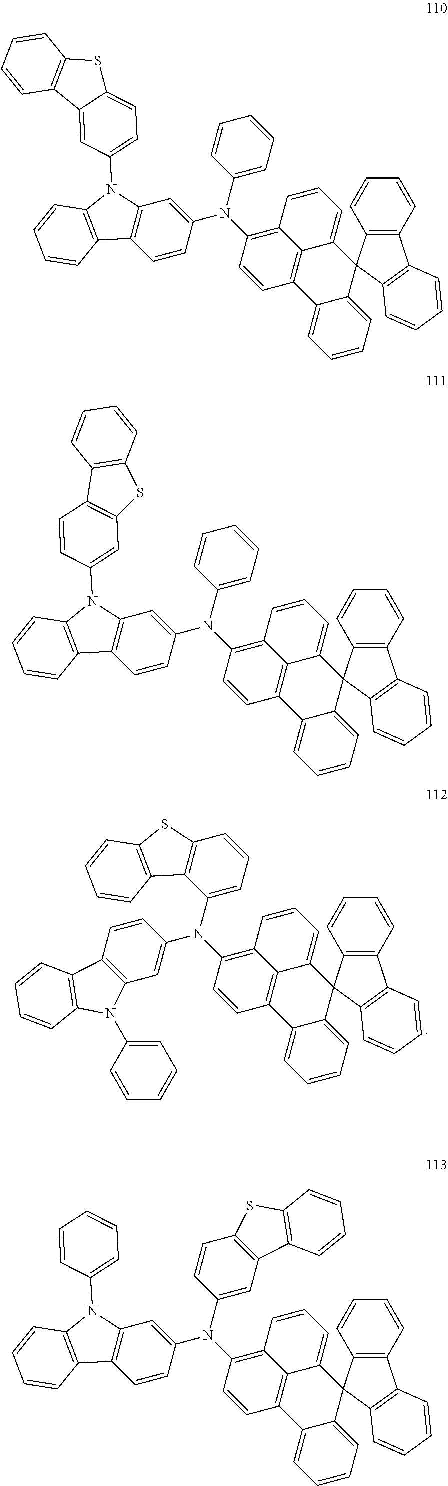 [SMILES: c1ccc(N(c2ccc3c4ccccc4n(-c4ccc5sc6ccccc6c5c4)c3c2)c2ccc3c4c(cccc24)C2(c4ccccc4-c4ccccc42)c2ccccc2-3)cc1 ; c1ccc(N(c2ccc3c4ccccc4n(-c4ccc5c(c4)sc4ccccc45)c3c2)c2ccc3c4c(cccc24)C2(c4ccccc4-c4ccccc42)c2ccccc2-3)cc1 ; c1ccc(-n2c3ccccc3c3ccc(N(c4ccc5c6c(cccc46)C4(c6ccccc6-c6ccccc64)c4ccccc4-5)c4cccc5sc6ccccc6c45)cc32)cc1 ; c1ccc(-n2c3ccccc3c3ccc(N(c4ccc5sc6ccccc6c5c4)c4ccc5c6c(cccc46)C4(c6ccccc6-c6ccccc64)c4ccccc4-5)cc32)cc1]

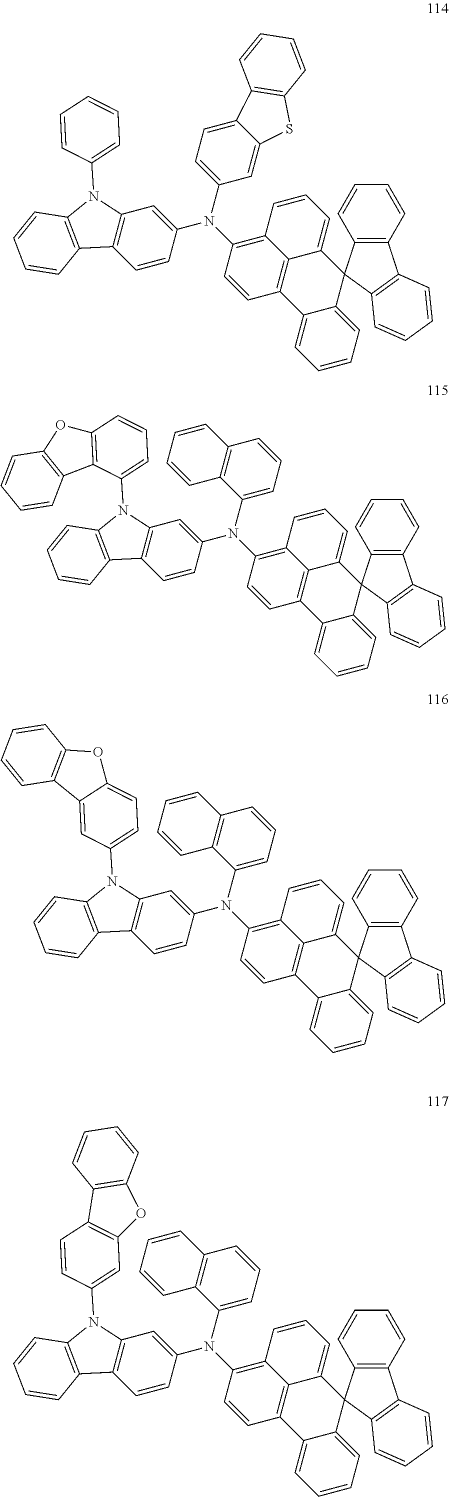 [SMILES: c1ccc(-n2c3ccccc3c3ccc(N(c4ccc5c(c4)sc4ccccc45)c4ccc5c6c(cccc46)C4(c6ccccc6-c6ccccc64)c4ccccc4-5)cc32)cc1 ; c1ccc2c(c1)-c1ccccc1C21c2ccccc2-c2ccc(N(c3ccc4c5ccccc5n(-c5cccc6oc7ccccc7c56)c4c3)c3cccc4ccccc34)c3cccc1c23 ; c1ccc2c(c1)-c1ccccc1C21c2ccccc2-c2ccc(N(c3ccc4c5ccccc5n(-c5ccc6oc7ccccc7c6c5)c4c3)c3cccc4ccccc34)c3cccc1c23 ; c1ccc2c(c1)-c1ccccc1C21c2ccccc2-c2ccc(N(c3ccc4c5ccccc5n(-c5ccc6c(c5)oc5ccccc56)c4c3)c3cccc4ccccc34)c3cccc1c23]

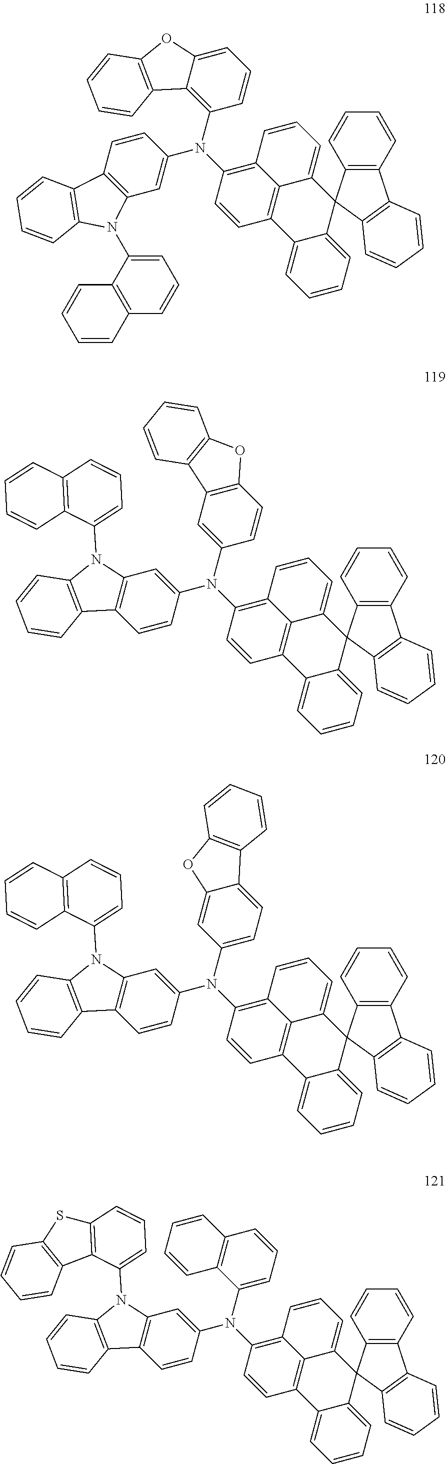 [SMILES: c1ccc2c(c1)-c1ccccc1C21c2ccccc2-c2ccc(N(c3ccc4c5ccccc5n(-c5cccc6ccccc56)c4c3)c3cccc4oc5ccccc5c34)c3cccc1c23 ; c1ccc2c(c1)-c1ccccc1C21c2ccccc2-c2ccc(N(c3ccc4oc5ccccc5c4c3)c3ccc4c5ccccc5n(-c5cccc6ccccc56)c4c3)c3cccc1c23 ; c1ccc2c(c1)-c1ccccc1C21c2ccccc2-c2ccc(N(c3ccc4c(c3)oc3ccccc34)c3ccc4c5ccccc5n(-c5cccc6ccccc56)c4c3)c3cccc1c23 ; c1ccc2c(c1)-c1ccccc1C21c2ccccc2-c2ccc(N(c3ccc4c5ccccc5n(-c5cccc6sc7ccccc7c56)c4c3)c3cccc4ccccc34)c3cccc1c23]

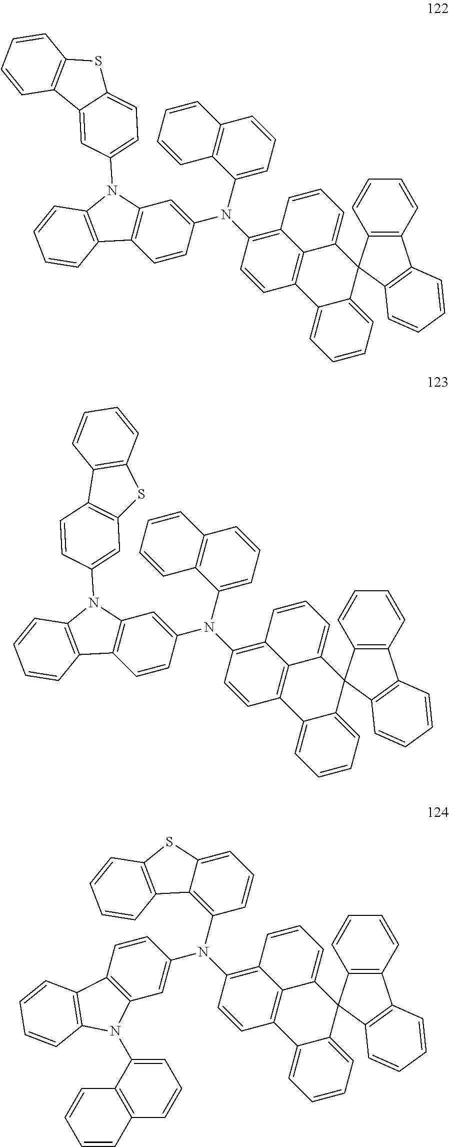 [SMILES: c1ccc2c(c1)-c1ccccc1C21c2ccccc2-c2ccc(N(c3ccc4c5ccccc5n(-c5ccc6sc7ccccc7c6c5)c4c3)c3cccc4ccccc34)c3cccc1c23 ; c1ccc2c(c1)-c1ccccc1C21c2ccccc2-c2ccc(N(c3ccc4c5ccccc5n(-c5ccc6c(c5)sc5ccccc56)c4c3)c3cccc4ccccc34)c3cccc1c23 ; c1ccc2c(c1)-c1ccccc1C21c2ccccc2-c2ccc(N(c3ccc4c5ccccc5n(-c5cccc6ccccc56)c4c3)c3cccc4sc5ccccc5c34)c3cccc1c23]

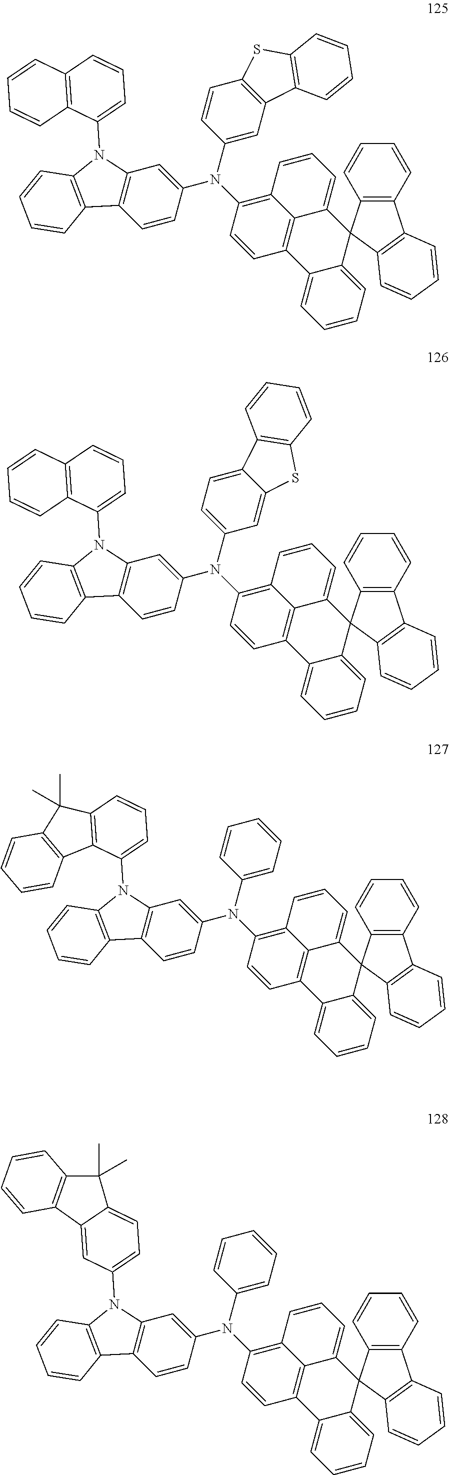 [SMILES: c1ccc2c(c1)-c1ccccc1C21c2ccccc2-c2ccc(N(c3ccc4sc5ccccc5c4c3)c3ccc4c5ccccc5n(-c5cccc6ccccc56)c4c3)c3cccc1c23 ; c1ccc2c(c1)-c1ccccc1C21c2ccccc2-c2ccc(N(c3ccc4c(c3)sc3ccccc34)c3ccc4c5ccccc5n(-c5cccc6ccccc56)c4c3)c3cccc1c23 ; CC1(C)c2ccccc2-c2c(-n3c4ccccc4c4ccc(N(c5ccccc5)c5ccc6c7c(cccc57)C5(c7ccccc7-c7ccccc75)c5ccccc5-6)cc43)cccc21 ; CC1(C)c2ccccc2-c2cc(-n3c4ccccc4c4ccc(N(c5ccccc5)c5ccc6c7c(cccc57)C5(c7ccccc7-c7ccccc75)c5ccccc5-6)cc43)ccc21]

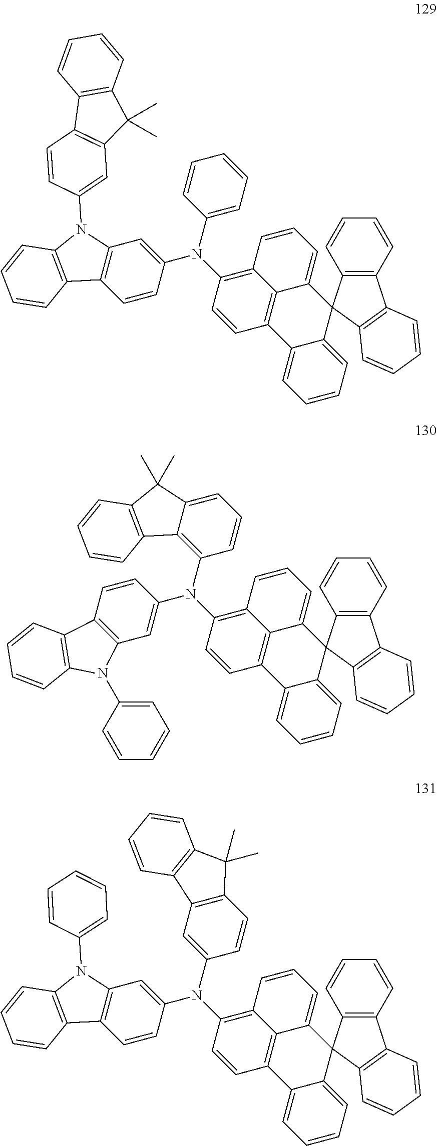 [SMILES: CC1(C)c2ccccc2-c2ccc(-n3c4ccccc4c4ccc(N(c5ccccc5)c5ccc6c7c(cccc57)C5(c7ccccc7-c7ccccc75)c5ccccc5-6)cc43)cc21 ; CC1(C)c2ccccc2-c2c(N(c3ccc4c5ccccc5n(-c5ccccc5)c4c3)c3ccc4c5c(cccc35)C3(c5ccccc5-c5ccccc53)c3ccccc3-4)cccc21 ; CC1(C)c2ccccc2-c2cc(N(c3ccc4c5ccccc5n(-c5ccccc5)c4c3)c3ccc4c5c(cccc35)C3(c5ccccc5-c5ccccc53)c3ccccc3-4)ccc21]

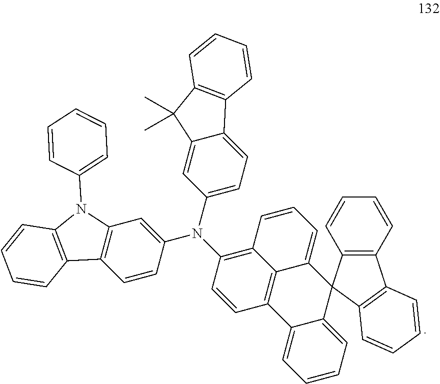 [SMILES: CC1(C)c2ccccc2-c2ccc(N(c3ccc4c5ccccc5n(-c5ccccc5)c4c3)c3ccc4c5c(cccc35)C3(c5ccccc5-c5ccccc53)c3ccccc3-4)cc21]

13. An amine compound represented by Formula 1 below: 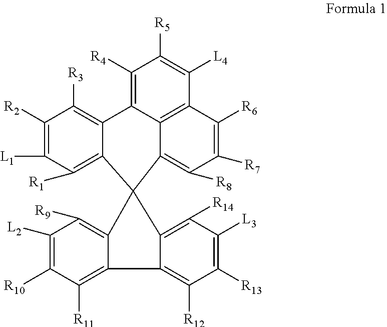
wherein, in Formula 1 above,
R₁ to R₁₄ are each independently a hydrogen atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms, and
L₁ to L₄ are each independently a hydrogen atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms, or a group represented by Formula 2 below,
where at least one selected from among L1 to L4 is represented by Formula 2 below: 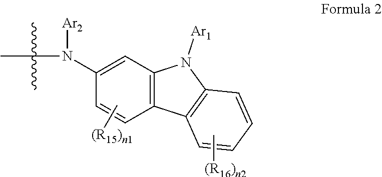
wherein, in Formula 2 above,
R₁₅ and R₁₆ are each independently a hydrogen atom, a deuterium atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms,
Ar₁ and Ar₂ are each independently a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms,
n1 is an integer of 0 to 3, and
n2 is an integer of 0 to 4.

14. The amine compound of claim 13, wherein Formula 1 above is represented by any one selected from among Formula 3-1 to Formula 3-4 below: 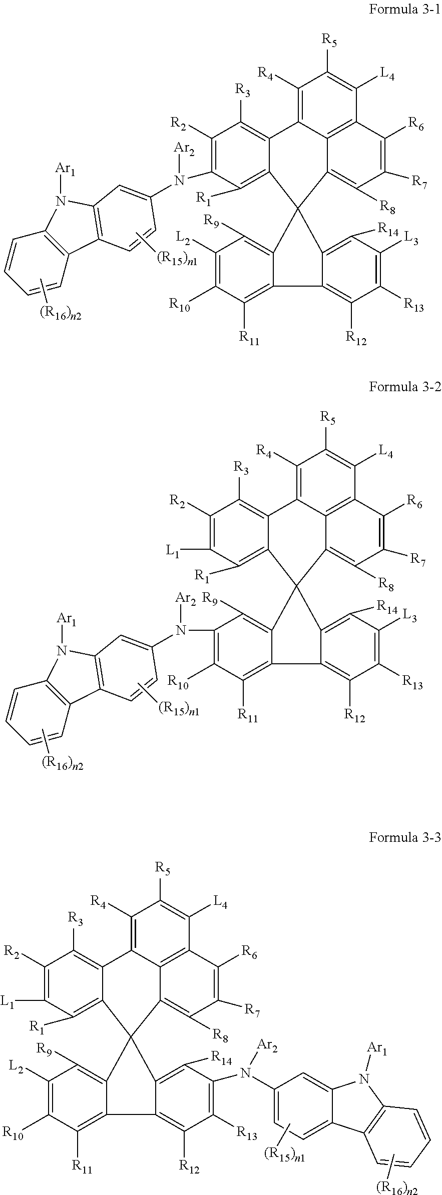 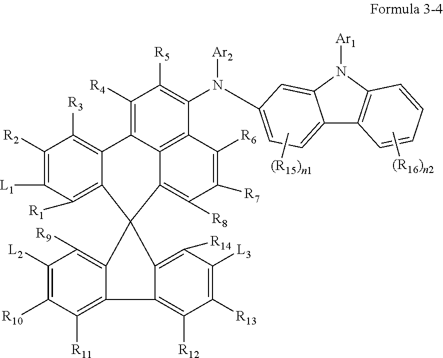
wherein, in Formula 3-1 to Formula 3-4 above,
R₁ to R₁₆, L₁ to L₄, Ar₁, Ar₂, n1, and n2 are the same as defined with respect to Formula 1 and Formula 2.

15. The amine compound of claim 13, wherein R₁ to R₁₄, Ar₁, and Ar₂ do not comprise a substituted or unsubstituted amine group in the amine compound represented by Formula 1 above.

16. The amine compound of claim 13, wherein the Ar₁ and Ar₂ are each independently a substituted or unsubstituted ring-forming aryl group having 6 to 18 carbon atoms.

17. The amine compound of claim 16, wherein the Ar₁ and Ar₂ are each independently represented by any one selected from among Formula 4-1 to Formula 4-5 below: 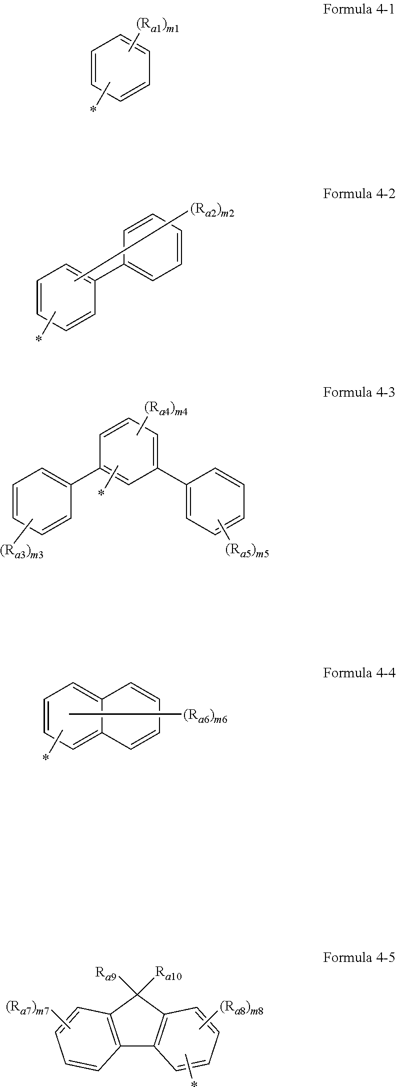
wherein, in Formula 4-1 to Formula 4-5 above,
Rₐ₁ to Rₐ₁₀ are each independently a hydrogen atom, a deuterium atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms,
m1, m3, and m5 are each independently an integer of 0 to 5,
m2 is an integer of 0 to 9,
m4 and m8 are each independently an integer of 0 to 3,
m6 is an integer of 0 to 7, and
m7 is an integer of 0 to 4.

18. The amine compound of claim 13, wherein the Ar₁ and Ar₂ are each independently a substituted or unsubstituted dibenzohetero group.

19. The amine compound of claim 18, wherein the Ar₁ and Ar₂ are each independently represented by Formula 5-1 or Formula 5-2 below: 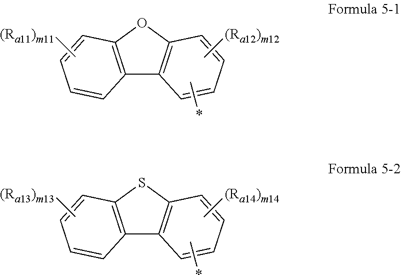
wherein, in Formula 5-1 and Formula 5-2 above,
Rₐ₁₁ to Rₐ₁₄ are each independently a hydrogen atom, a deuterium atom, a halogen atom, a substituted or unsubstituted alkyl group having 1 to 20 carbon atoms, a substituted or unsubstituted ring-forming aryl group having 6 to 30 carbon atoms, or a substituted or unsubstituted ring-forming heteroaryl group having 2 to 30 carbon atoms,
m11 and m13 are each independently an integer of 0 to 4, and
m12 and m14 are each independently an integer of 0 to 3.

20. The amine compound of claim 13, wherein the compound represented by Formula 1 above is any one selected from among the compounds represented in Compound Group 1 below: 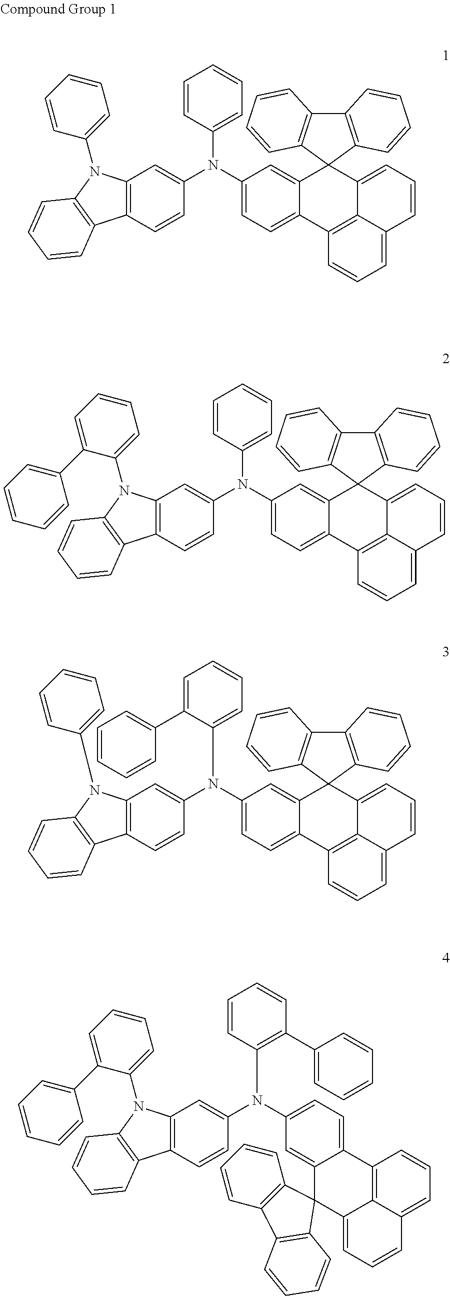 [SMILES: c1ccc(N(c2ccc3c(c2)C2(c4ccccc4-c4ccccc42)c2cccc4cccc-3c24)c2ccc3c4ccccc4n(-c4ccccc4)c3c2)cc1 ; c1ccc(-c2ccccc2-n2c3ccccc3c3ccc(N(c4ccccc4)c4ccc5c(c4)C4(c6ccccc6-c6ccccc64)c4cccc6cccc-5c46)cc32)cc1 ; c1ccc(-c2ccccc2N(c2ccc3c(c2)C2(c4ccccc4-c4ccccc42)c2cccc4cccc-3c24)c2ccc3c4ccccc4n(-c4ccccc4)c3c2)cc1 ; c1ccc(-c2ccccc2N(c2ccc3c(c2)C2(c4ccccc4-c4ccccc42)c2cccc4cccc-3c24)c2ccc3c4ccccc4n(-c4ccccc4-c4ccccc4)c3c2)cc1] 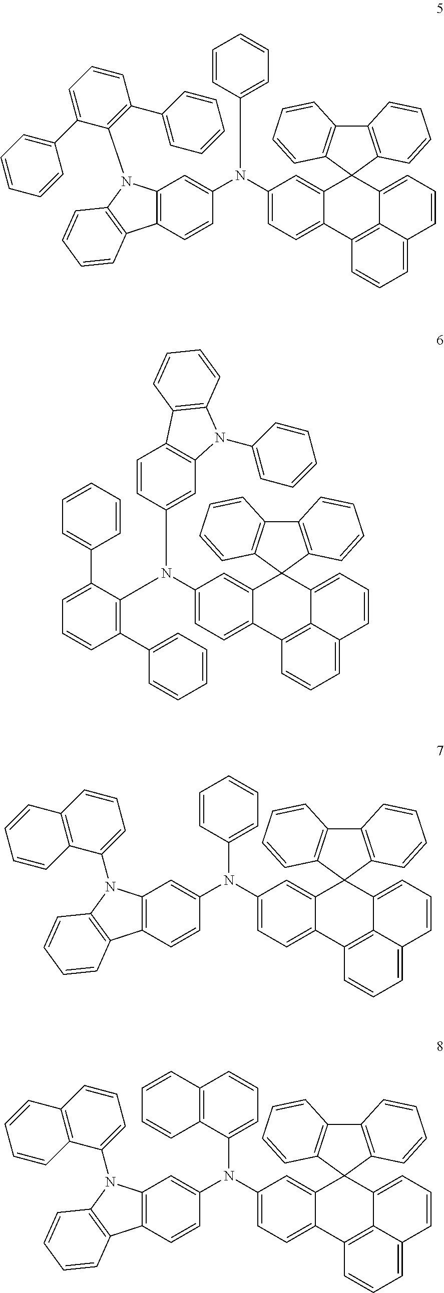 [SMILES: c1ccc(-c2cccc(-c3ccccc3)c2-n2c3ccccc3c3ccc(N(c4ccccc4)c4ccc5c(c4)C4(c6ccccc6-c6ccccc64)c4cccc6cccc-5c46)cc32)cc1 ; c1ccc(-c2cccc(-c3ccccc3)c2N(c2ccc3c(c2)C2(c4ccccc4-c4ccccc42)c2cccc4cccc-3c24)c2ccc3c4ccccc4n(-c4ccccc4)c3c2)cc1 ; c1ccc(N(c2ccc3c(c2)C2(c4ccccc4-c4ccccc42)c2cccc4cccc-3c24)c2ccc3c4ccccc4n(-c4cccc5ccccc45)c3c2)cc1 ; c1ccc2c(c1)-c1ccccc1C21c2cc(N(c3ccc4c5ccccc5n(-c5cccc6ccccc56)c4c3)c3cccc4ccccc34)ccc2-c2cccc3cccc1c23] 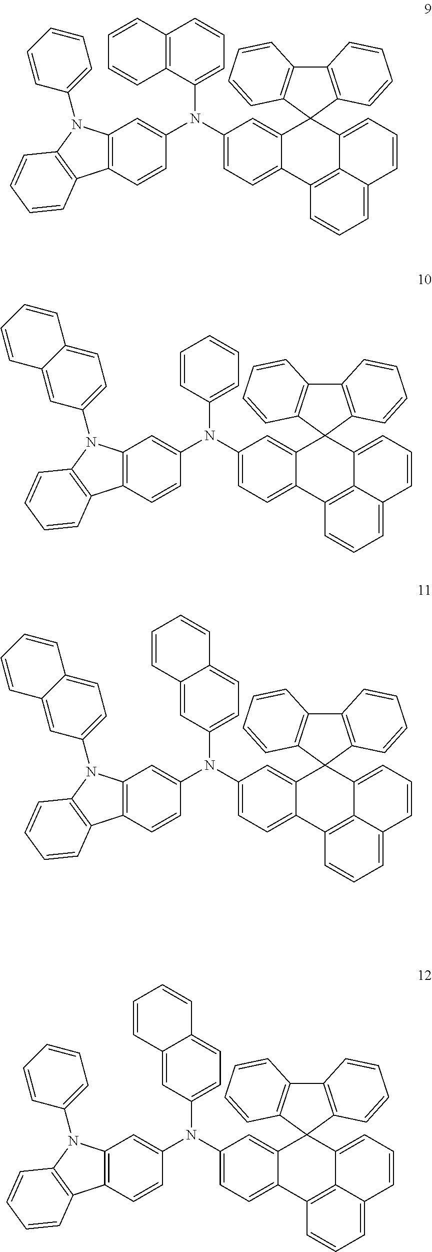 [SMILES: c1ccc(-n2c3ccccc3c3ccc(N(c4ccc5c(c4)C4(c6ccccc6-c6ccccc64)c4cccc6cccc-5c46)c4cccc5ccccc45)cc32)cc1 ; c1ccc(N(c2ccc3c(c2)C2(c4ccccc4-c4ccccc42)c2cccc4cccc-3c24)c2ccc3c4ccccc4n(-c4ccc5ccccc5c4)c3c2)cc1 ; c1ccc2c(c1)-c1ccccc1C21c2cc(N(c3ccc4ccccc4c3)c3ccc4c5ccccc5n(-c5ccc6ccccc6c5)c4c3)ccc2-c2cccc3cccc1c23 ; c1ccc(-n2c3ccccc3c3ccc(N(c4ccc5c(c4)C4(c6ccccc6-c6ccccc64)c4cccc6cccc-5c46)c4ccc5ccccc5c4)cc32)cc1] 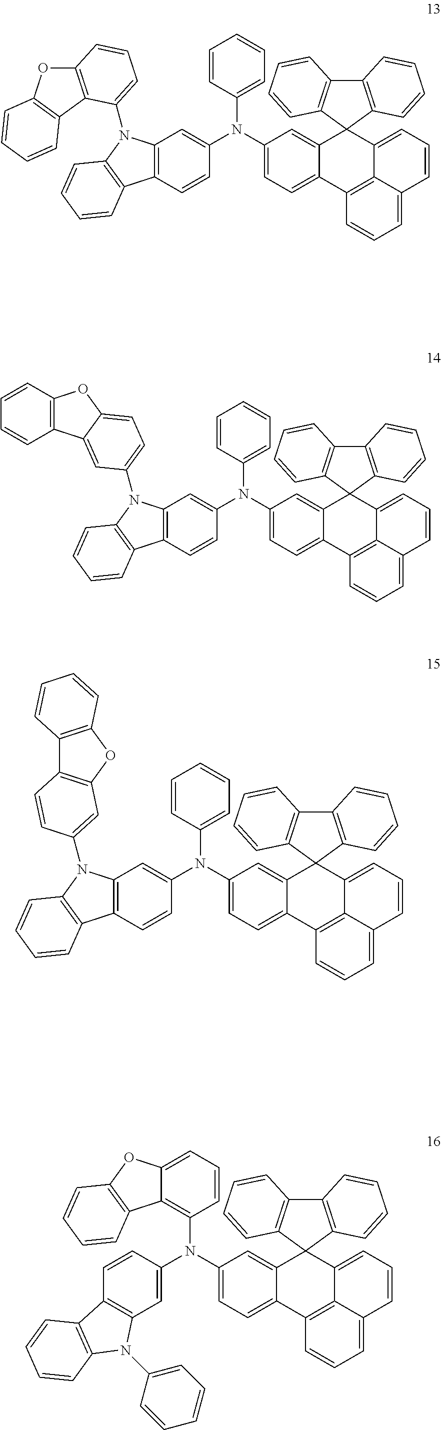 [SMILES: c1ccc(N(c2ccc3c(c2)C2(c4ccccc4-c4ccccc42)c2cccc4cccc-3c24)c2ccc3c4ccccc4n(-c4cccc5oc6ccccc6c45)c3c2)cc1 ; c1ccc(N(c2ccc3c(c2)C2(c4ccccc4-c4ccccc42)c2cccc4cccc-3c24)c2ccc3c4ccccc4n(-c4ccc5oc6ccccc6c5c4)c3c2)cc1 ; c1ccc(N(c2ccc3c(c2)C2(c4ccccc4-c4ccccc42)c2cccc4cccc-3c24)c2ccc3c4ccccc4n(-c4ccc5c(c4)oc4ccccc45)c3c2)cc1 ; c1ccc(-n2c3ccccc3c3ccc(N(c4ccc5c(c4)C4(c6ccccc6-c6ccccc64)c4cccc6cccc-5c46)c4cccc5oc6ccccc6c45)cc32)cc1] 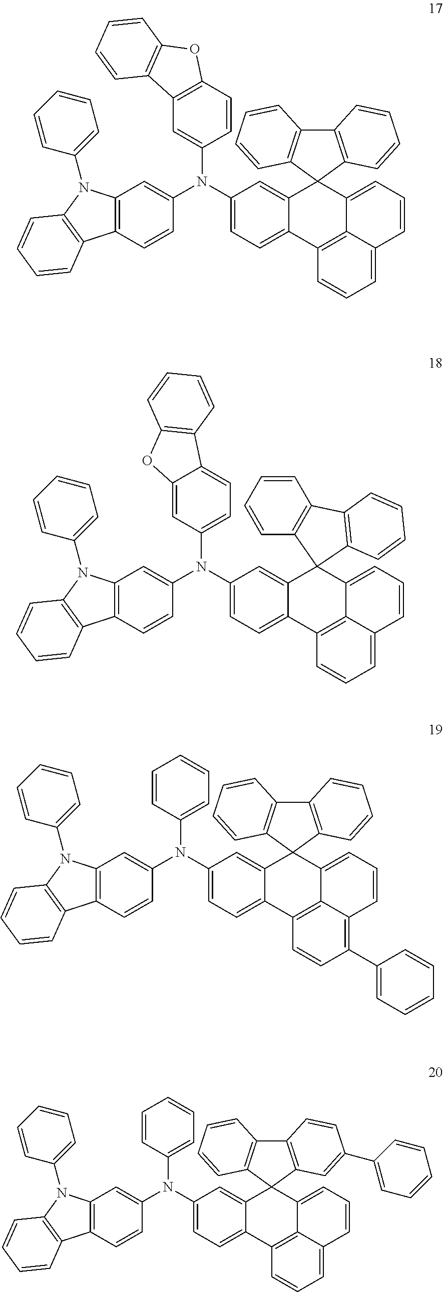 [SMILES: c1ccc(-n2c3ccccc3c3ccc(N(c4ccc5c(c4)C4(c6ccccc6-c6ccccc64)c4cccc6cccc-5c46)c4ccc5oc6ccccc6c5c4)cc32)cc1 ; c1ccc(-n2c3ccccc3c3ccc(N(c4ccc5c(c4)C4(c6ccccc6-c6ccccc64)c4cccc6cccc-5c46)c4ccc5c(c4)oc4ccccc45)cc32)cc1 ; c1ccc(-c2ccc3c4c(cccc24)C2(c4ccccc4-c4ccccc42)c2cc(N(c4ccccc4)c4ccc5c6ccccc6n(-c6ccccc6)c5c4)ccc2-3)cc1 ; c1ccc(-c2ccc3c(c2)C2(c4ccccc4-3)c3cc(N(c4ccccc4)c4ccc5c6ccccc6n(-c6ccccc6)c5c4)ccc3-c3cccc4cccc2c34)cc1] 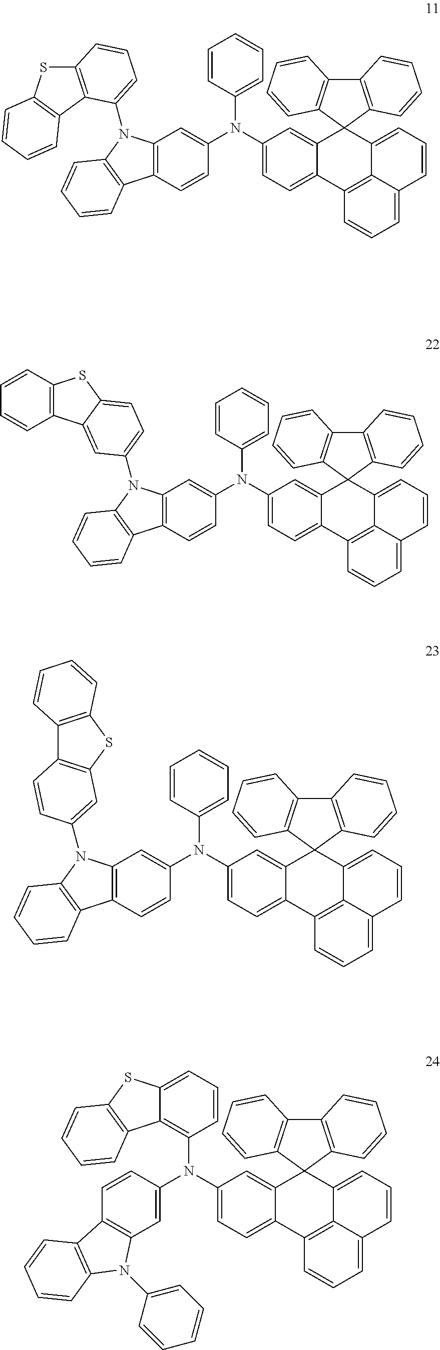 [SMILES: c1ccc(N(c2ccc3c(c2)C2(c4ccccc4-c4ccccc42)c2cccc4cccc-3c24)c2ccc3c4ccccc4n(-c4cccc5sc6ccccc6c45)c3c2)cc1 ; c1ccc(N(c2ccc3c(c2)C2(c4ccccc4-c4ccccc42)c2cccc4cccc-3c24)c2ccc3c4ccccc4n(-c4ccc5sc6ccccc6c5c4)c3c2)cc1 ; c1ccc(N(c2ccc3c(c2)C2(c4ccccc4-c4ccccc42)c2cccc4cccc-3c24)c2ccc3c4ccccc4n(-c4ccc5c(c4)sc4ccccc45)c3c2)cc1 ; c1ccc(-n2c3ccccc3c3ccc(N(c4ccc5c(c4)C4(c6ccccc6-c6ccccc64)c4cccc6cccc-5c46)c4cccc5sc6ccccc6c45)cc32)cc1] 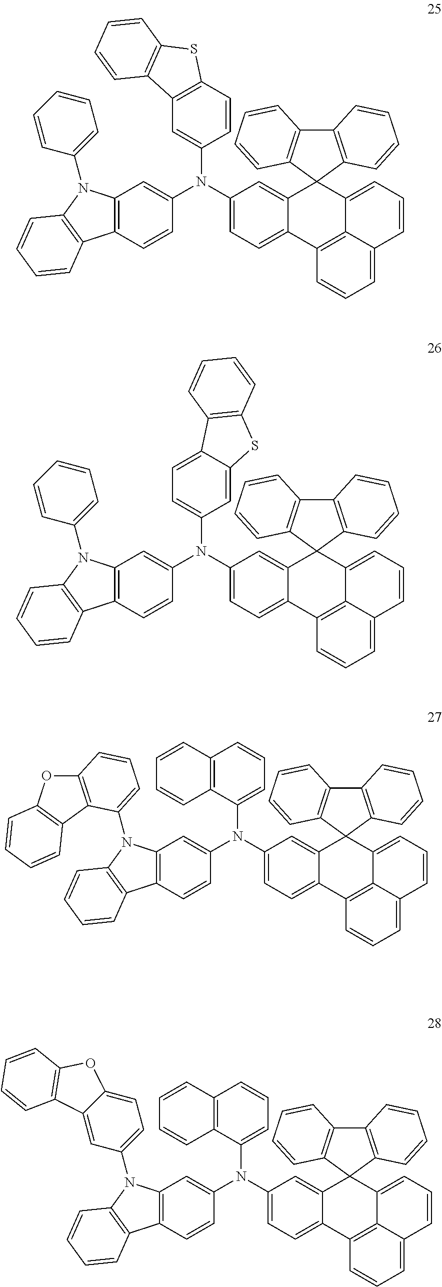 [SMILES: c1ccc(-n2c3ccccc3c3ccc(N(c4ccc5c(c4)C4(c6ccccc6-c6ccccc64)c4cccc6cccc-5c46)c4ccc5sc6ccccc6c5c4)cc32)cc1 ; c1ccc(-n2c3ccccc3c3ccc(N(c4ccc5c(c4)C4(c6ccccc6-c6ccccc64)c4cccc6cccc-5c46)c4ccc5c(c4)sc4ccccc45)cc32)cc1 ; c1ccc2c(c1)-c1ccccc1C21c2cc(N(c3ccc4c5ccccc5n(-c5cccc6oc7ccccc7c56)c4c3)c3cccc4ccccc34)ccc2-c2cccc3cccc1c23 ; c1ccc2c(c1)-c1ccccc1C21c2cc(N(c3ccc4c5ccccc5n(-c5ccc6oc7ccccc7c6c5)c4c3)c3cccc4ccccc34)ccc2-c2cccc3cccc1c23] 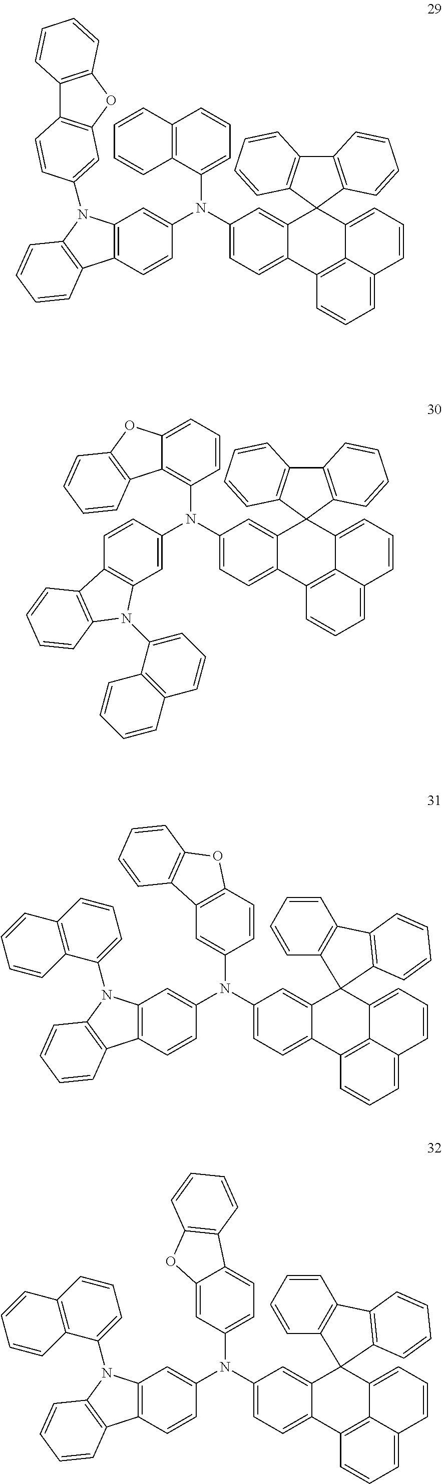 [SMILES: c1ccc2c(c1)-c1ccccc1C21c2cc(N(c3ccc4c5ccccc5n(-c5ccc6c(c5)oc5ccccc56)c4c3)c3cccc4ccccc34)ccc2-c2cccc3cccc1c23 ; c1ccc2c(c1)-c1ccccc1C21c2cc(N(c3ccc4c5ccccc5n(-c5cccc6ccccc56)c4c3)c3cccc4oc5ccccc5c34)ccc2-c2cccc3cccc1c23 ; c1ccc2c(c1)-c1ccccc1C21c2cc(N(c3ccc4oc5ccccc5c4c3)c3ccc4c5ccccc5n(-c5cccc6ccccc56)c4c3)ccc2-c2cccc3cccc1c23 ; c1ccc2c(c1)-c1ccccc1C21c2cc(N(c3ccc4c(c3)oc3ccccc34)c3ccc4c5ccccc5n(-c5cccc6ccccc56)c4c3)ccc2-c2cccc3cccc1c23] 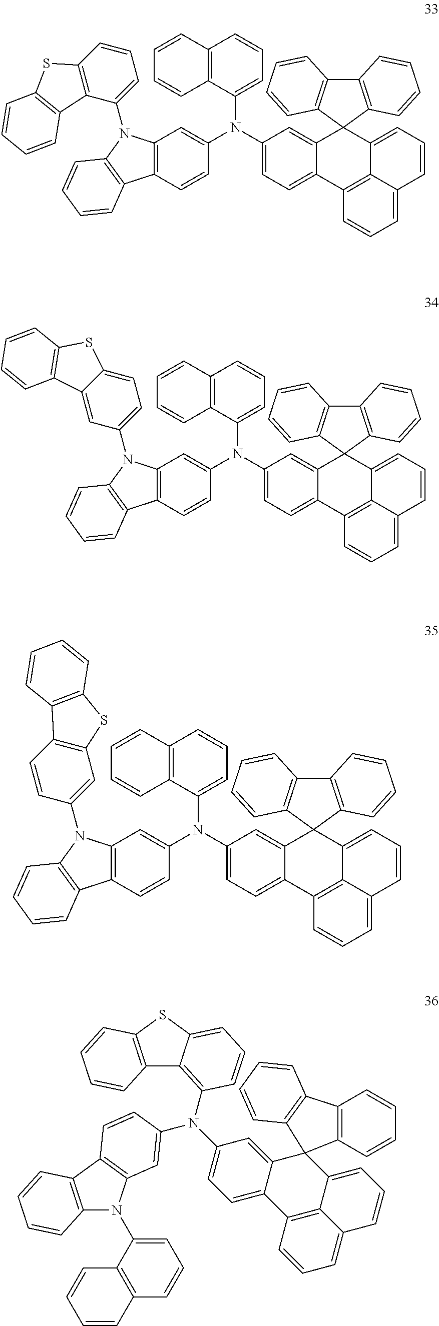 [SMILES: c1ccc2c(c1)-c1ccccc1C21c2cc(N(c3ccc4c5ccccc5n(-c5cccc6sc7ccccc7c56)c4c3)c3cccc4ccccc34)ccc2-c2cccc3cccc1c23 ; c1ccc2c(c1)-c1ccccc1C21c2cc(N(c3ccc4c5ccccc5n(-c5ccc6sc7ccccc7c6c5)c4c3)c3cccc4ccccc34)ccc2-c2cccc3cccc1c23 ; c1ccc2c(c1)-c1ccccc1C21c2cc(N(c3ccc4c5ccccc5n(-c5ccc6c(c5)sc5ccccc56)c4c3)c3cccc4ccccc34)ccc2-c2cccc3cccc1c23 ; c1ccc2c(c1)-c1ccccc1C21c2cc(N(c3ccc4c5ccccc5n(-c5cccc6ccccc56)c4c3)c3cccc4sc5ccccc5c34)ccc2-c2cccc3cccc1c23] 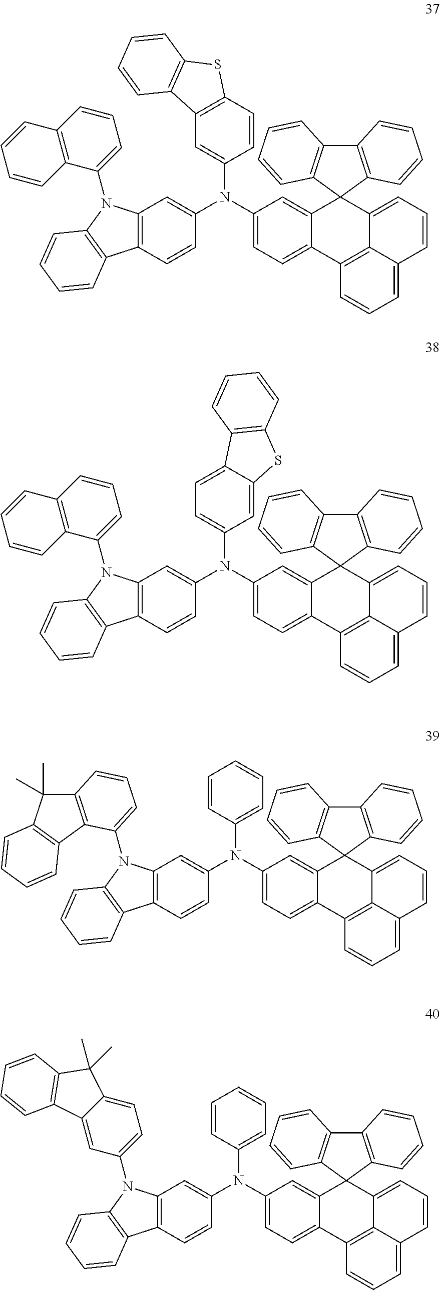 [SMILES: c1ccc2c(c1)-c1ccccc1C21c2cc(N(c3ccc4sc5ccccc5c4c3)c3ccc4c5ccccc5n(-c5cccc6ccccc56)c4c3)ccc2-c2cccc3cccc1c23 ; c1ccc2c(c1)-c1ccccc1C21c2cc(N(c3ccc4c(c3)sc3ccccc34)c3ccc4c5ccccc5n(-c5cccc6ccccc56)c4c3)ccc2-c2cccc3cccc1c23 ; CC1(C)c2ccccc2-c2c(-n3c4ccccc4c4ccc(N(c5ccccc5)c5ccc6c(c5)C5(c7ccccc7-c7ccccc75)c5cccc7cccc-6c57)cc43)cccc21 ; CC1(C)c2ccccc2-c2cc(-n3c4ccccc4c4ccc(N(c5ccccc5)c5ccc6c(c5)C5(c7ccccc7-c7ccccc75)c5cccc7cccc-6c57)cc43)ccc21] 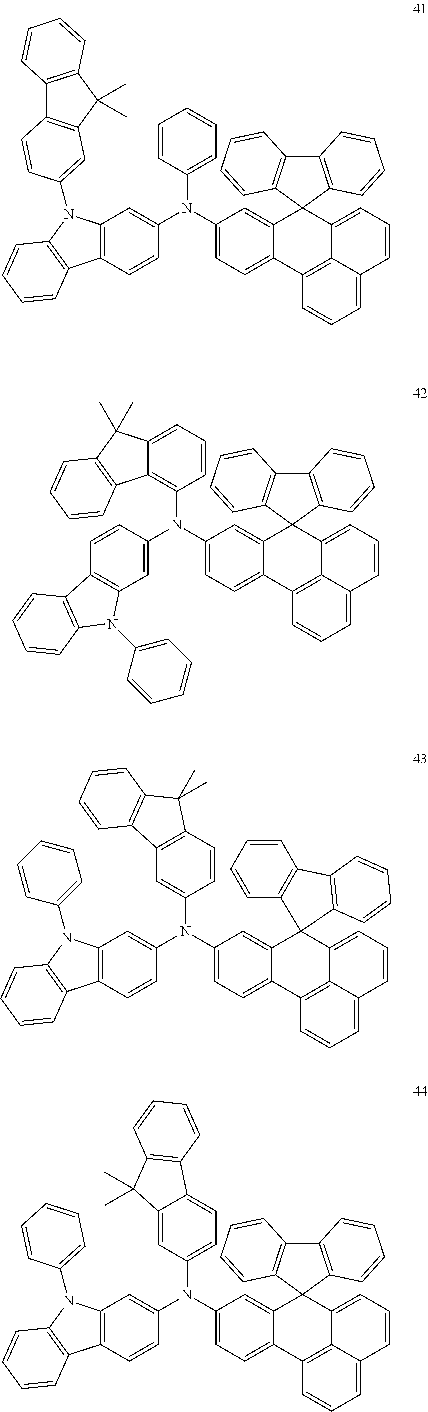 [SMILES: CC1(C)c2ccccc2-c2ccc(-n3c4ccccc4c4ccc(N(c5ccccc5)c5ccc6c(c5)C5(c7ccccc7-c7ccccc75)c5cccc7cccc-6c57)cc43)cc21 ; CC1(C)c2ccccc2-c2c(N(c3ccc4c(c3)C3(c5ccccc5-c5ccccc53)c3cccc5cccc-4c35)c3ccc4c5ccccc5n(-c5ccccc5)c4c3)cccc21 ; CC1(C)c2ccccc2-c2cc(N(c3ccc4c(c3)C3(c5ccccc5-c5ccccc53)c3cccc5cccc-4c35)c3ccc4c5ccccc5n(-c5ccccc5)c4c3)ccc21 ; CC1(C)c2ccccc2-c2ccc(N(c3ccc4c(c3)C3(c5ccccc5-c5ccccc53)c3cccc5cccc-4c35)c3ccc4c5ccccc5n(-c5ccccc5)c4c3)cc21] 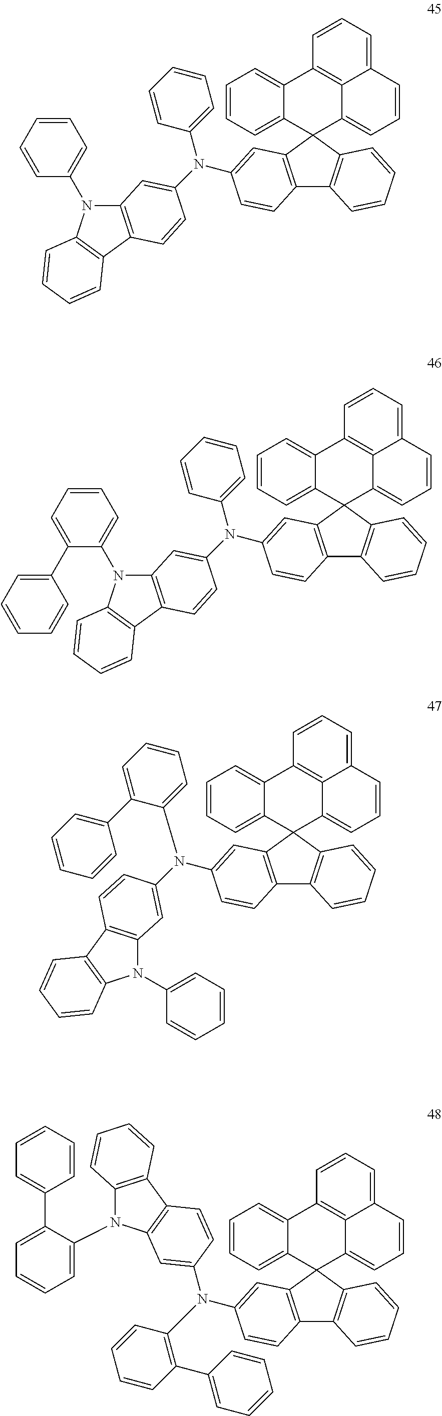 [SMILES: c1ccc(N(c2ccc3c(c2)C2(c4ccccc4-3)c3ccccc3-c3cccc4cccc2c34)c2ccc3c4ccccc4n(-c4ccccc4)c3c2)cc1 ; c1ccc(-c2ccccc2-n2c3ccccc3c3ccc(N(c4ccccc4)c4ccc5c(c4)C4(c6ccccc6-5)c5ccccc5-c5cccc6cccc4c56)cc32)cc1 ; c1ccc(-c2ccccc2N(c2ccc3c(c2)C2(c4ccccc4-3)c3ccccc3-c3cccc4cccc2c34)c2ccc3c4ccccc4n(-c4ccccc4)c3c2)cc1 ; c1ccc(-c2ccccc2N(c2ccc3c(c2)C2(c4ccccc4-3)c3ccccc3-c3cccc4cccc2c34)c2ccc3c4ccccc4n(-c4ccccc4-c4ccccc4)c3c2)cc1] 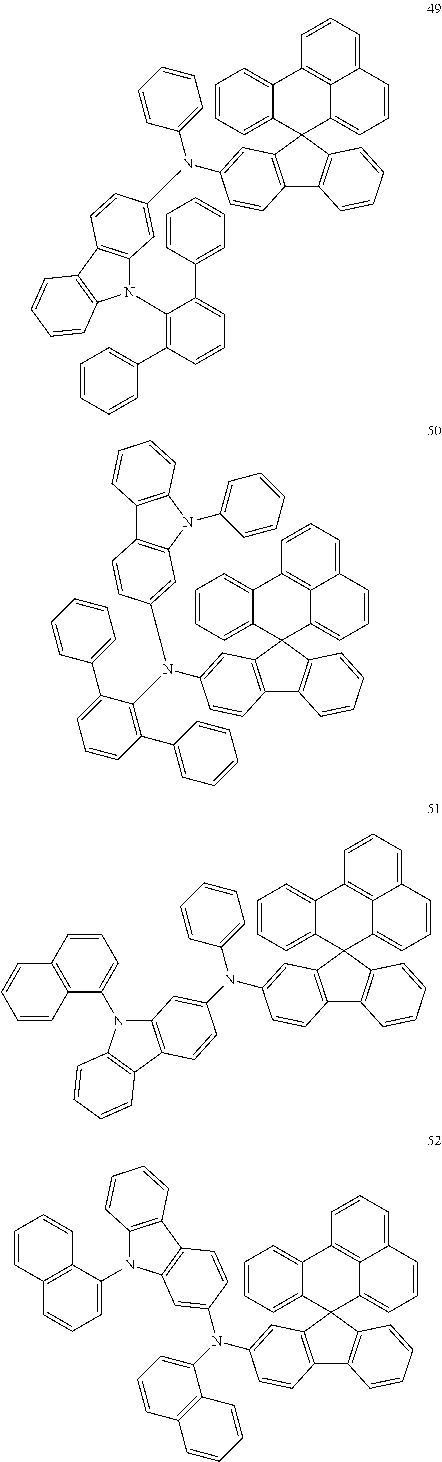 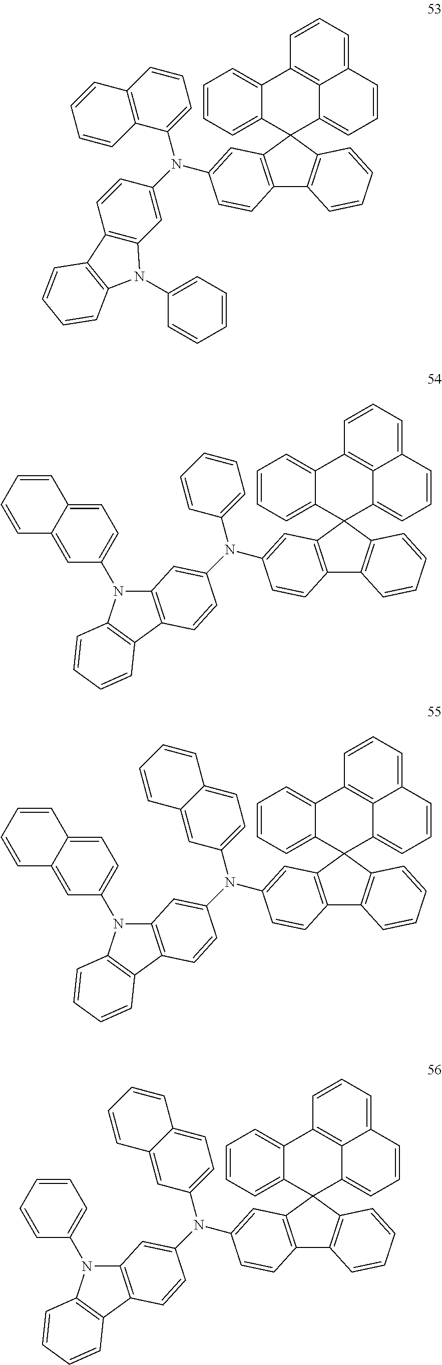 [SMILES: c1ccc(-n2c3ccccc3c3ccc(N(c4ccc5c(c4)C4(c6ccccc6-5)c5ccccc5-c5cccc6cccc4c56)c4cccc5ccccc45)cc32)cc1 ; c1ccc(N(c2ccc3c(c2)C2(c4ccccc4-3)c3ccccc3-c3cccc4cccc2c34)c2ccc3c4ccccc4n(-c4ccc5ccccc5c4)c3c2)cc1 ; c1ccc2c(c1)-c1ccc(N(c3ccc4ccccc4c3)c3ccc4c5ccccc5n(-c5ccc6ccccc6c5)c4c3)cc1C21c2ccccc2-c2cccc3cccc1c23 ; c1ccc(-n2c3ccccc3c3ccc(N(c4ccc5c(c4)C4(c6ccccc6-5)c5ccccc5-c5cccc6cccc4c56)c4ccc5ccccc5c4)cc32)cc1] 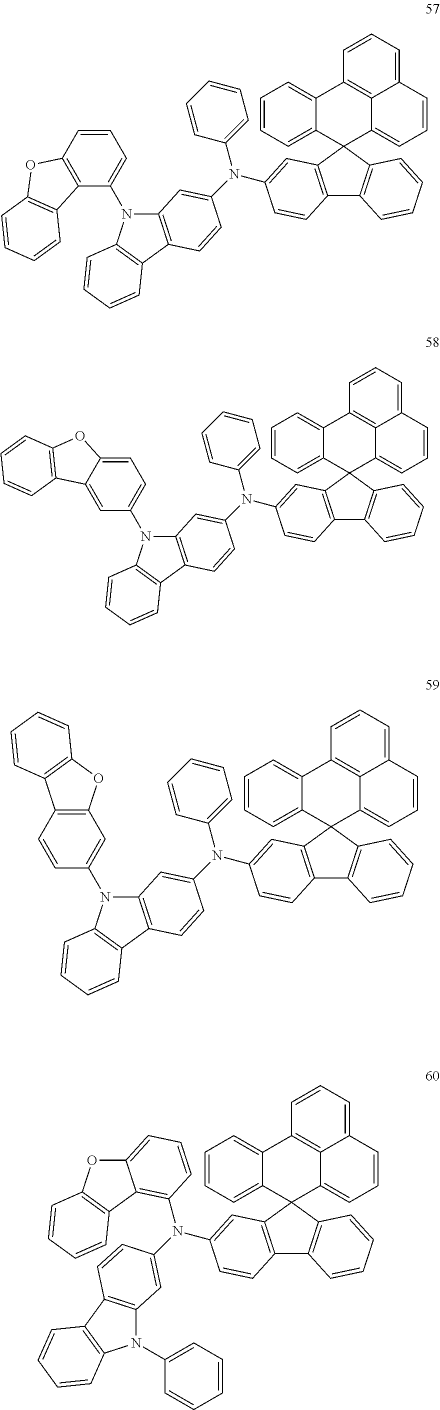 [SMILES: c1ccc(N(c2ccc3c(c2)C2(c4ccccc4-3)c3ccccc3-c3cccc4cccc2c34)c2ccc3c4ccccc4n(-c4cccc5oc6ccccc6c45)c3c2)cc1 ; c1ccc(N(c2ccc3c(c2)C2(c4ccccc4-3)c3ccccc3-c3cccc4cccc2c34)c2ccc3c4ccccc4n(-c4ccc5oc6ccccc6c5c4)c3c2)cc1 ; c1ccc(N(c2ccc3c(c2)C2(c4ccccc4-3)c3ccccc3-c3cccc4cccc2c34)c2ccc3c4ccccc4n(-c4ccc5c(c4)oc4ccccc45)c3c2)cc1 ; c1ccc(-n2c3ccccc3c3ccc(N(c4ccc5c(c4)C4(c6ccccc6-5)c5ccccc5-c5cccc6cccc4c56)c4cccc5oc6ccccc6c45)cc32)cc1] 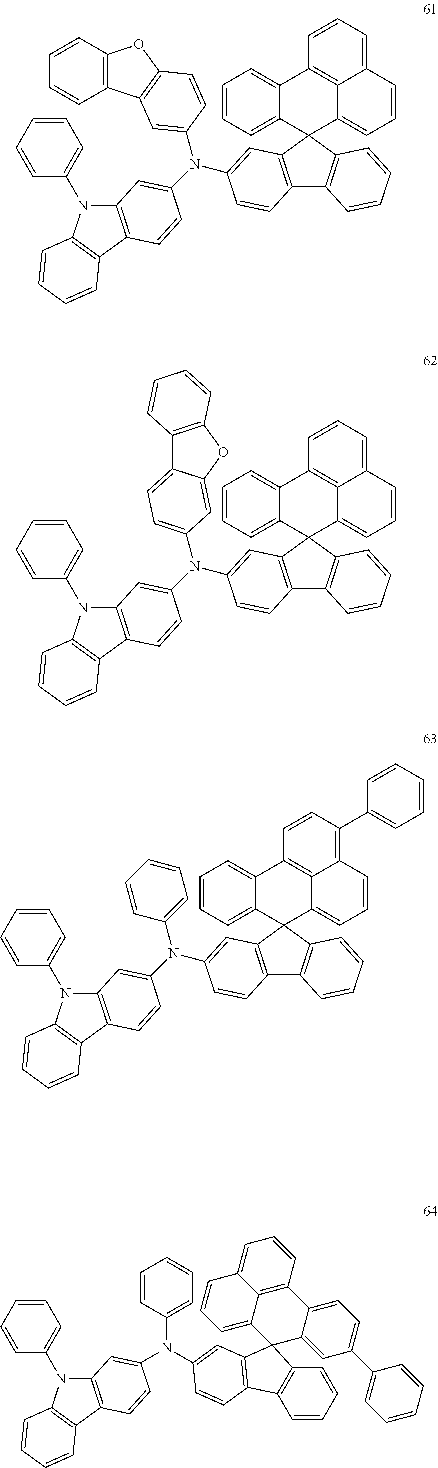 [SMILES: c1ccc(-n2c3ccccc3c3ccc(N(c4ccc5c(c4)C4(c6ccccc6-5)c5ccccc5-c5cccc6cccc4c56)c4ccc5oc6ccccc6c5c4)cc32)cc1 ; c1ccc(-n2c3ccccc3c3ccc(N(c4ccc5c(c4)C4(c6ccccc6-5)c5ccccc5-c5cccc6cccc4c56)c4ccc5c(c4)oc4ccccc45)cc32)cc1 ; c1ccc(-c2ccc3c4c(cccc24)C2(c4ccccc4-c4ccc(N(c5ccccc5)c5ccc6c7ccccc7n(-c7ccccc7)c6c5)cc42)c2ccccc2-3)cc1 ; c1ccc(-c2ccc3c(c2)C2(c4ccccc4-c4ccc(N(c5ccccc5)c5ccc6c7ccccc7n(-c7ccccc7)c6c5)cc42)c2cccc4cccc-3c24)cc1] 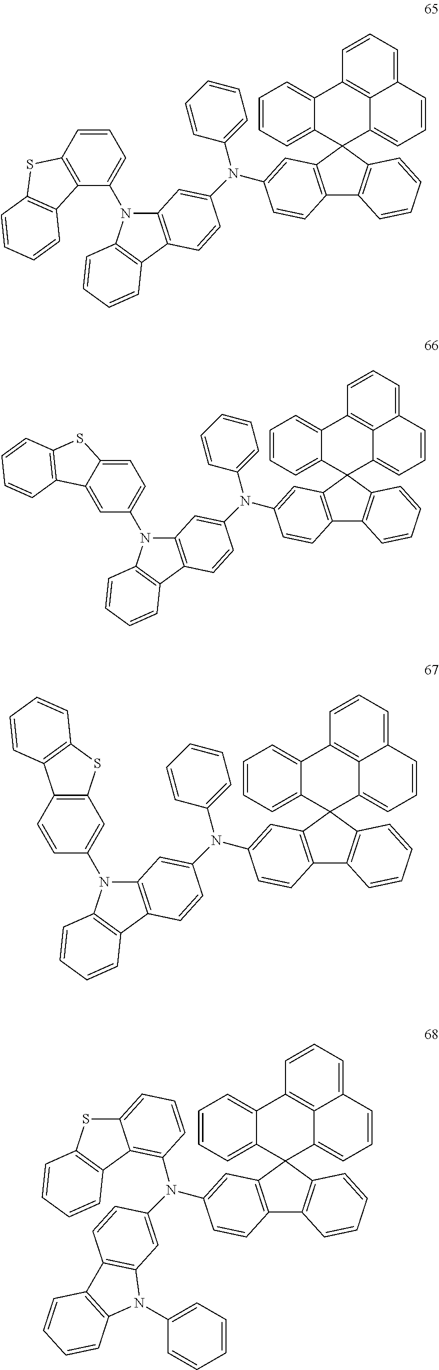 [SMILES: c1ccc(N(c2ccc3c(c2)C2(c4ccccc4-3)c3ccccc3-c3cccc4cccc2c34)c2ccc3c4ccccc4n(-c4cccc5sc6ccccc6c45)c3c2)cc1 ; c1ccc(N(c2ccc3c(c2)C2(c4ccccc4-3)c3ccccc3-c3cccc4cccc2c34)c2ccc3c4ccccc4n(-c4ccc5sc6ccccc6c5c4)c3c2)cc1 ; c1ccc(N(c2ccc3c(c2)C2(c4ccccc4-3)c3ccccc3-c3cccc4cccc2c34)c2ccc3c4ccccc4n(-c4ccc5c(c4)sc4ccccc45)c3c2)cc1 ; c1ccc(-n2c3ccccc3c3ccc(N(c4ccc5c(c4)C4(c6ccccc6-5)c5ccccc5-c5cccc6cccc4c56)c4cccc5sc6ccccc6c45)cc32)cc1] 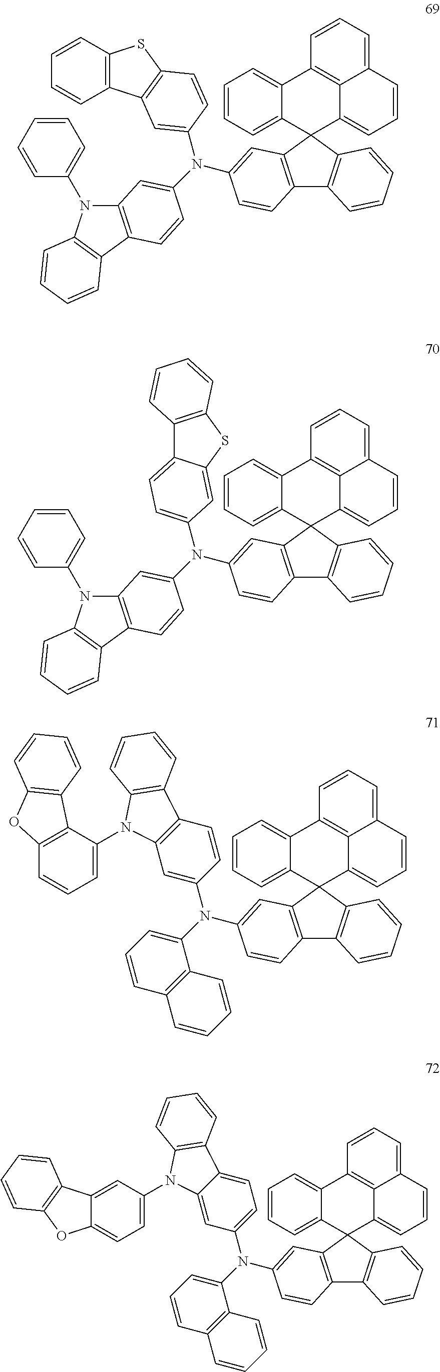 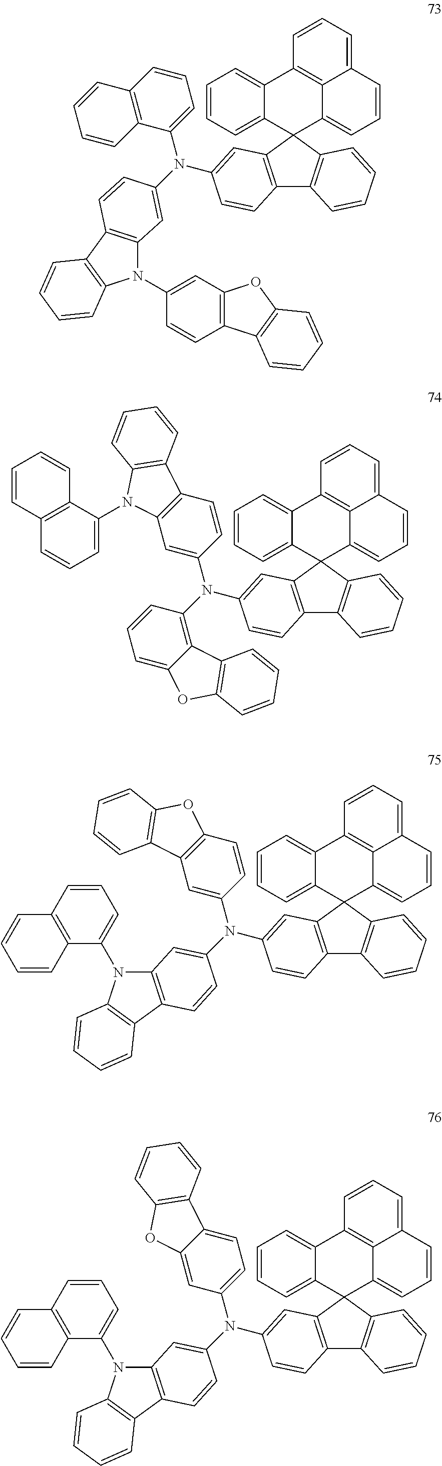 [SMILES: c1ccc2c(c1)-c1ccc(N(c3ccc4c5ccccc5n(-c5ccc6c(c5)oc5ccccc56)c4c3)c3cccc4ccccc34)cc1C21c2ccccc2-c2cccc3cccc1c23 ; c1ccc2c(c1)-c1ccc(N(c3ccc4c5ccccc5n(-c5cccc6ccccc56)c4c3)c3cccc4oc5ccccc5c34)cc1C21c2ccccc2-c2cccc3cccc1c23 ; c1ccc2c(c1)-c1ccc(N(c3ccc4oc5ccccc5c4c3)c3ccc4c5ccccc5n(-c5cccc6ccccc56)c4c3)cc1C21c2ccccc2-c2cccc3cccc1c23 ; c1ccc2c(c1)-c1ccc(N(c3ccc4c(c3)oc3ccccc34)c3ccc4c5ccccc5n(-c5cccc6ccccc56)c4c3)cc1C21c2ccccc2-c2cccc3cccc1c23] 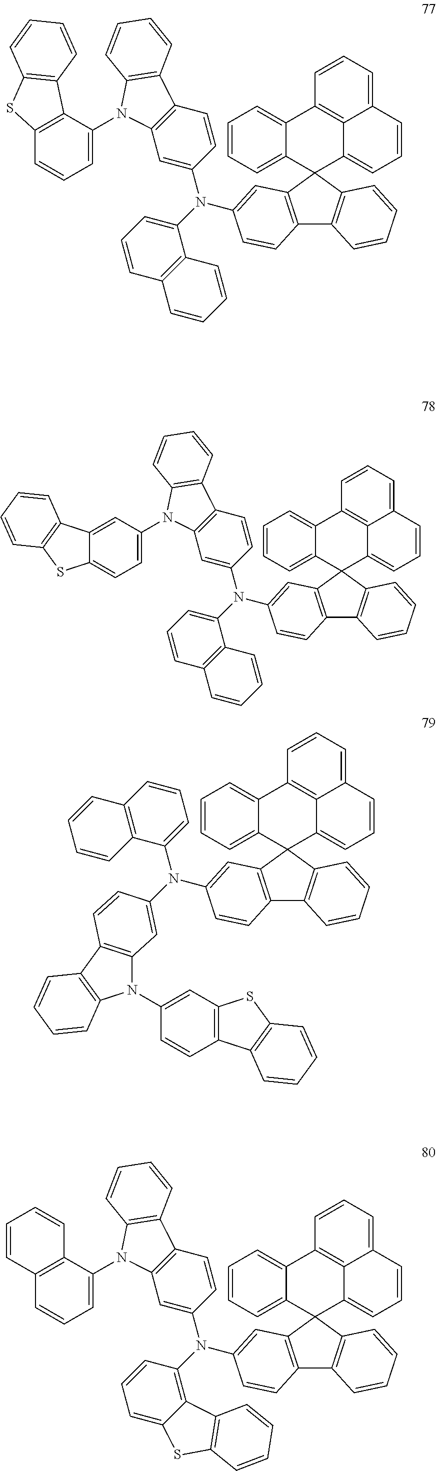 [SMILES: c1ccc2c(c1)-c1ccc(N(c3ccc4c5ccccc5n(-c5cccc6sc7ccccc7c56)c4c3)c3cccc4ccccc34)cc1C21c2ccccc2-c2cccc3cccc1c23 ; c1ccc2c(c1)-c1ccc(N(c3ccc4c5ccccc5n(-c5ccc6sc7ccccc7c6c5)c4c3)c3cccc4ccccc34)cc1C21c2ccccc2-c2cccc3cccc1c23 ; c1ccc2c(c1)-c1ccc(N(c3ccc4c5ccccc5n(-c5ccc6c(c5)sc5ccccc56)c4c3)c3cccc4ccccc34)cc1C21c2ccccc2-c2cccc3cccc1c23 ; c1ccc2c(c1)-c1ccc(N(c3ccc4c5ccccc5n(-c5cccc6ccccc56)c4c3)c3cccc4sc5ccccc5c34)cc1C21c2ccccc2-c2cccc3cccc1c23] 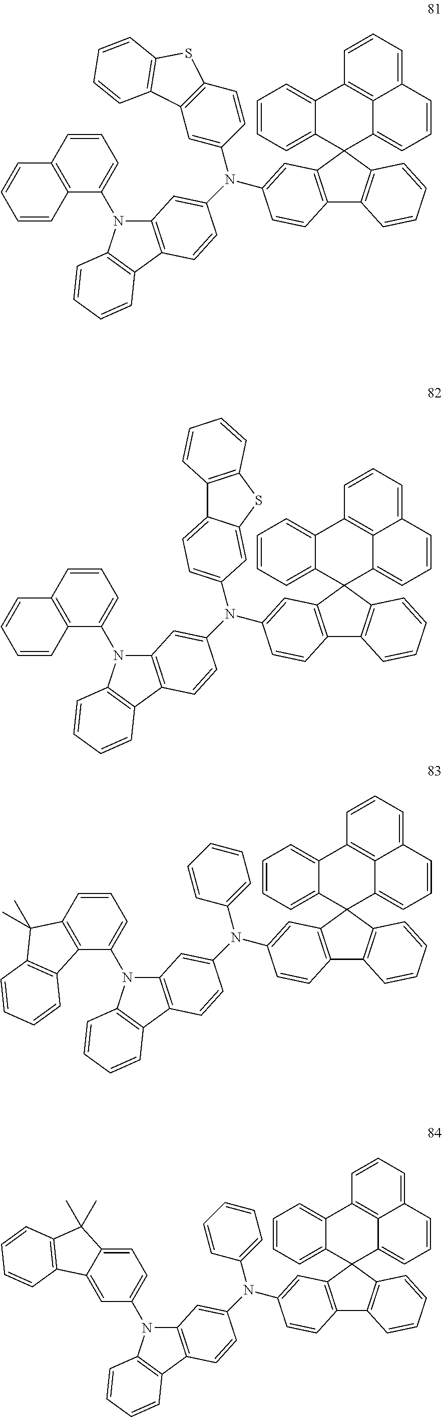 [SMILES: c1ccc2c(c1)-c1ccc(N(c3ccc4sc5ccccc5c4c3)c3ccc4c5ccccc5n(-c5cccc6ccccc56)c4c3)cc1C21c2ccccc2-c2cccc3cccc1c23 ; c1ccc2c(c1)-c1ccc(N(c3ccc4c(c3)sc3ccccc34)c3ccc4c5ccccc5n(-c5cccc6ccccc56)c4c3)cc1C21c2ccccc2-c2cccc3cccc1c23 ; CC1(C)c2ccccc2-c2c(-n3c4ccccc4c4ccc(N(c5ccccc5)c5ccc6c(c5)C5(c7ccccc7-6)c6ccccc6-c6cccc7cccc5c67)cc43)cccc21 ; CC1(C)c2ccccc2-c2cc(-n3c4ccccc4c4ccc(N(c5ccccc5)c5ccc6c(c5)C5(c7ccccc7-6)c6ccccc6-c6cccc7cccc5c67)cc43)ccc21] 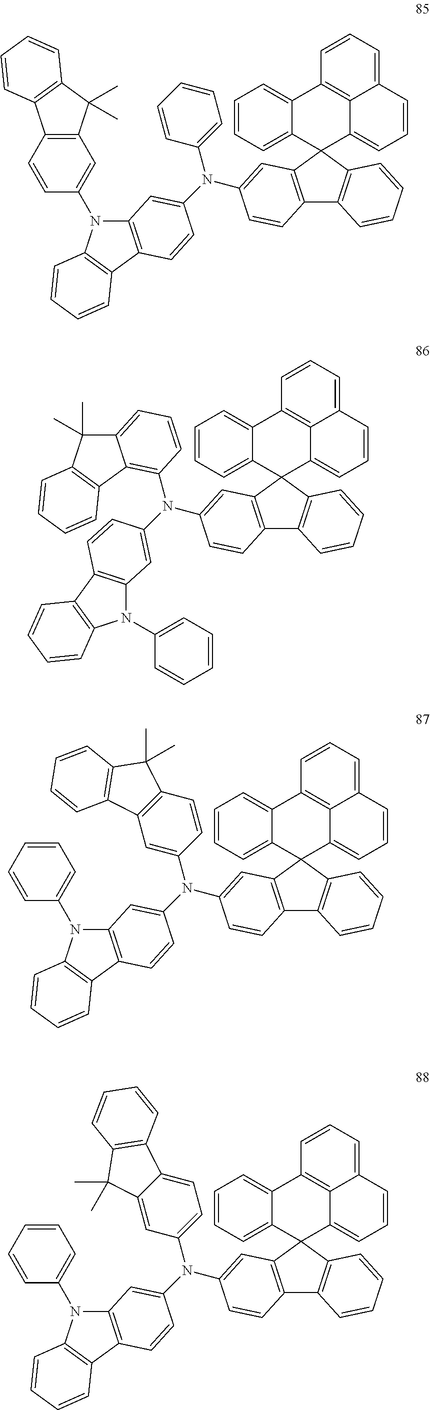 [SMILES: CC1(C)c2ccccc2-c2ccc(-n3c4ccccc4c4ccc(N(c5ccccc5)c5ccc6c(c5)C5(c7ccccc7-6)c6ccccc6-c6cccc7cccc5c67)cc43)cc21 ; CC1(C)c2ccccc2-c2c(N(c3ccc4c(c3)C3(c5ccccc5-4)c4ccccc4-c4cccc5cccc3c45)c3ccc4c5ccccc5n(-c5ccccc5)c4c3)cccc21 ; CC1(C)c2ccccc2-c2cc(N(c3ccc4c(c3)C3(c5ccccc5-4)c4ccccc4-c4cccc5cccc3c45)c3ccc4c5ccccc5n(-c5ccccc5)c4c3)ccc21 ; CC1(C)c2ccccc2-c2ccc(N(c3ccc4c(c3)C3(c5ccccc5-4)c4ccccc4-c4cccc5cccc3c45)c3ccc4c5ccccc5n(-c5ccccc5)c4c3)cc21] 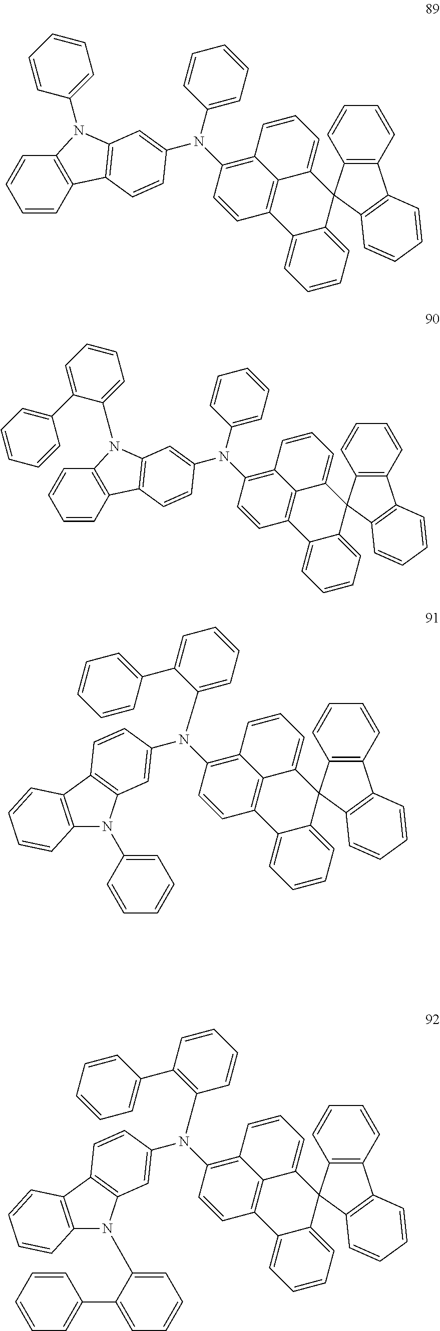 [SMILES: c1ccc(N(c2ccc3c4ccccc4n(-c4ccccc4)c3c2)c2ccc3c4c(cccc24)C2(c4ccccc4-c4ccccc42)c2ccccc2-3)cc1 ; c1ccc(-c2ccccc2-n2c3ccccc3c3ccc(N(c4ccccc4)c4ccc5c6c(cccc46)C4(c6ccccc6-c6ccccc64)c4ccccc4-5)cc32)cc1 ; c1ccc(-c2ccccc2N(c2ccc3c4ccccc4n(-c4ccccc4)c3c2)c2ccc3c4c(cccc24)C2(c4ccccc4-c4ccccc42)c2ccccc2-3)cc1 ; c1ccc(-c2ccccc2N(c2ccc3c4ccccc4n(-c4ccccc4-c4ccccc4)c3c2)c2ccc3c4c(cccc24)C2(c4ccccc4-c4ccccc42)c2ccccc2-3)cc1] 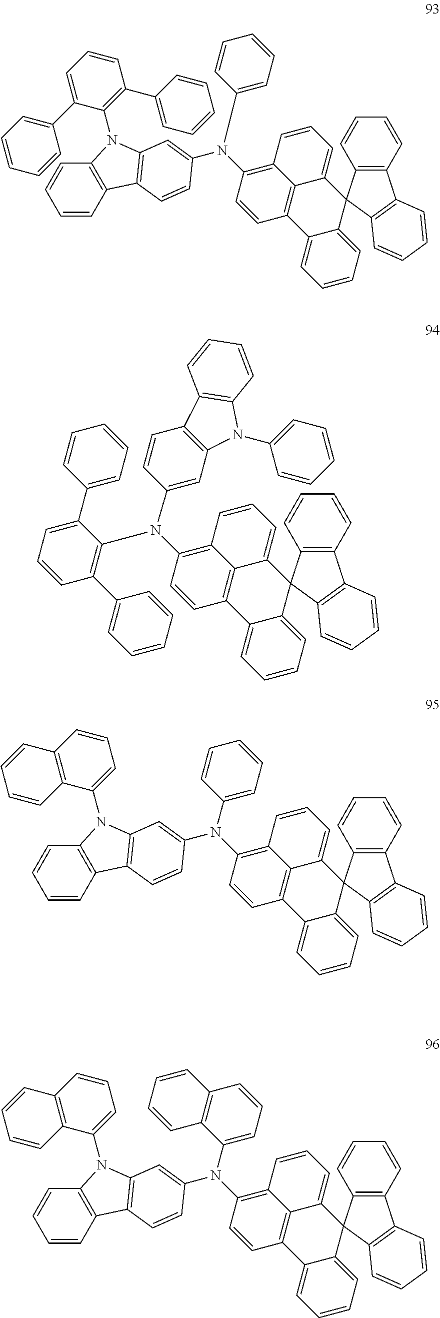 [SMILES: c1ccc(-c2cccc(-c3ccccc3)c2-n2c3ccccc3c3ccc(N(c4ccccc4)c4ccc5c6c(cccc46)C4(c6ccccc6-c6ccccc64)c4ccccc4-5)cc32)cc1 ; c1ccc(-c2cccc(-c3ccccc3)c2N(c2ccc3c4ccccc4n(-c4ccccc4)c3c2)c2ccc3c4c(cccc24)C2(c4ccccc4-c4ccccc42)c2ccccc2-3)cc1 ; c1ccc(N(c2ccc3c4ccccc4n(-c4cccc5ccccc45)c3c2)c2ccc3c4c(cccc24)C2(c4ccccc4-c4ccccc42)c2ccccc2-3)cc1 ; c1ccc2c(c1)-c1ccccc1C21c2ccccc2-c2ccc(N(c3ccc4c5ccccc5n(-c5cccc6ccccc56)c4c3)c3cccc4ccccc34)c3cccc1c23] 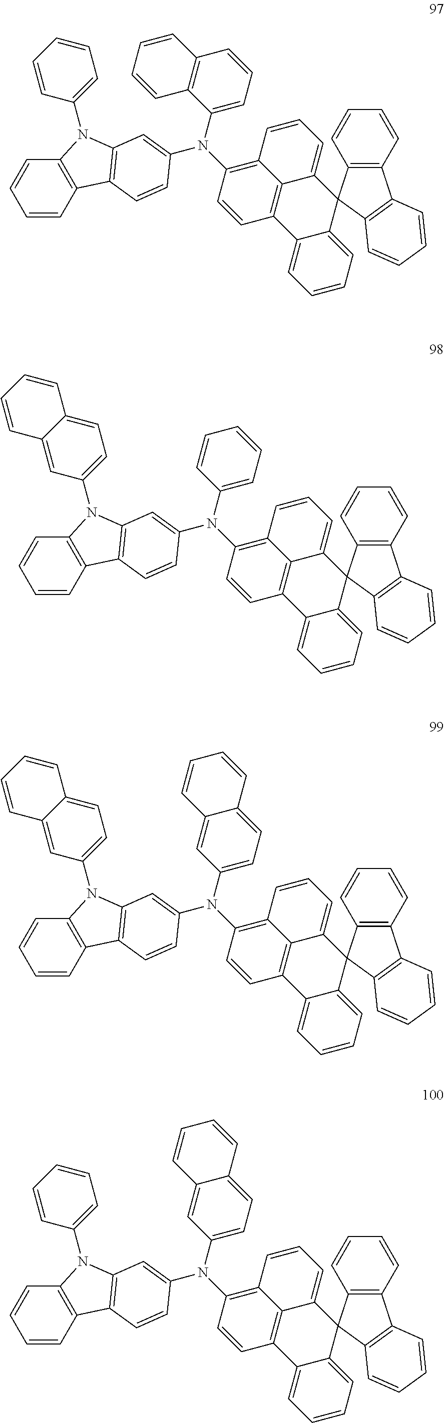 [SMILES: c1ccc(-n2c3ccccc3c3ccc(N(c4cccc5ccccc45)c4ccc5c6c(cccc46)C4(c6ccccc6-c6ccccc64)c4ccccc4-5)cc32)cc1 ; c1ccc(N(c2ccc3c4ccccc4n(-c4ccc5ccccc5c4)c3c2)c2ccc3c4c(cccc24)C2(c4ccccc4-c4ccccc42)c2ccccc2-3)cc1 ; c1ccc2c(c1)-c1ccccc1C21c2ccccc2-c2ccc(N(c3ccc4ccccc4c3)c3ccc4c5ccccc5n(-c5ccc6ccccc6c5)c4c3)c3cccc1c23 ; c1ccc(-n2c3ccccc3c3ccc(N(c4ccc5ccccc5c4)c4ccc5c6c(cccc46)C4(c6ccccc6-c6ccccc64)c4ccccc4-5)cc32)cc1] 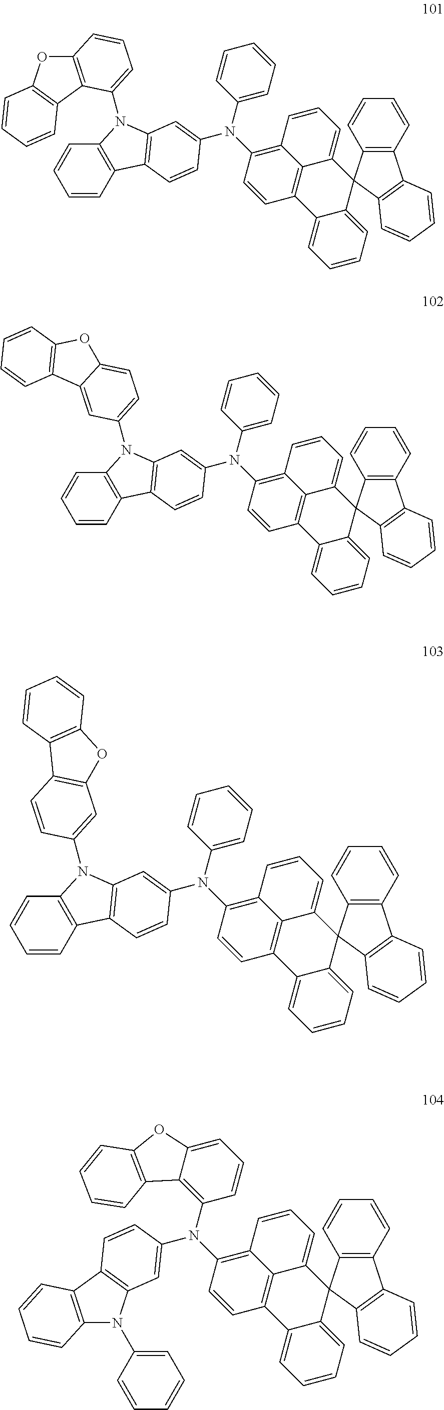 [SMILES: c1ccc(N(c2ccc3c4ccccc4n(-c4cccc5oc6ccccc6c45)c3c2)c2ccc3c4c(cccc24)C2(c4ccccc4-c4ccccc42)c2ccccc2-3)cc1 ; c1ccc(N(c2ccc3c4ccccc4n(-c4ccc5oc6ccccc6c5c4)c3c2)c2ccc3c4c(cccc24)C2(c4ccccc4-c4ccccc42)c2ccccc2-3)cc1 ; c1ccc(N(c2ccc3c4ccccc4n(-c4ccc5c(c4)oc4ccccc45)c3c2)c2ccc3c4c(cccc24)C2(c4ccccc4-c4ccccc42)c2ccccc2-3)cc1 ; c1ccc(-n2c3ccccc3c3ccc(N(c4ccc5c6c(cccc46)C4(c6ccccc6-c6ccccc64)c4ccccc4-5)c4cccc5oc6ccccc6c45)cc32)cc1] 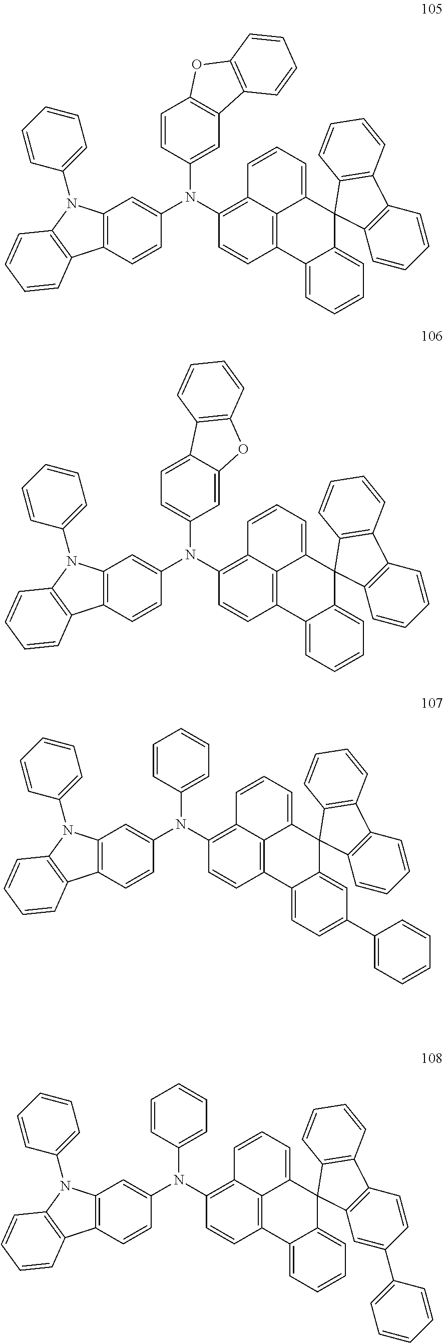 [SMILES: c1ccc(-n2c3ccccc3c3ccc(N(c4ccc5oc6ccccc6c5c4)c4ccc5c6c(cccc46)C4(c6ccccc6-c6ccccc64)c4ccccc4-5)cc32)cc1 ; c1ccc(-n2c3ccccc3c3ccc(N(c4ccc5c(c4)oc4ccccc45)c4ccc5c6c(cccc46)C4(c6ccccc6-c6ccccc64)c4ccccc4-5)cc32)cc1 ; c1ccc(-c2ccc3c(c2)C2(c4ccccc4-c4ccccc42)c2cccc4c(N(c5ccccc5)c5ccc6c7ccccc7n(-c7ccccc7)c6c5)ccc-3c24)cc1 ; c1ccc(-c2ccc3c(c2)C2(c4ccccc4-3)c3ccccc3-c3ccc(N(c4ccccc4)c4ccc5c6ccccc6n(-c6ccccc6)c5c4)c4cccc2c34)cc1] 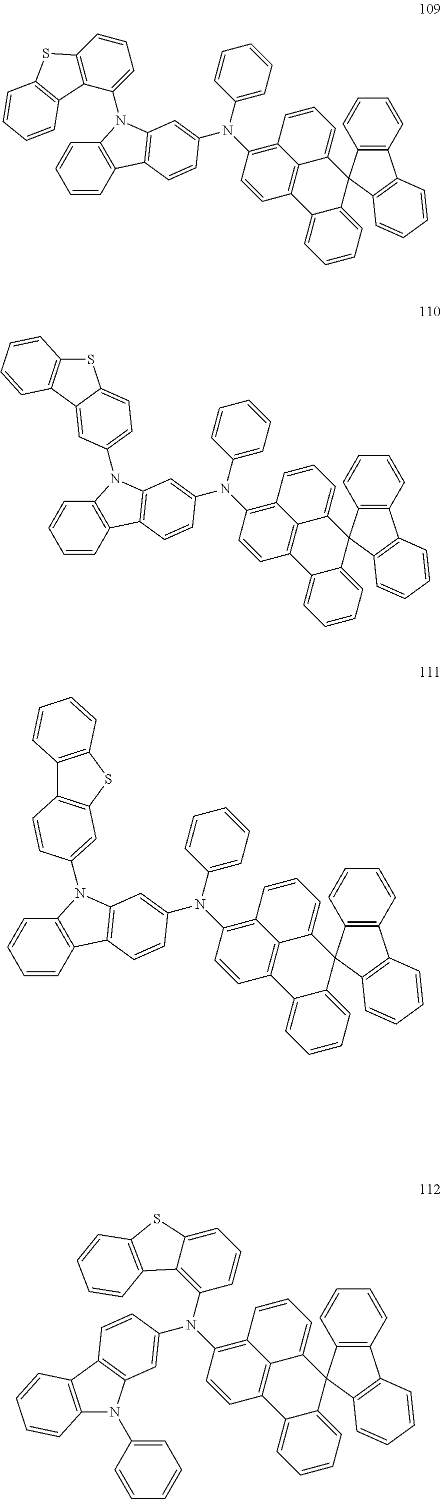 [SMILES: c1ccc(N(c2ccc3c4ccccc4n(-c4cccc5sc6ccccc6c45)c3c2)c2ccc3c4c(cccc24)C2(c4ccccc4-c4ccccc42)c2ccccc2-3)cc1 ; c1ccc(N(c2ccc3c4ccccc4n(-c4ccc5sc6ccccc6c5c4)c3c2)c2ccc3c4c(cccc24)C2(c4ccccc4-c4ccccc42)c2ccccc2-3)cc1 ; c1ccc(N(c2ccc3c4ccccc4n(-c4ccc5c(c4)sc4ccccc45)c3c2)c2ccc3c4c(cccc24)C2(c4ccccc4-c4ccccc42)c2ccccc2-3)cc1 ; c1ccc(-n2c3ccccc3c3ccc(N(c4ccc5c6c(cccc46)C4(c6ccccc6-c6ccccc64)c4ccccc4-5)c4cccc5sc6ccccc6c45)cc32)cc1] 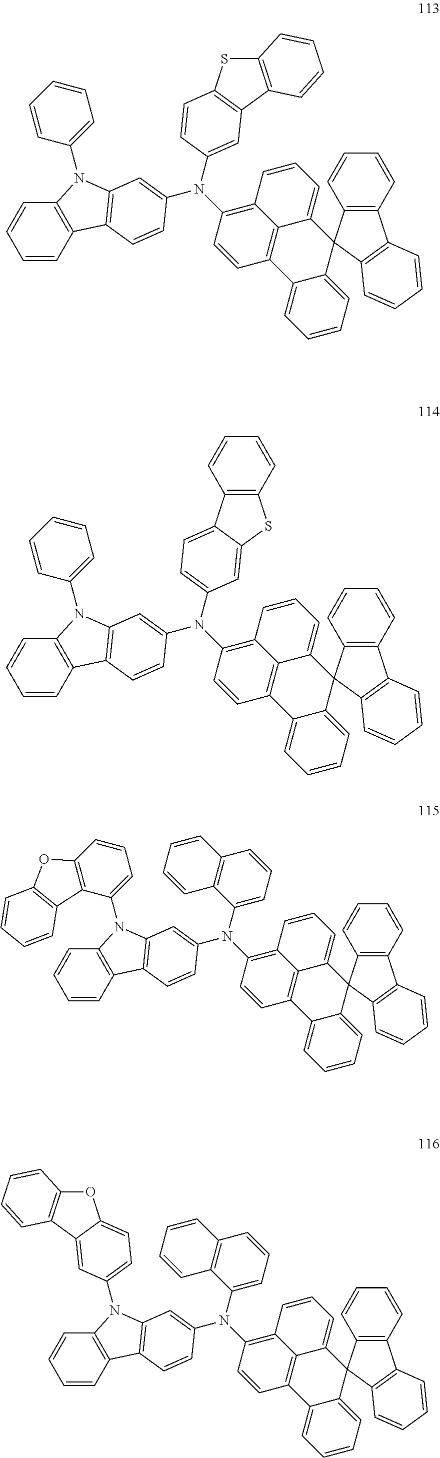 [SMILES: c1ccc(-n2c3ccccc3c3ccc(N(c4ccc5sc6ccccc6c5c4)c4ccc5c6c(cccc46)C4(c6ccccc6-c6ccccc64)c4ccccc4-5)cc32)cc1 ; c1ccc(-n2c3ccccc3c3ccc(N(c4ccc5c(c4)sc4ccccc45)c4ccc5c6c(cccc46)C4(c6ccccc6-c6ccccc64)c4ccccc4-5)cc32)cc1 ; c1ccc2c(c1)-c1ccccc1C21c2ccccc2-c2ccc(N(c3ccc4c5ccccc5n(-c5cccc6oc7ccccc7c56)c4c3)c3cccc4ccccc34)c3cccc1c23 ; c1ccc2c(c1)-c1ccccc1C21c2ccccc2-c2ccc(N(c3ccc4c5ccccc5n(-c5ccc6oc7ccccc7c6c5)c4c3)c3cccc4ccccc34)c3cccc1c23] 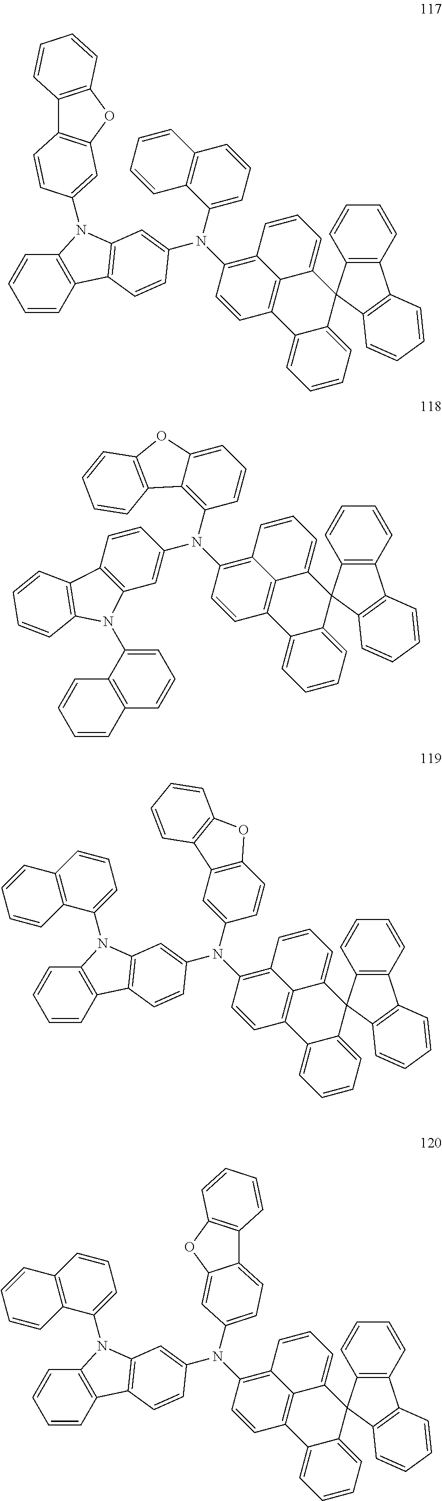 [SMILES: c1ccc2c(c1)-c1ccccc1C21c2ccccc2-c2ccc(N(c3ccc4c5ccccc5n(-c5ccc6c(c5)oc5ccccc56)c4c3)c3cccc4ccccc34)c3cccc1c23 ; c1ccc2c(c1)-c1ccccc1C21c2ccccc2-c2ccc(N(c3ccc4c5ccccc5n(-c5cccc6ccccc56)c4c3)c3cccc4oc5ccccc5c34)c3cccc1c23 ; c1ccc2c(c1)-c1ccccc1C21c2ccccc2-c2ccc(N(c3ccc4oc5ccccc5c4c3)c3ccc4c5ccccc5n(-c5cccc6ccccc56)c4c3)c3cccc1c23 ; c1ccc2c(c1)-c1ccccc1C21c2ccccc2-c2ccc(N(c3ccc4c(c3)oc3ccccc34)c3ccc4c5ccccc5n(-c5cccc6ccccc56)c4c3)c3cccc1c23] 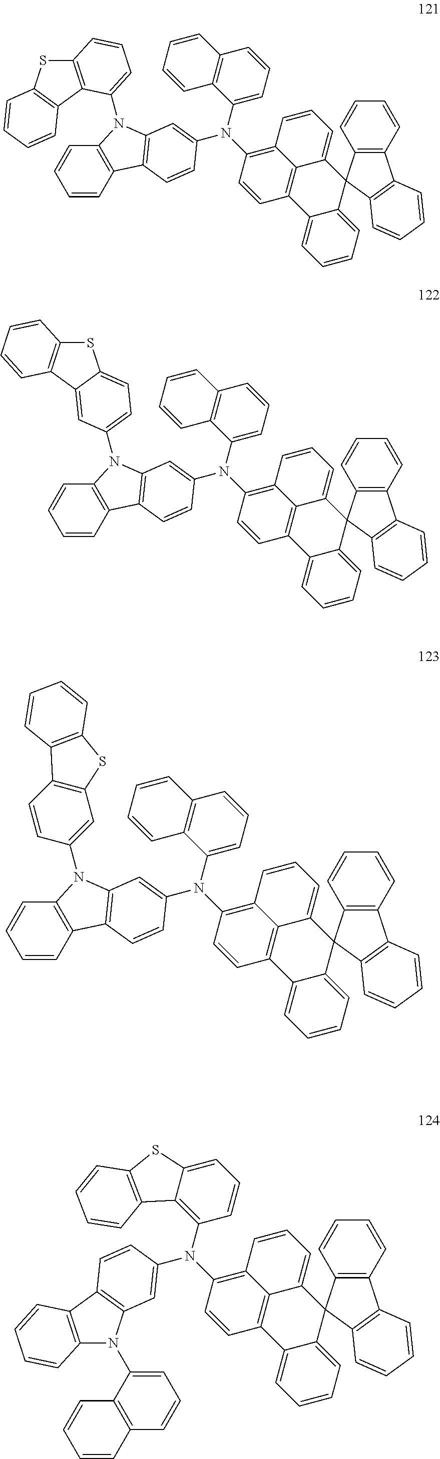 [SMILES: c1ccc2c(c1)-c1ccccc1C21c2ccccc2-c2ccc(N(c3ccc4c5ccccc5n(-c5cccc6sc7ccccc7c56)c4c3)c3cccc4ccccc34)c3cccc1c23 ; c1ccc2c(c1)-c1ccccc1C21c2ccccc2-c2ccc(N(c3ccc4c5ccccc5n(-c5ccc6sc7ccccc7c6c5)c4c3)c3cccc4ccccc34)c3cccc1c23 ; c1ccc2c(c1)-c1ccccc1C21c2ccccc2-c2ccc(N(c3ccc4c5ccccc5n(-c5ccc6c(c5)sc5ccccc56)c4c3)c3cccc4ccccc34)c3cccc1c23 ; c1ccc2c(c1)-c1ccccc1C21c2ccccc2-c2ccc(N(c3ccc4c5ccccc5n(-c5cccc6ccccc56)c4c3)c3cccc4sc5ccccc5c34)c3cccc1c23] 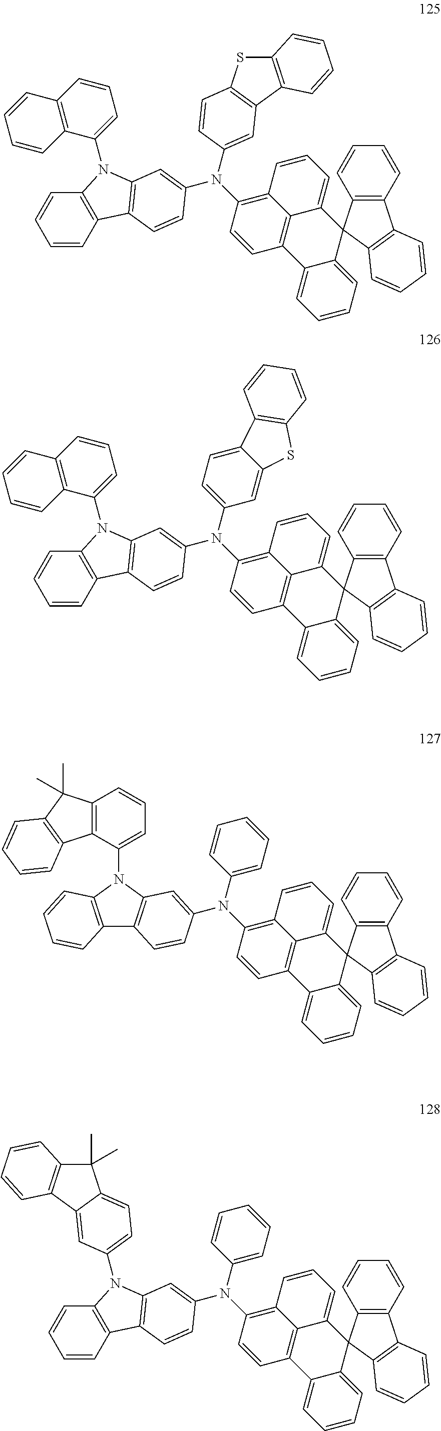 [SMILES: c1ccc2c(c1)-c1ccccc1C21c2ccccc2-c2ccc(N(c3ccc4sc5ccccc5c4c3)c3ccc4c5ccccc5n(-c5cccc6ccccc56)c4c3)c3cccc1c23 ; c1ccc2c(c1)-c1ccccc1C21c2ccccc2-c2ccc(N(c3ccc4c(c3)sc3ccccc34)c3ccc4c5ccccc5n(-c5cccc6ccccc56)c4c3)c3cccc1c23 ; CC1(C)c2ccccc2-c2c(-n3c4ccccc4c4ccc(N(c5ccccc5)c5ccc6c7c(cccc57)C5(c7ccccc7-c7ccccc75)c5ccccc5-6)cc43)cccc21 ; CC1(C)c2ccccc2-c2cc(-n3c4ccccc4c4ccc(N(c5ccccc5)c5ccc6c7c(cccc57)C5(c7ccccc7-c7ccccc75)c5ccccc5-6)cc43)ccc21] 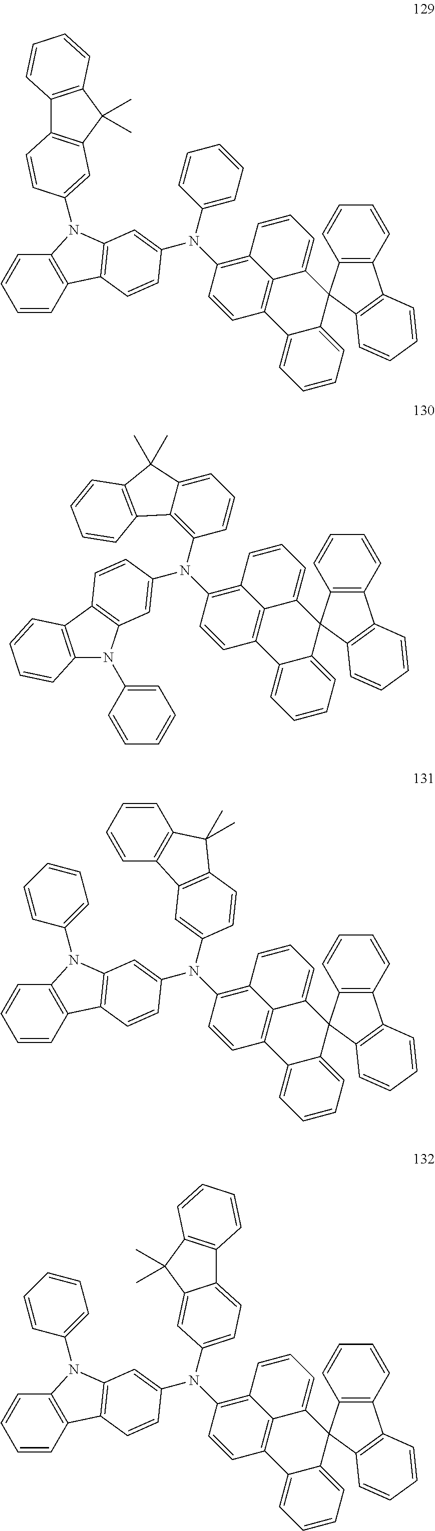 [SMILES: CC1(C)c2ccccc2-c2ccc(-n3c4ccccc4c4ccc(N(c5ccccc5)c5ccc6c7c(cccc57)C5(c7ccccc7-c7ccccc75)c5ccccc5-6)cc43)cc21 ; CC1(C)c2ccccc2-c2c(N(c3ccc4c5ccccc5n(-c5ccccc5)c4c3)c3ccc4c5c(cccc35)C3(c5ccccc5-c5ccccc53)c3ccccc3-4)cccc21 ; CC1(C)c2ccccc2-c2cc(N(c3ccc4c5ccccc5n(-c5ccccc5)c4c3)c3ccc4c5c(cccc35)C3(c5ccccc5-c5ccccc53)c3ccccc3-4)ccc21 ; CC1(C)c2ccccc2-c2ccc(N(c3ccc4c5ccccc5n(-c5ccccc5)c4c3)c3ccc4c5c(cccc35)C3(c5ccccc5-c5ccccc53)c3ccccc3-4)cc21]
